(19) United States
(12) Patent Application Publication
(10) Pub. No.: US 2015/0129861 A1
(43) Pub. Date: May 14, 2015

(54) ORGANIC MATERIAL FOR DEPOSITION, AND ORGANIC PHOTOELECTRIC CONVERSION ELEMENT, IMAGING ELEMENT, DEPOSITION METHOD, AND MANUFACTURING METHOD FOR ORGANIC PHOTOELECTRONIC ONVERSION ELEMENT OBTAINED USING THE SAME

(71) Applicant: FUJIFILM Corporation, Tokyo (JP)

(72) Inventors: Mitsumasa HAMANO, Ashigarakami-gun (JP); Eiji FUKUZAKI, Ashigarakami-gun (JP)

(73) Assignee: FUJIFILM CORPORATION, Tokyo (JP)

(21) Appl. No.: 14/604,486

(22) Filed: Jan. 23, 2015

Related U.S. Application Data
(63) Continuation of application No. PCT/JP2013/004236, filed on Jul. 9, 2013.

(30) Foreign Application Priority Data
Jul. 25, 2012 (JP) 2012-164658

(51) Int. Cl. H01L 51/00; H01L 27/30; H01L 51/52; H01L 27/32; H01L 51/56; H01L 51/44

(52) U.S. Cl. CPC H01L 51/0058; H01L 51/56; H01L 51/0002; H01L 51/442; H01L 51/5234; H01L 27/32; H01L 27/307; H01L 51/006; H01L 51/0072; H01L 51/0087; H01L 51/0061; H01L 51/0071
USPC 257/40; 438/82; 252/500

(57) ABSTRACT

An organic material for deposition that is used for dry deposition of an organic layer included in an organic photoelectric conversion element is provided in which the organic material contains an organic composition of the organic layer as a principal component, and a residual solvent content of the organic material for deposition is equal to or less than 3 mol %.

### CROSS REFERENCE TO RELATED APPLICATIONS

The present application is a Continuation of PCT International Application No. PCT/JP2013/004236 filed on Jul. 9, 2013, which claims priority under 35 U.S.C. §119(a) to Japanese Patent Application No. 2012-164658 filed on Jul. 25, 2012. Each of the above applications is hereby expressly incorporated by reference, in its entirety, into the present application.

### BACKGROUND OF THE INVENTION

1. Field of the Invention

The present invention relates to an organic material for deposition used for dry deposition of an organic photoelectric conversion element, and an organic photoelectric conversion element, an imaging element, and an organic electroluminescent element that are obtained using the organic material for deposition. The present invention also relates to a deposition method of an organic layer included in the organic photoelectric conversion element, and a manufacturing method for the organic photoelectric conversion element.

2. Description of the Related Art

Organic photoelectric conversion films having the characteristics that enables reduction in weight and increase in area thereof, with high flexibility, and which can be manufactured by printing process, as well as organic photoelectric conversion elements using the same are expected to be developed for various applications, including an image sensor (imaging element) used in a digital camera etc., an organic electroluminescent element (organic EL) used for a display, illumination etc., an organic thin film solar battery used in an electronic paper etc., an organic thin film transistor, and the like.

In the field of the imaging element, the area of a photodiode has recently been decreased together with reduction in size of a pixel due to an increase in number of pixels of the imaging element, which raises the problems of reduction in aperture ratio or light condensing efficiency, and also reduction in sensitivity together therewith. In general, a flat panel light receiving element is widely used as the imaging element. The flat panel light receiving element include pixels composed of photoelectric conversion parts two-dimensionally arranged in a semiconductor, and is designed to transfer and read out charges of a signal generated by photoelectric conversion in each pixel, through a CCD circuit or CMOS circuit.

The organic imaging element is increasingly expected to obtain a high aperture ratio, in addition to the characteristics described above, as compared to a conventional photoelectric conversion part with a photodiode formed therein using a PN junction in a semiconductor made of Si or the like.

The organic thin film solar battery is widely studied because it has advantages, including easy manufacturing steps and high possibility of the increase in area at low cost, as compared to an inorganic solar battery, typified by silicon or the like. However, the organic thin film solar battery has not reached the practical use level because of its low energy conversion efficiency.

The organic electroluminescent (EL) elements have been attracting attention as a display element or light emitting element because they can emit the light with high brightness at low voltage. The organic EL elements can significantly reduce power consumption and can easily achieve the reduction in size and increase in area, and therefore are aggressively studied for the practical use as the next-generation display element or light emitting element.

The applicants of the present invention have studied about an organic photoelectric conversion element including an organic layer in a light receiving layer or light emitting layer, and an imaging element, a light sensor, a solar battery, and an organic electroluminescent element including the same. In order to achieve the high S/N ratio in the organic photoelectric conversion element, it is necessary to have a high photoelectric conversion efficiency, and a low dark current performance.

For example, as for an improvement of the photoelectric conversion efficiency, the light receiving element preferably has good exciton dissociation effect in the light receiving layer, as well as good charge transport properties. In use of the imaging element, it is necessary to suppress the dark current, and preferable to control a carrier amount at the dark time.

Further, a high-speed signal response property needs to be satisfied.

The applicants of the invention submits an application regarding an organic photoelectric conversion element using a mixed layer of a p-type organic semiconductor and fullerene or fullerene derivative (a bulk hetero-structure layer formed of two materials by co-deposition) (see, for example, Japanese Unexamined Patent Application Publication No. 2007-123707 (hereinafter, Patent Document 1)).

### SUMMARY OF INVENTION

Patent Document 1 can provide an organic photoelectric conversion element having a high photoelectric conversion efficiency, and a good S/N ratio of photocurrent to dark current. In contrast, even though the high performance can be achieved, the organic photoelectric conversion element which cannot be stably manufactured is not considered to be of practical use.

When the applicants of the present invention have studies about the stability in manufacturing the organic photoelectric conversion element as disclosed in previous patent documents including Patent Document 1 and submitted by the applicants, the photoelectric conversion element having the same performance can be manufactured, but the manufacturing stability is desired to be improved.

The present invention has been made in view of the foregoing circumstances, and it is an object of the present invention to stably manufacture organic photoelectric conversion elements with the substantially same performance, and an imaging element with the same.

Further, it is another object of the present invention to provide an organic material for deposition that can form an organic layer by dry deposition to achieve the above-mentioned photoelectric conversion element.

It has been considered so far that in manufacture of the organic photoelectric conversion element, the influence of deposition material on the properties of a deposited film is made negligible by using an organic material for dry deposition whose HPLC purity is 95%, and more preferably 98% or more to form an organic layer, such as a light receiving layer, by a dry deposition method.

The inventors, however, have been dedicated themselves to studying about causes for reducing the manufacturing stability of the organic photoelectric conversion element, and as a result have found that the organic material for dry deposition is a main cause, and further that the main cause is due to residual solvent that have been considered negligible in terms of the HPLC purity.

That is, the organic material for deposition in the invention is an organic material for deposition used for dry deposition of an organic layer included in an organic photoelectric conversion element,

the organic layer contains an organic composition as a principal component, and a content of residual solvent in the organic material for deposition is 3 mol % or less.

The term “main component of the organic composition” as used in the specification means components other than inevitable impurities and residual solvent whose content is 3 mol % or less. The content of residual solvent is a value obtained by measuring using a nuclear magnetic resonance analysis (NMR), a gas chromatographic analysis, a Karl Fischer's method, or a detection method that can detect a solvent content with the same or higher accuracy than the accuracy of the above-mentioned methods.

The organic material for deposition in the invention is suitable for use when a vapor pressure of the residual solvent under a degree of vacuum of 1×10⁻³ Pa or less is higher than a sublimation pressure of the organic composition.

The use of the organic material for deposition in the invention can set a ratio of a film purity of the organic layer obtained after the continuous dry deposition for two hours to that of the organic layer in the initial stage of deposition to 0.9 or more.

The term “film purity in the initial stage of deposition” as used herein means a film purity directly after a deposition rate is stabilized. The organic material at the time when the deposition rate increases with increasing temperature, and then reaches the target rate is regarded as “stabilized”.

The use of the organic material for deposition in the invention can set a ratio of a film purity of the organic layer obtained when the dry deposition is continuously performed, resulting in a total thickness of the organic layer of 16000 Å taking into consideration the time for increasing the temperature of the material, to that of the organic layer in the initial stage of deposition to 0.9 or more. Here, the thickness of the organic layer is a value determined by multiplying the stabilized deposition rate by the time. This thickness is not limited to that of one single layer film, and may be a total thickness of a plurality of layers when depositing the layers.

The use of the organic material for deposition in the invention can set a ratio of a film purity of the organic layer obtained when the dry deposition is continuously performed, resulting in a total thickness of the organic layer of 16000 Å, to that of the organic layer for deposition to 0.9 or more.

The term “film purity” as used in the present specification means a peak area ratio of a principal component of the organic composition obtained by dissolving the deposited film in the solvent and detecting the principal component by the HPLC. Any part of the deposited film may be measured as long as the part is located within a range of about 15% of an in-plane thickness ratio from the substantially center of the film.

The term “purity of the organic material for deposition” as used in the present specification means a peak area ratio of a principal component of the organic composition that is detected by the HPLC by dissolving the organic material for deposition with solvent.

A deposition method of the invention is a method for forming an organic layer constituting an organic photoelectric conversion element, by the dry deposition. The deposition method includes the steps of: preparing an organic material for deposition which contains an organic composition of the organic layer as a principal component; removing a solvent contained in the organic material for deposition such that a solvent content is 3 mol % or less; and

depositing the organic layer by the dry deposition using the organic material for deposition whose residual solvent content is 3.0 mol % or less in the solvent removal step.

In the invention, the dry deposition can include a vacuum resistance heating vapor deposition method. The dry deposition vapor may be a co-deposition method.

The invention can be preferably applied to the case in which the organic layer is a photoelectric conversion layer, or an electron or hole blocking layer, and more preferably applied to the case in which the above-mentioned organic composition contains a component represented by the following general formula (D-I) or general formula (EB-1).

### [Chemical Formula 1]

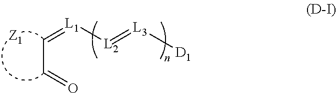

(In the general formula (D-I), Z₁ represents a group of atoms required to form a 5- or 6-membered ring. L₁, L₂ and L₃ independently represents a non-substituted methine group, or a substituted methine group. D₁ represents a group of atoms. n represents an integer number of 0 or more.)

### [Chemical Formula 29]

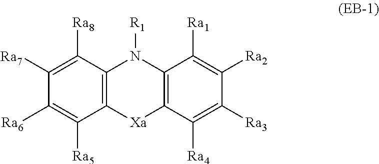

(In the general formula (EB-1), R₁ represents an alkyl group, an aryl group, or a heterocyclic group, which may have a substituent group. Ra₁ to Ra₈ each independently represents a hydrogen atom or substituent. At least two of R₁ and Ra₁ to Ra₈ may be bonded to each other to form a ring. Xa represents a single bond, an oxygen atom, a sulfur atom, or an alkylene group, a silylene group, an alkenylene group, a cycloalkylene group, a cycloalkenylene group, an arylene group, a divalent heterocyclic group, or an imino group, which may have a substituent group.)

A method for manufacturing an organic photoelectric conversion element according to the invention is a manufacturing method for an organic photoelectric conversion element that includes a pair of electrodes and a light receiving layer or light emitting layer including at least a photoelectric conversion layer sandwiched between the pair of the electrodes, in which the organic layer is deposited by the deposition method of the invention.

An organic photoelectric conversion element according to the invention is an organic photoelectric conversion element that includes a pair of electrodes and a light receiving layer or light emitting layer including at least a photoelectric conversion layer sandwiched between the pair of the electrodes, in which the organic layer is deposited by dry deposition using the organic material for deposition of the invention.

An imaging element according to the invention includes a plurality of photoelectric conversion elements of the invention; and

a circuit substrate having a signal reading circuit formed therein to read a signal corresponding to charges generated by the photoelectric conversion layer of the photoelectric conversion elements.

A light emitting element according to the invention includes a pair of electrodes, and at least a light emitting layer sandwiched between the pair of electrodes, in which light is emitted by applying a voltage to between the electrodes.

An organic material for deposition according to the invention is used for dry deposition of an organic layer forming an organic photoelectric conversion element, in which the organic material contains an organic composition of the organic layer as a principal component, and a residual solvent content is 3 mol % or less. With this arrangement, the organic material for deposition is a material for deposition that does not contain the residual solvent in the content that affects the film properties upon deposition and/or in the in-plane deposited film. Thus, the organic layer is deposited using the organic material for deposition according to the invention, or the organic layer is deposited by the deposition method that includes a residual solvent removal step of reducing the content of the solvent contained in the organic material for deposition down to 3 mol % or less, which can stably manufacture the organic photoelectric conversion elements with the same performance, and the imaging element and light emitting element including the same.

### BRIEF DESCRIPTION OF DRAWINGS

FIG. 1 is an exemplary cross-sectional view showing a schematic structure of an organic photoelectric conversion element in one embodiment of the invention;

FIG. 2 is an exemplary perspective view (vacuum heating deposition) showing a vapor deposition method of a light receiving layer;

FIG. 3 is an exemplary cross-sectional view showing a schematic structure of an imaging element in one embodiment of the invention; and

FIG. 4 is an exemplary cross-sectional view showing a schematic structure of an organic electroluminescent element in one embodiment of the invention.

### DETAILED DESCRIPTION OF THE PREFERRED EMBODIMENTS

“Organic Material for Deposition, Deposition Method for Organic Layer, and Photoelectric Conversion Element”

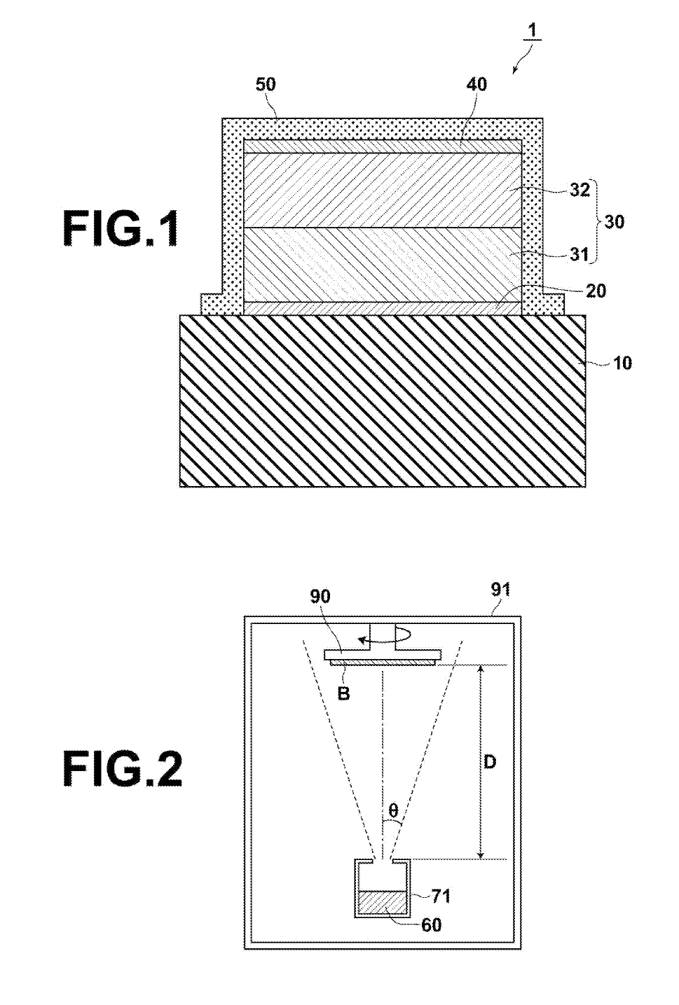

Referring to the accompanying drawings, a photoelectric conversion element in one embodiment of the invention will be described below. FIG. 1 is a schematic cross-sectional view showing the structure of the photoelectric conversion element in the present embodiment. For better understanding, in the drawings of the present specification, a scale reduction is adjusted for each component as appropriate.

As shown in FIG. 1, an organic photoelectric conversion element 1 (photoelectric conversion element 1) includes a substrate 10, a lower electrode 20 formed over the substrate 10, an electron blocking layer 31 formed over the lower electrode 20, an organic photoelectric conversion layer 32 (hereinafter referred to as a “photoelectric conversion layer”) formed over the electron blocking layer 31, an electrode 40 formed over the photoelectric conversion layer 32, and a sealing layer 50 formed over the electrode 40. The electron blocking layer 31 and the photoelectric conversion layer 32 forms a light receiving layer 30. The light receiving layer 30 may be a layer including at least the photoelectric conversion layer 32, and may be a layer including a layer other than the electron blocking layer 31 (for example, hole blocking layer).

The electron blocking layer 31 included in the light receiving layer 30 is a layer for suppressing injection of electrons from the lower electrode 20 into the photoelectric conversion layer 32, thereby inhibiting the flow of electrons generated in the photoelectric conversion layer 32 toward the side of the electrode 20. The electron blocking layer 31 contains organic or inorganic material, or both of them.

The electrode 40 is an electrode for collecting electrons from among the charges generated by the photoelectric conversion layer 32. The electrode 40 is formed using conductive material (e.g., ITO) having sufficient transparency for the light with a wavelength sensible by the photoelectric conversion layer 32 in order to allow the light to enter the photoelectric conversion layer 32. A bias voltage is applied to between the electrode 40 and the electrode 20, which can move holes among the charges generated by the photoelectric conversion layer 32 to the electrode 20, as well as electrons among them to the electrode 40.

The thus-structured photoelectric conversion element 1 has the upper electrode 40 as an electrode on a side of the incident light. Once the light enters the upper electrode 40 from above, the light transmits through the upper electrode 40 to be incident on the photoelectric conversion layer 32, generating charges. The holes among the generated charges are moved to the lower electrode 20. The holes moved to the lower electrode 20 are converted into a voltage signal according to the amount of the holes to be read out, whereby the light can be converted into the voltage signal to be taken out.

Alternatively, the bias voltage may also be applied so as to collect the electrons in the electrode 20, and also to collect the holes in the electrode 40. In this case, instead of the electron blocking layer 31, a hole blocking layer may be provided. The hole blocking layer may be formed as a layer made of organic material for suppressing the injection of holes from the electrode 20 into the photoelectric conversion layer 32, and for inhibiting the flow of holes generated by the photoelectric conversion layer 32 to the electrode 20 side. In either case, a part sandwiched between the electrode 20 and the electrode 40 serves as the light receiving layer 30.

In the above photoelectric conversion element 1, the light receiving layer 30 containing the photoelectric conversion layer 32 is a layer containing organic material (organic layer), and includes the organic layer deposited by the dry deposition using the organic material 60 for deposition. Note that in the following, the organic layer deposited using the organic material 60 for deposition will be described as a layer forming the light receiving layer 30. If an organic layer is deposited by the dry deposition, the organic layer is not limited to the light receiving layer 30.

The dry deposition is not specifically limited. The dry depositions can include physical vapor deposition, sputtering, chemical vapor deposition, and the like. However, the vacuum resistance vapor deposition method can be preferably used.

FIG. 2 shows an example of an exemplary diagram of the state of vapor deposition by the vacuum resistance heating deposition. As shown in FIG. 2, normally, the vapor deposition of the light receiving layer is performed while a substrate holder 90 is provided above an opening of a vapor deposition cell 71 disposed in a vapor deposition chamber 91, and a substrate B is installed on the holder 90. The organic material for deposition (vapor deposition material) 60 is set in the vapor deposition cell 71 with a heating function. Since a degree of vacuum within the vapor deposition chamber 91 is high, the material for vapor deposition evaporating from the vapor deposition cell 71 is emitted from the opening and goes straight, and is then deposited on the substrate B. By adjusting a diameter of the opening of the vapor deposition cell 71, a maximum emission angle θ of the vapor deposition material evaporating can be adjusted.

The vapor deposition cell 71 and the substrate B may be preferably spaced apart from each other by 10 cm or more if possible. The evaporating vapor deposition raw material is incident on the substrate surface at an incident angle of 0° to θ to expand substantially in a conical shape.

The organic material 60 for deposition is provided as a vapor deposition source having a boat-like, basket-like, hair-pin like, pot-like shape, or the like, but is not specifically limited.

In the item of “means for solving problems”, the inventors have described that even if the organic layer is deposited using the organic material for dry deposition whose HPLC purity is 95% or more, and preferably 98% or more, it exhibit variations in properties of the deposited film, which makes it difficult to stably manufacture the photoelectric conversion elements with the same performance.

The inventors had considered that even if there is such a little residual solvent that cannot be detected by the HPLC, most of solvents to be used for preparation of the organic material for deposition, especially, in the final stage do not affect the film properties. This is because in the dry deposition to be performed at a degree of vacuum of 1·10⁻³ Pa or less, a vapor pressure of the solvent is higher than a sublimation pressure of the organic composition, so that the solvent sufficiently vaporizes in a stage before sublimation of the organic material.

However, as can be seen from examples to be described later and comparative examples, it has been confirmed that in the examples using the organic material for deposition having 3 mol % or less of the residual solvent, the film properties obtained after the continuous deposition for 1 hour or two hours hardly changes, whereas in the comparative examples using the organic material for deposition having more than 3 mol % of the residual solvent, the film properties is worsened over time.

Such a result shows that the main cause for worsening the film properties is the existence of residual solvent that had been considered negligible in terms of HPLC purity. Further, it is found that the content of the residual solvent in the organic material for the deposition is equal to or less than 3 mol %, which can suppress variations in film properties, thus making it possible to stably manufacture the photoelectric conversion elements with the same performance.

Although this mechanism is uncertain, the inventors have supposed that the decomposition of the residual solvent slightly contained is promoted due to a thermal load, or the residual solvent reacts with the organic material due to the increase in the thermal load over time, which might adversely affect the partial decomposition of the organic material, causing the organic material to sublimate during the deposition. Over the time, the film properties are observed to be degraded.

In the examples to be described later, in the dry deposition of the deposition material for the organic photoelectric conversion layer having a high photoelectric conversion efficiency, the organic material 60 for deposition having 3 mol % or less of the residual solvent was used to deposit the photoelectric conversion layer 32 or the electron blocking layer 31, and then the film properties of the deposited layer, the photoelectric conversion efficiency (sensitivity) and the response speed (rise time) thereof when the photoelectric conversion element 1 was fabricated were evaluated.

Table 1 shows the change in film properties over time in the continuous deposition. It is confirmed that the deposition using the organic material 60 for deposition having 3 mol % or less of the residual solvent can set a ratio of a film purity of the photoelectric conversion layer 32 deposited after the continuous deposition for 2 hours, to a film purity of the photoelectric conversion layer (organic layer) 32 in the initial stage of deposition (direct after stabilizing the deposition speed), to 0.9 or more, that is, that such deposition can suppress the degradation of the film purity down to 10% or less.

Table 2 shows that the use of the organic material 60 for deposition can suppress the variations in film properties, and further can stably deposit the photoelectric conversion element 1 having a high photoelectric conversion efficiency (sensitivity) and a high response speed (rise time).

That is, the organic material 60 for deposition contains the organic composition of the light receiving layer 30 (electron blocking layer 31, photoelectric conversion layer 32) of the organic photoelectric conversion element 1, as a principal component, and the residual solvent content is 3 mol % or less.

In the example, the organic material 60 for deposition is used as the organic material for deposition of the light receiving layer 30. The influence of the residual solvent on the film properties is caused not only in the photoelectric layer material and electron blocking material, but also unlimitedly in the organic material for deposition in the dry deposition for the organic photoelectric conversion element, even though the degree of influence, such as easiness of decomposition or the like, differs depending on the material.

The organic material 60 for deposition can be preferably applied to the photoelectric conversion layer 32 and electron blocking layer 31 included in the light receiving layer 30, or to the hole blocking layer or the like (not shown) because of its large influence on the element characteristics of the photoelectric conversion element.

The kind of the residual solvent is not limited even though the influence varies in level from small to large. Solvents can include, for example, water, alcohol, ether, ketone, sulfoxide, carbonate, amide, carboxylic acid, ester, nitrile, halogen, aromatic series, and the like. More specifically, when two or more kinds of the solvents are contained, the total amount of two or more solvents is 3 mol % or less.

For example, the following solvents are considered. Specifically, suitable solvents include methanol, ethanol, propanol, isopropanol, butanol, isobutanol, t-butyl alcohol, ethylene glycol, propylene glycol, glycerin, dimethyl ether, diethyl ether, 1,2-dimethoxy ethane, diglyme, triglyme, oligoethylene oxide, oligopropylene oxide, polyethylene oxide, polypropylene oxide, anisole, diphenyl ether, THF, dioxane, 1,3-dioxolne, acetone, MEK, syclohexanone, cyclopentanone, dimethyl sulfoxide, dimethyl sulfone, sulfolane, dimethyl carbonate, diethyl carbonate, ethylene carbonate, propylene carbonate, N,N-dimethyleformamide, N,N-dimethyl acetamide, N-methyl pyrrolidone, N-ethyl pyrrolidone, acetic acid, ethyl acetate, acetonitrile, benzonitrile, benzene, o-,m-,p-xylene, toluene, o-,m-,p-TMB, chlorobenzene, o-,m-,p-dichlorobenzen, nitrobenzene, chloroform, methylene chloride, and the like, but not limited to the above solvents.

When the organic material for deposition is commercially available, the deposition of the light receiving layer 30 (electron blocking layer 31, photoelectric conversion layer 32) is performed by preparing the commercially-available organic material for deposition, then performing a solvent removal step of removing a solvent contained in the organic material for deposition so as to measure 3 mol % or less of solvent, thereby producing the organic material 60 for deposition, and performing deposition by a predetermined dry deposition method.

When the organic material for deposition is synthesized, in a refinement step of the synthesized organic material, or after the above solvent removal step, following the refinement step, the light receiving layer 30 (electron blocking layer 31, photoelectric conversion layer 32) may be deposited by a predetermined deposition method.

When the organic material for deposition is a molded body formed by being pressurized into a specific shape, a sintered body produced by burning the molded body, or a granulated material produced therefrom, or further a granular sintered body obtained by burning the granulated material, the solvent removal step may be performed after the molding, granulation, and sintering.

Methods for performing the solvent removal step are not specifically limited, but can include sublimation refinement, recystallization refinement, column chromatography refinement, reslurrying, vacuum drying, reprecipitaion refinement, separation, washing with water or solvent, filtration, filtering, ion-exchange resin chromatography, adsorption with activated coal, diatom earth, ion-exchange resin, resin, or inorganic porous material (zeolite), air drying, heating drying, freezing and drying, and the like.

The organic material 60 for deposition is used for dry deposition of an organic layer forming the organic photoelectric conversion element 1 (light receiving layer 30; electron blocking layer 31, photoelectric conversion layer 32), and contains an organic composition of the organic layer as a principal component, and a residual solvent content is 3 mol % or less. With this arrangement, the organic material 60 for deposition is a material for deposition that does not contain the residual solvent in the content that affects the film properties upon deposition and/or in the in-plane deposited film. Thus, the light receiving layer 30 is deposited using the organic material 60 for deposition, or the light receiving layer 30 is deposited by the deposition method that includes a residual solvent removal step of reducing the content of the solvent contained in the organic material 60 for deposition down to 3 mol % or less, which can stably manufacture the organic photoelectric conversion elements 1 with the same performance.

A description will be given below of the structure of the photoelectric conversion element 1 shown in FIG. 1. As mentioned above, with the above arrangement, the light receiving layer 30, including the photoelectric conversion layer 32 and electron blocking layer 31 which are organic layers, is deposited using the organic material 60 for deposition by the dry deposition, whereby the light receiving layer 30 has little variation in film properties, so that the photoelectronic element 1 with high photoelectric conversion efficiency and high response speed can be stably manufactured.

### <Substrate and Electrode>

The substrate 10 is not specifically limited, but can be a silicon substrate, a glass substrate, and the like in use.

The lower electrode 20 is an electrode for collecting holes from among the charges generated by the photoelectric conversion layer 32. The lower electrode 20 is not specifically limited as long as its conductivity is good. Depending on applications, the lower electrode 20 is used by being made transparent in some cases, and conversely by being formed of material that allows the light to be reflected without transparency in other cases. Specifically, suitable materials for the lower electrode can include conductive metal oxide, such as tin oxide (ATO, FTO) doped with antimony, fluorine, or the like, tin oxide, zinc oxide, indium oxide, indium tin oxide (ITO), indium zinc oxide (IZO), and the like; metal, such as gold, silver, chromium, nickel, titanium, tungsten, alumina, and the like; a conductive compound made of metal oxide or nitride of any of these metals (e.g., titanium nitride (TiN)); a mixture or laminate of any of these metals and a conductive metal oxide; inorganic conductive material, such as copper iodide or copper sulfide; organic conductive material, such as polyaniline, polythiophene, or polypyrrole; a laminate of any of these materials and ITO or titanium nitride; and the like.

The upper electrode 40 is an electrode for collecting electrons from among the charges generated by the photoelectric conversion layer 32. The electrode 40 is formed of any conductive material, which is not specifically limited, as long as the material has sufficient transparency for the light with a wavelength sensible by the photoelectric conversion layer 32 in order to allow the light to enter the photoelectric conversion layer 32. Specifically, suitable materials for the upper electrode can include conductive metal oxide, such as tin oxide (ATO, FTO) doped with antimony, fluorine, or the like, tin oxide, zinc oxide, indium oxide, indium tin oxide (ITO), indium zinc oxide (IZO), and the like; a metal thin film made of gold, silver, chromium, nickel, or the like; a mixture or laminate of any of these metals and a conductive metal oxide; inorganic conductive material, such as copper iodide or copper sulfide; organic conductive material, such as polyaniline, polythiophene, or polypyrrole; a laminate of any of these materials and ITO; and the like. Among them, the conductive metal oxide is preferable in terms of high conductivity, transparency, and the like.

A method for forming the above-mentioned electrode is not specifically limited, and can be appropriately selected taking into consideration the suitability for the material for the electrode. Specifically, the electrode can be formed by a printing method, a wet type method, such as a coating method, a physical method, such as a vacuum vapor deposition method, a sputtering method, and an ion plating method, or a chemical method, such as a CVD, and a plasma CVD method.

When the material for the electrode is ITO, the electrode can be formed by an electron beam method, a sputtering method, a resistance heating deposition method, a chemical reaction method (sol-gel method etc.), a coating method of dispersed material, such as indium tin oxide, and the like. Further, a film fabricated using ITO can be subjected to UV-ozone treatment, plasma treatment, and the like. When the material for the electrode is TiN, various methods, including a reactive sputtering method, can be used, and further a UV-ozone treatment, a plasma treatment, and the like can be performed.

The upper electrode 40 is deposited on the organic photoelectric conversion layer 32, and thus is preferably deposited by a method that does not degrade the properties of the organic photoelectric conversion layer 32. Hence, the upper electrode 40 is preferably deposited while being free from the plasma. Here, the term “plasma free” means that no plasma is generated during the deposition of the upper electrode 40, or that a distance from a plasma generating source to a base is 2 cm or more, preferably 10 cm or more, and more preferably 20 cm or more to reduce the amount of plasma reaching the base.

A device that does not generate plasma during deposition of the upper electrode 40 is, for example, an electron-beam deposition device (EB deposition device), or a pulse laser deposition device. As the EB deposition device or pulse laser deposition device, can be used a device described in Y. Sawada, “New Development of Transparent Conductive Film”, (CMC article, 1999); Y. Sawada, “New Development II of Transparent Conductive Film”, (CMC article, 2002); “Technologies of Transparent Conductive Films” written by Japan Society for the Promotion of Science (Ohm, 1999), and cited references attached to them. Hereinafter, a method for depositing a transparent electrode film using the EB deposition device is referred to as an “EB deposition method”, while a method for depositing a transparent electrode film using the pulse laser deposition device is referred to as a “pulse laser deposition method”.

In a device that can achieve the distance of 2 cm or more from the plasma generating source to the base to decrease the amount of plasma reaching the base (hereinafter referred to as a plasma free deposition device), for example, an opposed target sputtering device or an arc plasma deposition method can be considered to be used. These devices can be used as described in Y. Sawada, “New Development of Transparent Conductive Film”, (CMC article, 1999); Y. Sawada, “New Development II of Transparent Conductive Film”, (CMC article, 2002); “Technologies of Transparent Conductive Films” written by Japan Society for the Promotion of Science (Ohm, 1999), and cited references attached to them.

When the transparent conductive film, such as TCO, is used as the upper electrode 40, DC short or increase in leak current might be caused in some cases. One of the reasons is that fine cracks introduced in the photoelectric conversion layer 32 are covered with a fine film made of TCO or the like, increasing conduction with the lower electrode 20 on the opposite side. For this reason, in the case of an electrode made of Al or the like and having relatively degraded film properties, the leak current is less likely to be increased. The thickness of the upper electrode 40 is controlled with respect to the thickness of the photoelectric conversion layer 32 (that is, the depth of the crack), which can significantly suppress the increase in leak current. It is desirable that the thickness of the upper electrode 40 is one fifth or less of the thickness of the photoelectric conversion layer 32, and preferably one tenth or less thereof.

Normally, the conductive film whose thickness is set thinner than a certain range leads to a drastic increase in resistance. In a solid imaging element incorporating therein the photoelectric conversion element of this embodiment, a sheet resistance may be preferably in a range of 100 to 10000Ω/□. The imaging element has a high degree of flexibility in a range that can decrease its thickness. As the thickness of the upper electrode 40 is decreased, the amount of absorbed light becomes smaller, generally resulting in an increase in light transmittance. The increase in light transmittance increases the light absorption in the photoelectric conversion layer 32, thereby increasing the photoelectric conversion capacity, which is very preferable. Taking into consideration the suppression of the leak current, the increase in resistance of the thin film and the increase in transmittance together with the decrease in thickness of the film, the thickness of the upper electrode 40 is preferably in a range of 5 to 100 nm, and more preferably in a range of 5 to 20 nm.

The bias voltage is applied to between the upper electrode 40 and the lower electrode 20, which can move holes among the charges generated by the photoelectric conversion layer 32 to the lower electrode 20, as well as electrons among them to the upper electrode 40.

### <Light Receiving Layer>

The light receiving layer 30 is an organic layer containing at least the photoelectric conversion layer 32, which includes an organic layer deposited by the dry deposition using the organic material 60 for deposition. In this embodiment, the light receiving layer 30 is composed of the electron blocking layer 31 and the photoelectric conversion layer 32, either or both of these can be deposited using the organic material 60 for deposition by the dry deposition. To further suppress the variations in film properties, as many organic layers as possible, which are included in the light receiving layer 30, are preferably deposited using the organic material 60 for deposition.

The light receiving layer 30 can be formed by the dry deposition or wet deposition method. The dry deposition method is preferable because of easiness of formation of a uniform film and difficulty for impurities to contaminate, and also because of easiness of control of the film thickness and lamination of different materials.

Specific examples of the dry deposition can include a physical vapor deposition, such as vacuum vapor deposition, sputtering, ion plating, and MBE method, or a CVD method such as plasma polymerization. Preferably, the dry deposition is the vacuum vapor deposition. When depositing a film by the vacuum vapor deposition, manufacturing conditions including a degree of vacuum, a deposition temperature, and the like can be set in an usual way. When forming the light receiving layer 30 by the vapor deposition, as the decomposition temperature is higher than the vapor depositable temperature, the thermal decomposition during the vapor deposition can be preferably suppressed.

When the light receiving layer 30 is formed by the dry deposition, the degree of vacuum upon formation is preferably equal to or less than 1·10⁻³ Pa, more preferably equal to or less than 4·10⁻⁴ Pa, and most preferably equal to or less than 1·10⁻⁴ Pa, taking into consideration the prevention of degradation of the element properties as forming the light receiving layer.

The thickness of the light receiving layer 30 is preferably not less than 10 nm nor more than 1000 nm, more preferably, not less than 50 nm nor more than 800 nm, and most preferably not less than 100 nm nor more than 600 nm. The light receiving layer 30 with a thickness of 10 nm or more can have a preferable effect of suppressing the dark current, while the light receiving layer with a thickness of 1000 nm or less can have a preferable photoelectric conversion efficiency.

### <<Photoelectric Conversion Layer>>

The photoelectric conversion layer 32 receives light and generates charges corresponding to the amount of light, and contains an organic photoelectric conversion material.

The photoelectric conversion element 1 of this embodiment includes a mixed layer composed of a mixture of a p-type organic semiconductor (p-type organic compound) and an n-type organic semiconductor in the photoelectric conversion layer 32. The mixed layer is preferably formed by co-depositing the organic material 60 for deposition which is the p-type organic semiconductor material, and the organic material 60 for deposition which is the n-type organic semiconductor material.

The mixed layer is a layer in which a plurality of materials are mixed or dispersed. In this embodiment, the mixed layer is a layer formed by co-depositing the p-type organic semiconductor and the n-type organic semiconductor.

The n-type organic semiconductor (compound) forming the photoelectric conversion layer 32 is not specifically limited, but is preferably a fullerene or fullerene derivative. Fullerenes can include fullerene C₆₀, fullerene C₇₀, fullerene C₇₆, fullerene C₇₈, fullerene C₈₀, fullerene C₈₂, fullerene C₈₄, fullerene C₉₀, fullerene C₉₆, fullerene C₂₄₀, fullerene ₅₄₀, mixed fullerene, fullerene nanotube, and the like.

A fullerene derivative represents a compound with a substituent group attached thereto. A substituent group of the fullerene derivative is preferably an alkyl group, an aryl group, or a heterocyclic group. More preferably, the alkyl group is an alkyl group with the number of carbon atoms of 1 to 12. Preferably, the aryl group and the heterocyclic group is a benzene ring, a naphthalene ring, an anthracene ring, a phenanthrene ring, a fluorene ring, a tripheylene ring, a naphthacene ring, a biphenyl ring, a pyrrole ring, a furan ring, a thiophene ring, an imidazole ring, an oxazole ring, a thiazole ring, a pyridine ring, a pyrazine ring, a pyrimidine ring, a pyridazine ring, an indolizine ring, an indole ring, a benzofuran ring, a benzothiophene ring, an isobenzofuran ring, a benzimidazole ring, an imidazo pyridine ring, a quinolizine ring, a quinoline ring, a phthalazin ring, a naphthylidine ring, a quinoxaline ring, a quinoxazoline ring, an isoquinoline ring, a carbazole ring, a phenantolidine ring, an acridine ring, a phenanthroline ring, a thianthrene ring, a chromene ring, a xanthene ring, a phenoxathiin ring, a phenothiazine ring, or a phenazine ring, more preferably, a benzene ring, a naphthalene ring, an anthracene ring, a phenanthrene ring, a pyridine ring, an imidazole ring, an oxazole ring, or a thiazole ring, and most preferably, a benzene ring, a naphthalene ring, or a pyridine ring. These substituents may further have another substitutent group. The substituent group may be bonded to form a ring as much as possible. Note that the fullerene derivative may have a plurality of substituent groups, which may be the same or different from each other. The substituent groups may be bonded together as much as possible to form a ring.

The p-type organic semiconductor (compound) forming the photoelectronic conversion layer 32 preferably has an appropriate particle diameter that does not significantly affect the stability of the deposition rate. The average particle diameter of each of the p-type organic semiconductor and the n-type organic semiconductor is preferably in a range of 10 to 800 μm, and more preferably 20 to 700 μm.

The p-type organic semiconductor (compound) forming the photoelectric conversion layer 32 is a donor organic semiconductor (compound). The organic semiconductor is an organic compound mainly typified by a hole transport organic compound, and that tends to supply electrons. More in detail, when using two organic materials in contact with each other, the donor organic compound corresponds to one of the organic compounds having a smaller ionization potential. Therefore, any donor organic compound can be used as long as the organic compound has an electron donating property.

For example, the donor organic compounds can include a triarylamine compound, a pyran compound, a quinacridon compound, a benzidine compound, a pyrazoline compound, a styrylamine compound, a hydrazone compound, a triphenylmethane compound, a carbazole compound, a polysilane compound, a thiophene compound, a phthalocyanine compound, a cyanine compound, a merocyanine compound, an oxonol compound, a polyamine compound, an indole compound, a pyrrole compound, a pyrazole compound, a polyarylene compound, a condensed aromatic carbocyclic compound (a naphthalin derivative, an anthracenes derivative, a phenantherene derivative, a tetracene derivative, a pyrene derivative, a perylene derivative, a fluoranthene derivative), and a metallic complex including a nitrogen containing heterocyclic compound as a ligand. Note that the donor organic compound is not limited thereto, and may be used as long as the organic compound has a smaller ionization potential than that of the organic compound used as the n-type organic semiconductor.

Among the above elements, a triarylamine compound, a pyran compound, a quinacridon compound, a pyrrole compound, a phthalocyanine compound, a merocyanine compound, and a condensed aromatic carbocyclic compound are preferable.

As the P-type organic semiconductor, any organic dye may be used. Preferably, examples of the organic dyes include a cyanine dye, a styryl dye, a hemicyanine dye, a merocyanine dye (including zero methine merocyanine (simple merocyanine)), trinuclear merocyanine dyes, tetranuclear merocyanine dyes, rhodacyanine dyes, complex cyanine dyes, complex merocyanine dyes, allopolar dyes, oxonol dyes, hemioxonol dyes, squarylium dyes, croconium dyes, azamethine dyes, coumarin dyes, arylidene dyes, anthraquinone dyes, triphenylmethane dyes, azo dyes, azomethine dyes, spiro compound, metallocene dyes, fluorenone dyes, fulgide dye, perylene dyes, perynone dyes, phenazine dyes, phenothiazine dyes, quinone dyes, diphenylmethane dyes, polyene dyes, acridine dyes, acridinone dyes, diphenylamine dyes, quinacridone dyes, quinophthalone dyes, phenoxazine dyes, phthaloperylene dye, diketopyrrolopyrrole dyes, dioxane dyes, porphyrin dyes, chlorophyll dyes, phthalocyanine dyes, metal complex dyes, fused aromatic carbocyclic dyes (naphthalene derivatives, anthracene derivatives, phenanthrene derivatives, tetracene derivatives, pyrene derivatives, perylene derivatives, fluoranthene derivatives).

When the ratio of the n-type organic semiconductor to the photoelectric conversion layer 32 is too large, the amount of absorption of the incident light is reduced to decrease the photoelectric conversion efficiency. Thus, for example, when the n-type organic semiconductor is a fullerene or a fullerene derivative, the ratio of the fullerene or fullerene derivative contained in the photoelectric conversion layer 32 is preferably 85% or less.

The p-type organic semiconductor is preferable when the n-type organic semiconductor is fullerene or a fullerene derivative, and is preferably deposited using the organic material 60 for deposition. The p-type organic semiconductor will be described later.

### <<Electron Blocking Layer>>

The electron blocking layer 31 included in the light receiving layer 30 is a layer for suppressing injection of electrons from the lower electrode 20 into the photoelectric conversion layer 32, thereby inhibiting the flow of electrons generated in the photoelectric conversion layer 32 toward the side of the electrode 20. The electron blocking layer 31 contains organic or inorganic material, or both of them.

The electron blocking layer 31 may be composed of a plurality of layers. In this way, there occur interfaces between the respective layers forming the electron blocking layer 31, so that the discontinuity is caused by an intermediate level existing in each layer. As a result, electric charges are less likely to transfer via the intermediate level or the like, which can enhance the electron blocking effect. Note that when the respective layers constituting the electron blocking layer 31 are made of the same material, the intermediate levels existing in the respective layer can be the same. Thus, in order to further enhance the electron blocking effect, the materials constituting the respective layers are preferably different.

The electron blocking layer 31 can be formed using an electron-donating organic material. Specifically, low-molecular materials can include aromatic diamine compounds, such as N, N′-bis(3-methylphenyl)-(1,1′-biphenyl)-4,4′-diamine (TPD), 4,4′-bis[N-(naphthyl)-N-phenyl-amino]biphenyl (α-NPD), or the like; oxazole, oxadiazole, triazole, imidazole, imidazolone, a stilbene derivative, a pyrazoline derivative, tetrahydroimidazole, polyaryl alkane, butadiene, 4,4′,4″-tris(N-(3-methylphenyl)N-phenylamino)triphenylamine (m-MTDATA), porphine, tetraphenyl porphin copper, phthalocyanine, copper phthalocyanine, a porphyrin compound, such as titanium phthalocyanine oxide, a triazole derivative, an oxa-diazole derivative, an imidazole derivative, a polyaryl alkane derivative, a pyrazoline derivative, a pyrazolone derivative, a phenylenediamine derivative, an arylamine derivative, a fluorene derivative, an amino-substituted chalcone derivative, an oxazole derivative, a styrylanthracene derivative, a fluorenone derivative, a hydrazone derivative, or a silazane derivative. Polymeric materials for use can include polymers, such as phenylenevinylene, fluorene, carbazole, indole, pyrene, pyrrole, picoline, thiophene, acetylene, or diacetylene; and derivatives thereof. The material for the electron blocking layer is not limited to the electron-donating compound, but can be a compound that has the sufficient hole transport property.

The electron blocking layer 31 can be formed using an inorganic material. In general, the inorganic material has a larger dielectric constant than the organic material. Therefore, in use of the inorganic material for the electron blocking layer 31, much voltage is applied to the photoelectric conversion layer 32, which can enhance the photoelectric conversion efficiency. Suitable materials for the electron blocking layer 31 can include calcium oxide, chromium oxide, chromium copper oxide, manganese oxide, cobalt oxide, nickel oxide, copper oxide, gallium copper oxide, strontium copper oxide, niobium oxide, molybdenum oxide, indium copper oxide, indium silver oxide, iridium oxide, and the like.

In the electron blocking layer 31 made of a plurality of layers, the layer adjacent to the photoelectric conversion layer 32 among the layers is preferably made of the same material as the p-type organic semiconductor included in the photoelectric conversion layer 32. The use of the same p-type organic semiconductor in the electron blocking layer 31 can suppress the formation of an intermediate level at the interface with the layer adjacent to the photoelectric conversion layer 32, thereby further preventing the dark current.

When the electron blocking layer 31 is a single layer, the layer can be made of inorganic material. When the electron blocking layer 31 is made of a plurality of layers, one or two or more of them can be made of inorganic material.

The electron-donating organic material is preferable when the n-type organic semiconductor is fullerene or a fullerene derivative, and is preferably deposited using the organic material 60 for deposition. The electron-donating organic material will be described later.

When the bias voltage is applied so as to collect electrons in the lower electrode 20 and also to collect holes in the upper electrode 40, the hole blocking layer may be provided instead of the electron blocking layer 31. The hole blocking layer may be formed as a layer made of organic material for suppressing the injection of holes from the lower electrode 20 into the photoelectric conversion layer 32, and for inhibiting the flow of holes generated in the photoelectric conversion layer 32 to the lower electrode 20 side. The hole blocking layer can be made of a plurality of layers to enhance the hole blocking effect.

### <<Hole Blocking Layer>>

The electrons or holes collected by the upper electrode 40 may be converted into the voltage signal corresponding to the amount of the electrons or holes to be taken out. In this case, the electron blocking layer or hole blocking layer may be provided between the upper electrode 40 and the photoelectric conversion layer 32. In either case, a part sandwiched between the lower electrode 20 and the upper electrode 40 serves as the light receiving layer 30.

The hole blocking layer can be formed using an electron-acceptor organic material. The electron-acceptor materials can include an oxadiazole derivative, such as 1,3-bis(4-tert-butyl phenyl-1,3,4-oxadiazolyl)phenylene (OXD-7), or the like; an anthraquinodimethane derivative; a diphenylquinon derivative; bathocuproine; bathophenanthroline, and a derivative thereof; a triazole compound; a tris (8-hydroxyquinolinate) aluminum complex; a bis(4-methyl-8-quinolinate)aluminum complex; a distyrylarylene derivative; and a silole compound. The hole blocking layer can be formed using any material with sufficient electron transport property even though the material is not the electron acceptor organic material. A porphyrin-based compound, a styryl-based compound, such as DCM (4-dicyanomethylene-2-methyl-6-(4-(dimethylaminostyryl))-4H pyran), and a 4H pyran-based compound can be used.

The hole blocking layer is preferably deposited using the organic material 60 for deposition.

### <Sealing Layer>

The sealing layer 50 is a layer that prevents factors for degrading the organic material, such as water or oxygen, from invading the light receiving layer containing the organic material. The sealing layer 50 is formed to cover the lower electrode 20, the electron blocking layer 31, the photoelectric conversion layer 32, and the upper electrode 40.

In the photoelectric conversion element 1, the incident light reaches the photoelectric conversion layer 32 via the sealing layer 50. In order to allow the light to be incident on the photoelectric conversion layer 32, the photoelectric conversion layer 32 needs to be sufficiently transparent for the light with the wavelength sensitive to the photoelectric conversion layer 32. Materials for such a sealing layer 50 can include fine ceramics, such as a metal oxide, a metal nitride, or a metal oxynitride, that does not permit the water molecule to transmit therethrough, and a diamond-like carbon (DLC). Conventionally, aluminum oxide, silicon oxide, silicon nitride, silicon oxynitride, or a laminated film thereof, and a laminated film of the above film and an organic polymer are used.

The sealing layer 50 can be formed of a thin film made of a single material, but can be formed of a multilayered structure with the respective layers having different functions, which can be expected to have effects of relaxing the entire stress of the sealing layer 50, suppressing the occurrence of defects, such as cracks or pin holes, due to dust or the like during a manufacturing process, and easily optimizing the development of material. For example, the sealing layer 50 can form a two-layered structure in which a “sealing auxiliary layer” is laminated on a layer for achieving the inherent object for inhibiting the penetration of degrading factors, such as water molecules so as to have the function that cannot be easily achieved by the layer. The sealing layer 50 can be configured of three or more layers, but the number of layers is preferably small in terms of manufacturing cost.

A method for forming the sealing layer 50 is not specifically limited, but the sealing layer 50 is preferably deposited by a method that hardly degrades the performance or properties of the photoelectric conversion layer 32 already deposited. In the related art, the sealing layer is generally deposited by various vacuum vapor deposition techniques. In the conventional sealing layer, a thin film is difficult to grow in stepped portions that are caused by a structure on a substrate surface, fine defects on the substrate surface, particles attached to the substrate surface, and the like (because the stepped portion becomes a diagonally part), so that the sealing layer in the stepped portion is much thinner than that in a flat portion. Thus, the stepped portion might serve as a route through which the degrading factors pass. In order to completely cover the stepped portion with the sealing layer, it is necessary to increase the thickness of the entire sealing layer by depositing the sealing layer over the flat portion in a thickness of 1 μm or more. The vacuum degree in formation of the sealing layer is preferably 1·10³ Pa or less, and more preferably 5·10² Pa or less.

However, in an imaging element having a pixel dimension of less than 2 μm, especially, about 1 μm, when the thickness of the sealing layer 50 is large, the distance between a color filter and the photoelectric conversion layer becomes large, which might cause the incident light to be diffracted/scattered within the sealing layer, inducing a mixed colored light. Thus, when applied to the imaging element with the pixel dimension of about 1 μm, the material and manufacturing method for the sealing layer 50 that does not degrade the element performance is required even though the thickness of the sealing layer 50 is decreased.

An atomic layer deposition (ALD) method is one of the CVD methods, and a technique for forming a thin film by alternately repeating adsorption/reaction of an organic metal compound molecule, a metal halide compound molecule, or a metal hydride molecule as the material for the thin film onto a substrate surface, and decomposition of unreacted group contained therein. When the material for the thin film reaches the substrate surface, the material is in the state of low-molecule, and if there is a slight space into which the low-molecule material can enter, the thin film can grow. Thus, the stepped portion which is difficult to handle in the conventional thin film forming method is completely covered (the thickness of a thin film having grown in the stepped portion is the same as that of the thin film having grown in the flat portion). That is, this embodiment has very excellent stepped-portion covering property. Thus, the stepped portions caused by the structure on the substrate surface, the fine defects on the substrate surface, and particles attached to the substrate surface can be completely covered. Those stepped portions cannot serve as a route which the degrading factors of the photoelectric conversion material invade. When the sealing layer 50 is formed by the atomic layer deposition, the necessary sealing layer can be more effectively thinned than that in the related art.

When the sealing layer 50 is formed by the atomic layer deposition, the material corresponding to ceramic that is preferable for the above-mentioned sealing layer 50 can be selected for the sealing layer as appropriate. However, since the photoelectric conversion layer of the invention uses an organic photoelectric conversion material, it is limited to material that allows the growth of the thin film at a relatively low temperature which does not degrade the organic photoelectric conversion material. By the atomic layer deposition using alkylaluminum or aluminum halide as the material, a fine aluminum oxide thin film can be formed at a temperature of less than 200° C. that does not degrade the organic photoelectric conversion material. In particular, in the use of the trimethyl aluminum, preferably, an aluminum oxide thin film can be formed even at about 100° C. Preferably, silicon dioxide or titanium oxide can be appropriately selected as the material, whereby the fine thin film can be formed at a temperature of less than 200° C. in the same way as aluminum oxide.

Note that the thin film formed by the atomic layer deposition can achieve the good thin film at a low temperature matchlessly in terms of the stepped portion covering property and extreme precision. However, the properties of the thin film material might be degraded with chemicals used in a photolithography process. For example, the aluminum oxide thin film deposited by the atomic layer deposition is amorphous, and thus might have its surface eroded with an alkaline solution, such as a development solution or removal solution.

In many cases, most of thin films formed by the CVD method, such as the atomic layer deposition, have tensile stress with a very large internal stress. Such a thin film can be degraded due to cracks formed in the thin film itself by a discontinuous repetition of heating and cooling, like a semiconductor manufacturing process, storage/usage under high-temperature/high-humidity atmosphere for a long term.

Thus, in use of the sealing layer 50 deposited by the atomic layer deposition, a sealing auxiliary layer having excellent chemical resistance and which can offset the internal stress of the sealing layer 50 is preferably formed.

Such an auxiliary sealing layer can includes a layer containing at least one of ceramics with excellent chemical resistance which is deposited by a physical vapor deposition (PVD) method, such as sputtering, and which includes a metal oxide, a metal nitride, and a metal nitride oxide. The ceramics deposited by the PVD method, such as sputtering, often has a large compressive stress, which can cancel the tensile stress of the sealing layer 50 formed by the atomic layer deposition method.

The sealing layer 50 formed by the atomic layer deposition preferably contains any one of aluminum oxide, silicon oxide, and titanium oxide. The sealing auxiliary layer is preferably a sputtering film containing any one of aluminum oxide, silicon oxide, silicon nitride, and silicon nitride oxide. In this case, the thickness of the sealing layer 50 is preferably not less than 0.05 μm nor more than 0.5 μm.

In the way above, the photoelectric conversion element 1 is structured.

When the n-type organic semiconductor is fullerene or a fullerene derivative, preferable p-type organic semiconductor material will be described below. These materials are compounds with a shallower HOMO level than that of fullerene or a fullerene derivative when using the fullerene or a fullerene derivative as the n-type semiconductor, that is, a dye having an absorption peak in a visible light region (in a wavelength of 400 nm to 700 nm).

Subsequently, when the n-type organic semiconductor is fullerene or a fullerene derivative, preferable materials for the electron blocking layer (electron-donating organic material) will also be described below.

These compounds are preferable as the p-type organic semiconductor material, or electron-donating organic material when using the fullerene or a fullerene derivative as the n-type semiconductor. In other structures, these compounds may be used as another functional layer. As mentioned above, the layers of these organic compounds are preferably deposited using the organic material 60 for deposition.

### <<P-Type Organic Semiconductor Material>>

Suitable material for the p-type organic semiconductor is, preferably, for example, a compound represented by the following general formula (D-I).

### [Chemical Formula 1]

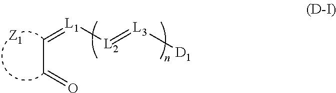

(In the general formula (D-I), Z₁ represents a group of atoms required to form a 5- or 6-membered ring. L₁, L₂ and L₃ independently represents a non-substituted methine group, or a substituted methine group. D₁ represents a group of atoms. n represents an integer number of 0 or more.)

In the general formula (D-I), Z₁ represents a ring including at least two carbon atoms, and represents a condensed ring containing the 5-membered ring, the 6-membered ring, or at least one of the 5- and 6-membered rings. The condensed ring containing the 5-membered ring, the 6-membered ring, or at least one of the 5- and 6-membered rings is preferably a merocyanine dye normally used as an acid nucleus. Specific examples can include the following, for example.

(a) 1,3-dicarbonyl nucleus: e.g. 1,3-indaneddione nucleus, 1,3-cyclohexanedione, 5-5-dimethyl-1,3-cyclohexane dione, 1,3-dioxane-4,6-dione, and the like.

(b) Pyrazolinone nucleus: e.g. 1-phenyl-2-pyrazoline-5 one, 3-methyl-1-phenyl-2-phyrazoline-5-one, 1-(2-benzo thiazoyle)-3-methyl-2-pyrazoline-5-one, etc.

(c) Isoxazolinon nucleus: e.g. 3-phenyl-2-isoxazoline-5-one, 3-methyl-2-isoxazoline-5-one, etc.

(d) Oxindole nucleus: e.g. 1-alkyl-2,3-dihydro-2-oxindole, etc.

(e) 2,4,6-triketohexahydropyrimidin nucleus: e.g. barbituric acid, or 2-thiobarbituric acid, and its derivative, etc. The derivatives can include, for example, 1-alkyl, such as 1-methyl, or 1-ethyl; 1,3-dialkyl, such as 1,3-dimethyl, 1,3-diethyl, or 1,3-dibutyl; 1,3-diaryl, such as 1,3-diphenyl, 1,3-di(p-chlorophenyl), 1,3-di(p-ethoxycarbonyl phenyl); 1-alkyl-1-aryl, such as 1-ethyl-3-phenyl; 1,3-dihetero ring substitute, such as 1,3-di(2-pyridyl).

(f) 2-thio-2,4-thiazolidinedione nucleus: e.g. rhodanine, and its derivative, etc. The derivatives can include, for example, 3-alkyl rhodanine, such as 3-methyl rhodanine, 3-ethyl rhodanine, or 3-aryl rhodanine; 3-aryl rhodanine, such as 3-phenyl rhodanine; 3 hetero ring substituted rhodarinine, such as 3-(2-pyridyl)rhodanine etc.

(g) 2-thio-2,4-oxazolidinedione (2-thio-2,4-(3H,5H)-oxazole dione nucleus: e.g. 3-ethyl-2-thio-2,4-oxazolidinedione etc.

(h) chianafutenon nucleus: e.g. 3(2H)-chianafutenon-1,1-dioxide etc.

(i) 2-thio-2,5 thiazolidinedione nucleus: e.g. 3-ethyl-2-thio-2,5-thiazolidinedione etc.

(j) 2,4-thiazolidinedione nucleus: e.g. 2,4-thiazolidinedione, 3-ethyl-2,4-thiazolidinedione, 3-phenyl-2,4-thiazolidinedione etc.

(k) thiazoline-4-one nucleus: e.g. 4-chiazorinon, 2-ethyl-4 chiazorinon etc.

(l) 2,4-imidazolidinedione(hydantoin) nucleus: e.g. 2,4-imidazolidinedione, 3-ethyl-2,4-imidazolidinedione etc.

(m) 2-thio-2,4-imidazolidinedione(2-thiohydantoin) nucleus: e.g. 2-thio-2,4-imidazolidinedione, 3-ethyl-2-thio-2,4-imidazolidinedione etc.

(n) imidazoline-5-one nucleus: e.g. 2-propyl-mercapto-2-imidazoline-5-one etc.

(o) 3,5-pyrazolidinedione nucleus: e.g. 1,2-diphenyl-3,5-pyrazolidinedione, 1,2-dimethyl-3,5-pyrazolidinedione etc.

(p) benzothiophen-3-one nucleus: e.g. a benzothiophen-3-one, oxo benzothiophene-3-one, di-oxo-benzothiophene-3-one etc.

(q) indanone nucleus: e.g. 1-indanone, 3-phenyl-1-indanone, 3-methyl-1-indanone, 3,3-diphenyl-1-indanone, 3,3-dimethyl-1-indanone etc.

Examples of a ring represented by Z₁ include, preferably, 1,3-dicarbonyl nucleus, pyrazolinone nucleus, 2,4,6-triketohexahydropyrimidine nucleus (including thioketone, e.g. barbituric acid nucleus, 2-thiobarbituric nucleus), 2-thio-2,4-thiazolidinedione nucleus, 2-thio-2,4-oxazolidinedione nucleus, 2-thio-2,5-thiazolidinedione nucleus, 2,4-thiazolidinedione nucleus, 2,4-imidazolidine dione nucleus, 2-thio-2,4-imidazolidine dione nucleus, 2-imidazoline-5-one nucleus, 3,5 pyrazolidinedione nucleus, benzothiophene-3-one nucleus, indanone nucleus, more preferably, 1,3-dicarbonyl nucleus, 2,4,6-triketohexahydropyrimidine nucleus (including a thioketone body, such as barbituric acid nucleus, or 2-thiobarbituric acid nucleus), 3,5 pyrazolidinedione nucleus, a benzothiophen-3-one nucleus, indanone nucleus, even more preferably 1,3-dicarbonyl nucleus, 2,4,6-triketohexahydropyrimidine nucleus (including thioketone body, such as barbituric acid nucleus, and 2-thiobarbituric acid nucleus), particularly most preferably, 1,3-indanedione nucleus, barbituric acid nucleus, 2-thiobarbituric acid nucleus, and a derivative thereof.

The ring represented by Z₁ is preferably represented by the following general formula (Z₁).

### [Chemical Formula 2]

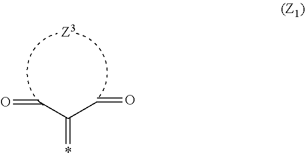 [SMILES: *=C(C=O)C=O]

(Z³ is a ring containing at least 3 carbon atoms, and represents a 5-membered ring, a 6-membered ring, or a condensed ring containing at least one of the 5- and 6-membered rings. * represents a binding position with L₁ in the general formula (D-I).)

Z³ can be selected from the rings formed by the above-mentioned Z₁, and is preferably 1,3-dicarbonyl nucleus, 2,4,6-triketohexahydropyrimidine nucleus (including a thioketone body), particularly preferably, 1,3-indanedione nucleus, barbituric acid nucleus, 2-thiobarbituric acid nucleus, and their derivatives.

In the general formula (D-I), the ring represented by Z₁ serves as an acceptor part in some cases. The inventors have found that by controlling the interaction between the acceptor parts, the high hole transport property can be exhibited as a film co-deposited with fullerene. Specifically, the interaction can be controlled by arranging the structure of the acceptor part, and introducing a substituent group as a three-dimensional inhibitor. In barbituric acid nucleus, and 2-thiobarbituric acid nucleus, two hydrogen atoms in the N-position, preferably, both of two hydrogen atoms, are replaced by a substituent group, which can preferably control the intermolecular interaction. The substituent group can be a substituent group w to be described later, more preferably, an alkyl group, and most preferably, a methyl group, an ethyl group, a propyl group, or a butyl group.

When the ring represented by Z₁ is preferably represented by the following general formula (D-V).

### [Chemical Formula 3]

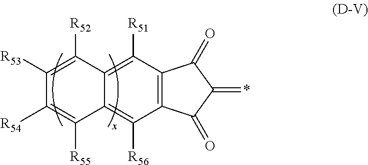

(In the general formula (D-V), R₅₁ to R₅₆ each independently represent a hydrogen atom or a substituent group. Any adjacent two of R₅₁ to R₅₆ may be bonded to each other to form a ring. * represents the binding position with the L₁. x represents 0 or 1.)

In the group represented by the general formula (D-V), R₅₁ to R₅₆ each independently represent a hydrogen atom or a substituent group. The substituent group that can be applied is the substituent W described below for example, preferably an alkyl group, and more preferably an alkyl group having 1 to 6 carbon atoms. Also, any adjacent two of R₅₁ to R₅₆ may be bonded together to form a ring. In formation of the ring, preferably, R₅₃ and R₅₄ are bonded together to form a ring (e.g. a benzene ring, a pyridine ring, and a pyrazine ring).

It is preferred that all of R₅₁ to R₅₆ are a hydrogen atom.

When the ring represented by Z₁ is a 2,4,6-triketohexahydropyrimidine nucleus (including a thioketone body), it is preferred that the Z₁ is the group represented by the general formula (D-VI).

### [Chemical Formula 4]

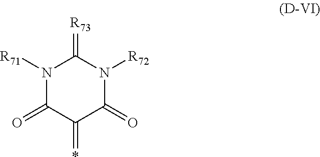

(In the general formula (D-VI), R₇₁ and R₇₂ each independently represent a hydrogen atom or a substituent group. R₇₃ represents an oxygen atom, a sulfur atom, or a substituent group. * represents the binding position with the L₁.)

In the general formula (D-VI), R₇₁ and R₇₂ each independently represent a hydrogen atom or a substituent group. The substituent group that can be applied is, for example, the substituent W described below. R₇₁ and R₇₂ each independently represent preferably an alkyl group, an aryl group, or a heterocyclic group (preferably, 2-pyridyl, etc.), and more preferably, an alkyl group having 1 to 6 carbon atoms (e.g. methyl, ethyl, n-propyl, t-butyl).

R₇₃ represents an oxygen atom, a sulfur atom or a substituent group. R₇₃ preferably represents an oxygen atom, or sulfur atom. A bonding part of the substituent group is preferably a nitrogen atom or carbon atom. In the case where the bonding part is the nitrogen atom, R₇₃ other than the bonding part preferably include an alkyl group (having 1 to 12 carbon atoms) or an aryl group (6 to 12 carbon atoms). Specifically, examples of R₇₃ include methylamino group, ethylamino group, butylamino group, hexylamino group, phenylamino group, and naphthylamino group. In the case where the bonding part is the carbon atom, preferably, R₇₃ is further substituted with at least one electron-attracting group. Examples of the electron-attracting group include a carbonyl group, a cyano group, a sulfoxide group, a sulfonyl group, and a phosphoryl group, and preferably, further has a substituent group. This substituent group is, for example, the substituent group W to be described later. R₇₃ is preferable to form a 5- or 6-membered ring containing a carbon atom, and specifically includes the following structures. Note that, Ph in the group represents a phenyl group.

### [Chemical Formula 5]

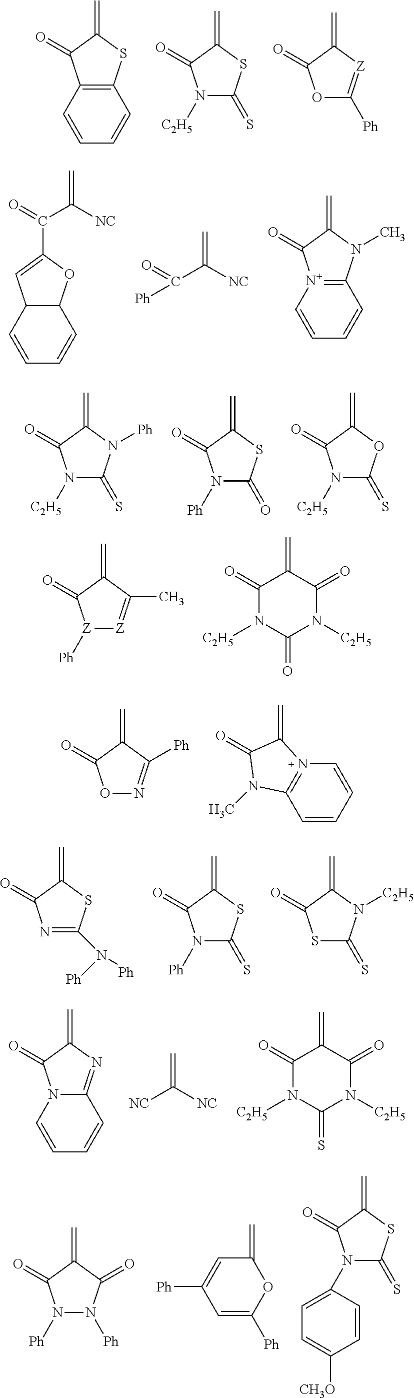

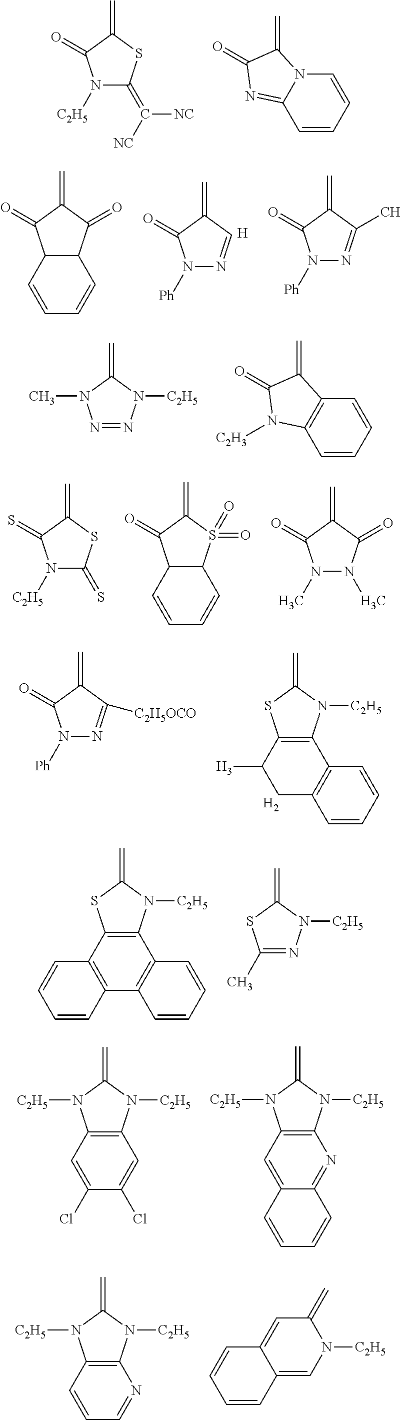

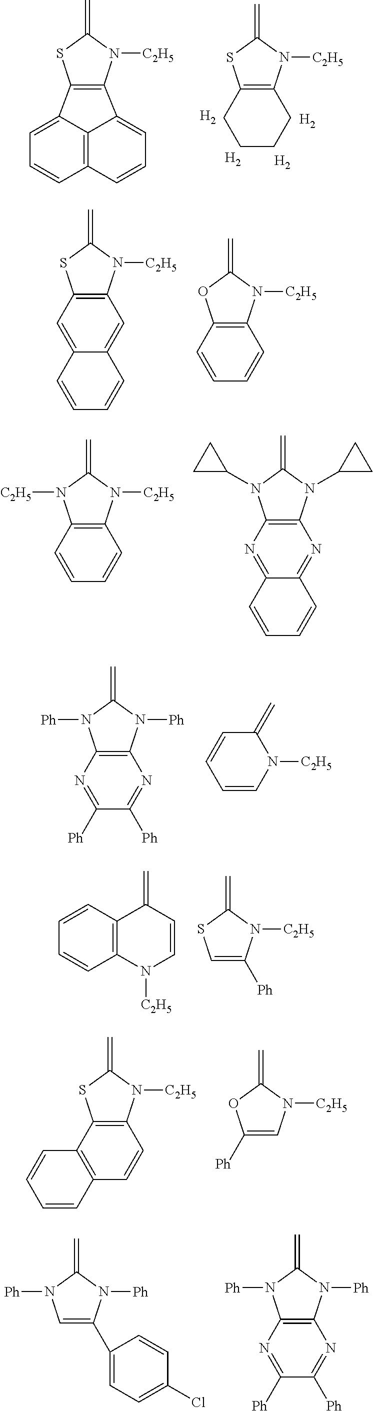

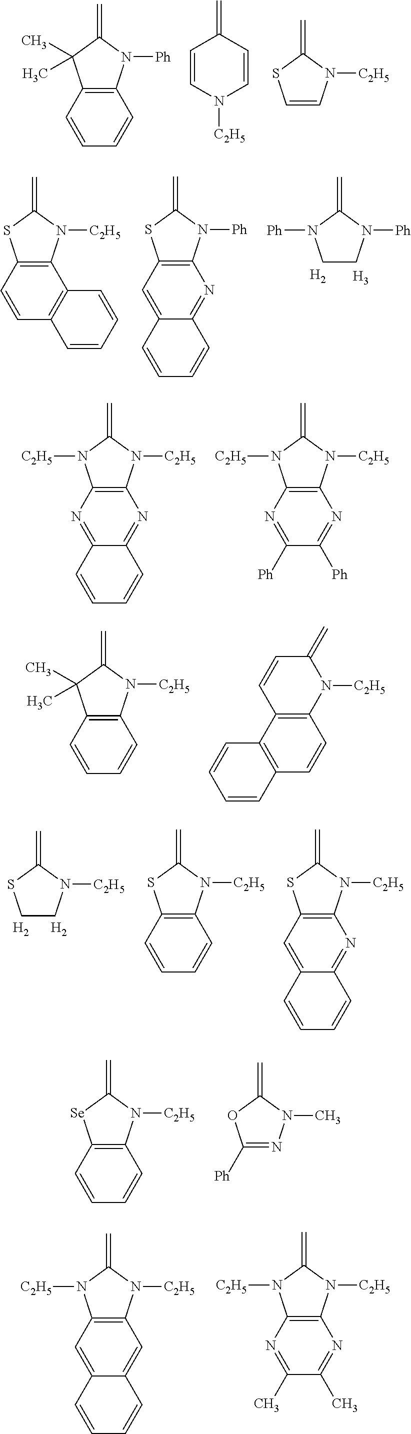

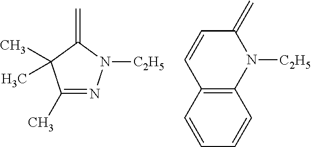

In the general formula (D-I), L₁, L₂ and L₃ each independently represent an unsubstituted methine group or a substituted methine group. Substituted methine groups may be bonded together to form a ring. An example of the ring is a 6-membered ring (e.g. benzene ring, etc.) Although the substituent of the substituted methine group is the substituent W described below, all L₁, L₂ and L₃ are preferably unsubstituted methine group.

In the general formula (D-I), n represents an integer number of 0 or more, preferably an integer number of not less than 0 nor more than 3, and more preferably 0. If n is increased, an absorption wavelength range can be shifted to a longer wavelength, but a thermal decomposition temperature becomes lower. Here, n=0 is preferable because of a proper absorption in the visible region in terms of suppressing the thermal decomposition during vapor deposition.

In the general formula (D-I), D₁ represents a group of atoms. D₁ is preferably a group containing —NR^{a}(R^{b}). Further the D₁ preferably represents an aryl group substituted with —NR^{a}(R^{b}) (preferably, a phenyl group or a naphthyl group that may have a substituent group). R^{a} and R^{b} each independently represent a hydrogen atom, or a substituent group, but examples of the substituent group include the substituent W described below, preferably an aliphatic hydrocarbon group (preferably, an alkyl or alkenyl group that may have a subs tituent group), an aryl group, or a heterocyclic group.

The hetero ring is preferably a 5-membered ring, such as a furan ring, a thiophene ring, a pyrrole ring, and an oxadiazole ring.

When R^{a} and R^{b} are substituent groups (preferably, an alkyl group, or an alkenyl group), the substituent groups may be bonded to a hydrogen atom or a substitute group of an aromatic ring skeleton of the aryl group substituted with —NR^{a}(R^{b}) to form a ring (preferably, a 6-membered ring).

R^{a} and R^{b} may have their substituent groups bonded to each other to form a ring (preferably a 5-membered or 6-membered ring, more preferably a 6-membered ring). Alternatively, R^{a} and R^{b} may be respectively bonded to a substituent group of L (which indicates any one of L₁, L₂, L₃) to form a ring (preferably a 5-membered or 6-membered ring, and more preferably a 6-membered ring).

D₁ is preferably an aryl group (preferably, a phenyl group, a naphthyl group) substituted with an amino group in the para-position. Examples of the substituent group of the amino group include the substituent W described below, but preferably, an aliphatic hydrocarbon group (preferably, an alkyl group that may be substituted), an aryl group (preferably, a phenyl group or naphthyl group that may be substituted), and a heterocyclic group. The amino group is preferably the so-called diaryl group-substituted amino group which is substituted with two aryl groups. Further, the substituent group of the amino group (preferably, an alkyl group or an alkenyl group which may be substituted) may be bonded to a hydrogen atom or a substitute group of an aromatic ring skeleton of the aryl group (preferably, a benzene ring, or a naphthyl group) to form a ring (preferably, a 6-membered ring).

Examples of R^{a} and R^{b}, which are substituent groups of an aliphatic hydrocarbon group, an aryl group, or a heterocyclic group, include an alkyl group, an alkenyl group, an aryl group, an alkoxy group, an aryloxy group, an acyl group, an alkoxycarbonyl group, an aryloxycarbonyl group, an acylamino group, a sulfonylamino group, a sulfonyl group, a silyl group, an aromatic heterocyclic group, more preferably an alkyl group, an alkenyl group, an aryl group, an alkoxy group, an aryloxy group, a silyl group, an aromatic heterocyclic group, and most preferably, an alkyl group, an aryl group, an alkoxy group, an aryloxy group, a silyl group, and an aromatic heterocyclic group. Specifically, the substituent group W to be described later can be applied to R^{a} and R^{b}.

R^{a} and R^{b} are preferably an alkyl group, an aryl group, or an aromatic heterocyclic group. Examples of R^{a} and R^{b} preferably include an alkyl group, an alkylene group which is coupled with L to form a ring, or an aryl group, more preferably, an alkyl group having 1 to 8 carbon atoms, an alkylene group which is coupled with L to form a 5- or 6-membered ring, or a substituted or unsubstituted aryl group, even more preferably, an alkyl group having 1 to 8 carbon atoms, or a substituted or unsubstituted aryl group, and particularly preferably, a substituted or unsubstituted phenyl group or naphthyl group.

D₁ is preferably also represented by the following general formula (D-II).

### [Chemical Formula 6]

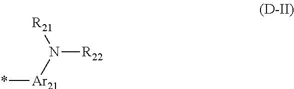

(In the general formula (D-II), R₂₁ and R₂₂ each independently represent a hydrogen atom or a substituent group. Ar₂₁ represents an aromatic hydrocarbon ring group or an aromatic heterocyclic group. * Represents the binding position. Ar₂₁ and R₂₁, Ar₂₁ and R₂₂, and R₂₁ and R₂₂ may be respectively bonded to each other to form a ring.)

R₂₁, and R₂₂ each independently represent a hydrogen atom or a substituent group, and examples of the substituent group can include the substituent W described below. These may further have a substituent group. Specific examples of the substituent groups include the substituent group W described below, preferably, a halogen atom, an alkyl group, an aryl group, a heterocyclic group, a hydroxyl group, an amino group, or a mercapto group, more preferably a halogen atom, an alkyl group, an aryl group, a heterocyclic group, more preferably, a fluorine atom, an alkyl group, an aryl group, particularly preferably, an alkyl group, an aryl group, and most preferably, an alkyl group. The alkyl group preferably has a linear or branched structure, and preferably, has 1 to 20 carbon atoms, more preferably, 1 to 10, and further preferably, 1 to 5.

Examples of R₂₁ and R₂₂ preferably include an alkyl group, an alkenyl group, an aryl group, an alkoxy group, an aryloxy group, an acyl group, an alkoxycarbonyl group, an aryloxycarbonyl group, an acylamino group, a sulfonylamino group, a sulfonyl group, a silyl group, an aromatic heterocyclic group, more preferably an alkyl group, an aryl group, an alkoxy group, an aryloxy group, a silyl group, an aromatic heterocyclic group (preferably a furan ring, a thiophene ring, a pyridine ring, a pyridazine ring, a pyrimidine ring, a pyrazine ring, an oxadiazole ring, a triazole ring, an imidazole ring, a pyrazole ring, a thiazole ring), more preferably an alkyl group, an aryl group, or an aromatic heterocyclic group (preferably a furan ring, a thiophene ring, a pyridine ring, an oxadiazole ring, an imidazole ring, a pyrazole ring, a thiazole ring), particularly preferably, an alkyl group or an aryl group, among them, preferably an alkyl group having 1 to 8 carbon atoms, or a phenyl group, an alkyl-substituted phenyl group, a phenyl substituted phenyl group, a naphthyl group, a phenanthryl group, an anthryl group, or a fluorenyl group (preferably 9,9′-dimethyl-2-fluorenyl group), particularly preferably a substituted or unsubstituted aryl group, most preferably a substituted or unsubstituted phenyl group or naphthyl group. Also, these substituents may be bonded to each other to form a ring.

Ar₂₁ represents an aromatic hydrocarbon ring or an aromatic heterocyclic group, and these may have the substituent group W described below as a substituent group. Preferably, examples of Ar₂₁ include a benzene ring, a naphthalene ring, an indane ring, an anthracene ring, a fluorene ring, a pyrene ring, a phenanthrene ring, a perylene ring, a pyridine ring, a quinoline ring, an isoquinoline ring, a phenanthridine ring, a pyrimidine ring, a pyrazine ring, a pyridazine ring, a triazine ring, a cinnoline ring, an acridine ring, a phthalazine ring, a quinazoline ring, a quinoxaline ring, a naphthyridine ring, a pteridine ring, a pyrrole ring, a pyrazole ring, a triazole ring, an indole ring, a carbazole ring, an indazole ring, a benzimidazole ring, an oxazole ring, a thiazole ring, an oxadiazole ring, a thiadiazole ring, benzoxazole ring, a benzothiazole ring, an imidazopyridine ring, a thiophene ring, a benzothiophene ring, a furan ring, a benzofuran ring, a phosphole ring, a phosphinine ring, a silole ring, more preferably, a benzene ring, a naphthalene ring, a fluorene ring, an indane ring, an anthracene ring, a pyrene ring, a phenanthrene ring, a perylene ring, a pyrrole ring, an indole ring, a carbazole ring, an indazole ring, a thiophene ring, a benzothiophene ring, a furan ring, a benzofuran ring, more preferably, a benzene ring, a naphthalene ring, a fluorene ring, an indane ring, an indole ring, a carbazole ring, an indazole ring, particularly preferably, a benzene ring, a naphthalene ring, a fluorene ring, an indane ring, an anthracene ring. Among them, a benzene ring, a naphthalene ring, and a fluorene ring are preferable, and a benzene ring, and a naphthalene ring are most preferable.

Ar₂₁ may further have a substituent group. Specific example of the further substituent group includes the substituent W described below, preferably a halogen atom, an alkyl group, an aryl group, a heterocyclic group, a hydroxyl group, an amino group, or a mercapto group, more preferably, a halogen atom, an alkyl group, an aryl group, a heterocyclic group, more preferably, a fluorine atom, an alkyl group, an aryl group, particularly preferably, an alkyl group, an aryl group, and most preferably an alkyl group.

A compound (dye) having a structure in which D₁ of the general formula (D-I) is represented by the general formula (D-II) is used in combination with fullerenes when a donor represented by the D₁ has a triarylamine skeleton, which can achieve high charge collection efficiency and high-speed response, while maintaining the heat resistance of the element.

One preferred form of the general formula (D-II) is a formula (II-a).

### [Chemical Formula 7]

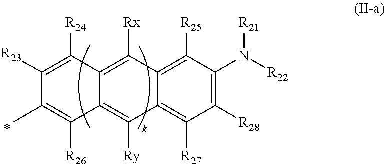

(In the general formula (II-a), R₂₁ and R₂₂ each independently represent a hydrogen atom or a substituent group. R₂₃ to R₂₈ each independently represent a hydrogen atom or a substituent group. k represents an integer number of 0 or more. Rx and Ry each independently represent a hydrogen atom or a substituent group. When k represents 2 or more, a plurality of Rxs and Rys may be the same as or different from each other. In addition, R₂₃ and R₂₄, R₂₄ and Rx, Ry and R₂₅, R₂₅ and R₂₁, R₂₆ and Rx, Ry and R₂₇, R₂₇ and R₂₈, R₂₈ and R₂₂, and R₂₁ and R₂₂ may be respectively bonded to each other to form a ring. * represents the binding position.)

In the general formula (II-a), k is preferably 0 or 1, and more preferably 0. R₂₁ and R₂₂ have the same meanings as those of R₂₁ and R₂₂ in the general formula (D-II), and the preferred range thereof is also the same.

When R₂₃ to R₂₈, Rx, and Ry each represent a substituent group, examples of the substituent group include the substituent W described below. They may further have a substituent group. Specific examples of the further substituent group include the substituent W described below, preferably a halogen atom, an alkyl group, an aryl group, a heterocyclic group, a hydroxyl group, an amino group, and a mercapto group, more preferably, a halogen atom, an alkyl group, an aryl group, a heterocyclic group, more preferably a fluorine atom, an alkyl group, an aryl group, particularly preferably, an alkyl group, an aryl group, and most preferably an alkyl group.

Each of R₂₃ to R₂₈ is preferably a hydrogen atom. In addition, both Rx and Ry are preferable hydrogen atom. R₂₃ to R₂₈ are hydrogen atoms, and Rx and Ry are more preferably a hydrogen atom.

D₁ also preferably be a group represented by the following general formula (II-b) or (II-c).

### [Chemical Formula 8]

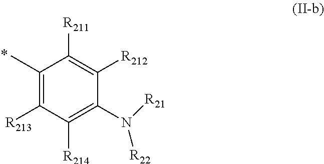

(In the general formula (II-b), R₂₁ and R₂₂ each independently represent a hydrogen atom or a substituent group. R₂₁₁ to R₂₁₄ each independently represent a hydrogen atom or a substituent group. R₂₁₁ and R₂₁₂, R₂₁₃ and R₂₁₄, R₂₁ and R₂₂, R₂₁₂ and R₂₁, and R₂₁₄ and R₂₂ may be respectively bonded to each other to form a ring. * represents the binding position.)

R₂₁ and R₂₂ have the same meanings as those of R₂₁ and R₂₂ in the general formula (D-II), and the preferred range thereof is also the same.

When each of R₂₁₁ to R₂₁₄ represents a substituent group, examples of the substituent group include the substituent W described below, preferably, R₂₁₁ to R₂₁₄ each represent a hydrogen atom, or a combination of R₂₁₂ and R₂₁ or a combination of R₂₁₄ and R₂₂ forms 5- or 6-membered ring, and more preferably, each of the R₂₁₁ to R₂₁₄ is a hydrogen atom.

When R₂₁₁ and R₂₁₂, R₂₁₃ and R₂₁₄, R₂₁ and R₂₂, R₂₁₂ and R₂₁, and R₂₁₄ and R₂₂ are bonded to each other to form a ring, the ring formed include the ring R to be described later. Preferably, examples of the ring include a benzene ring, a naphthalene ring, an anthracene ring, a pyridine ring, a pyrimidine ring, and the like.

### [Chemical Formula 9]

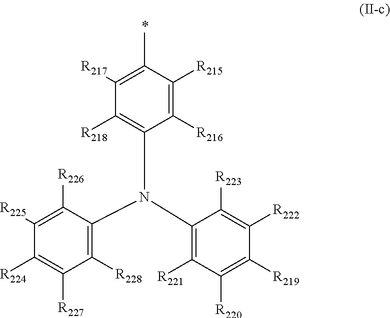

(In the general formula (II-c), R₂₁₅ to R₂₁₈, R₂₁₉ to R₂₂₃, and R₂₂₄ to R₂₂₈ each independently represent a hydrogen atom or a substituent group. R₂₁₅ and R₂₁₆, R₂₁₇ and R₂₁₈, R₂₂₃ and R₂₂₂, R₂₂₂ and R₂₁₉, R₂₁₉ and R₂₂₀, R₂₂₀ and R₂₂₁, R₂₂₈ and R₂₂₇, R₂₂₇ and R₂₂₄, R₂₂₄ and R₂₂₅, and R₂₂₅ and R₂₂₆ may be respectively bonded to each other to form a ring. * represents a binding position.)

R₂₁₅ and R₂₁₆, R₂₁₇ and R₂₁₈, R₂₂₃ and R₂₂₂, R₂₂₂ and R₂₁₉, R₂₁₉ and R₂₂₀, R₂₂₀ and R₂₂₁, R₂₂₈ and R₂₂₇, R₂₂₇ and R₂₂₄, R₂₂₄ and R₂₂₅, and R₂₂₅ and R₂₂₆ may be respectively bonded to each other to form a ring. The formed ring includes the ring R to be described later. Preferably, examples of the ring include a benzene ring, a naphthalene ring, an anthracene ring, a pyridine ring, a pyrimidine ring, and the like.

R₂₁₆ and R₂₂₃, R₂₁₈ and R₂₂₆, and R₂₂₈ and R₂₂₁ may be respectively bonded to each other. R₂₁₆ and R₂₂₃, R₂₁₈ and R₂₂₆, and R₂₂₈ and R₂₂₁ may be respectively bonded to each other to form a 5- to 10-membered ring (preferably 5 to 6-membered ring). Each of the bonding between the R₂₁₆ and R₂₂₃, R₂₁₈ and R₂₂₆, R₂₂₈ and R₂₂₁ may be a single bond.

When each of the R₂₁₅ to R₂₁₈, R₂₁₉ to R₂₂₃, and R₂₂₄ to R₂₂₈ represents a substituent group, examples of the substituent group include the substituent W to be described later. R₂₁₅ to R₂₁₈, R₂₁₉ to R₂₂₃, and R₂₂₄ to R₂₂₈ each preferably represent a hydrogen atom, a halogen atom, an alkyl group having 1 to 18 carbon atoms, an aryl group having 6 to 18 carbon atoms, a heterocyclic group having 4 to 16 carbon atoms, more preferably, a hydrogen atom, an alkyl group having 1 to 12 carbon atoms, an aryl group having 6 to 14 carbon atoms, a fluorine atom, and even more preferably, a hydrogen atom, an alkyl group having 1 to 6 carbon atoms, an aryl group having 6 to 10 carbon atoms. Among them, a hydrogen atom, a fluorine atom, a methyl group, an ethyl group, a propyl group, a butyl group, a hexyl group, a cyclohexyl group, a phenyl group, and a naphthyl group are preferable. A hydrogen atom, a methyl group, a butyl group, a hexyl group, and a phenyl group are particularly preferred. The alkyl group may have a branch.

These may further have a substituent group. Specific examples of the further substituent group include the substituent W described below, preferably a halogen atom, an alkyl group, an aryl group, a heterocyclic group, a hydroxyl group, an amino group, and a mercapto group, more preferably a halogen atom, an alkyl group, an aryl group, a heterocyclic group, more preferably a fluorine atom, an alkyl group, an aryl group, particularly preferably an alkyl group, an aryl group, and most preferably an alkyl group.

The general formula (D-I) is preferably the following formula (II-d).

### [Chemical Formula 10]

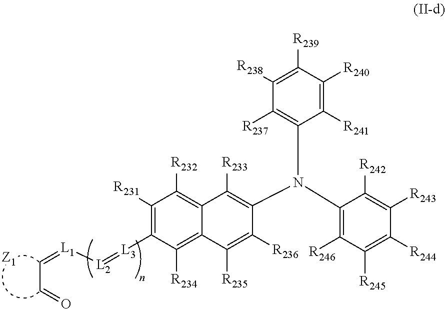

(In the general formula (II-d), Z₁ represents a group of atoms required to form a 5 or 6-membered ring. L₁, L₂ and L₃ each independently represent an unsubstituted methine group or a substituted methine group. R₂₃₁ to R₂₃₆ each independently represent a hydrogen atom or a substituent group. The R₂₃₁ and R₂₃₂, the R₂₃₂ and R₂₃₃, R₂₃₄ and R₂₃₅, and R₂₃₅ and R₂₃₆ may be respectively bonded to each other to form a ring. R₂₃₇ to R₂₄₁ and R₂₄₂ to R₂₄₆ each independently represent a hydrogen atom or a substituent group. Among the R₂₃₇ to R₂₄₁ and R₂₄₂ to R₂₄₆, the adjacent atoms or groups may be bonded to each other to form a ring. The R₂₃₃ and R₂₃₇, and R₂₃₆ and R₂₄₆ may be independently bonded to each other to form a ring.)

In the general formula (II-d), Z₁, L₁, L₂, L₃ have the same meanings as Z₁, L₁, L₂, L₃ in the general formula (D-I), and preferred ranges thereof are also the same.

When R₂₃₁ to R₂₃₆ represent a substituent group, examples of the substituent include the substituent W described below. R₂₃₁ to R₂₃₆ are preferably a hydrogen atom, a halogen atom, an alkyl group having 1 to 18 carbon atoms, an aryl group having 6 to 18 carbon atoms, a heterocyclic group having 4 to 16 carbon atoms, more preferably, a hydrogen atom, an alkyl group having 1 to 12 carbon atoms, an aryl group having 6 to 14 carbon atoms, a fluorine atom, and even more preferably, a hydrogen atom, an alkyl group having 1 to 6 carbon atoms, an aryl group having 6 to 10 carbon atoms. Among them, a hydrogen atom, a fluorine atom, methyl group, an ethyl group, a propyl group, a butyl group, a hexyl group, a cyclohexyl group, a phenyl group, and a naphthyl group are preferable, and a hydrogen atom, a methyl group, a butyl group, a hexyl group, and a phenyl group are particularly preferable. The alkyl group may have a branch.

They may further have a substituent group. Specific examples of the further substituent group include the substituent W described below, preferably a halogen atom, an alkyl group, an aryl group, a heterocyclic group, a hydroxyl group, an amino group, or a mercapto group, more preferably, a halogen atom, an alkyl group, an aryl group, a heterocyclic group, more preferably a fluorine atom, an alkyl group, an aryl group, particularly preferably an alkyl group, an aryl group, and most preferably an alkyl group.

R₂₃₁ and R₂₃₂, R₂₃₂ and R₂₃₃, R₂₃₄ and R₂₃₅, and R₂₃₅ and R₂₃₆ may be respectively bonded to each other to form a ring. Examples of the ring formed include the ring R to be described later. Preferably, examples of the rings include a benzene ring, a naphthalene ring, an anthracene ring, a pyridine ring, a pyrimidine ring, and the like.

R₂₃₇ to R₂₄₁ and R₂₄₂ to R₂₄₆ each independently, represent a hydrogen atom or a substituent group. When R₂₃₇ to R₂₄₁ and R₂₄₂ to R₂₄₆ represent a substituent group, examples of the substituent include the substituent W described below. R₂₃₇ to R₂₄₁ and R₂₄₂ to R₂₄₆ are preferably a hydrogen atom, a halogen atom, an alkyl group having 1 to 18 carbon atoms, an aryl group having 6 to 18 carbon atoms, a heterocyclic group having 4 to 16 carbon atoms, more preferably, a hydrogen atom, an alkyl group having 1 to 12 carbon atoms, an aryl group having 6 to 14 carbon atoms, a fluorine atom, further preferably, a hydrogen atom, an alkyl group having 1 to 6 carbon atoms, an aryl group having 6 to 10 carbon atoms. Among them, a hydrogen atom, a fluorine atom, a methyl group, an ethyl group, a propyl group, a butyl group, a hexyl group, a cyclohexyl group, a phenyl group, and a naphthyl group are preferred, and particularly, a hydrogen atom, a methyl group, a butyl group, a hexyl group, and a phenyl group are preferred. The alkyl group may have a branch.

They may further have a substituent group. Specific examples of the further substituent group include the substituent W described below, preferably a halogen atom, an alkyl group, an aryl group, a heterocyclic group, a hydroxyl group, an amino group, or a mercapto group, more preferably a halogen atom, an alkyl group, an aryl group, a heterocyclic group, even more preferably a fluorine atom, an alkyl group, an aryl group, particularly preferably an alkyl group, an aryl group, and most preferably an alkyl group.

Also, adjacent groups among R₂₃₇ to R₂₄₁ and R₂₄₂ to R₂₄₆ may be bonded to each other to form a ring. Examples of the ring formed include a ring R described later. Preferably, the ring formed include a benzene ring, a naphthalene ring, an anthracene ring, a pyridine ring, a pyrimidine ring, and the like.

In addition, R₂₃₃ and R₂₃₇, and R₂₃₆ and R₂₄₆ may be respectively bonded to each other. If the R₂₃₃ and R₂₃₇ or R₂₃₆ and R₂₄₆ are bonded together, four or more membered fused ring is provided which contains a naphthylene group and a phenyl group. Connection between the R₂₃₃ and R₂₃₇ or R₂₃₆ and R₂₄₆ may be a single bond.

In addition, the compound represented by the general formula (D-I) is preferably a compound represented by the following general formula (D-III) or (D-IV).

### [Chemical Formula 11]

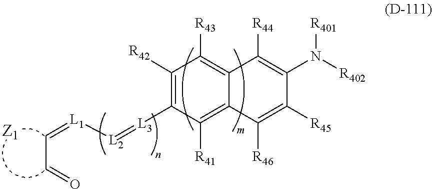

(in the general formula (D-III), Z₁ represents a group of atoms requested to form a 5 or 6-membered ring. L₁, L₂ and L₃ each independently represent an unsubstituted methine group or a substituted methine group. n represents an integer number of 0 or more. m represents 0 or 1. R₄₁ to R₄₆ each independently represent a hydrogen atom or a substituent group. R₄₂ and R₄₃, R₄₃ and R₄₄, R₄₅ and R₄₆, and R₄₁ and R₄₆ may respectively form a ring independently. R₄₀₁ and R₄₀₂ each represent a single bond, or a divalent or trivalent coupling group. R₄₀₁ and any one of R₄₁ to R₄₆, R₄₀₁ and R₄₀₂, and R₄₀₂ and any one of R₄₁ to R₄₆ may be respectively bonded to each other to form a ring.)

### [Chemical Formula 12]

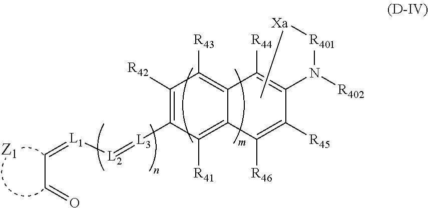

(In the general formula (D-IV), Z₁ represents a group of atoms required to form a 5 or 6-membered ring. L₁, L₂, and L₃ each independently represent an unsubstituted methine group or a substituted methine group. n represents an integer number of 0 or more. m represents 0 or 1. R₄₁ to R₄₆ independently represent a hydrogen atom or a substituent group. R₄₂ and R₄₃, R₄₃ and R₄₄, R₄₅ and R₄₆, and R₄₁ and R₄₆ may respectively form a ring independently. R₄₀₁ represents a single bond or a divalent coupling group. R₄₀₂ independently represents a hydrogen atom or a substitutent group. Xa represents a single bond, an oxygen atom, a sulfur atom, an alkylene group, a silylene group, an alkenylene group, a cycloalkylene group, a cycloalkenylene group, an arylene group, a divalent heterocyclic group, or an imino group, which may further have a substituent group to be bonded with any one of R₄₁ to R₄₆. R₄₀₁ and R₄₀₂, and R₄₀₂ and any one of R₄₁ to R₄₆ may be respectively bonded to each other to form a ring.)

The compound represented by the general formula (D-I) can be manufactured according to the synthesis method described in Japanese Unexamined Patent Publication No. 2000-297068.

Specific examples of the compound represented by the general formula (D-I) are as follows. However, the present invention is not limited thereto.

### [Chemical Formula 13]

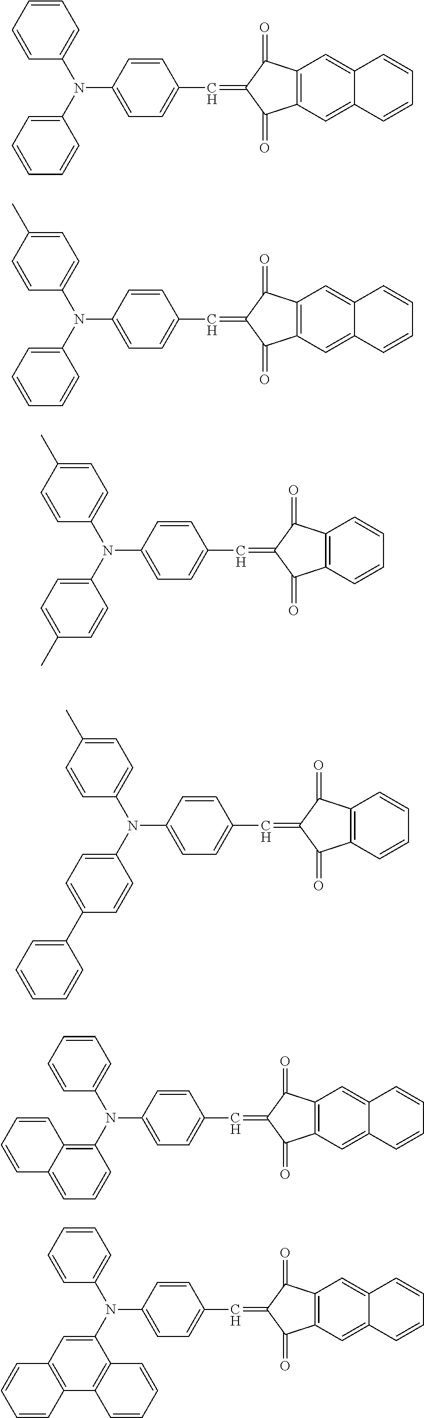

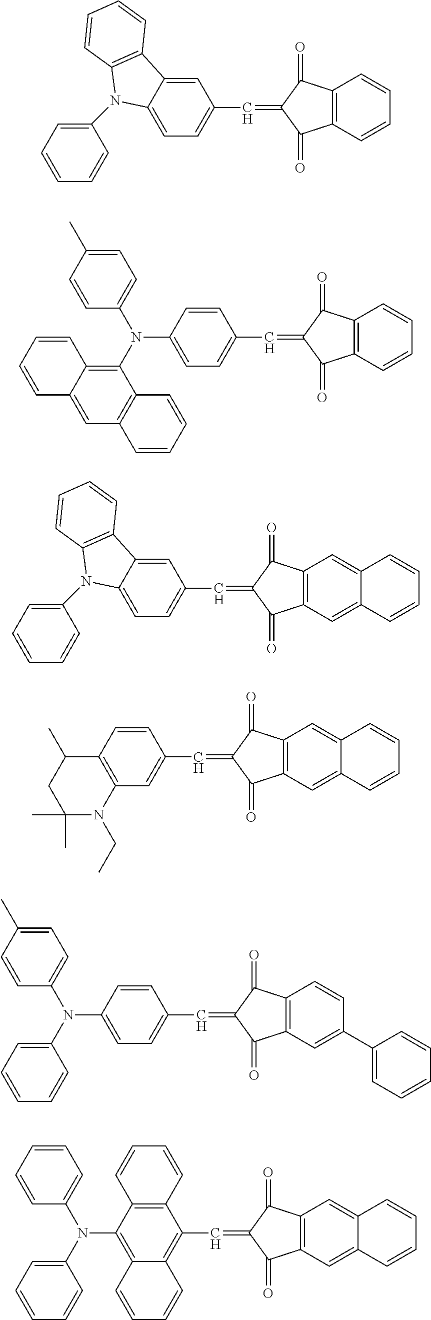

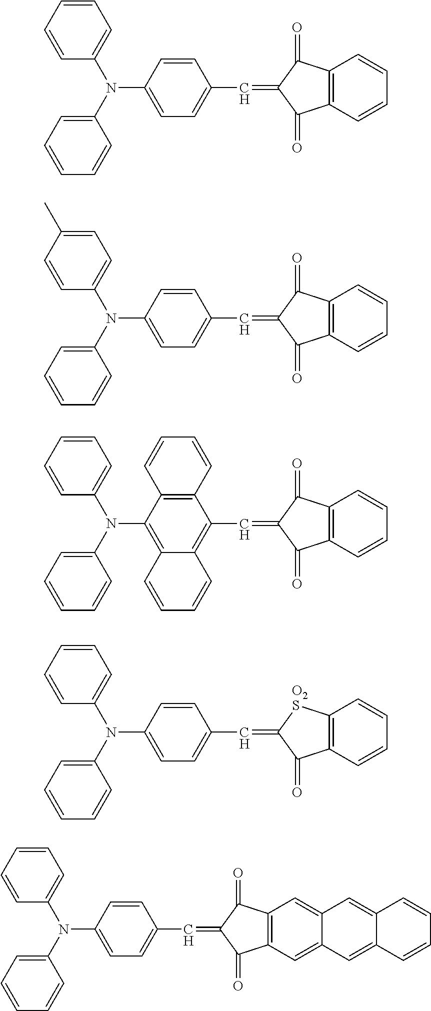

### [Chemical Formula 14]

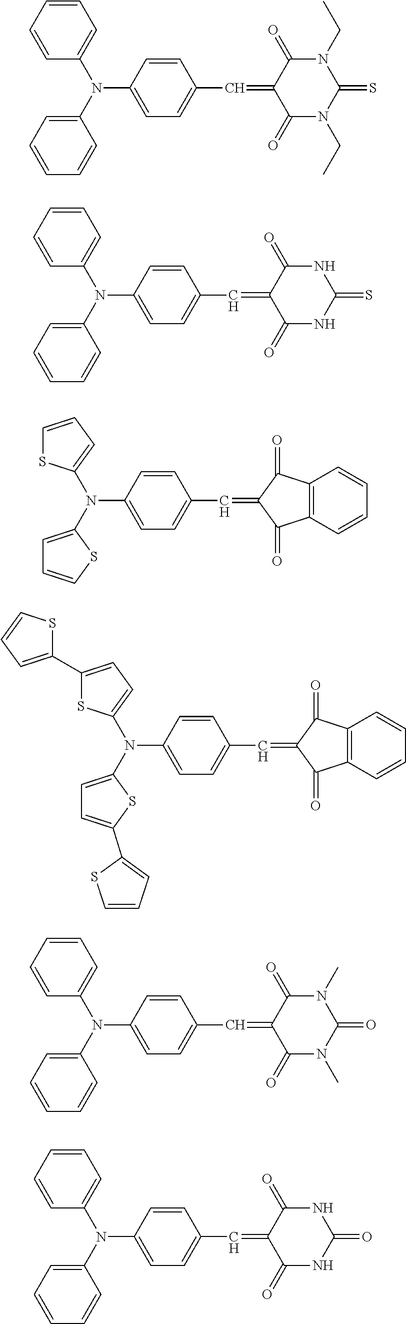

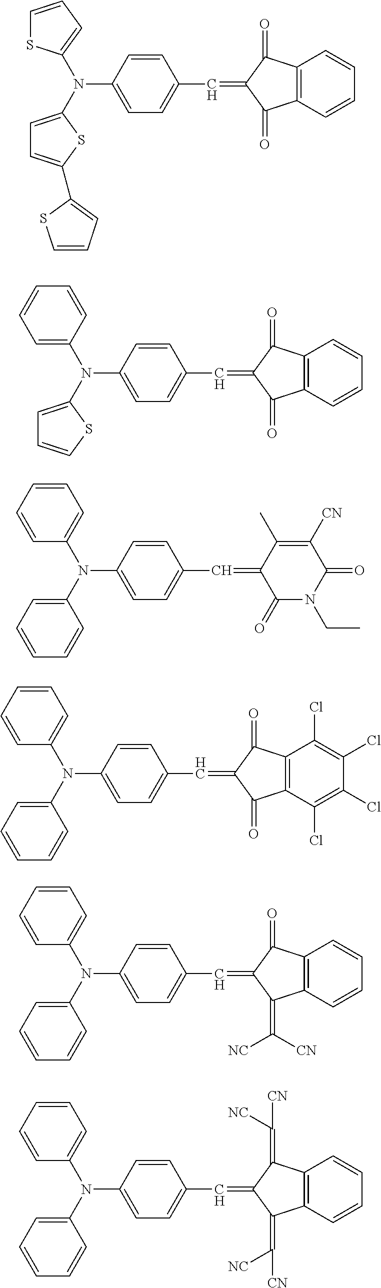

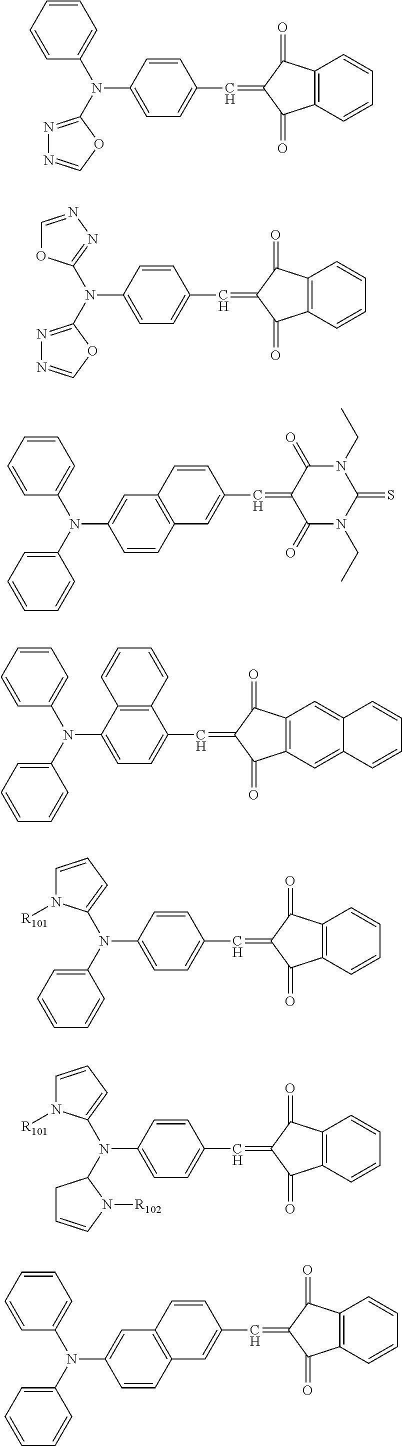

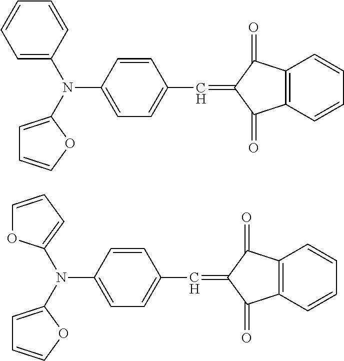

### [Chemical Formula 15]

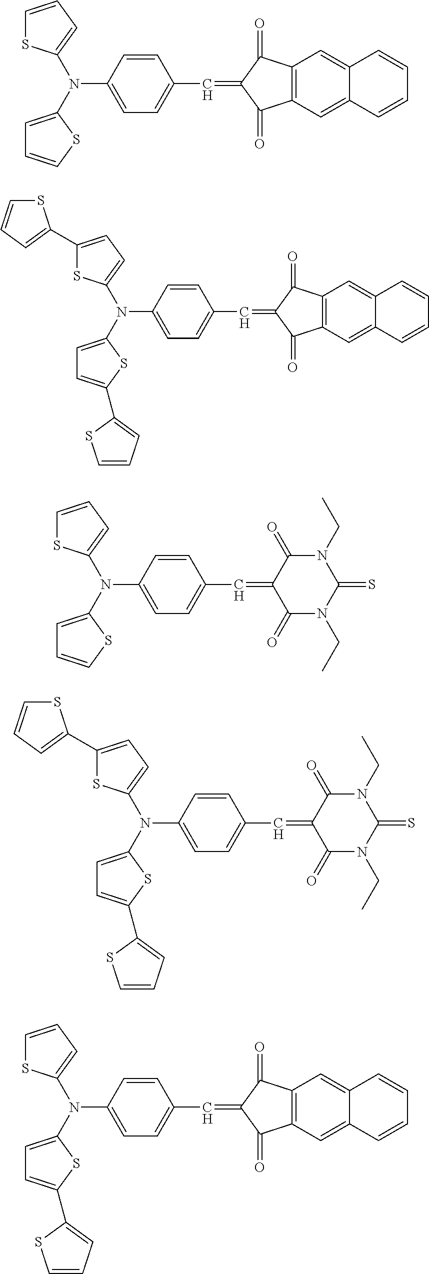

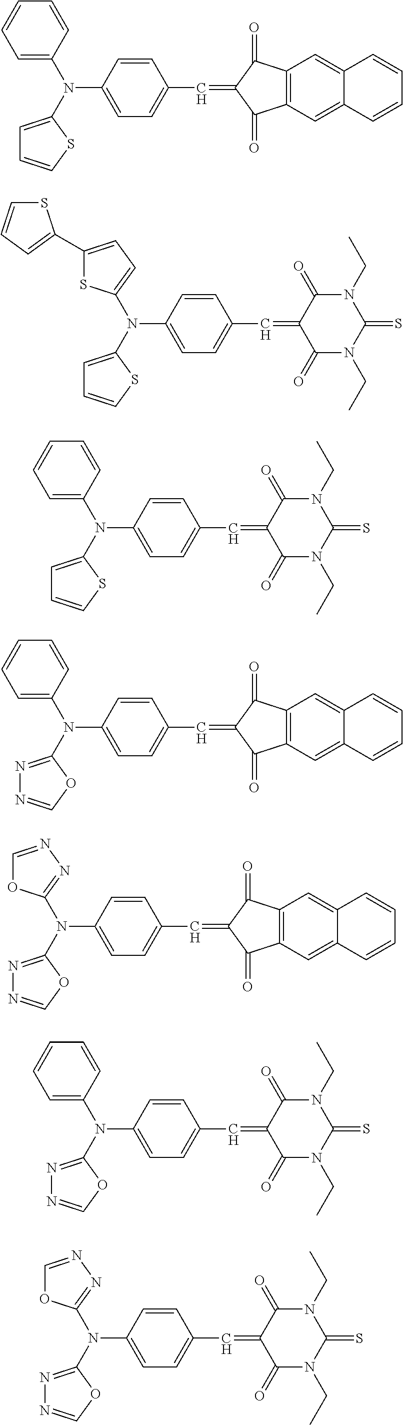

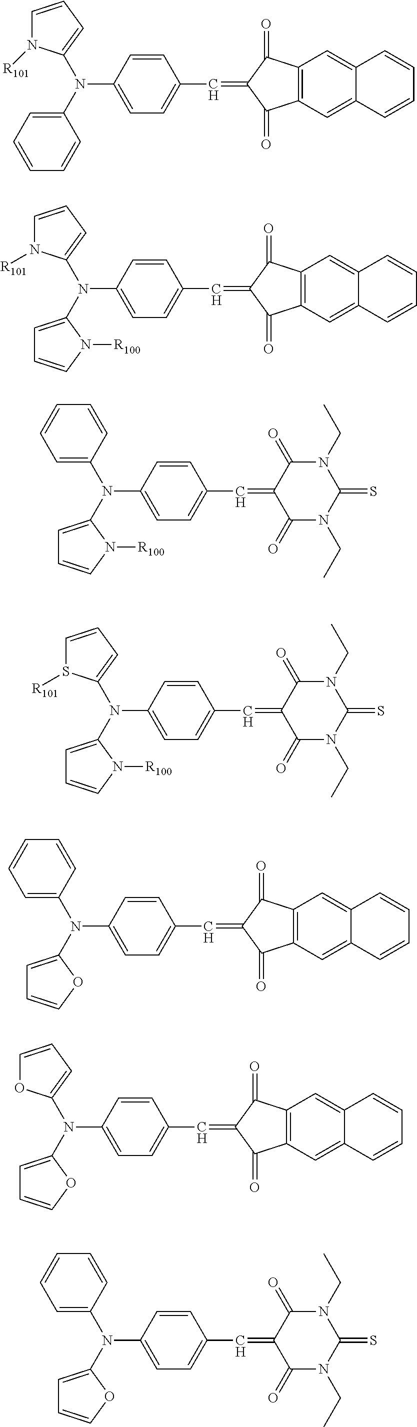

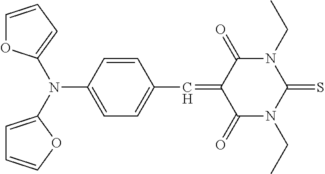 [SMILES: CCN1C(=O)C(=Cc2ccc(N(c3ccco3)c3ccco3)cc2)C(=O)N(CC)C1=S]

### [Chemical Formula 16]

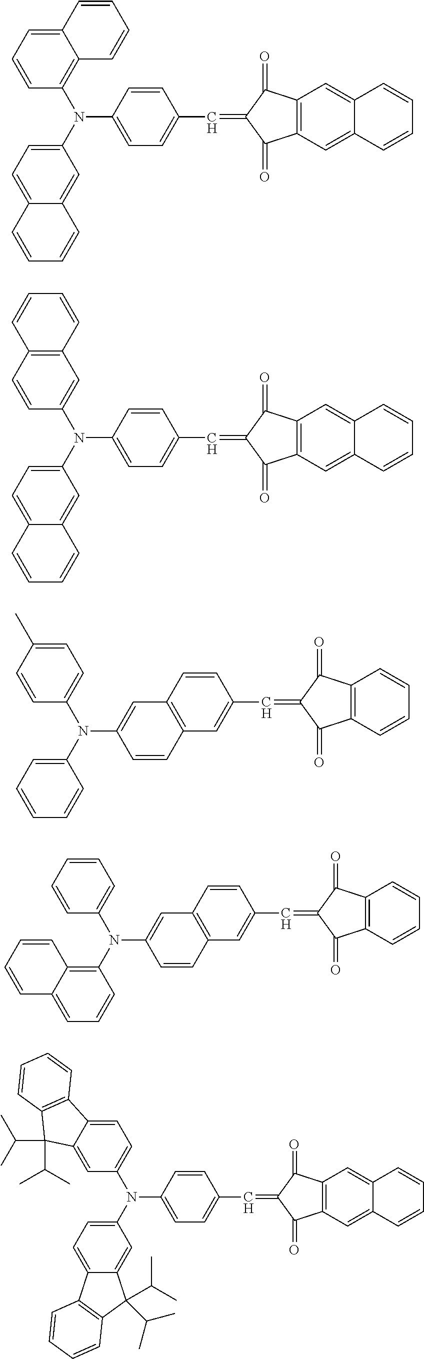

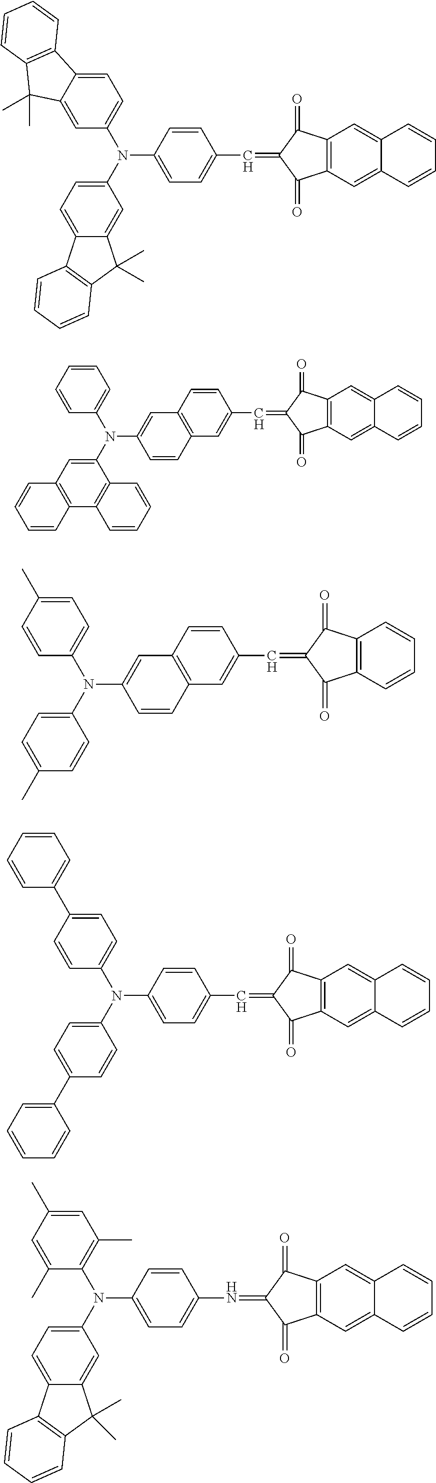

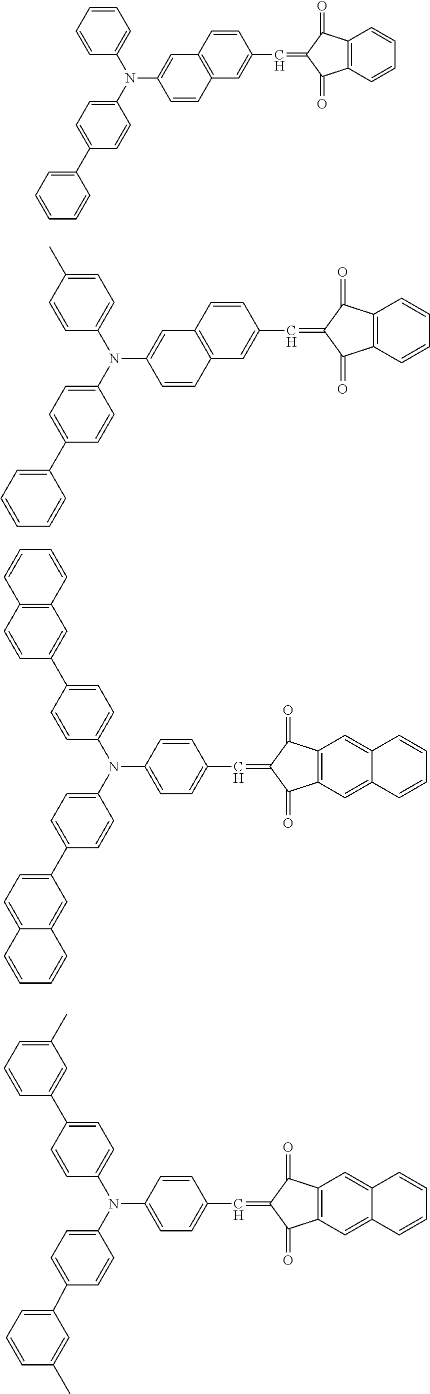

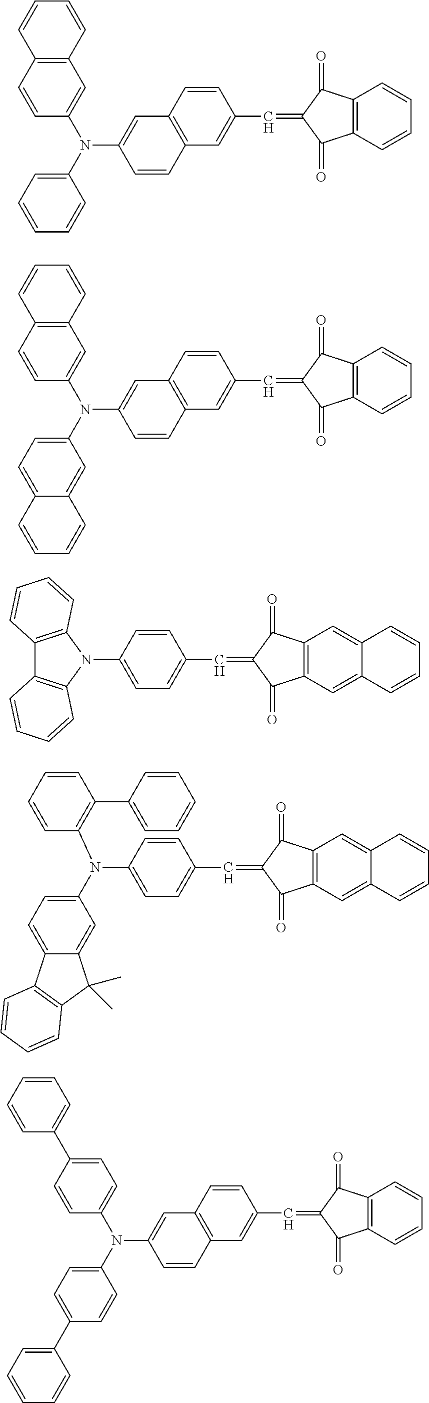

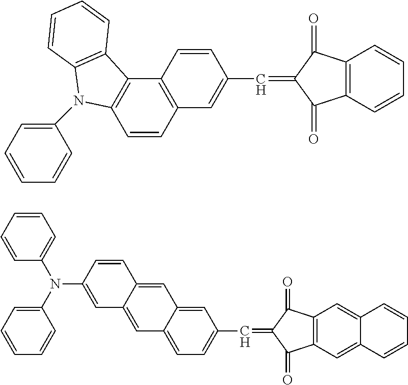

### [Chemical Formula 17]

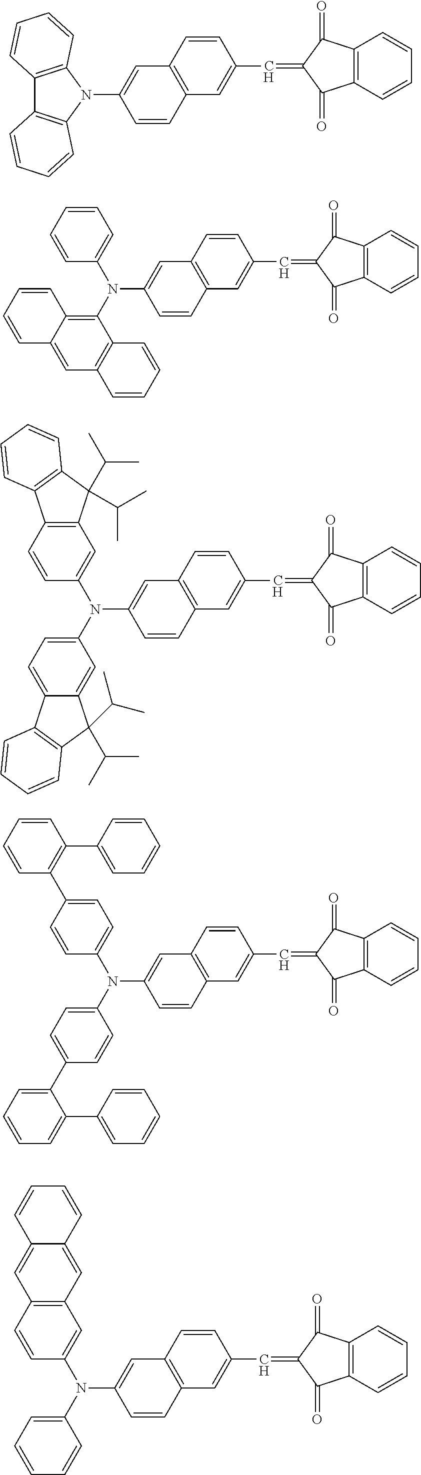

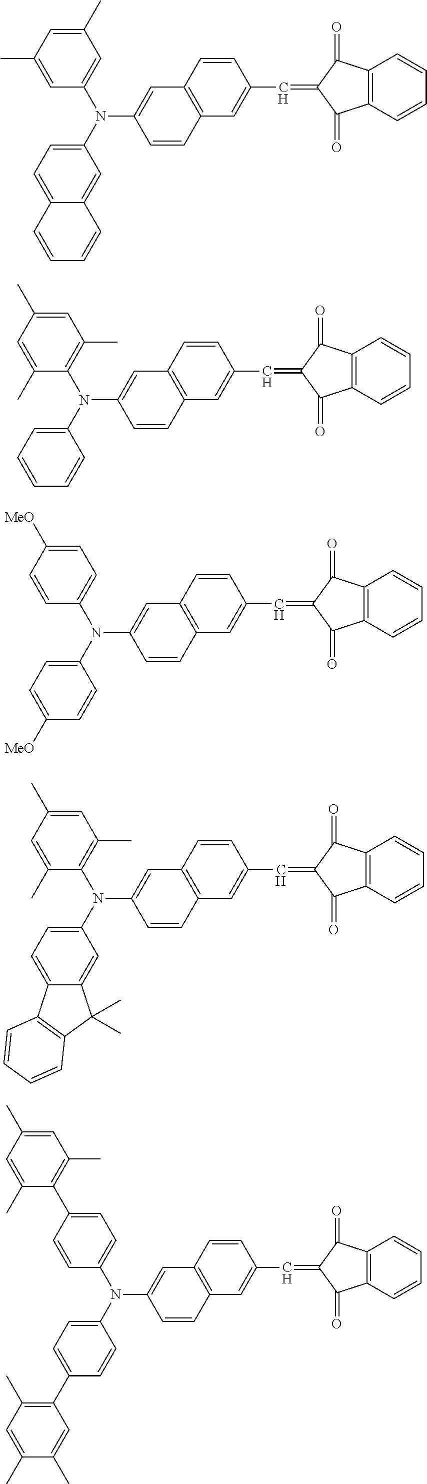

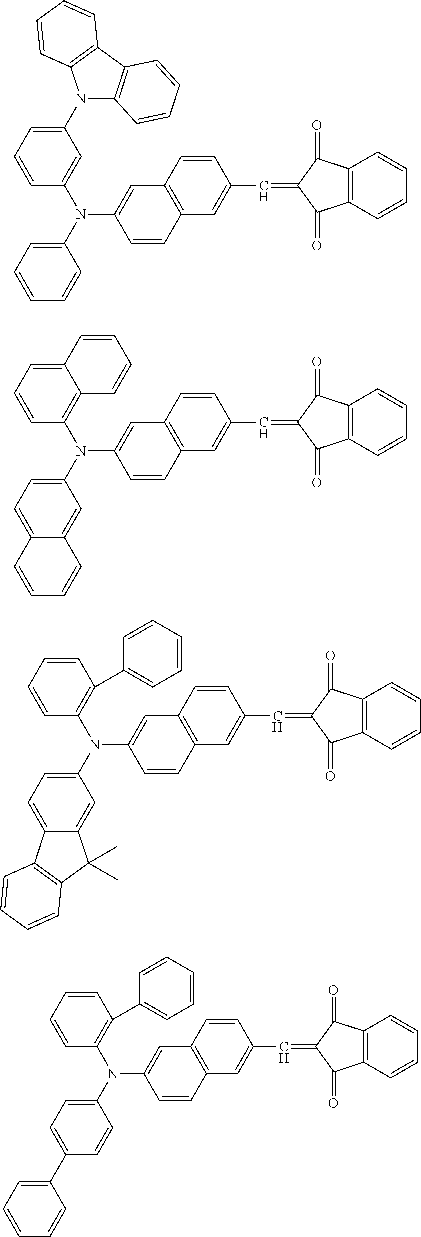

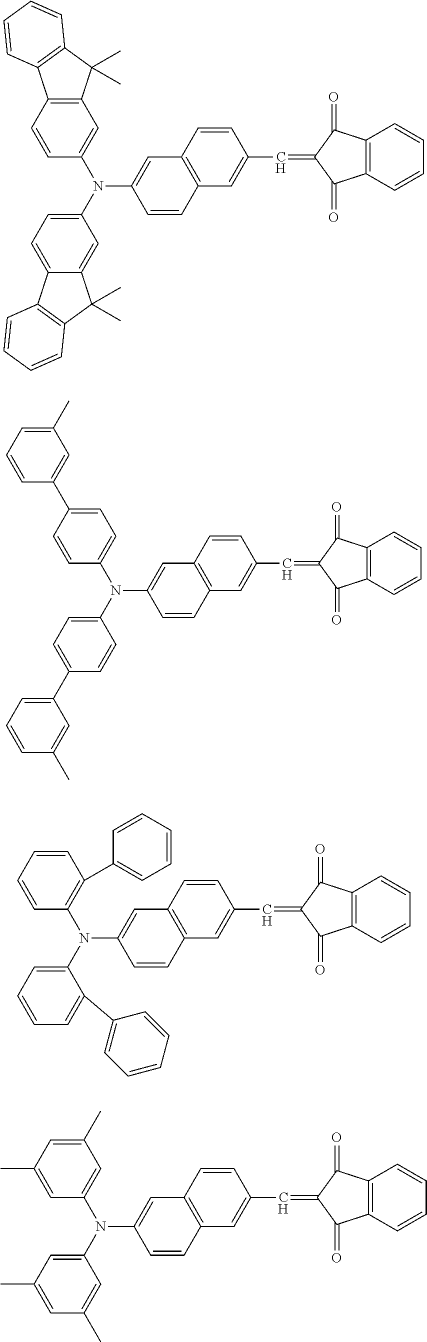

### [Chemical Formula 18]

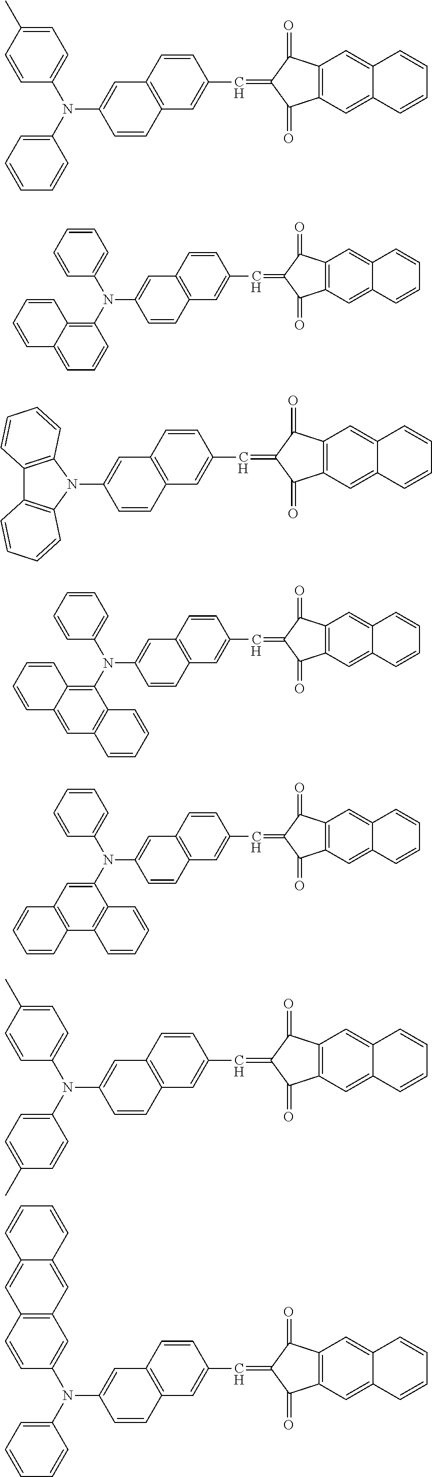

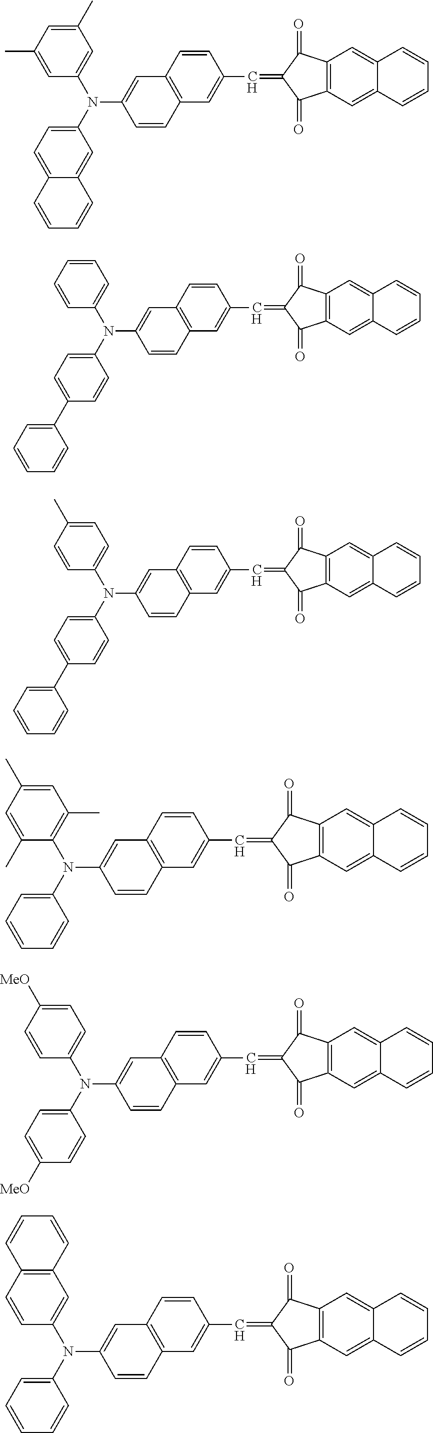

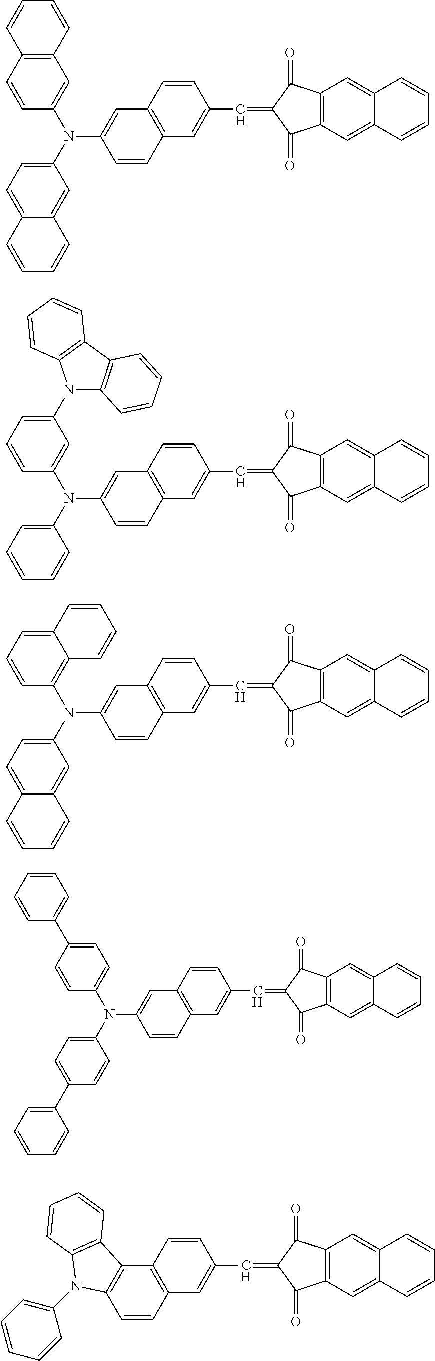

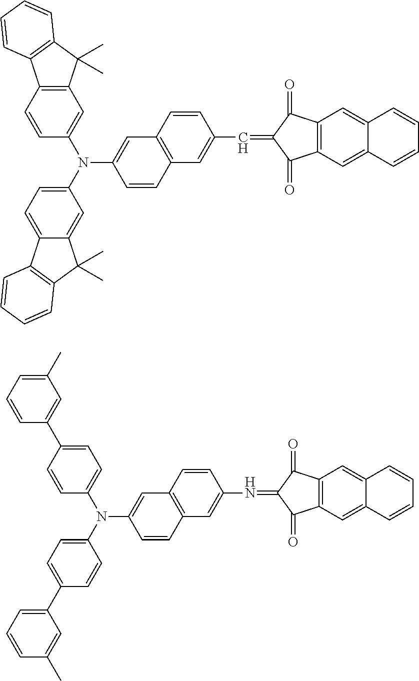

### [Chemical Formula 19]

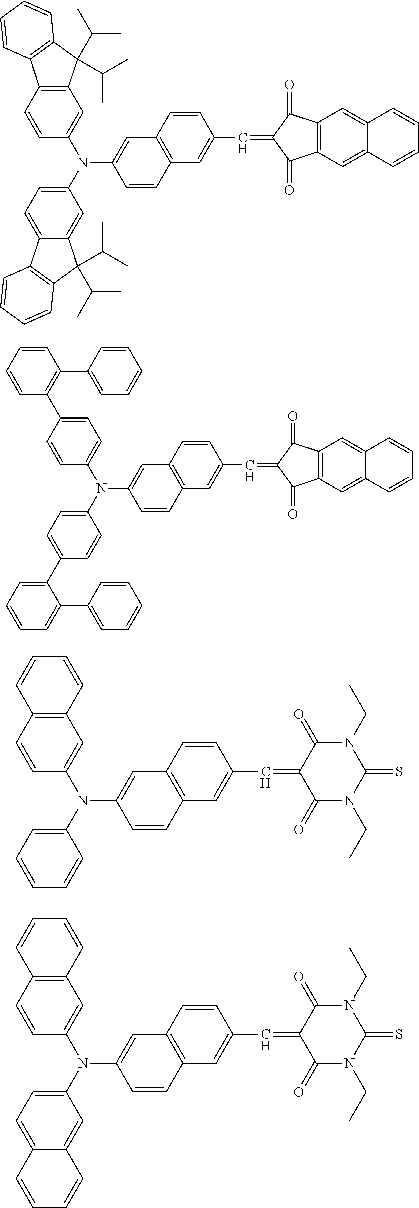

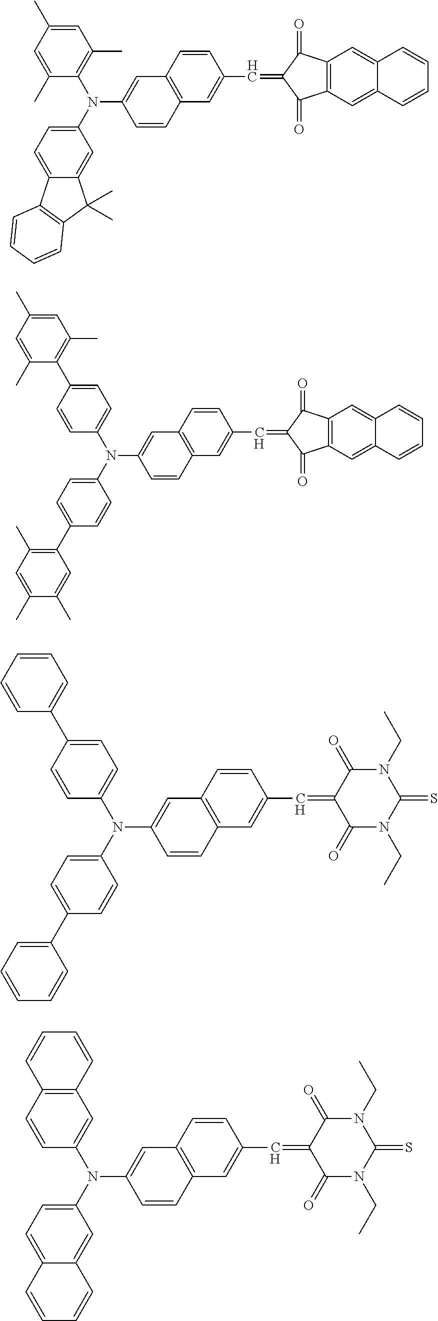

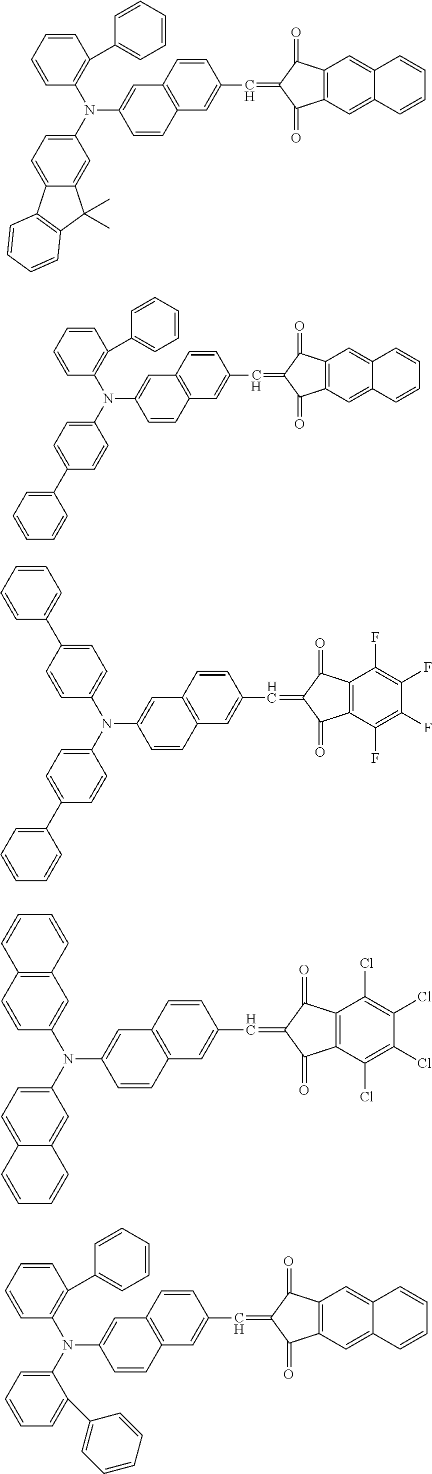

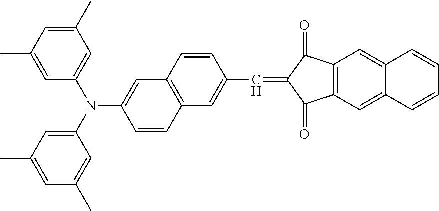 [SMILES: Cc1cc(C)cc(N(c2cc(C)cc(C)c2)c2ccc3cc(C=C4C(=O)c5cc6ccccc6cc5C4=O)ccc3c2)c1]

### [Chemical Formula 20]

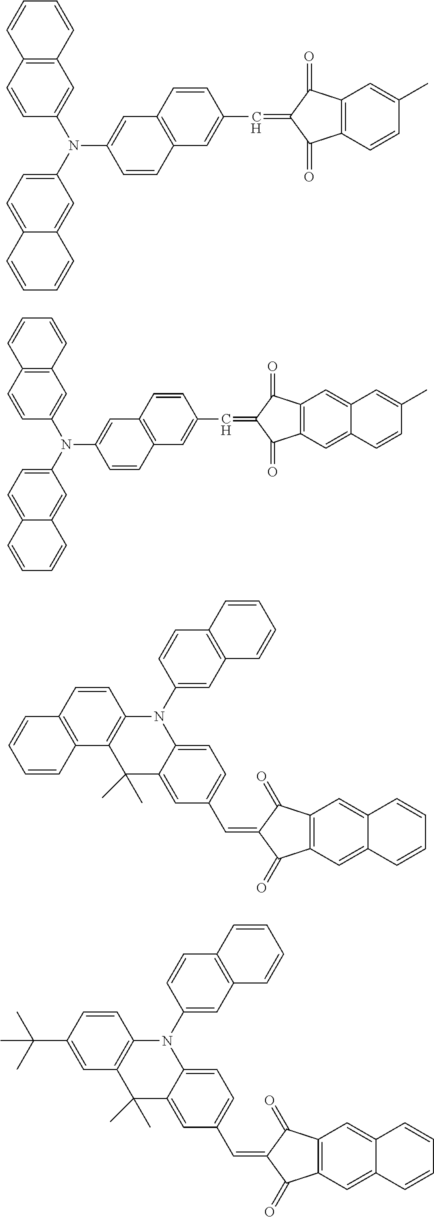

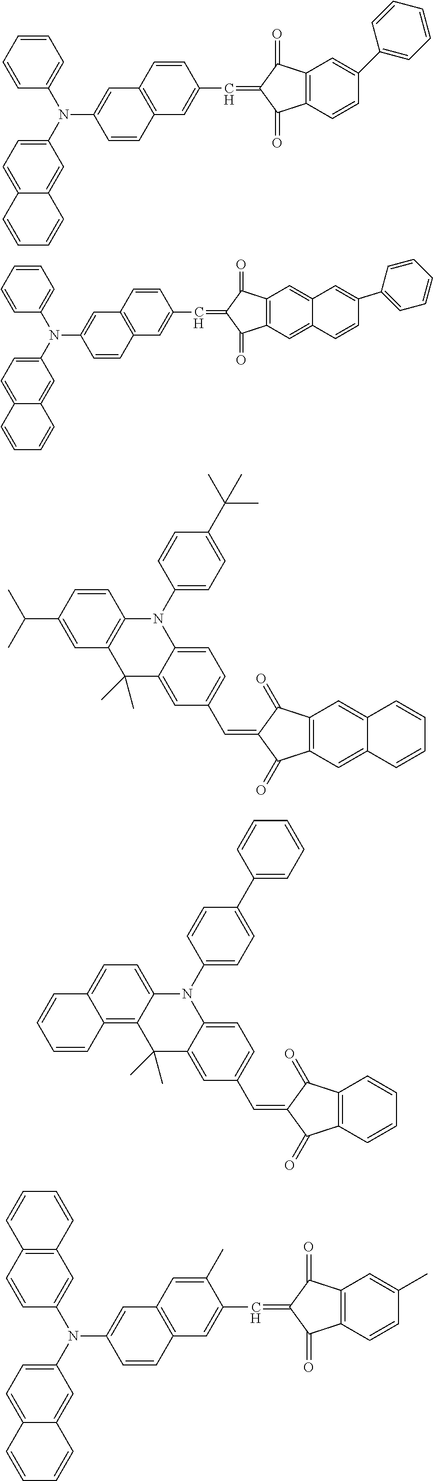

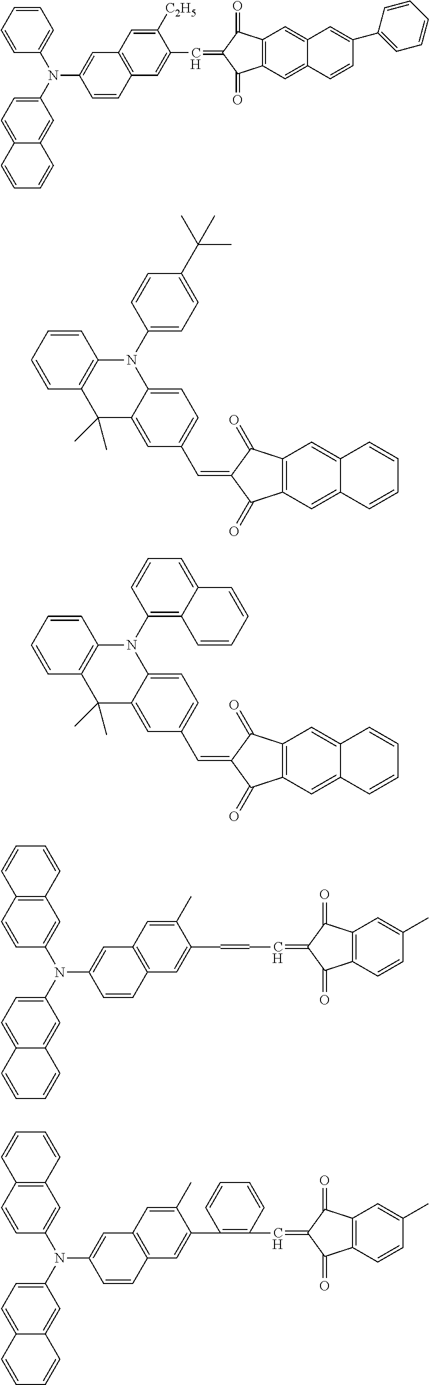

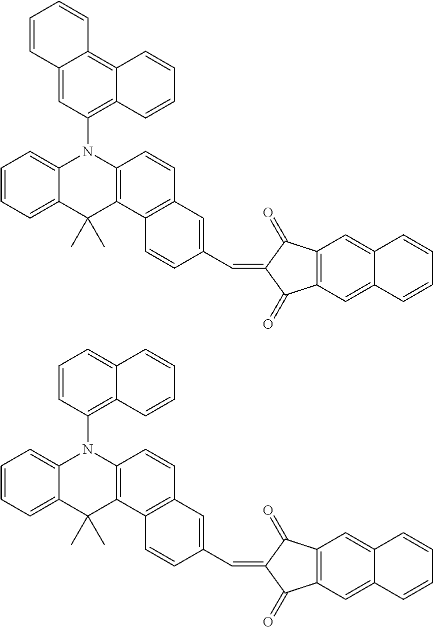 [SMILES: CC1(C)c2ccccc2N(c2cc3ccccc3c3ccccc23)c2ccc3cc(C=C4C(=O)c5cc6ccccc6cc5C4=O)ccc3c21 ; CC1(C)c2ccccc2N(c2cccc3ccccc23)c2ccc3cc(C=C4C(=O)c5cc6ccccc6cc5C4=O)ccc3c21]

### [Chemical Formula 21]

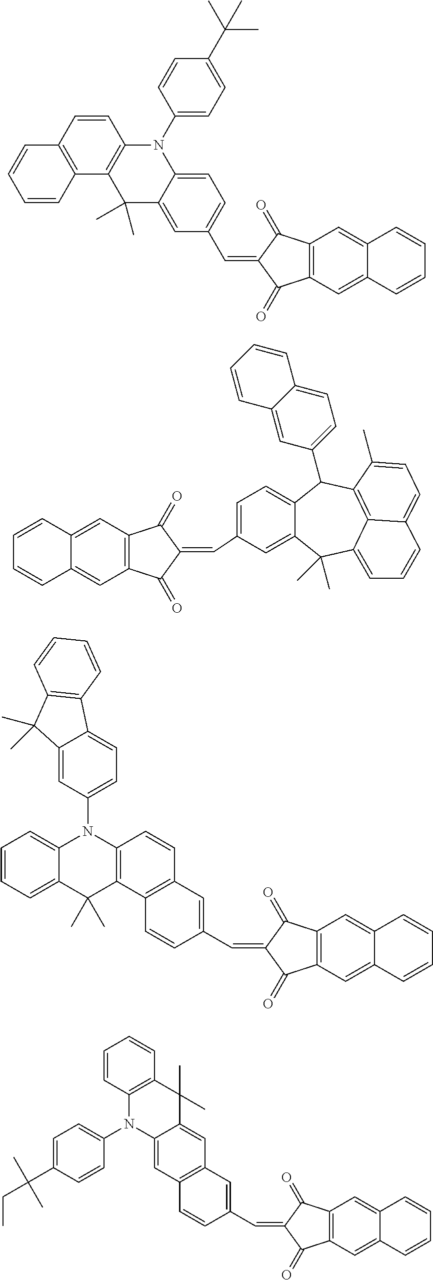

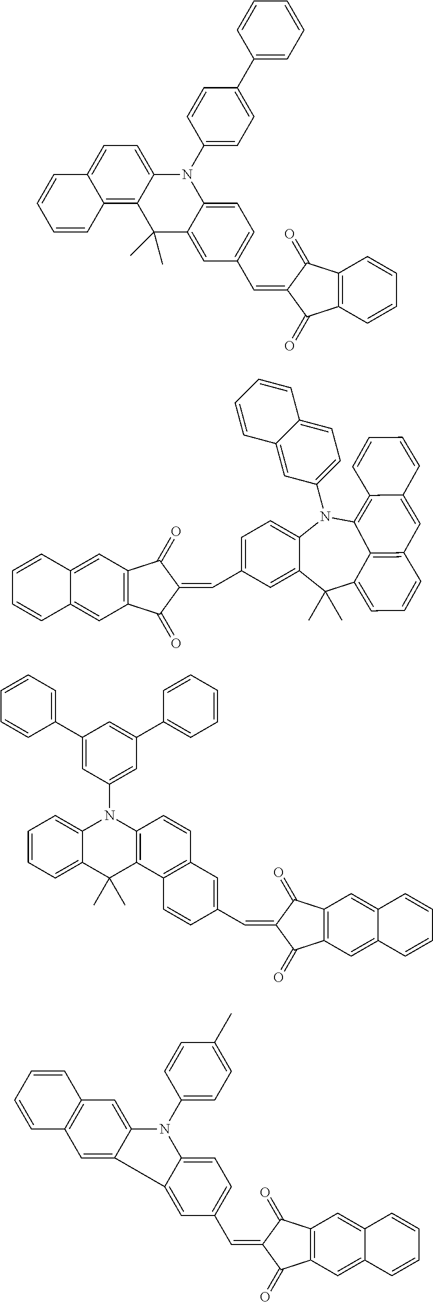

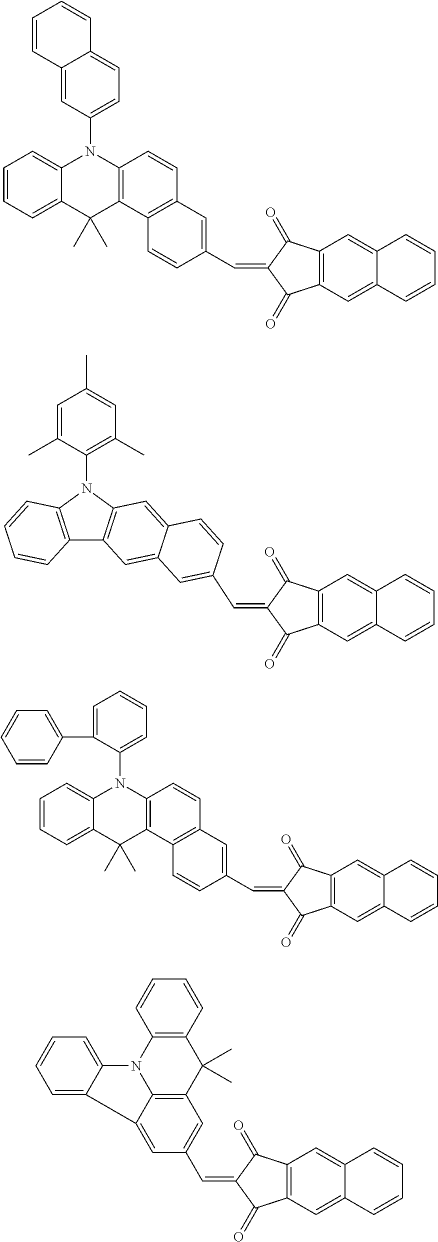 [SMILES: CC1(C)c2ccccc2N(c2ccc3ccccc3c2)c2ccc3cc(C=C4C(=O)c5cc6ccccc6cc5C4=O)ccc3c21 ; Cc1cc(C)c(-n2c3ccccc3c3cc4cc(C=C5C(=O)c6cc7ccccc7cc6C5=O)ccc4cc32)c(C)c1 ; CC1(C)c2ccccc2N(c2ccccc2-c2ccccc2)c2ccc3cc(C=C4C(=O)c5cc6ccccc6cc5C4=O)ccc3c21 ; CC1(C)c2ccccc2-n2c3ccccc3c3cc(C=C4C(=O)c5cc6ccccc6cc5C4=O)cc1c32]

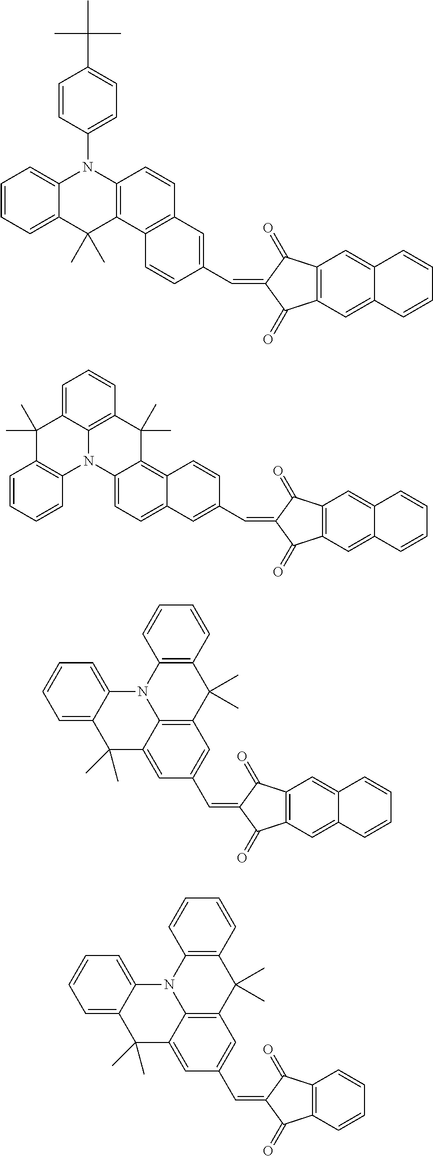

### [Chemical Formula 22]

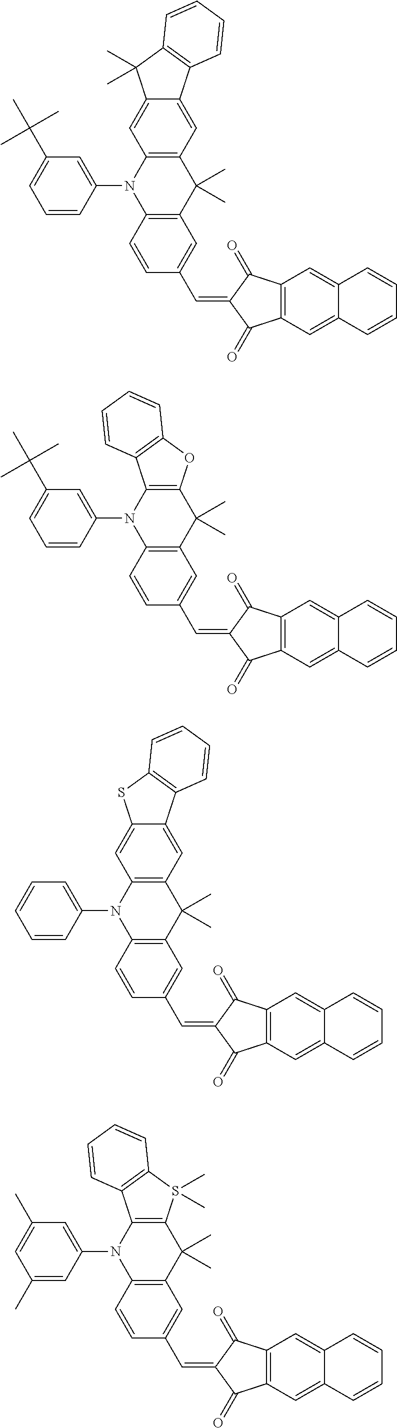 [SMILES: CC(C)(C)c1cccc(N2c3ccc(C=C4C(=O)c5cc6ccccc6cc5C4=O)cc3C(C)(C)c3cc4c(cc32)C(C)(C)c2ccccc2-4)c1 ; CC(C)(C)c1cccc(N2c3ccc(C=C4C(=O)c5cc6ccccc6cc5C4=O)cc3C(C)(C)c3oc4ccccc4c32)c1 ; CC1(C)c2cc(C=C3C(=O)c4cc5ccccc5cc4C3=O)ccc2N(c2ccccc2)c2cc3sc4ccccc4c3cc21 ; Cc1cc(C)cc(N2C3=C(C(C)(C)c4cc(C=C5C(=O)c6cc7ccccc7cc6C5=O)ccc42)S(C)(C)c2ccccc23)c1]

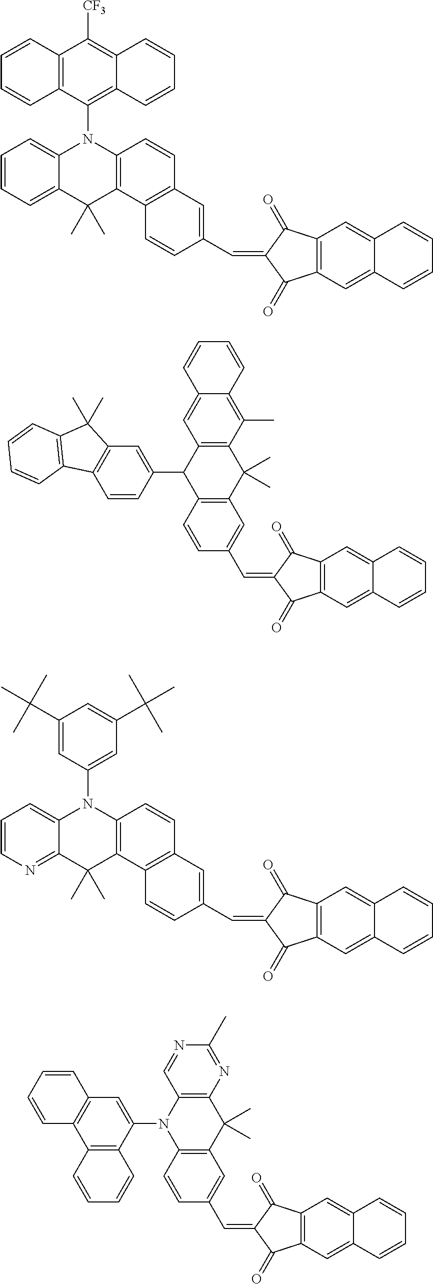 [SMILES: CC1(C)c2ccccc2N(c2c3ccccc3c(C(F)(F)F)c3ccccc23)c2ccc3cc(C=C4C(=O)c5cc6ccccc6cc5C4=O)ccc3c21 ; Cc1c2c(cc3ccccc13)C(c1ccc3c(c1)C(C)(C)c1ccccc1-3)c1ccc(C=C3C(=O)c4cc5ccccc5cc4C3=O)cc1C2(C)C ; CC(C)(C)c1cc(N2c3cccnc3C(C)(C)c3c2ccc2cc(C=C4C(=O)c5cc6ccccc6cc5C4=O)ccc32)cc(C(C)(C)C)c1 ; Cc1ncc2c(n1)C(C)(C)c1cc(C=C3C(=O)c4cc5ccccc5cc4C3=O)ccc1N2c1cc2ccccc2c2ccccc12]

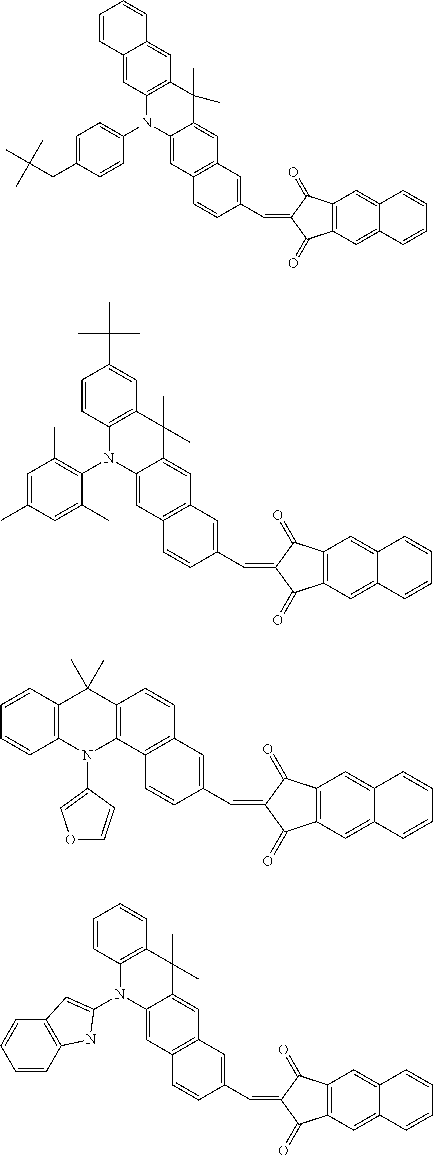 [SMILES: CC(C)(C)Cc1ccc(N2c3cc4ccccc4cc3C(C)(C)c3cc4cc(C=C5C(=O)c6cc7ccccc7cc6C5=O)ccc4cc32)cc1 ; Cc1cc(C)c(N2c3ccc(C(C)(C)C)cc3C(C)(C)c3cc4cc(C=C5C(=O)c6cc7ccccc7cc6C5=O)ccc4cc32)c(C)c1 ; CC1(C)c2ccccc2N(c2ccoc2)c2c1ccc1cc(C=C3C(=O)c4cc5ccccc5cc4C3=O)ccc21 ; CC1(C)c2ccccc2N(c2cc3ccccc3[nH]2)c2cc3ccc(C=C4C(=O)c5cc6ccccc6cc5C4=O)cc3cc21]

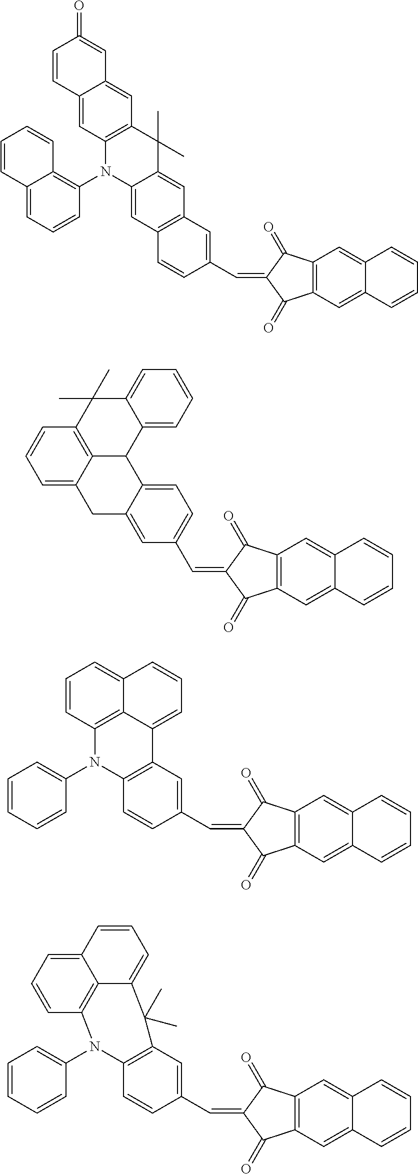

### [Chemical Formula 23]

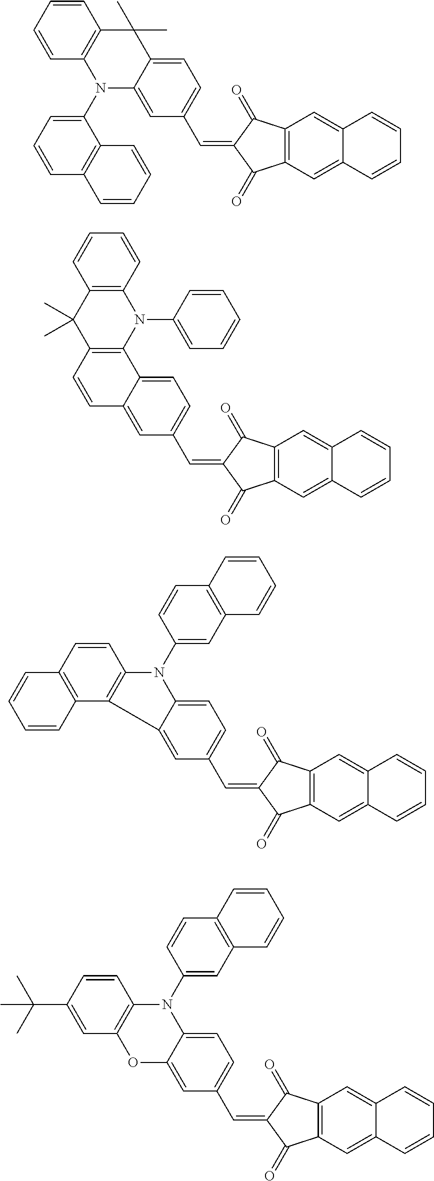

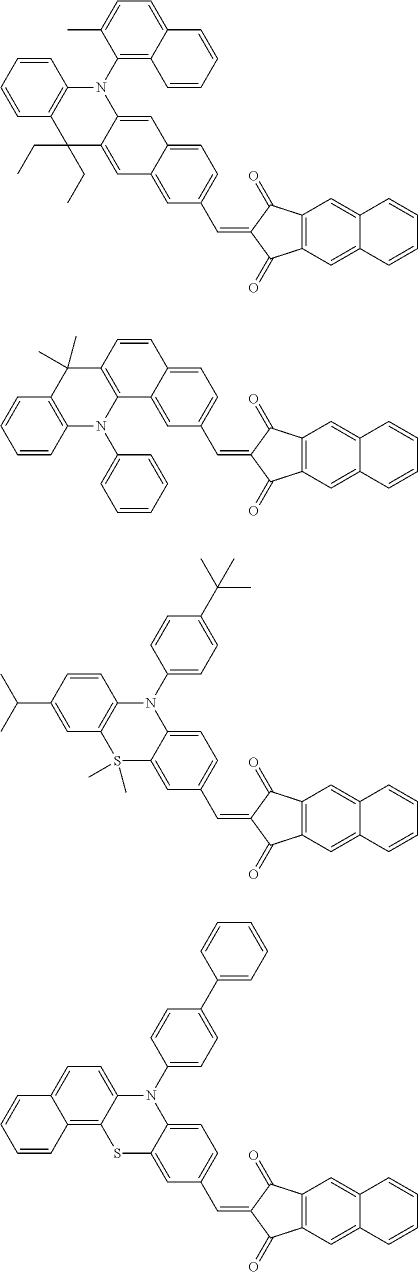 [SMILES: CCC1(CC)c2ccccc2N(c2c(C)ccc3ccccc23)c2cc3ccc(C=C4C(=O)c5cc6ccccc6cc5C4=O)cc3cc21 ; CC1(C)c2ccccc2N(c2ccccc2)c2c1ccc1ccc(C=C3C(=O)c4cc5ccccc5cc4C3=O)cc21 ; CC(C)c1ccc2c(c1)S(C)(C)c1cc(C=C3C(=O)c4cc5ccccc5cc4C3=O)ccc1N2c1ccc(C(C)(C)C)cc1 ; O=C1C(=Cc2ccc3c(c2)Sc2c(ccc4ccccc24)N3c2ccc(-c3ccccc3)cc2)C(=O)c2cc3ccccc3cc21]

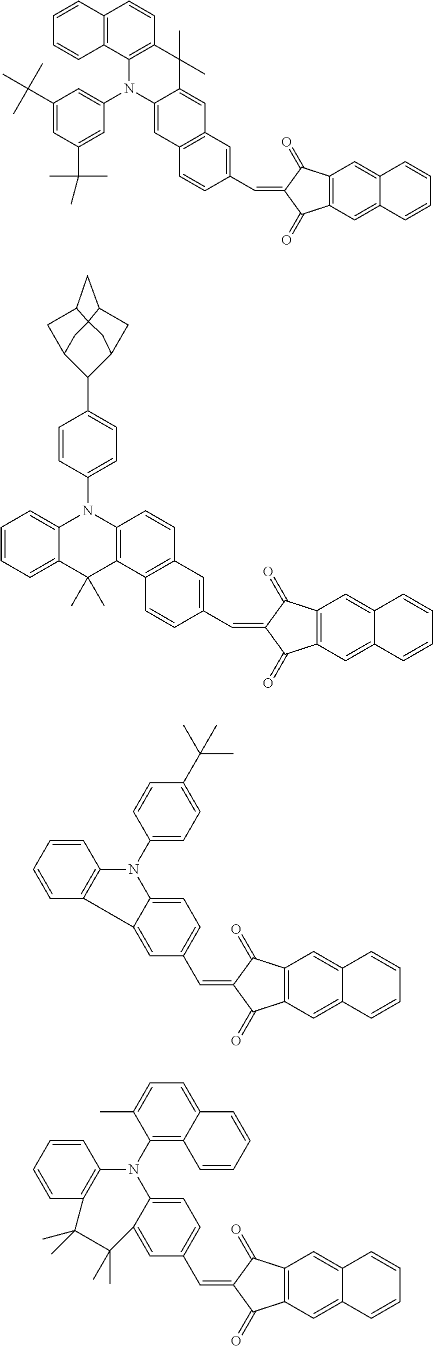 [SMILES: CC(C)(C)c1cc(N2c3cc4ccc(C=C5C(=O)c6cc7ccccc7cc6C5=O)cc4cc3C(C)(C)c3ccc4ccccc4c32)cc(C(C)(C)C)c1 ; CC1(C)c2ccccc2N(c2ccc(C3C4CC5CC6CC3CC56C4)cc2)c2ccc3cc(C=C4C(=O)c5cc6ccccc6cc5C4=O)ccc3c21 ; CC(C)(C)c1ccc(-n2c3ccccc3c3cc(C=C4C(=O)c5cc6ccccc6cc5C4=O)ccc32)cc1 ; Cc1ccc2ccccc2c1N1c2ccccc2C(C)(C)C(C)(C)c2cc(C=C3C(=O)c4cc5ccccc5cc4C3=O)ccc21]

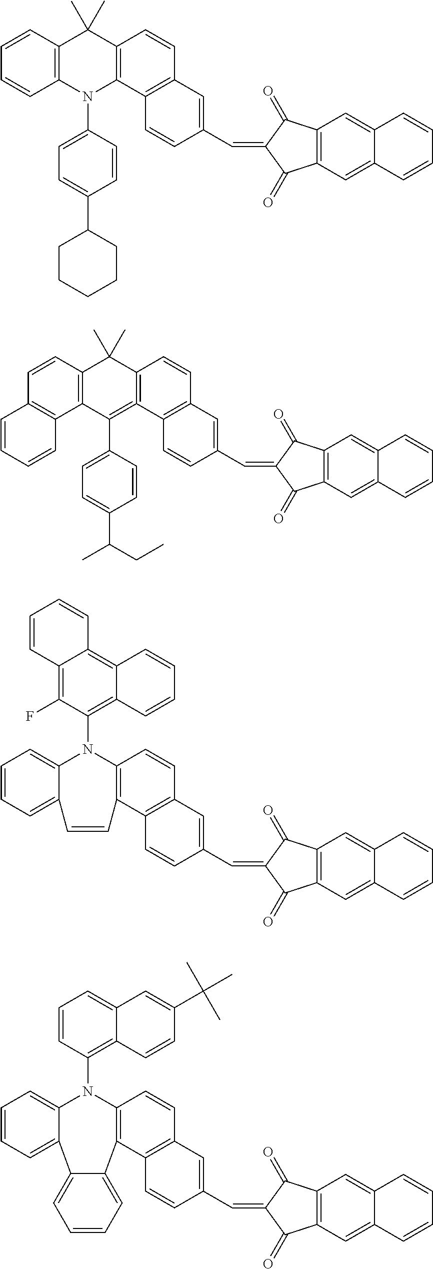

### [Chemical Formula 24]

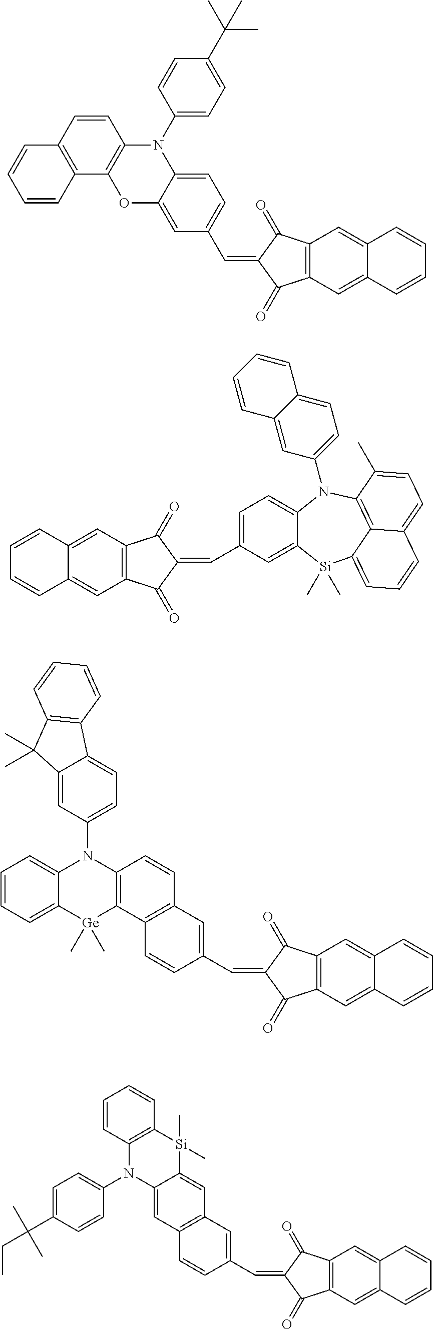

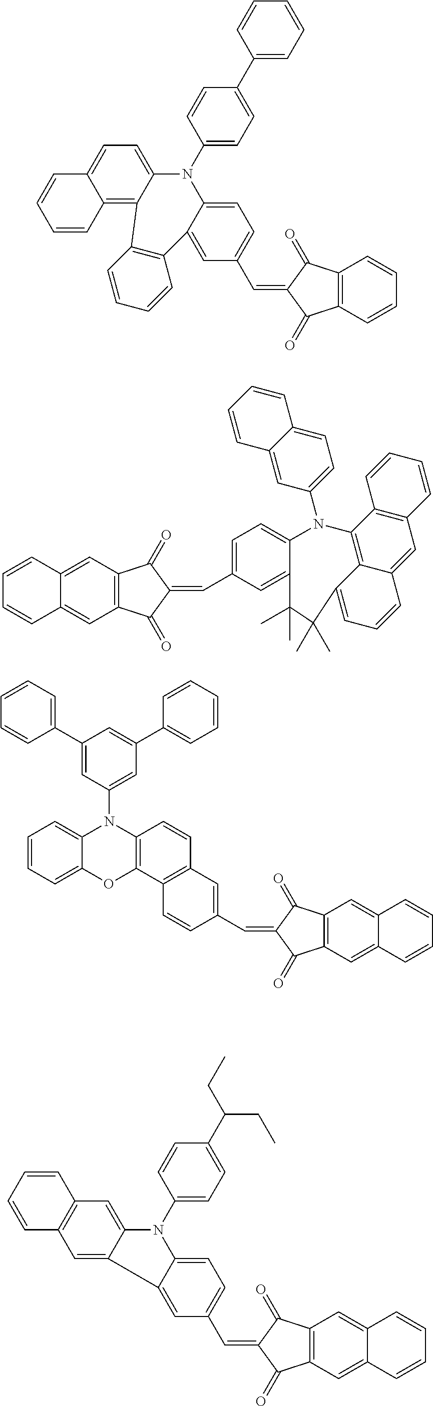

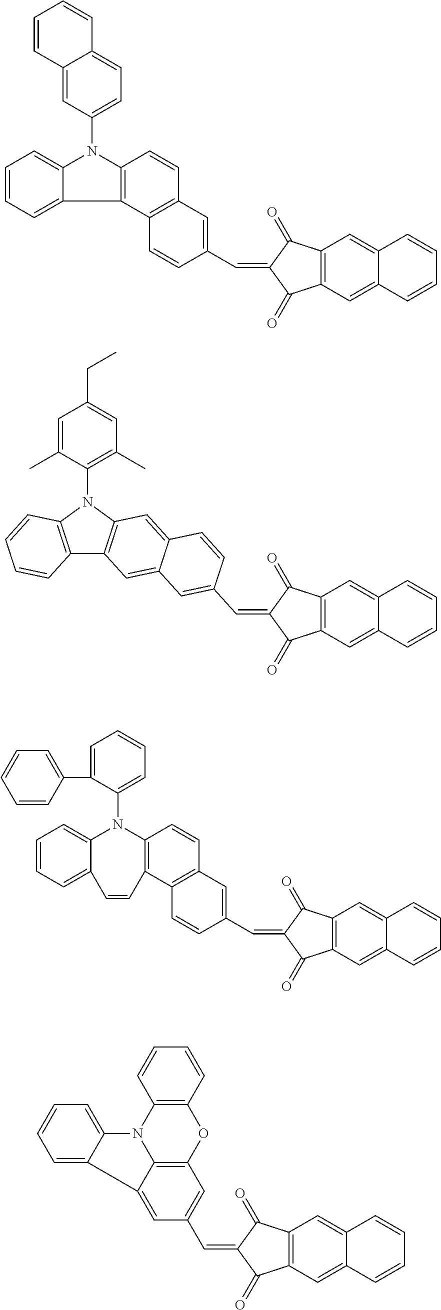 [SMILES: O=C1C(=Cc2ccc3c(ccc4c3c3ccccc3n4-c3ccc4ccccc4c3)c2)C(=O)c2cc3ccccc3cc21 ; CCc1cc(C)c(-n2c3ccccc3c3cc4cc(C=C5C(=O)c6cc7ccccc7cc6C5=O)ccc4cc32)c(C)c1 ; O=C1C(=Cc2ccc3c4c(ccc3c2)N(c2ccccc2-c2ccccc2)c2ccccc2C=C4)C(=O)c2cc3ccccc3cc21 ; O=C1C(=Cc2cc3c4c(c2)c2ccccc2n4-c2ccccc2O3)C(=O)c2cc3ccccc3cc21]

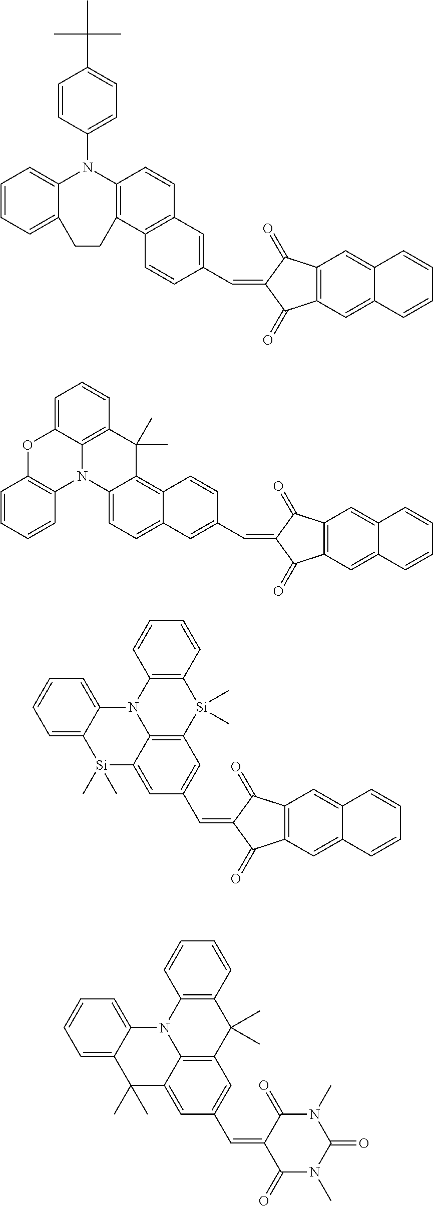 [SMILES: CC(C)(C)c1ccc(N2c3ccccc3CCc3c2ccc2cc(C=C4C(=O)c5cc6ccccc6cc5C4=O)ccc32)cc1 ; CC1(C)c2cccc3c2N(c2ccccc2O3)c2ccc3cc(C=C4C(=O)c5cc6ccccc6cc5C4=O)ccc3c21 ; C[Si]1(C)c2ccccc2N2c3ccccc3[Si](C)(C)c3cc(C=C4C(=O)c5cc6ccccc6cc5C4=O)cc1c32 ; CN1C(=O)C(=Cc2cc3c4c(c2)C(C)(C)c2ccccc2N4c2ccccc2C3(C)C)C(=O)N(C)C1=O]

### [Chemical Formula 25]

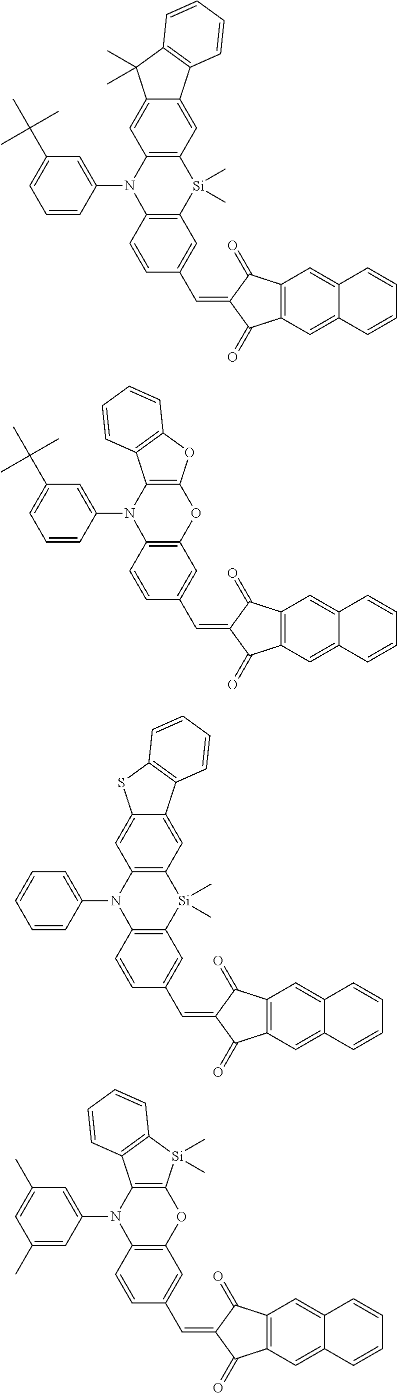 [SMILES: CC(C)(C)c1cccc(N2c3ccc(C=C4C(=O)c5cc6ccccc6cc5C4=O)cc3[Si](C)(C)c3cc4c(cc32)C(C)(C)c2ccccc2-4)c1 ; CC(C)(C)c1cccc(N2c3ccc(C=C4C(=O)c5cc6ccccc6cc5C4=O)cc3Oc3oc4ccccc4c32)c1 ; C[Si]1(C)c2cc(C=C3C(=O)c4cc5ccccc5cc4C3=O)ccc2N(c2ccccc2)c2cc3sc4ccccc4c3cc21 ; Cc1cc(C)cc(N2C3=C(Oc4cc(C=C5C(=O)c6cc7ccccc7cc6C5=O)ccc42)[Si](C)(C)c2ccccc23)c1]

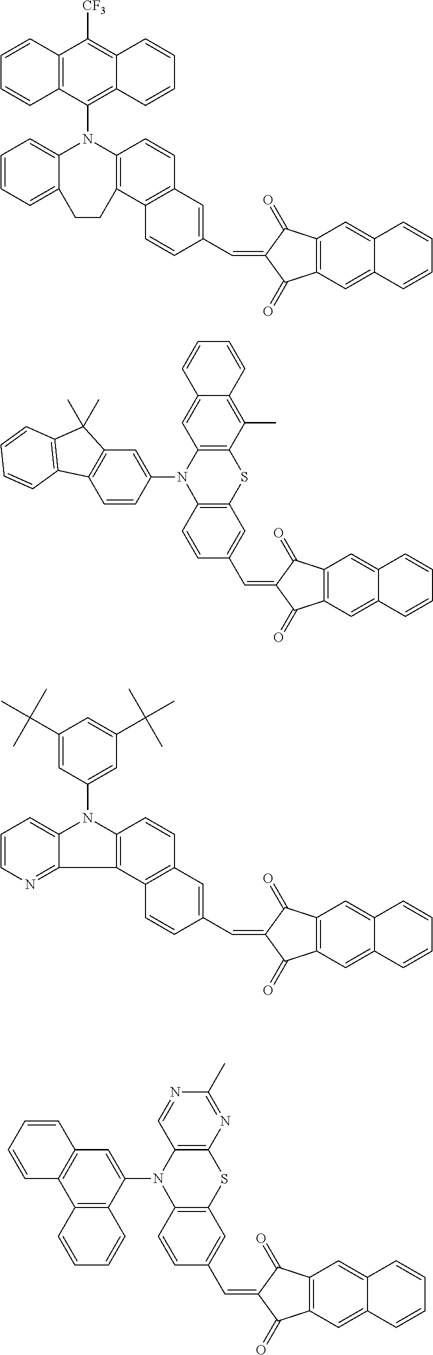 [SMILES: O=C1C(=Cc2ccc3c4c(ccc3c2)N(c2c3ccccc3c(C(F)(F)F)c3ccccc23)c2ccccc2CC4)C(=O)c2cc3ccccc3cc21 ; Cc1c2c(cc3ccccc13)N(c1ccc3c(c1)C(C)(C)c1ccccc1-3)c1ccc(C=C3C(=O)c4cc5ccccc5cc4C3=O)cc1S2 ; CC(C)(C)c1cc(-n2c3cccnc3c3c4ccc(C=C5C(=O)c6cc7ccccc7cc6C5=O)cc4ccc32)cc(C(C)(C)C)c1 ; Cc1ncc2c(n1)Sc1cc(C=C3C(=O)c4cc5ccccc5cc4C3=O)ccc1N2c1cc2ccccc2c2ccccc12]

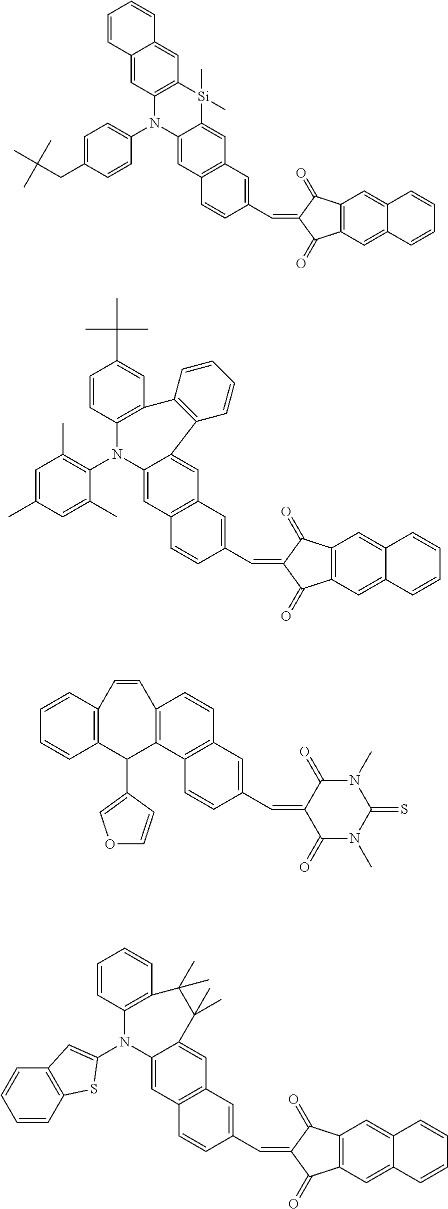 [SMILES: CC(C)(C)Cc1ccc(N2c3cc4ccccc4cc3[Si](C)(C)c3cc4cc(C=C5C(=O)c6cc7ccccc7cc6C5=O)ccc4cc32)cc1 ; Cc1cc(C)c(N2c3ccc(C(C)(C)C)cc3-c3ccccc3-c3cc4cc(C=C5C(=O)c6cc7ccccc7cc6C5=O)ccc4cc32)c(C)c1 ; CN1C(=O)C(=Cc2ccc3c4c(ccc3c2)C=Cc2ccccc2C4c2ccoc2)C(=O)N(C)C1=S ; CC1(C)c2ccccc2N(c2cc3ccccc3s2)c2cc3ccc(C=C4C(=O)c5cc6ccccc6cc5C4=O)cc3cc2C1(C)C]

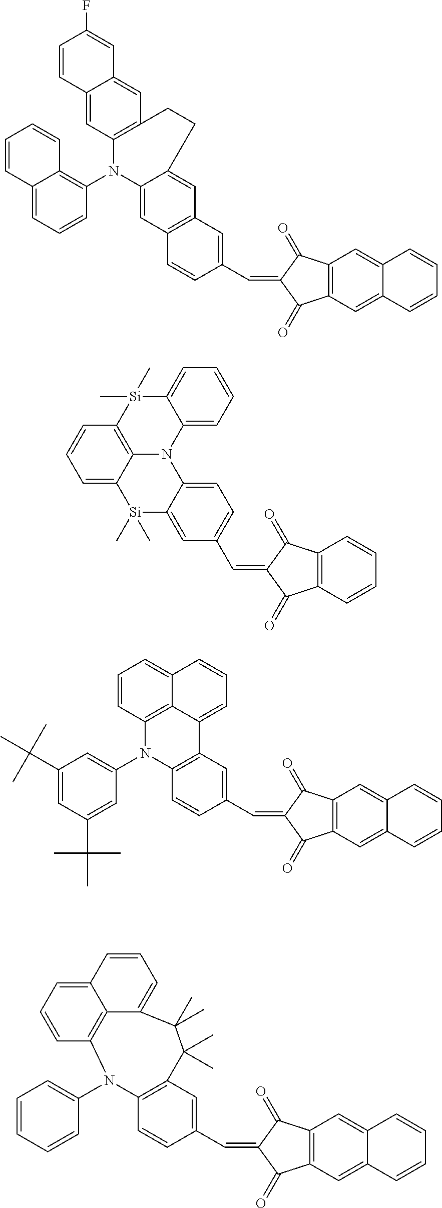 [SMILES: O=C1C(=Cc2ccc3cc4c(cc3c2)CCc2cc3cc(F)ccc3cc2N4c2cccc3ccccc23)C(=O)c2cc3ccccc3cc21 ; C[Si]1(C)c2ccccc2N2c3ccc(C=C4C(=O)c5ccccc5C4=O)cc3[Si](C)(C)c3cccc1c32 ; CC(C)(C)c1cc(N2c3ccc(C=C4C(=O)c5cc6ccccc6cc5C4=O)cc3-c3cccc4cccc2c34)cc(C(C)(C)C)c1 ; CC1(C)c2cc(C=C3C(=O)c4cc5ccccc5cc4C3=O)ccc2N(c2ccccc2)c2cccc3cccc(c23)C1(C)C]

### [Chemical Formula 26]

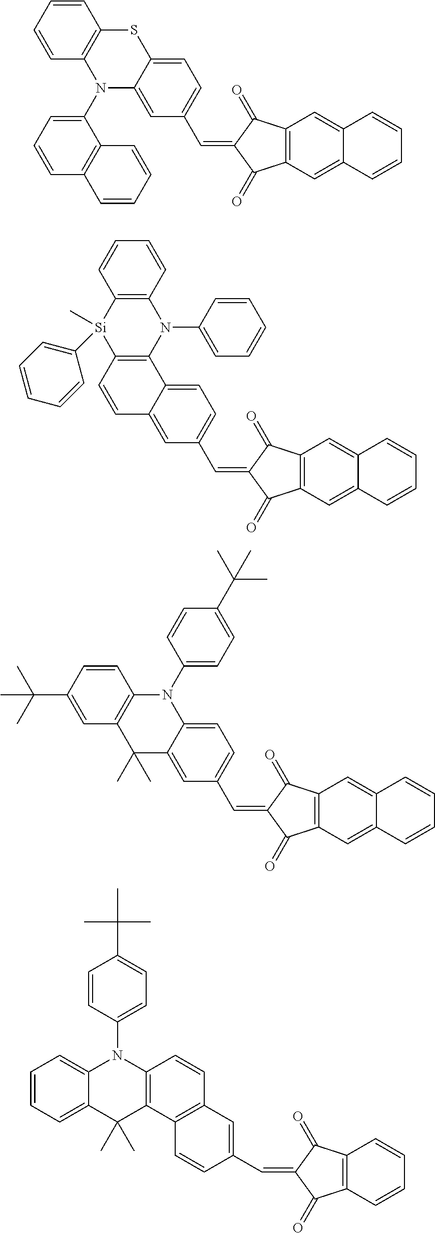

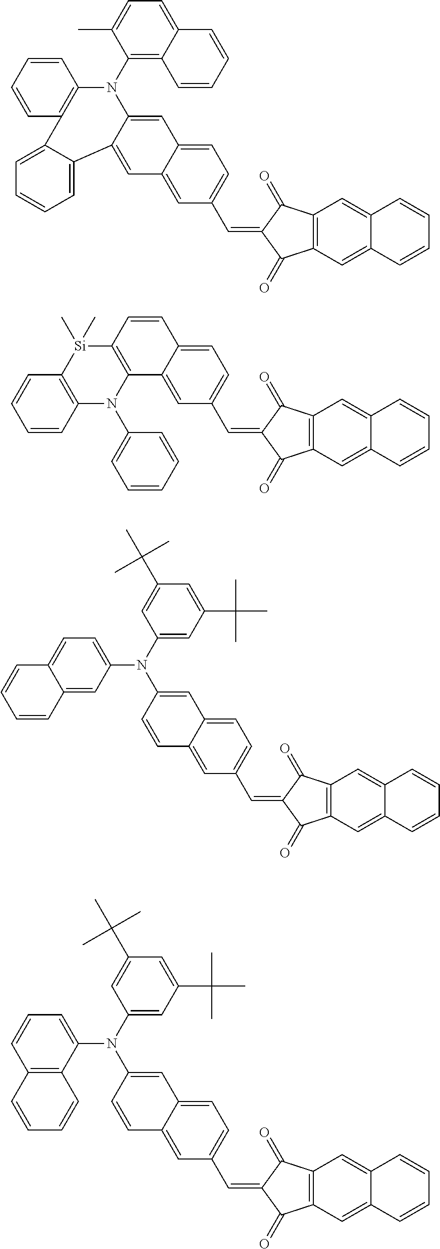

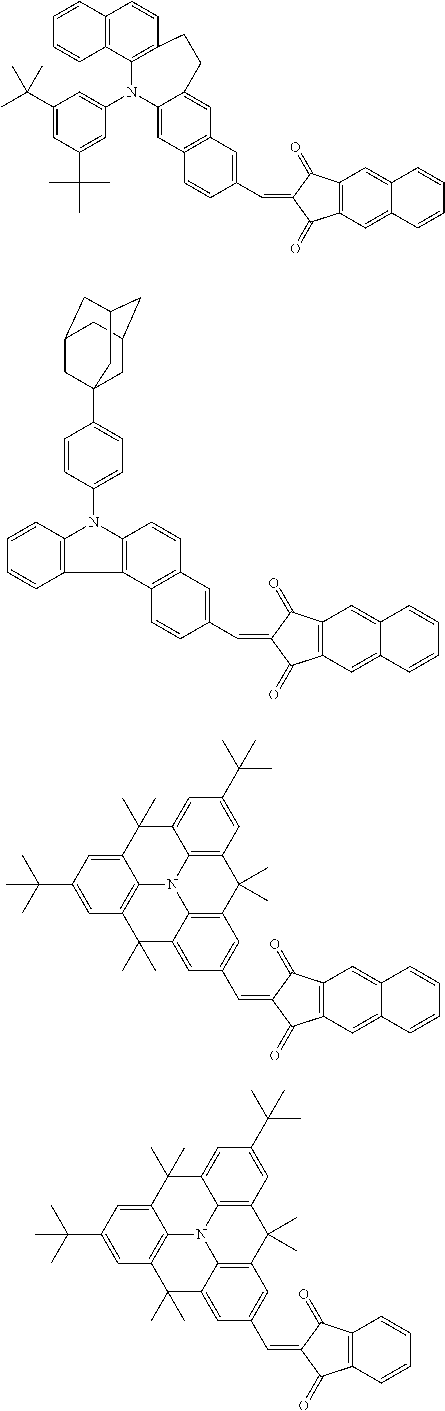

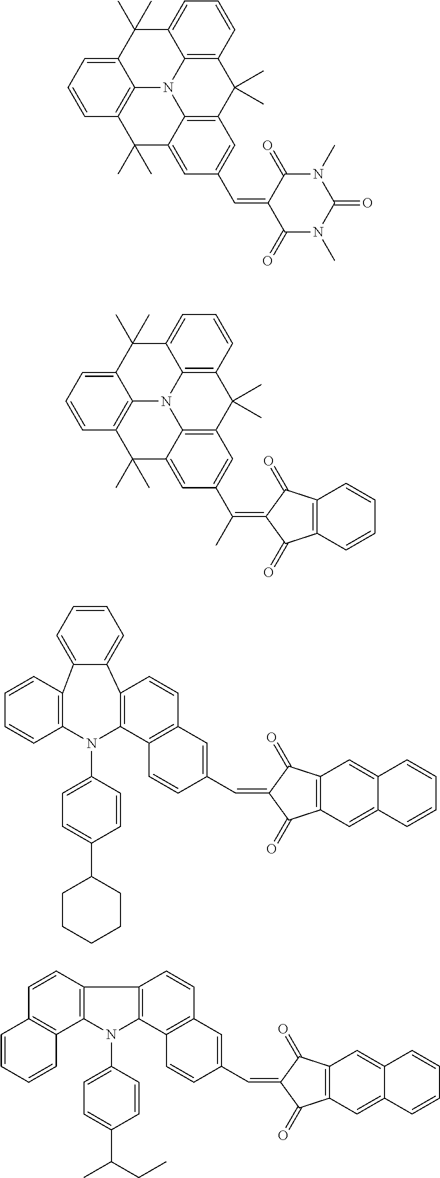

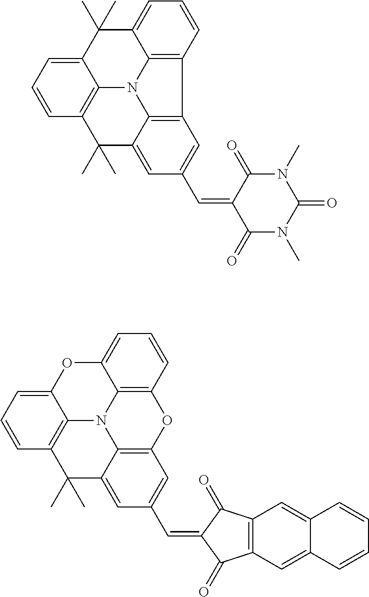 [SMILES: CN1C(=O)C(=Cc2cc3c4c(c2)c2cccc5c2n4-c2c(cccc2C3(C)C)C5(C)C)C(=O)N(C)C1=O ; CC1(C)c2cccc3c2N2c4c(cccc4Oc4cc(C=C5C(=O)c6cc7ccccc7cc6C5=O)cc1c42)O3]

### [Chemical Formula 27]

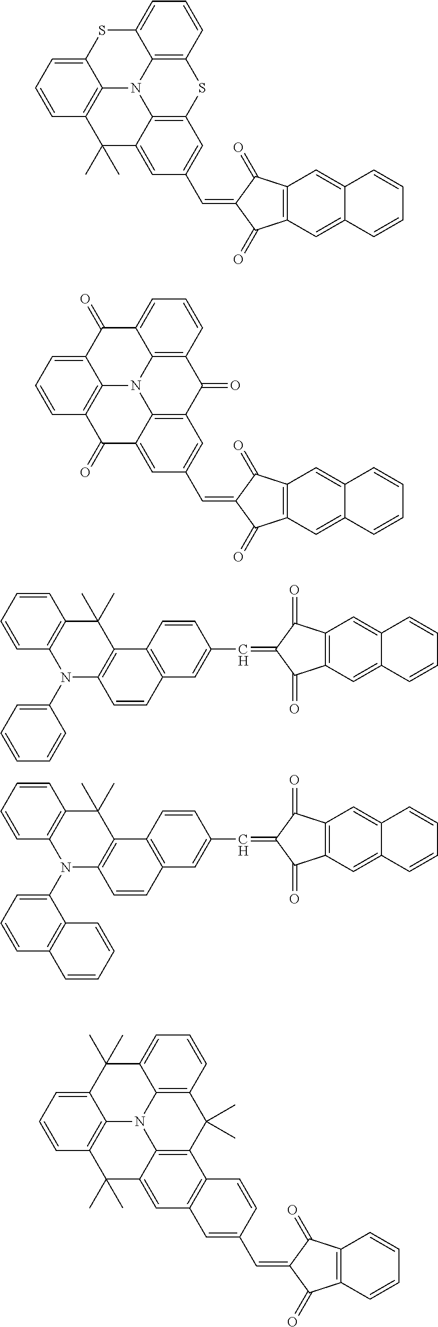

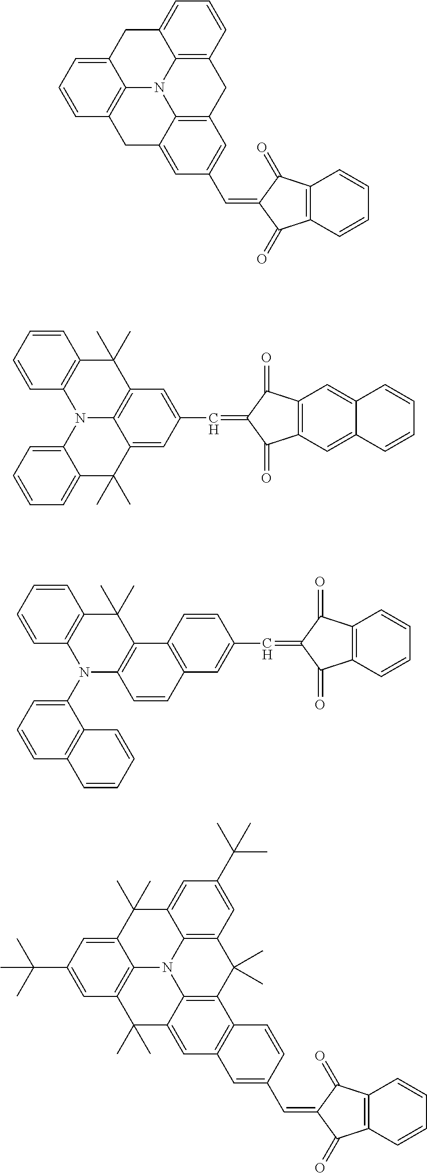 [SMILES: O=C1C(=Cc2cc3c4c(c2)Cc2cccc5c2N4c2c(cccc2C3)C5)C(=O)c2ccccc21 ; CC1(C)c2ccccc2N2c3ccccc3C(C)(C)c3cc(C=C4C(=O)c5cc6ccccc6cc5C4=O)cc1c32 ; CC1(C)c2ccccc2N(c2cccc3ccccc23)c2ccc3cc(C=C4C(=O)c5ccccc5C4=O)ccc3c21 ; CC(C)(C)c1cc2c3c(c1)C(C)(C)c1cc4cc(C=C5C(=O)c6ccccc6C5=O)ccc4c4c1N3c1c(cc(C(C)(C)C)cc1C4(C)C)C2(C)C]

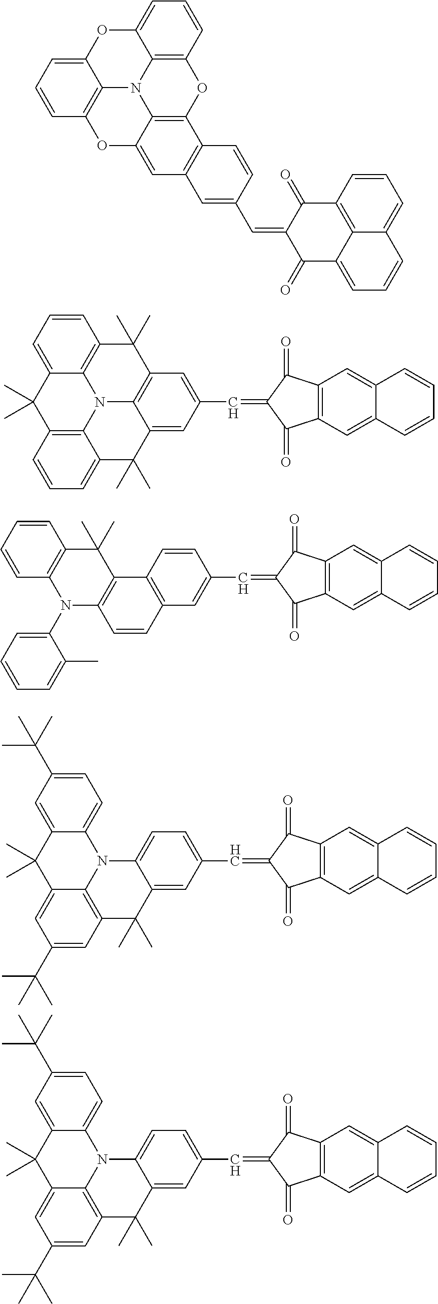

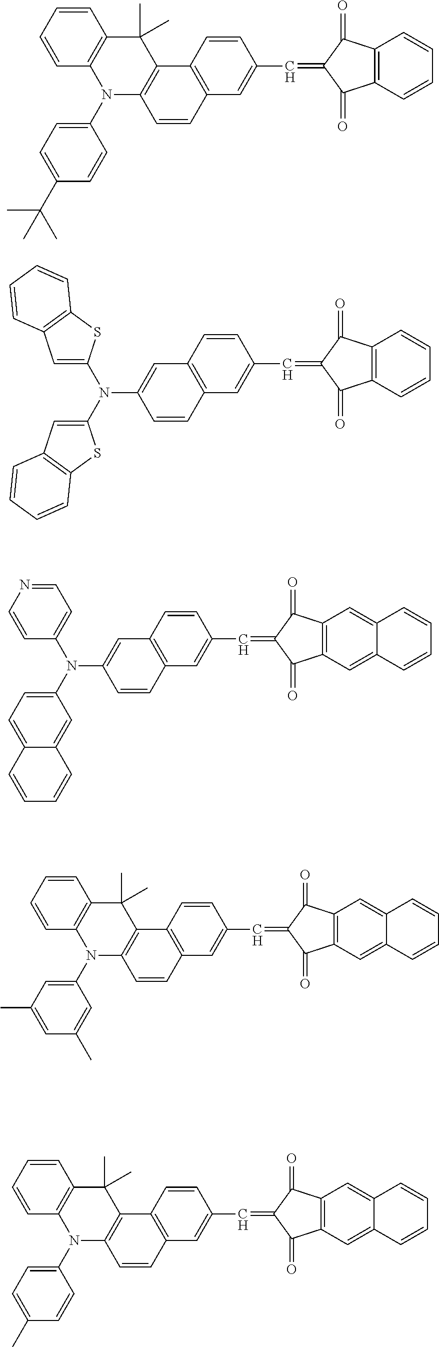

The compounds represented by the general formula (D-I) are particularly useful as a photoelectric conversion material used in photosensors and photocells. Other applications can include coloring material, liquid crystal material, organic semiconductor material, organic light-emitting element material, charge transport material, pharmaceutical material, fluorescent diagnostic agent material, and the like.

In addition, the compounds represented by the general formula (D-I), for example, can be synthesized according to the following reaction.

### [Chemical Formula 28]

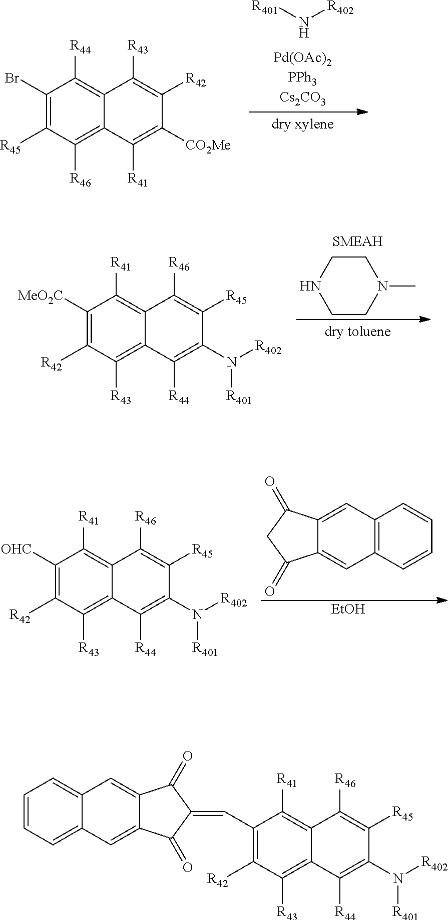

(In the above-mentioned formula, the R₄₁ to R₄₆, R₄₀₁, and R₄₀₂ have the same meanings as those of R₄₁ to R₄₆, R₄₀₁, and R₄₀₂ in the general formula (D-IV)).

Note that in the above-mentioned synthesis example, while Z₁ of the compound represented by the general formula (D-I) has 1,3-benzoin Dan-dione nucleus, even when Z₁ has another structure, the same synthesis can be carried out by changing the 1-3-benzoin Dan-dione to another compound.

### <<Electron-Donating Organic Material>>

When fullerene or a fullerene derivative is used as an n-type organic semiconductor of the photoelectric conversion layer 32, preferred electron donating organic materials as an electron blocking layer 31 will be described below.

The electron blocking layer 31 include compounds represented by the following general formula (EB-1), or compounds represented by the following general formula (EB-2).

The compound represented by the general formula (EB-2) can improve the element performance, while maintaining the heat resistance of the element because of a high moving velocity of charges. Specifically, the photoelectric conversion element can achieve the high charge collection efficiency and high-speed response. The organic electroluminescent element can achieve light emission with high efficiency. The organic transistor can achieve a high On/Off ratio.

On the other hand, the compound represented by the general formula (EB-1) which has a condensed diarylamine structure has a high glass transition temperature and a high heat resistance to the element because free rotation of molecules thereof by thermal motion is suppressed.

### [Chemical Formula 29]

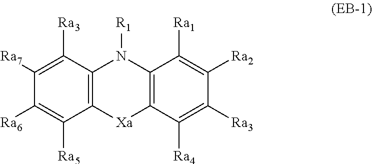

(In the formula, R₁ may be an alkyl group, an aryl group, or a heterocyclic group, which may have a substituent group. Ra₁ to Ra₈ each independently represents a hydrogen atom or a substituent group. R₁ and at least two of the Ra₁ to Ra₈ may be bonded to form a ring. Xa may have a single bond, an oxygen atom, a sulfur atom, or an alkylene group, a silylene group, an alkenylene group, a cycloalkylene group, cycloalkenylene group, an arylene group, a divalent heterocyclic group, or an imino group, which may have a substituent group.)

### [Chemical Formula 30]

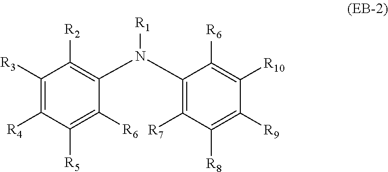

(In the formula, R₁ may be an alkyl group, an aryl group, or a heterocyclic group that may have a substituent group. R₀ and R₂ to R₁₀ independently represent a hydrogen atom or a substituent.)

Furthermore, the compound represented by the following general formula (EB-3) that is obtained by connecting a condensed diarylamine (substituent represented by the following general formula (A-1)) to the divalent coupling group (C-1) is useful as electric blocking material for a photoelectric conversion element. The use of the coupling group (C-1) leads to an increase in molecular weight, and can improve the heat resistance as compared to the use of the coupling group (C-2). Since the bond between the skeletons is twisted to cut a conjugated system, a layer using the above material (for example, electron blocking layer) and an adjacent layer (for example, a photoelectric conversion layer) do not interact with each other. Therefore, it is estimated that the dark current of the photoelectric conversion element is kept low. Since the diaryl amine structure, which is a charge-transport unit, is introduced not into the inside of the molecule, but to both ends thereof, this material is considered to have a high charge transport properties.

Furthermore, through the studies of the present applicants, it has been found that in the general formula (EB-3), a coupling position of the coupling group (C-1), a binding position of the substituent group represented by the general formula (A-1), a substituent position of the substituent group (S₁₁), and the kind of the substituent group (S₁₁) are selected, which can improve the heat resistance of the electron blocking layer without causing a reduction in the response speed of the photoelectric conversion element. By finding the optimum points of the coupling position of the coupling group (C-1), the binding position of the substituent group represented by the general formula (A-1), the substituent position of the substituent group (S₁₁), and the substituent group (S₁₁), it is considered that the effects of suppressing the interaction with the photoelectric conversion layer, and increasing an intermolecular force between the compounds represented by the general formula (EB-3) due to an increase in molecular weight are significantly exhibited, thereby achieving the high heat resistance.

### [Chemical Formula 31]

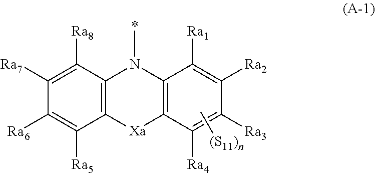

(In the general formula (A-1), Ra₁ to Ra₈ each independently represent a hydrogen atom, a halogen atom, an alkyl group, an aryl group, a heterocyclic group, or an alkoxy group, and these may have a further substituent group. At least two of Ra₁ to Ra₈ may be bonded to each other to form a ring. * represents a binding position. Xa may have a single bond, an oxygen atom, a sulfur atom, or an alkylene group, a silylene group, an alkenylene group, a cycloalkylene group, a cycloalkenylene group, an arylene group, a divalent heterocyclic group, or an imino group, which may have a substituent group. S₁₁ each independently indicates the following substituent group (S₁₁), and substitutes as any one of Ra₁ to Ra₈. n each independently represents an integer number of 1 to 4.)

### [Chemical Formula 32]

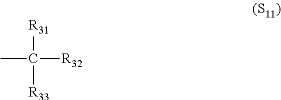

(Rs₁ to Rs₃ each independently represent a hydrogen atom or an alkyl group. At least two of Rs₁ to Rs₃ may be bonded to each other to form a ring.)

### [Chemical Formula 33]

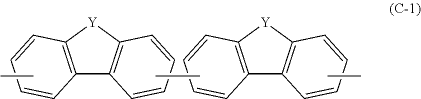

### [Chemical Formula 34]

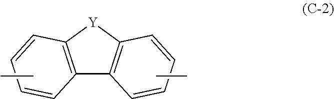

(In the following general formulas (C-1) and (C-2), Y independently represents —C(R₂₁) (R₂₂)—, —Si(R₂₃)(R₂₄)—, —N(R₂₀)—, an oxygen atom, or a sulfur atom, and R₂₀ to R₂₄ each independently represent a hydrogen atom, a halogen atom, an alkyl group, an aryl group, a heterocyclic group, a hydroxyl group, an amino group or a mercapto group.)

### [Chemical Formula 35]

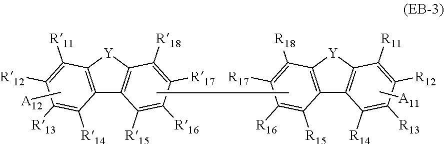

(In the general formula (EB-3), R₁₁ to R₁₈, and R′₁₁ to R′₁₈ independently represent a hydrogen atom, a halogen atom, an alkyl group, an aryl group, a heterocyclic group, a hydroxyl group, an amino group or a mercapto group, which may have a further substituent group. Note that any one of R₁₅ to R₁₈ is coupled to any one of R′₁₅ to R′₁₈ to form a single bond. A₁₁ and A₁₂ each independently represents a substituent group represented by the general formula (A-1), and which substitutes as any one of R₁₁ to R₁₄, and any one of R′₁₁ to R′₁₄. Y each independently represents a carbon atom, a nitrogen atom, an oxygen atom, a sulfur atom, or a silicon atom, which may have a further substituent group.)

Furthermore, through the studies of the present inventors, it has been found that in the general formula (EB-3), a coupling position of the coupling group (C-1), a bonding position of a substituent group represented by the general formula (A-1), a substituent position of the substituent group (S₁₁) below, and the kind of the substituent group (S₁₁) are selected, which can enhance the heat resistance of the electron blocking layer without causing a reduction in the response speed of the photoelectric conversion element. By finding the optimum points of the coupling position of the coupling group (C-1), the binding position of the substituent group represented by the general formula (A-1), the substituent position of the substituent group (S₁₁), and the substituent group (S₁₁), it is considered that the effects of suppressing the interaction with the photoelectric conversion layer, and increasing an intermolecular force between the compounds represented by the general formula (EB-3) due to an increase in molecular weight are significantly exhibited, thereby achieving the high heat resistance.

In the following, the compound represented by each general formula will be described below.

First, the compound represented by the formula (EB-1) will be described.

In the general formula (EB-1), R₁ represents an alkyl group, an aryl group, or a heterocyclic group, and may have a substituent group. Specific examples of the substituent group include the substituent group W described below, preferably a halogen atom, an alkyl group, an aryl group, a heterocyclic group, a hydroxyl group, an amino group, and a mercapto group, more preferably a halogen atom, an alkyl group, an aryl group, a heterocyclic group, an amino group, even more preferably a fluorine atom, an alkyl group, an aryl group, an amino group, particularly preferably an alkyl group, an aryl group, an amino group, and most preferably an alkyl group, and an amino group. When having a plurality of the substituent groups, the substituent groups may be coupled to each other to form a ring. The formed ring includes the ring R to be described later.

When R₁ is an alkyl group, the alkyl group may be linear, branched alkyl group, and a cyclic alkyl group (cycloalkyl group), and be preferably a cycloalkyl group. The number of carbon atoms, when it does not include the carbazole skeleton in R₁, is preferably in a range of 4 to 20, and more preferably 5 to 16. The number of carbon atoms, when it contains a carbazole skeleton in R₁, is preferably in a range of 19-35, and more preferably 20-31. Specifically, examples of the cycloalkyl group include a cycloalkyl group (cyclopiropiru group, cyclopentyl group, cyclohexyl group, etc.), a cycloalkenyl group (2-cyclohexen-1-yl group etc.), and the like.

When R₁ is an aryl group, R₁ is a substituted or unsubstituted aryl group in which, when it does not include the carbazole skeleton in R₁, the number of carbon atoms is preferably in a range of 6 to 20, and more preferably 6 to 16, and in which, when it includes the carbazole skeleton in R₁, the number of carbon atoms is preferably in a range of 21 to 35, and more preferably 21-31. More specifically, examples of the aryl group include a phenyl group, a naphthyl group, an anthryl group, a fluorenyl group, and the like.

When R₁ is a heterocyclic group, the heterocyclic group includes a 5-membered or 6-membered heterocyclic group. Specifically, examples of the heterocyclic groups include a furyl group, a thienyl group, a pyridyl group, a quinolyl group, a thiazolyl group, an oxazolyl group, azepinyl group, a carbazolyl group, and the like. An aryl group or heterocyclic group may contain a condensed ring of two to four single rings.

Preferably, R₁ is an aryl group or a heterocyclic group, more preferably an aryl group, and most preferably a phenyl group. Another preferred aspect of R₁ is an aryl group or a heterocyclic group having a skeleton represented by the general formula (C-2).

The group having a skeleton represented by the general formula (C-2) may further have a substituent group. A specific example of the substituent group includes the substituent group W described below. As a substituent group, an aryl group or a heterocyclic group which further has the skeleton represented by the general formula (C-2) (in which these groups may have the substituent group W described below) is preferably provided. Also, the substituent groups may be coupled to each other to form a ring, and an example of the ring formed includes the ring R described later.

In other more preferred embodiments of R₁, two or more of the aryl or heterocyclic groups having a skeleton represented by the general formula (C-2) are single-bonded, or coupled together via a substituent group (more preferably, two of these groups are coupled together). In a particularly preferred embodiment, two of the aryl and/or heterocyclic groups having the skeleton represented by the general formula (C-2) are coupled together via a single bond.

In the general formula (EB-1), Ra₁ to Ra₈ independently represent a hydrogen atom or a substituent group. A specific example of the substituent group is the substituent W described below. Examples of the substituent group preferably include a halogen atom, an alkyl group, an aryl group, a heterocyclic group, a hydroxyl group, an amino group, a mercapto group, or an alkoxy group, more preferably a halogen atom, an alkyl group, an aryl group, a heterocyclic group, an alkoxy group, more preferably a halogen atom, an alkyl group, an aryl group, a heterocyclic group, more preferably a fluorine atom, an alkyl group, an aryl group, particularly preferably an alkyl group, an aryl group, and most preferably an alkyl group.

Preferred specific examples of Ra₁ to Ra₈ include a hydrogen atom, a fluorine atom, a methyl group, an ethyl group, a propyl group, a butyl group, a hexyl group, a cyclohexyl group, a phenyl group, and a naphthyl group.

In addition, it is preferred that at least one of Ra₃ and Ra₆ is a hydrogen atom or an alkyl group having 1 to 10 carbon atoms, and each of Ra₁, Ra₂, Ra₄, Ra₅, Ra₇ and Ra₈ is a hydrogen atom. Alternatively, it is preferred that at least one of the Ra₂ and Ra₇ is a hydrogen atom or an alkyl group having 1 to 10 carbon atoms, and each of Ra₁, Ra₃, Ra₄, Ra₅, Ra₆ and Ra₈ is a hydrogen atom. In particular, it is more preferred that Ra₃ and Ra₆ is a hydrogen atom or an alkyl group having 1 to 6 carbon atoms, and each of Ra₁, Ra₂, Ra₄, Ra₅, Ra₇ and Ra₈ is a hydrogen atom.

At least two of R₁ and Ra₁ to Ra₈ may be bonded to each other to form a ring. As the ring formed, a ring R will be described later. Examples of the formed ring include a cycloalkyl ring having 5 to 18 carbon atoms, a benzene ring, a naphthalene ring, an indane ring, an anthracene ring, a pyrene ring, a phenanthrene ring, a perylene ring, a pyridine ring, a quinoline ring, an isoquinoline ring, a phenanthridine ring, a pyrimidine ring, a pyrazine ring, a pyridazine ring, a triazine ring, a cinnoline ring, an acridine ring, a phthalazine ring, a quinazoline ring, a quinoxaline ring, a naphthyridine ring, a pteridine ring, a pyrrole ring, a pyrazole ring, a triazole ring, an indole ring, a carbazole ring, an indazole ring, a benzimidazole ring, an oxazole ring, a thiazole ring, an oxadiazole ring, a thiadiazole ring, a benzoxazole ring, a benzothiazole ring, an imidazopyridine ring, a thiophene ring, a benzothiophene ring, a furan ring, a benzofuran ring, a phosphole ring, a phosphinine ring, a silole ring and the like. Preferably, the cycloalkyl ring having 5 to 18 carbon atoms, a benzene ring, a naphthalene ring, an indane ring, an anthracene ring, a pyrene ring, a phenanthrene ring, a perylene ring, a pyrrole ring, an indole ring, a carbazole ring, an indazole ring, a thiophene ring, a benzothiophene ring, a furan ring, a benzofuran ring, more preferably a cycloalkyl ring having 5 to 18 carbon atoms, a benzene ring, a naphthalene ring, an indane ring, an indole ring, a carbazole ring, an indazole ring, and particularly preferably, a cycloalkyl ring having 5 to 10 carbon atoms, a benzene ring, a naphthalene ring, an indane ring, an anthracene ring. Among them, a cycloalkyl ring having 5 to 10 carbon atoms, a benzene ring, a naphthalene ring, and an indane ring are more preferable, and a cycloalkyl ring having 5-6 carbon atoms, a benzene ring, and an indane ring are most preferable. These rings may further have a substituent W described below.

Xa represents a single bond, an oxygen atom, or a sulfur atom, an alkylene group, a silylene group, an alkenylene group, a cycloalkylene group, a cycloalkenylene group, an arylene group, a divalent heterocyclic group, or an imino group, each of which may have a substituent group. Specific examples of the substituent group include the substituent W, and is preferably an alkyl group or an aryl group.

Xa is preferably a single bond, an alkylene group having 1 to 12 carbon atoms, an alkenylene group having 2 to 12 carbon atoms, an arylene group having 6 to 14 carbon atoms, a heterocyclic group having 4 to 13 carbon atoms, an oxygen atom, a sulfur atom, an imino group (e.g. phenylimino group, methylimino group, t-butylimino group) with a hydrocarbon group having 1 to 12 carbon atoms (preferably an aryl group or an alkyl group), a silylene group, more preferably, a single bond, an oxygen atom, an alkylene group having a carbon atoms of 1-6 (for example, a methylene group, 1,2-ethylene group, 1,1-dimethylmethylene group), an alkenylene group having 2 carbon atoms (for example, —CH₂═CH₂—), an arylene group having 6 to 10 carbon atoms (for example, 1,2-phenylene group, 2,3-naphthylene group), a silylene group, and further preferably, a single bond, an oxygen atom, an alkylene group having 1 to 6 carbon atoms (for example, a methylene group, 1,2-ethylene group, 1,1-dimethyl-methylene group).

The general formula (EB-3) indicates a condensed diaryl amine (the above substituent group represented by the general formula (A-1)) connected by a divalent coupling group (C-1). In EB-3, R₁₁ to R₁₈ and R′₁₁ to R′₁₈ each independently represent a hydrogen atom, a halogen atom, an alkyl group, an aryl group, a heterocyclic group, a hydroxyl group, an amino group or a mercapto group, and these may further have a substituent group.

Specific examples of the further substituent group include the substituent W described below, preferably a halogen atom, an alkyl group, an aryl group, a heterocyclic group, a hydroxyl group, an amino group, a mercapto group, more preferably a halogen atom, an alkyl group, an aryl group, a heterocyclic group, more preferably a fluorine atom, an alkyl group, an aryl group, particularly preferably an alkyl group, an aryl group, and most preferably an alkyl group.

R₁₁ to R₁₈ and R′₁₁ to R′₁₈ each represent a hydrogen atom, or an alkyl group, aryl group, or heterocyclic group in which these groups may have a substituent group in terms of chemical stability, electric charge mobility, and heat resistance, more preferably, a hydrogen atom, an alkyl group having 1 to 18 carbon atoms, an aryl group having 6 to 18 carbon atoms, or a heterocyclic group having 4 to 16 carbon atoms in which these groups may have a substitutent group. Among them, in terms of the charge mobility and heat resistance, preferably, the substituent group represented by the general formula (A-1) independently substitutes for R₁₂ and R′₁₂, respectively. More preferably, the substituent group represented by the general formula (A-1) independently substitutes for R₁₂ and R′₁₂, and R₁₁, R₁₃ to R₁₈, R′₁₁, and R′₁₃ to R′₁₈ each are a hydrogen atom, or an alkyl group having 1 to 18 carbon atoms that may have a substituent group. In particular, preferably, the substituent group represented by the general formula (A-1) independently substitutes for R₁₂ and R′₁₂, and R₁₁, R₁₃ to R₁₈, R′₁₁, and R′₁₃ to R′₁₈ each are a hydrogen atom.

Y each independently represents a carbon atom, a nitrogen atom, an oxygen atom, a sulfur atom or a silicon atom, which may have a substituent group. That is, Y represents a carbon atom, a nitrogen atom, an oxygen atom, a sulfur atom, or a divalent coupling group consisting of a silicon atom. Examples of the substituent group include the substituent W described below.

Y independently represents, —C(R₂₁) (R₂₂)—, —Si(R₂₃) (R₂₄)—, —N(R₂₀)—, an oxygen atom, or sulfur atom, and R₂₀ to R₂₄ each independently represents a hydrogen atom, a halogen atom, an alkyl group, an aryl group, a heterocyclic group, a hydroxyl group, an amino group, or a mercapto group. Among these, in terms of chemical stability, electric charge mobility, and heat resistance, —C(R₂₁) (R₂₂)—, —Si(R₂₃) (R₂₄)—, and —N(R₂₀)— are preferred, and —C(R₂₁) (R₂₂)—, and —N(R₂₀)— are more preferred, and —C(R₂₁) (R₂₂)— is particularly preferred.

In the —C(R₂₁) (R₂₂)—, the R₂₁ and R₂₂, each independently represent a hydrogen atom, a halogen atom, an alkyl group, an aryl group, a heterocyclic group, a hydroxyl group, an amino group or a mercapto group. R₂₁ and R₂₂ may further have a substituent group. Examples of the further substituent include the substituent W described below, preferably an alkyl group, an aryl group, or an alkoxy group.

Preferably, R₂₁ and R₂₂ is a hydrogen atom, or an alkyl group, an aryl group, and a heterocyclic group, in which these groups may have a substituent group, more preferably a hydrogen atom, or an alkyl group having 1 to 18 carbon atoms, an aryl group having 6 to 18 carbon atoms, or a heterocyclic group having 4 to 16 carbon atoms in which these groups may have a substituent group, even more preferably, a hydrogen atom, or an alkyl group having 1 to 18 carbon atoms which may have a substituent group, and particularly preferably, an alkyl group having 1 to 18 carbon atoms.

In —Si(R₂₃)(R₂₄)—, R₂₃ and R₂₄ each independently represent a hydrogen atom, a halogen atom, an alkyl group, an aryl group, a heterocyclic group, a hydroxyl group, an amino group or a mercapto group. R₂₃ and R₂₄ may further have a substituent group, specific examples of further substituents include substituents W, preferably an alkyl group, an aryl group, or an alkoxy group.

Preferably, R₂₃ and R₂₄ may be a hydrogen atom, or an alkyl group, an aryl group, and a heterocyclic group which may have a substituent group, more preferably a hydrogen atom, or an alkyl group having 1 to 18 carbon atoms, an aryl group having 6 to 18 carbon atoms, or a heterocyclic group having 4 to 16 carbon atoms, which may have a substituent group, even more preferably a hydrogen atom, or an alkyl group having 1 to 18 carbon atoms which may have a substituent group, and particularly preferably an alkyl group having 1 to 18 carbon atoms.

Also, R₂₃ and R₂₄ may be bonded to form a ring. The ring is preferably an aliphatic hydrocarbon ring, more preferably an aliphatic hydrocarbon ring having 4 to 10 carbon atoms.

In —N(R₂₀)—, R₂₀ preferably represents an alkyl group, an aryl group, and a heterocyclic group. R₂₀ may further have a substituent group. Examples of the further substituent group include the substituent W, preferably an alkyl group or an aryl group.

More preferably, examples of R₂₀ include a hydrogen atom, an alkyl group having 1 to 18 carbon atoms, an aryl group having 6 to 18 carbon atoms, or a heterocyclic group having 4 to 16 carbon atoms, which may have a substituent, more preferably a hydrogen atom, an alkyl group having 1 to 18 carbon atoms which may have a substituent group, and particularly preferably an alkyl group having 1 to 18 carbon atoms.

Ra₁ to Ra₈ and Xa in the above general formula (A-1) are the same as Ra₁ to Ra₈ and Xa as described by the general formula (EB-1).

In a substituent group (S₁₁), Rs₁ represents a hydrogen atom or an alkyl group. In terms of chemical stability, electric charge mobility, and heat resistance, preferably, examples of Rs₁ include an alkyl group having 1 to 10 carbon atoms, more preferably an alkyl group having 1 to 6 carbon atoms, specifically, preferably, a methyl group, an ethyl group, a propyl group, an iso-propyl group, a butyl group or a tert-butyl group, more preferably a methyl group, an ethyl group, a propyl group, an iso-propyl group, or a tert-butyl group, even more preferably a methyl group, an ethyl group, an iso-propyl group or a tert-butyl group, and particularly preferably a methyl group, an ethyl group, or a tert-butyl group.

Rs₂ represents a hydrogen atom or an alkyl group. In terms of chemical stability, electric charge mobility, and heat resistance, Rs₂ preferably includes a hydrogen atom, or an alkyl group having 1 to 10 carbon atoms, more preferably a hydrogen atom or an alkyl group having 1 to 6 carbon atoms, specifically, a hydrogen atom, a methyl group, an ethyl group, a propyl group, an iso-propyl group, a butyl group, or a tert-butyl group, further preferably a hydrogen atom, a methyl group, an ethyl group, or a propyl group, more preferably a hydrogen atom, a methyl group, particularly preferably a methyl group.

Rs₃ represents a hydrogen atom or an alkyl group. In terms of chemical stability, electric charge mobility, and heat resistance, Rs₃ preferably includes a hydrogen atom, or an alkyl group having 1 to 10 carbon atoms, more preferably a hydrogen atom or an alkyl group having 1 to 6 carbon atoms, specifically, is a hydrogen atom, or a methyl group, and more preferably a methyl group.

At least two of Rs₁ to Rs₃ may be bonded to each other to form a ring. The ring preferably include an aliphatic hydrocarbon ring. The number of ring members is not particularly limited, but the ring is preferably 5 to 12-membered ring, more preferably 5 or 6-membered ring, and even more preferably a 6-membered ring. The rings preferably includes cyclopentane ring, cyclohexane ring, adamantane rings.

S₁₁ shows the above-mentioned substituent group (S₁₁), to substitute for any one of Ra₁ to Ra_{b}. At least one of the Ra₁ and Ra_{b} in the general formula (A-1) preferably independently represents a substituent group (S₁₁).

Preferably, examples of the substituent (S₁₁) can include the following groups (a) to (x), more preferably the groups (a) to (j), even more preferably the groups (a) to (h), particularly preferably the groups (a) to (f), further preferably the groups (a) to (c), and the most preferably the group (a). In the following (a) to (x), “*” represents the position substituted with the general formula (A-1).

### [Chemical Formula 36]

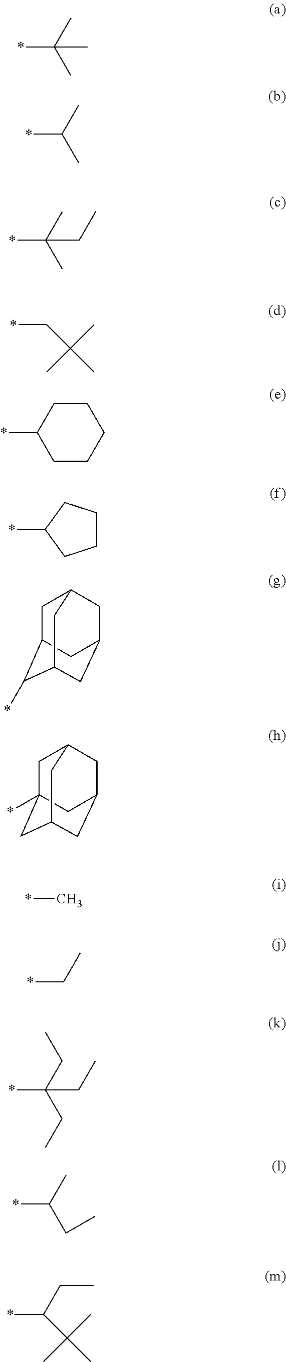

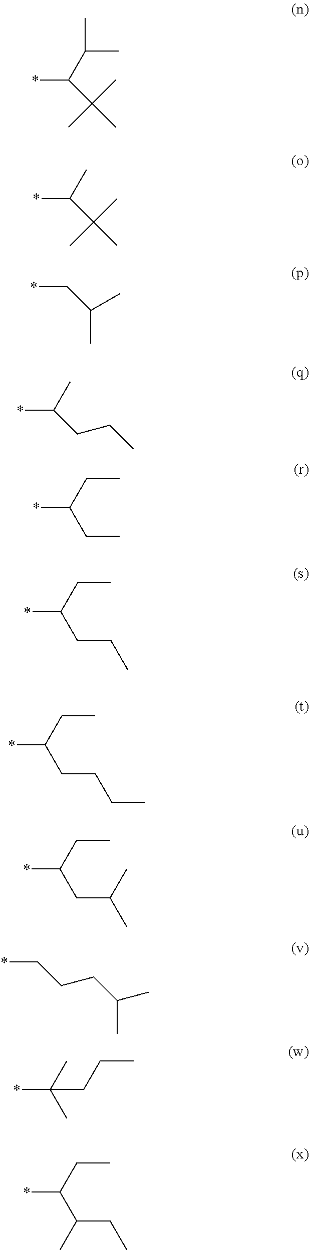

n each independently represents an integer number of 1-4, preferably 1-3, more preferably 1 or 2, and particularly preferably, 2. By introducing a substituent group represented by S₁₁, in use of the compound represented by the general formula (EB-3) for the electron blocking layer of the photoelectric conversion element, the interaction with the photoelectric conversion layer is suppressed, whereby the dark current becomes smaller. The intermolecular force between the compounds represented by the general formula (EB-3) is increased due to the increase in molecular weight, so that the element has a high heat resistance.

In one preferred embodiment of the present invention, Ra₁ to Ra₈ in the group represented by formula (A-1) each independently represent a hydrogen atom, a halogen atom, or an alkyl group.

When Ra₁ to Ra₈ each independently represent a hydrogen atom, a halogen atom, or an alkyl group in the group represented by the general formula (A-1), in one preferred embodiment, the general formula (A-1) is a group represented by the following general formulas (A-3) to (A-5).

### [Chemical Formula 37]

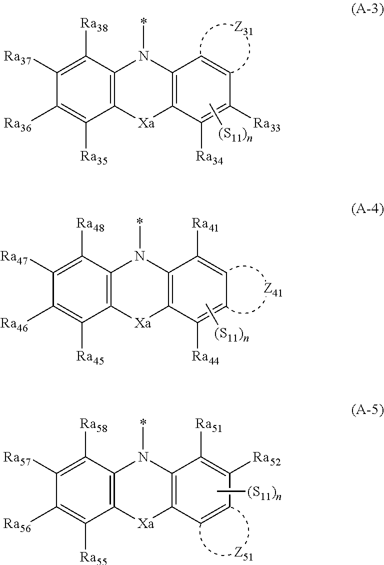

(In the general formulas (A-3) to (A-5), the Ra₃₃ to Ra₃₈, Ra₄₁, Ra₄₄ to Ra₄₈, Ra₅₁, Ra₅₂, and Ra₅₅ to Ra₅₈, each independently represent a hydrogen atom, a halogen atom, or an alkyl group. * represents the binding position. Xa is a single bond, an oxygen atom, a sulfur atom, an alkylene group, a silylene group, an alkenylene group, a cycloalkylene group, a cycloalkenylene group, an arylene group, a divalent heterocyclic group, or an imino group. S₁₁ independently represents the substituent (S₁₁), and substitutes for any one of Ra₃₃ to Ra₃₈, Ra₄₁, Ra₄₄ to Ra₄₈, Ra₅₁, Ra₅₂, Ra₅₅ to Ra₅₈. Z₃₁, Z₄₁ and Z₅₁ represent a cycloalkyl ring, an aromatic hydrocarbon ring, or an aromatic heterocyclic ring. n represents an integer number of 1-4.)

Xa, S₁₁ and n in the general formulas (A-3) to (A-5) have the same meanings as Xa, S₁₁ and n in the general formula (A-1), and preferable ones are also the same. In the general formulas (A-3) to (A-5), Ra₃₃ to Ra₃₈, Ra₄₁, Ra₄₄ to Ra₄₈, Ra₅₁, Ra₅₂, and Ra₅₅ to Ra₅₈ have the same meanings as a hydrogen atom, a halogen atom, or an alkyl group represented by Ra₂₁ to Ra₂₈ of the general formula (A-1), and preferable ones are also the same.

Z₃₁, Z₄₁ and Z₅₁ each represent a cycloalkyl ring, an aromatic hydrocarbon ring, or an aromatic heterocyclic ring. Examples of the ring represented by Z₃₁, Z₄₁ and Z₅₁ preferably include the cycloalkyl ring having 5 to 18 carbon atoms, a benzene ring, a naphthalene ring, an indane ring, an anthracene ring, a pyrene ring, a phenanthrene ring, a perylene ring, a pyridine ring, a quinoline ring, an isoquinoline ring, a phenanthridine ring, a pyrimidine ring, a pyrazine ring, a pyridazine ring, a triazine ring, a cinnoline ring, an acridine ring, a phthalazine ring, quinazoline ring, a quinoxaline ring, a naphthyridine ring, a pteridine ring, a pyrrole ring, a pyrazole ring, a triazole ring, an indole ring, a carbazole ring, an indazole ring, a benzimidazole ring, an oxazole ring, a thiazole ring, an oxadiazole ring, a thiadiazole ring, a benzoxazole ring, a benzothiazole ring, an imidazopyridine ring, a thiophene ring, a benzothiophene ring, a furan ring, a benzofuran ring, a phosphole ring, a phosphinine ring, a silole ring and the like. More preferably, examples of the ring include the cycloalkyl ring having 5 to 18 carbon atoms, a benzene ring, a naphthalene ring, an indane ring, an anthracene ring, a pyrene ring, a phenanthrene ring, a perylene ring, a pyrrole ring, an indole ring, a carbazole ring, an indazole ring, a thiophene ring, a benzothiophene ring, a furan ring, a benzofuran ring, even more preferably a cycloalkyl ring having 5 to 18 carbon atoms, a benzene ring, a naphthalene ring, an indane ring, an indole ring, a carbazole ring, an indazole ring, and particularly preferably, a cycloalkyl ring having 5 to 10 carbon atoms, a benzene ring, a naphthalene ring, an indane ring, an anthracene ring. Among them, examples of the ring preferably include a cycloalkyl ring having 5 to 10 carbon atoms, a benzene ring, a naphthalene ring, an indane ring, and most preferably a cycloalkyl ring having 5 to 6 carbon atoms, a benzene ring, an indane ring. These rings may further have a substituent W described below.

Specific examples of the group represented by the general formula (A-1) include groups represented by the following N-1 to N-135. However, the invention is not limited thereto. The preferred group represented by the general formula (A-1) is an N-1 to N-93, more preferably N-1 to N-79, even more preferably N-1 to N-37. Among them, N-1 to N-3, N-12 to N-22 and N-24 to N-35 are preferable, N-1 to N-3, N-17 to N-22 and N-30 to N-35 is especially preferable, and N-1 to N-3, N-17 to N-19 and N-30 to N-32 are most preferable. In the figure (S) represents the above-mentioned substituent group (S₁₁), n′ and n″ each independently represent an integer number of 1 to 4, and n′+n″ is an integer number of 1 to 4.

### [Chemical Formula 38]

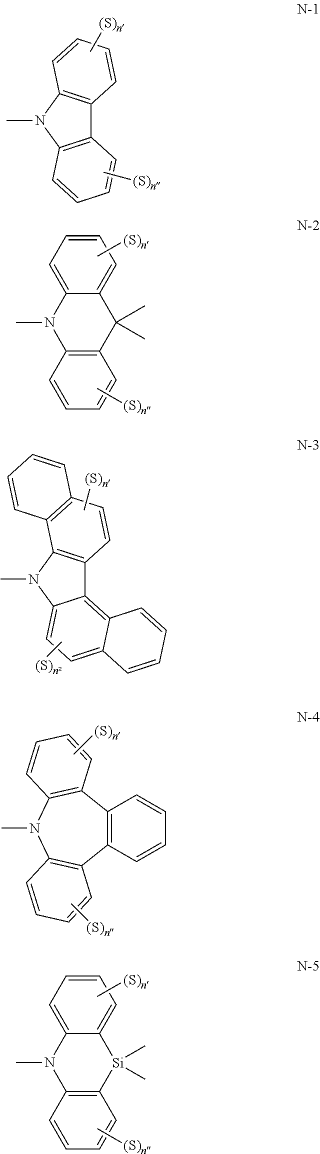

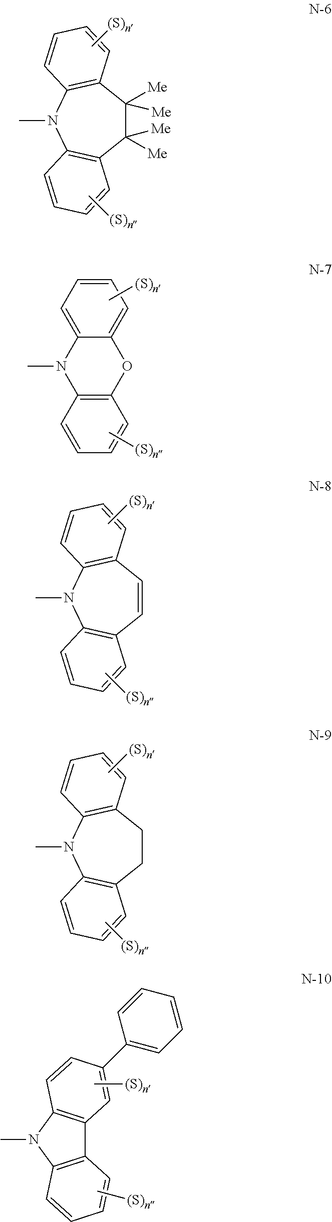

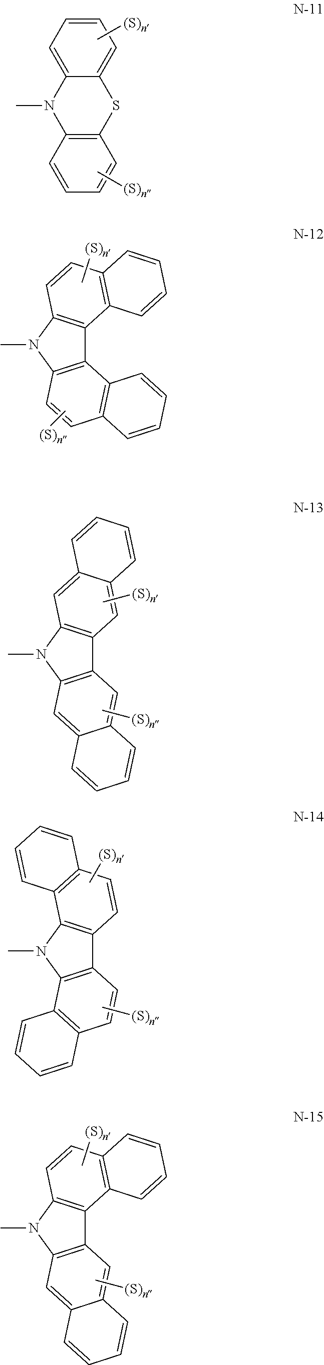

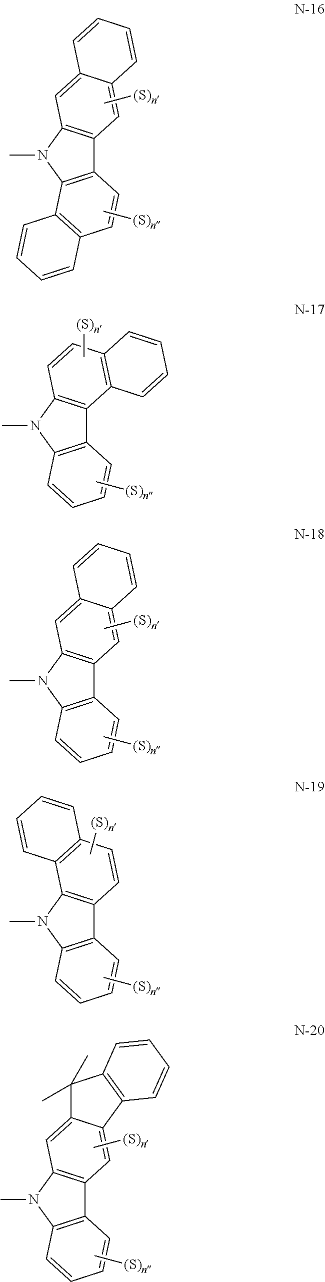

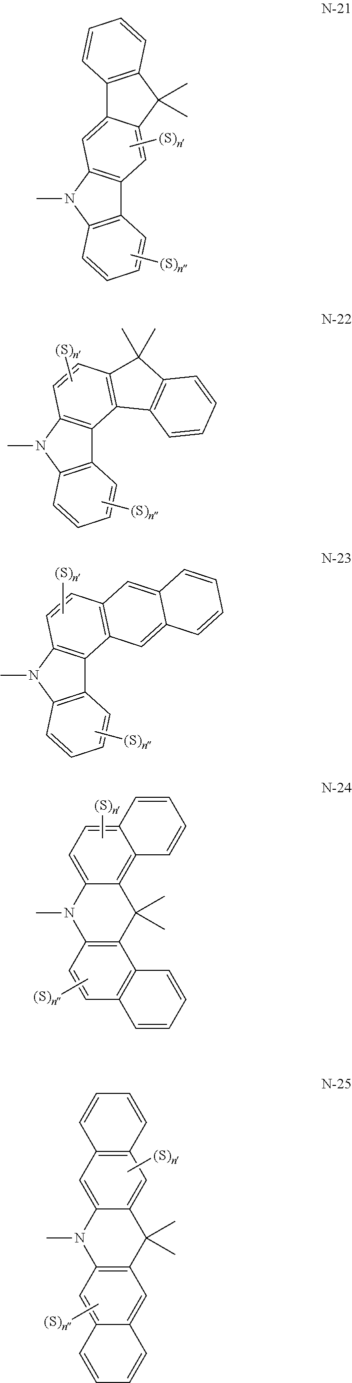

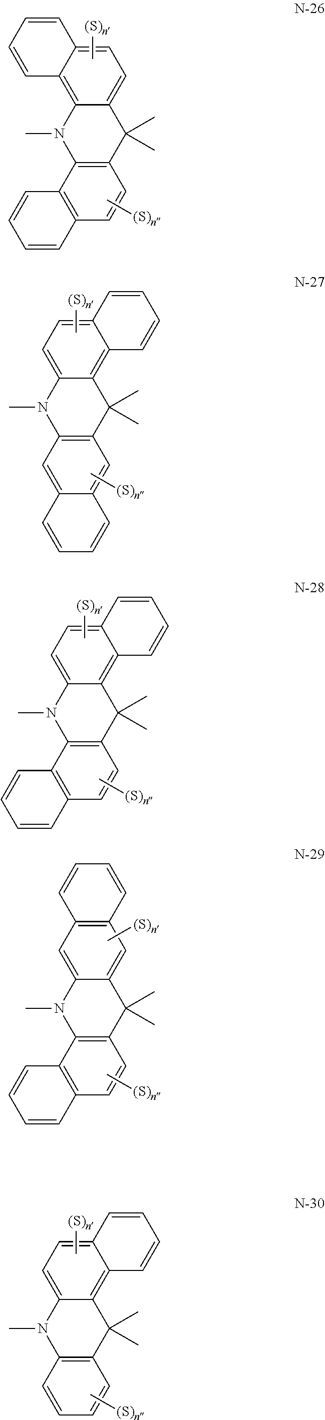

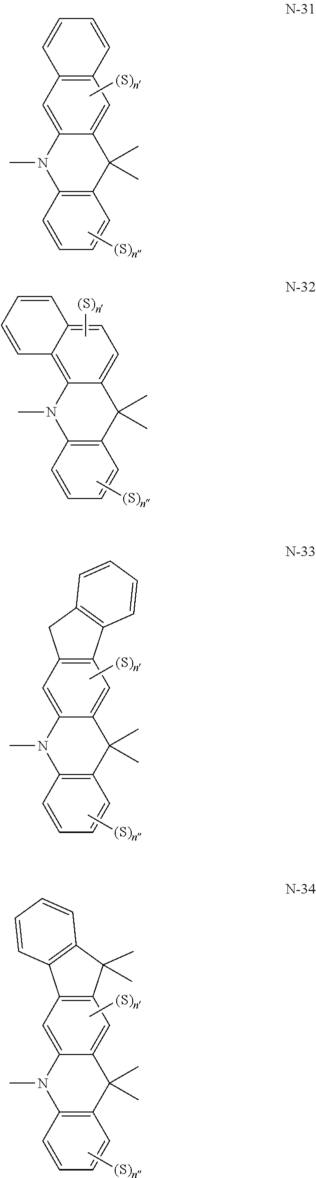

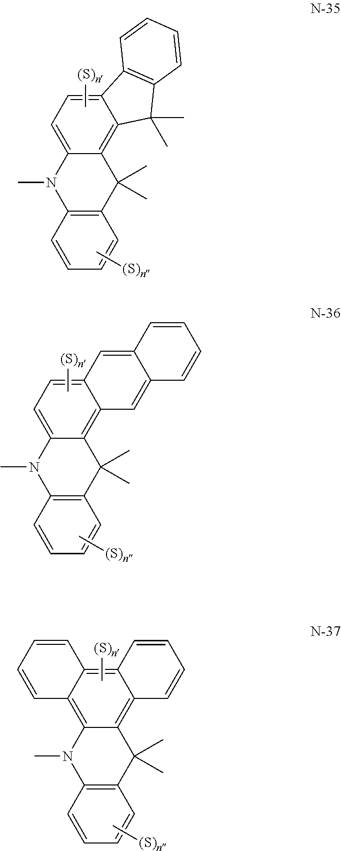

### [Chemical Formula 39]

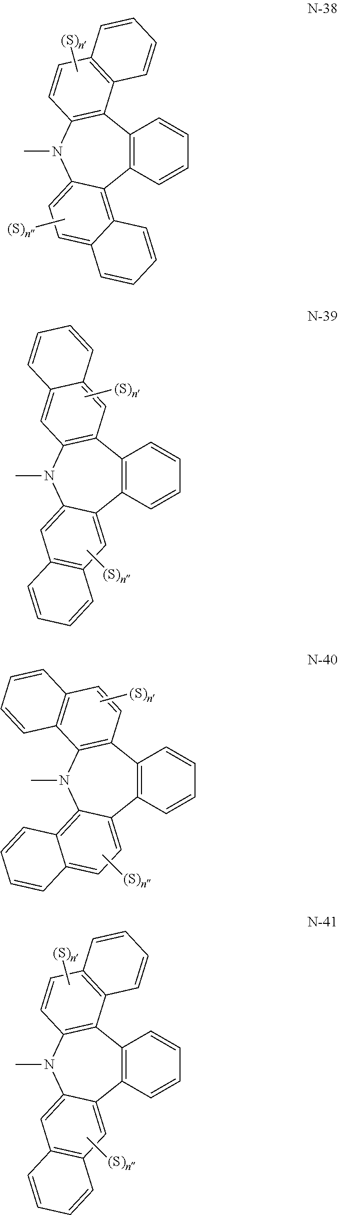

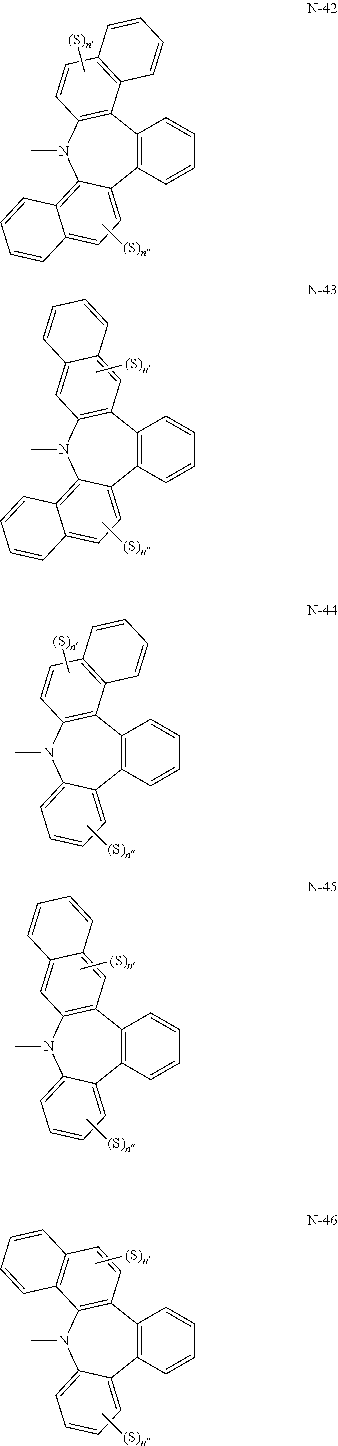

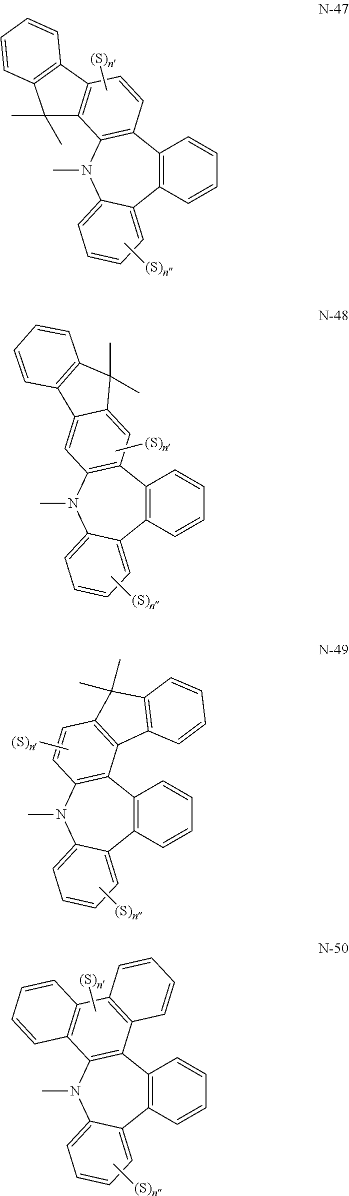

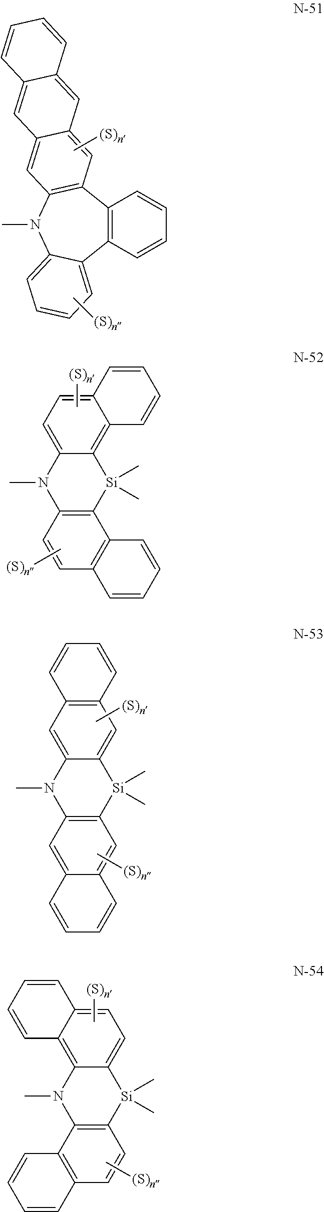

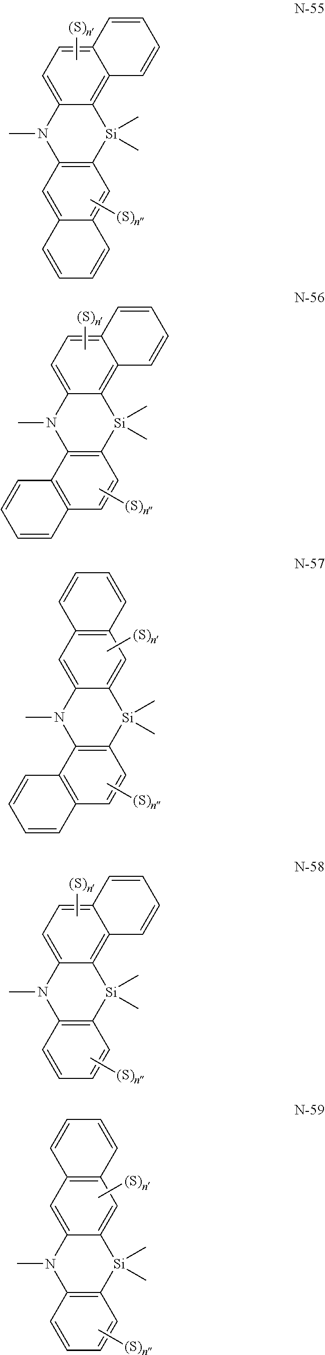

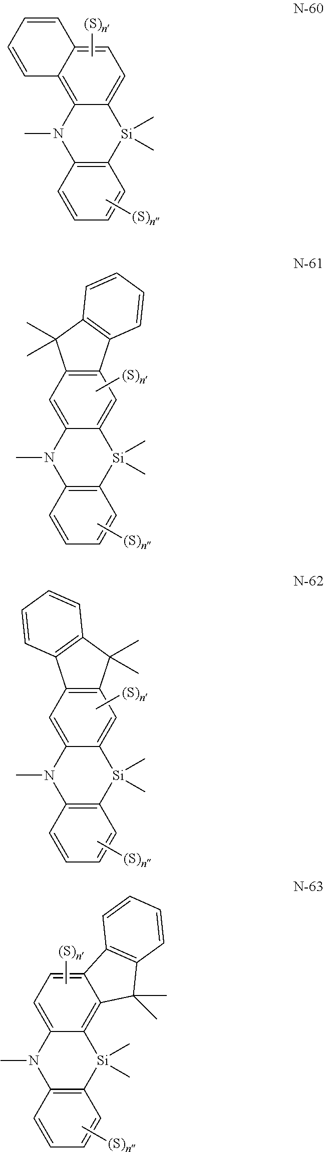

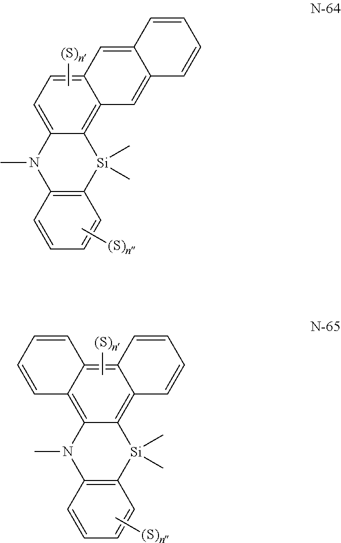

### [Chemical Formula 40]

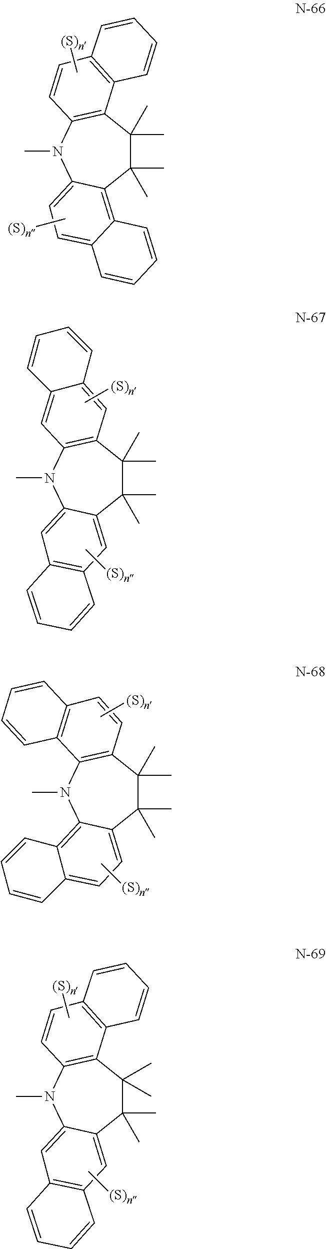

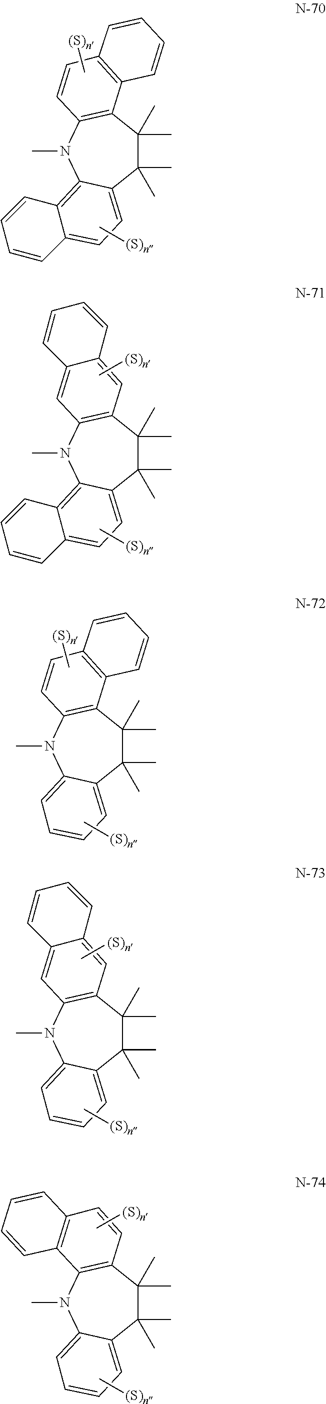

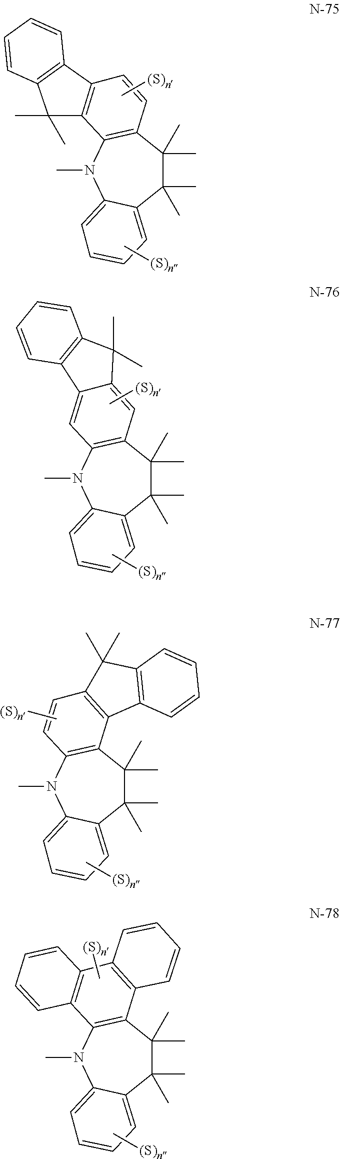

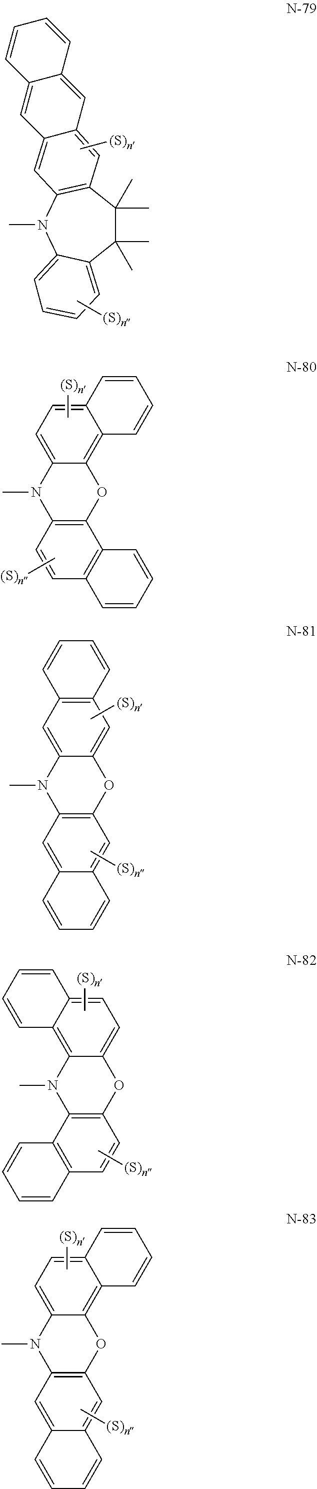

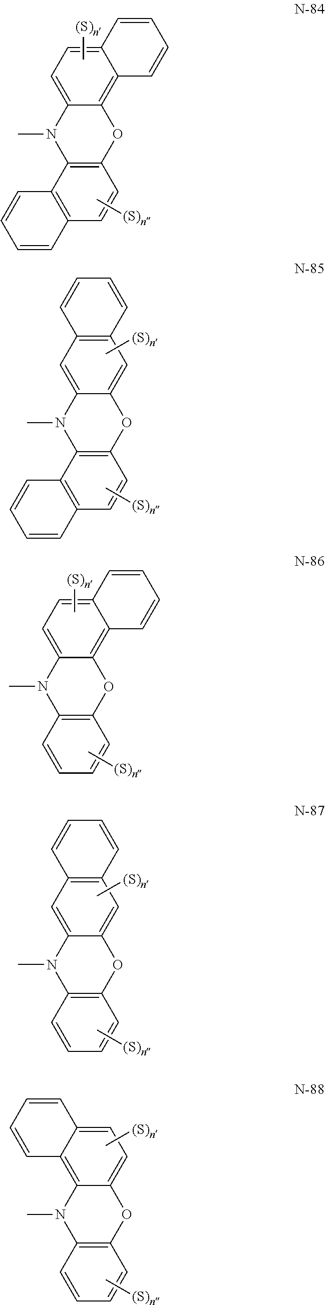

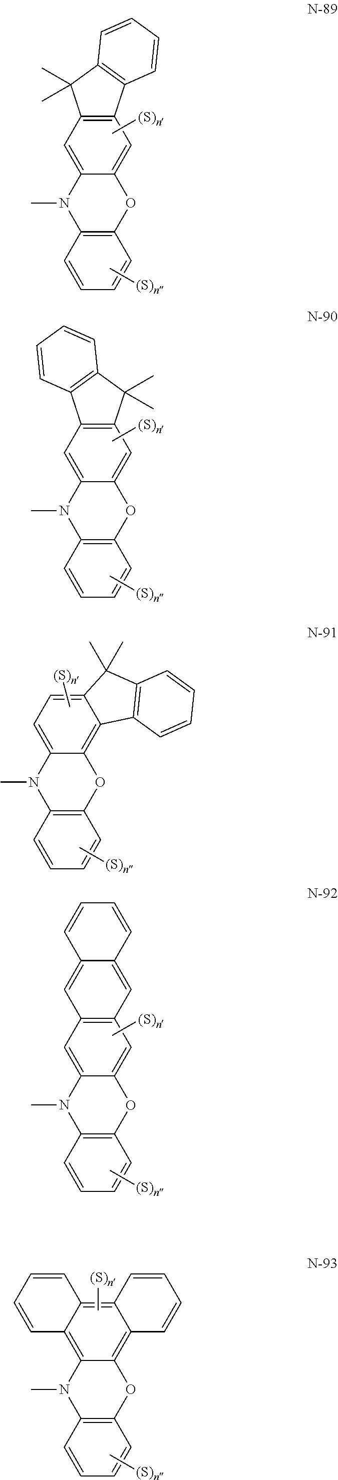

### [Chemical Formula 41]

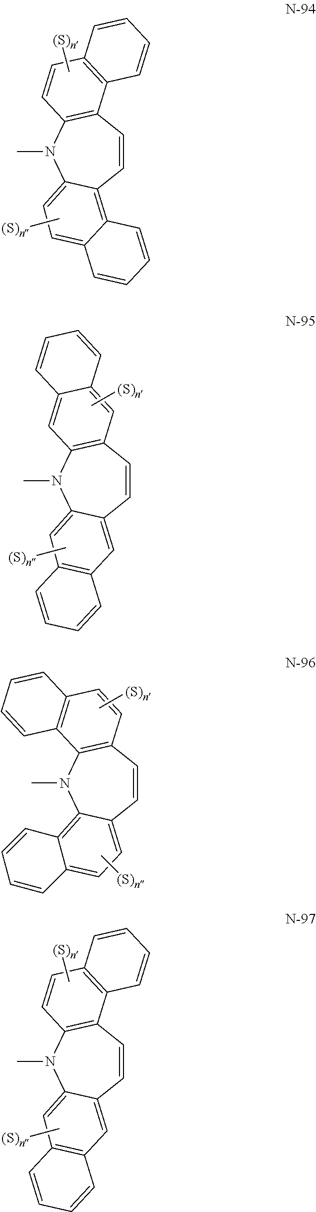

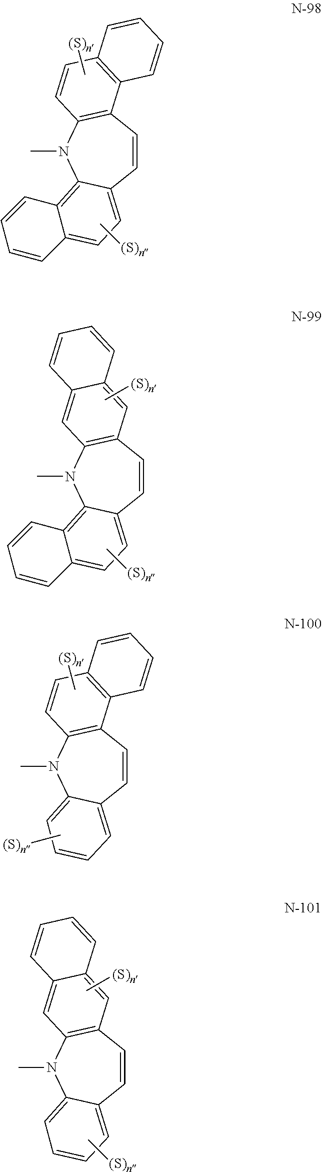

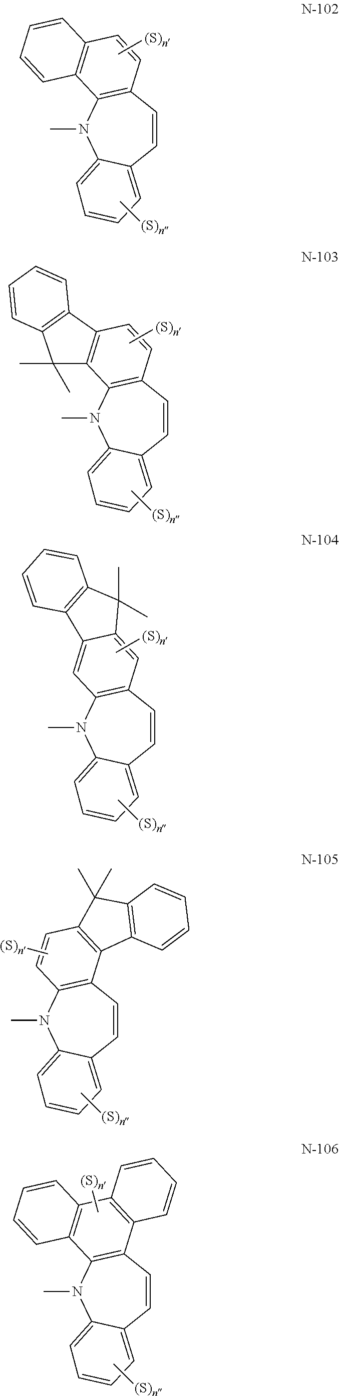

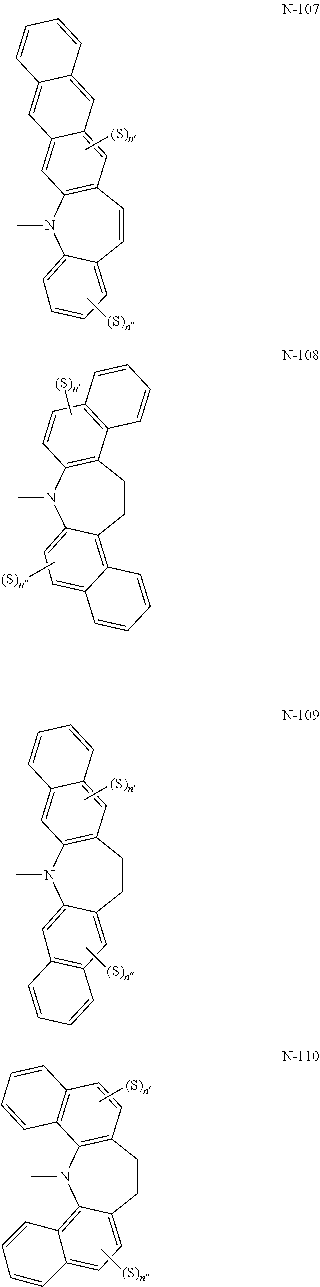

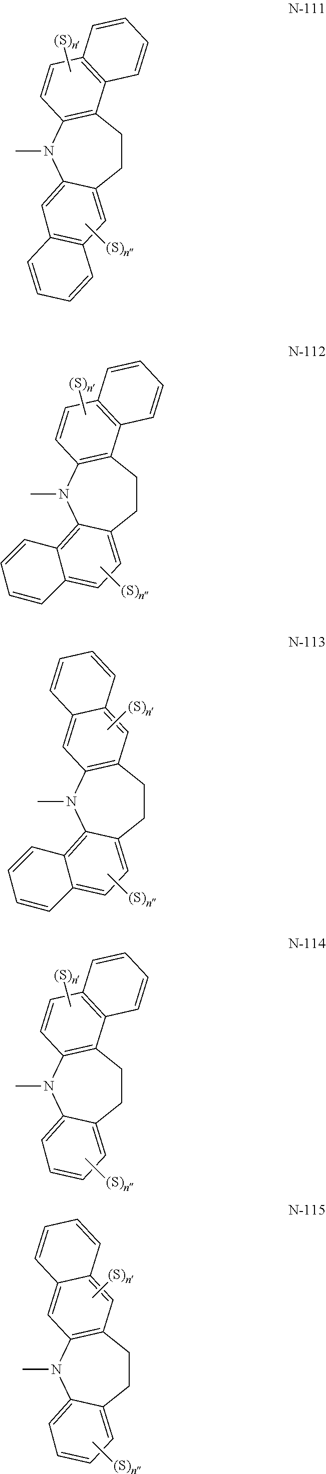

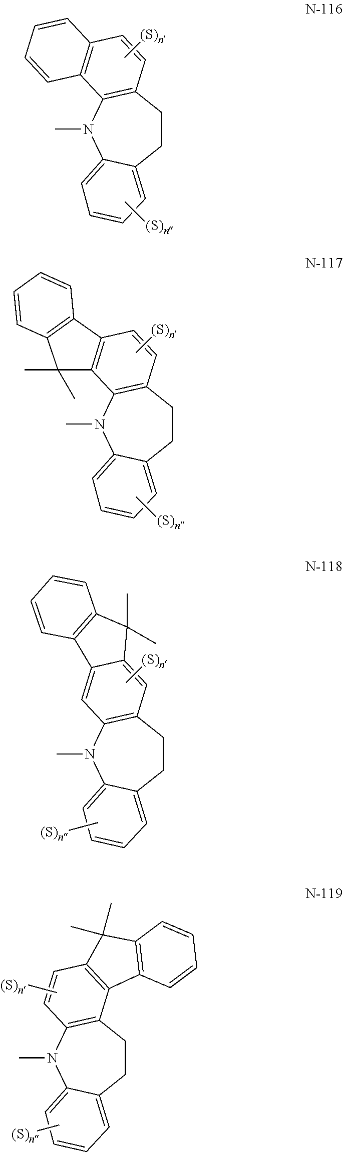

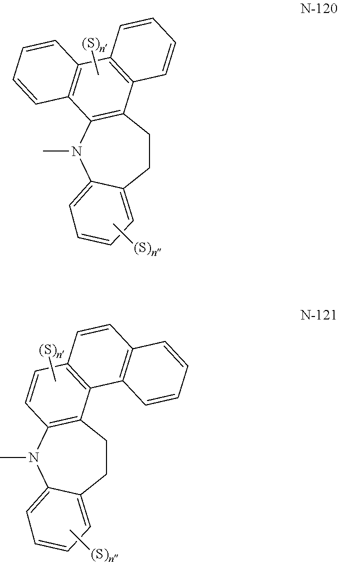

### [Chemical Formula 42]

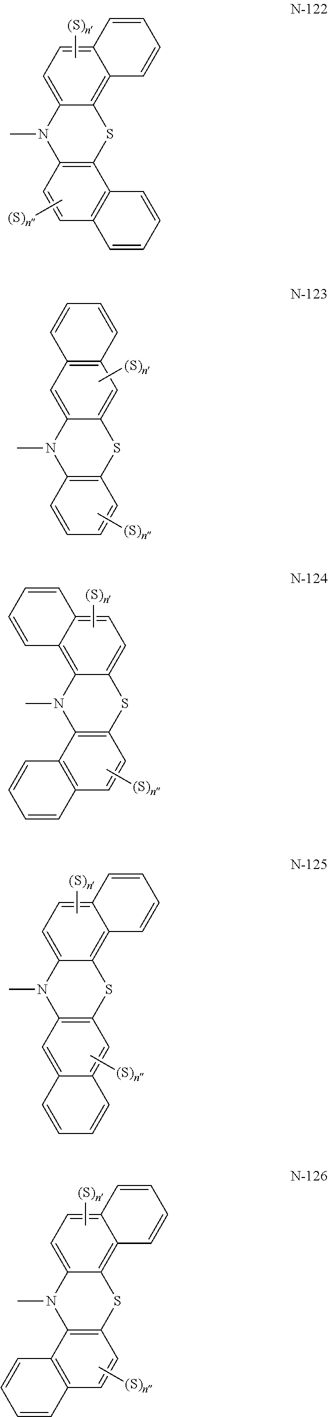

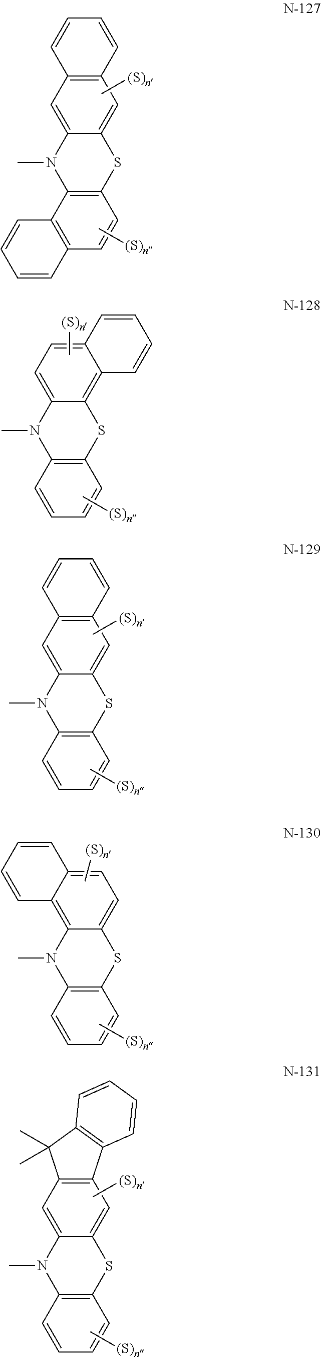

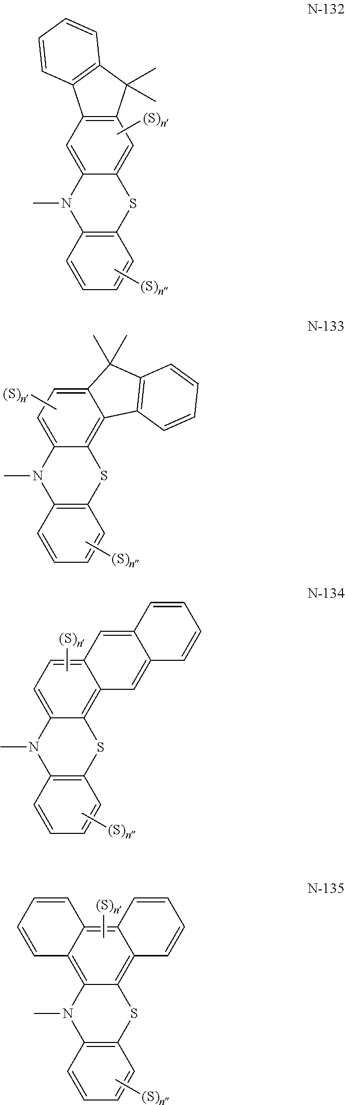

One preferred form of the compound represented by the general formula (EB-3) is a compound represented by the following general formula (EB-4). With such structure, in the case of using the compound in the electron blocking layer of the photoelectric conversion elements, the interaction with the photoelectric conversion layer is suppressed, the dark current is reduced, and the intermolecular force increases due to the increase in molecular weight, whereby the element has high heat resistance.

### [Chemical Formula 43]

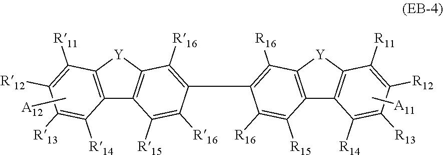

(In the general formula (EB-4), R₁₁ to R′₁₆, R₁₈, R′₁₁ to R′₁₆, and R′₁₈ each independently represent a hydrogen atom, a halogen atom, an alkyl group, an aryl group, a heterocyclic group, a hydroxyl group, an amino group, or a mercapto group, which may have a further substituent group. A₁₁ and A₁₂ each independently represent a substituent group represented by the general formula (A-1), and substitute as any one of R₁₁ to R₁₄, and any one of R′₁₁ to R′₁₄. Y respectively independently represents a carbon atom, a nitrogen atom, an oxygen atom, a sulfur atom, or a silicon atom, which may have a further substituent group.)

In the general formula (EB-4), R₁₁ to R′₁₆, R₁₈, R′₁₁ to R′₁₆, R′₁₈, Y, A₁₁, and A₁₂ have the same meanings as those of R₁₁ to R′₁₆, R₁₈, R′₁₁ to R′₁₆, R′₁₈, Y, A₁₁, and A₁₂ in the general formula (EB-3), and the preferred ranges thereof are also the same.

In one preferred form of the compound represented by the formula (EB-3), in the general formula (EB-3), Y each independently represents, —C(R₂₁) (R₂₂)—, —Si(R₂₃) (R₂₄)—, an oxygen atom, or sulfur atom, and in the group represented by the general formula (A-1), Ra₁ to Ra₈ each independently represent a hydrogen atom, a halogen atom, or an alkyl group. The use of the compounds of this form in the electron blocking layer of the photoelectric conversion elements suppresses the interaction with the photoelectric conversion layer, the dark current is reduced, and the intermolecular force is increased due to the increase in molecular weight, whereby the element has a high heat resistance. In particularly, it is preferred that Y each independently represents —C(R₂₁)(R₂₂)—, and that R₂₁ and R₂₂ each independently represent an alkyl group, an aryl group, or a heterocyclic group.

In another embodiment of the compounds represented by the general formula (EB-3) and the formula (EB-4), in the general formula (EB-3), Y each independently represents —N(R₂₀)—, R₂₀ is preferably an alkyl group, an aryl group, or a heterocyclic group. The use of the compounds of this form in the electron blocking layer can have the effect of obtaining the element with the high response speed.

Further, in one preferred form of the compound represented by the general formula (EB-3), and the compound represented by the general formula (EB-4), the substituent group represented by the general formula (A-1) independently substitutes for each of the R₁₂ and R′₁₂. Symmetry of the molecule is enhanced, and the melting point and glass transition point thereof is higher.

It is preferred that in the formula (A-1), n is 1 or 2. The use of the compounds of this form in the electron blocking layer of the photoelectric conversion elements suppresses the interaction with the photoelectric conversion layer, the dark current is reduced, and the intermolecular force is increased by the high molecular weight, whereby the element has high heat resistance.

In particular, it is preferred that in the general formula (A-1), at least one of Ra₁ and Ra₆ respectively independently represents the substituent group (S₁₁). Thus, an active site is protected, thereby improving the chemical stability of the compound.

The ionization potential (Ip) of the compounds represented by the general formulas (EB-3) and (EB-4) needs to be smaller than Ip of material that serves to transport holes in the photoelectric conversion layer in use for the electron blocking layer because the holes are required to be accepted from the material for hole transport of the photoelectric conversion layer without obstacle. In particular, when selecting absorption material having sensitivity in the visible region, in order to meet the more materials, an ionization potential of the compound according to the present invention is preferably 5.8 eV or less. By setting Ip to 5.8 eV or less, the effects of achieving the high charge collection efficiency, and the high speed response is obtained without generating an obstacle against charge transport.

Also, Ip is preferably equal to or more than 4.9 eV, and more preferably equal to or more than 5.0 eV. By setting Ip to 4.9 eV or more, a higher dark-current suppression effect can be obtained. In addition, Ip of each compound can be measured by ultraviolet photoelectron spectroscopy (UPS) or atmospheric photoelectron spectrometer (for example, AC-2 etc. made by Riken Keiki).

The Ip of the compound of the present invention can be in the above range by changing the substituent group bonded to the skeleton, or the like.

Next, a description will be given the compound represented by the formula (EB-2).

### [Chemical Formula 30]

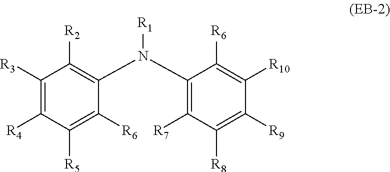

(In the formula, R₁ represents an alkyl group, an aryl group, or a heterocyclic group, which may have a substituent group. R₀ and R₂ to R₁₀ each independently represent a hydrogen atom or a substituent group.)

In the general formula (EB-2), R₁ is preferably an aryl group, like (EB-1).

R₀ and R₂ to R₁₀ each independently represent a hydrogen atom or a substituent group. A specific example of the substituent group includes the substituent W described below. Preferably examples of the substituent group include a halogen atom, an alkyl group, an aryl group, a heterocyclic group, a hydroxyl group, an amino group, or a mercapto group, more preferably a halogen atom, an alkyl group, an aryl group, or a heterocyclic group, further preferably a fluorine atom, an alkyl group, an aryl group, particularly preferably an alkyl group, an aryl group, and most preferably an alkyl group.

At least two of R₀ and R₂ to R₁₀ may be bonded to each other to form a ring. An example of the formed ring is the ring R to be described later.

In the following, specific examples of the compounds represented by the above-mentioned general formula (EB-1), (EB-2), (EB-3), or (EB-4) are described. However, the present invention is not limited to the specific examples below.

### [Chemical Formula 44]

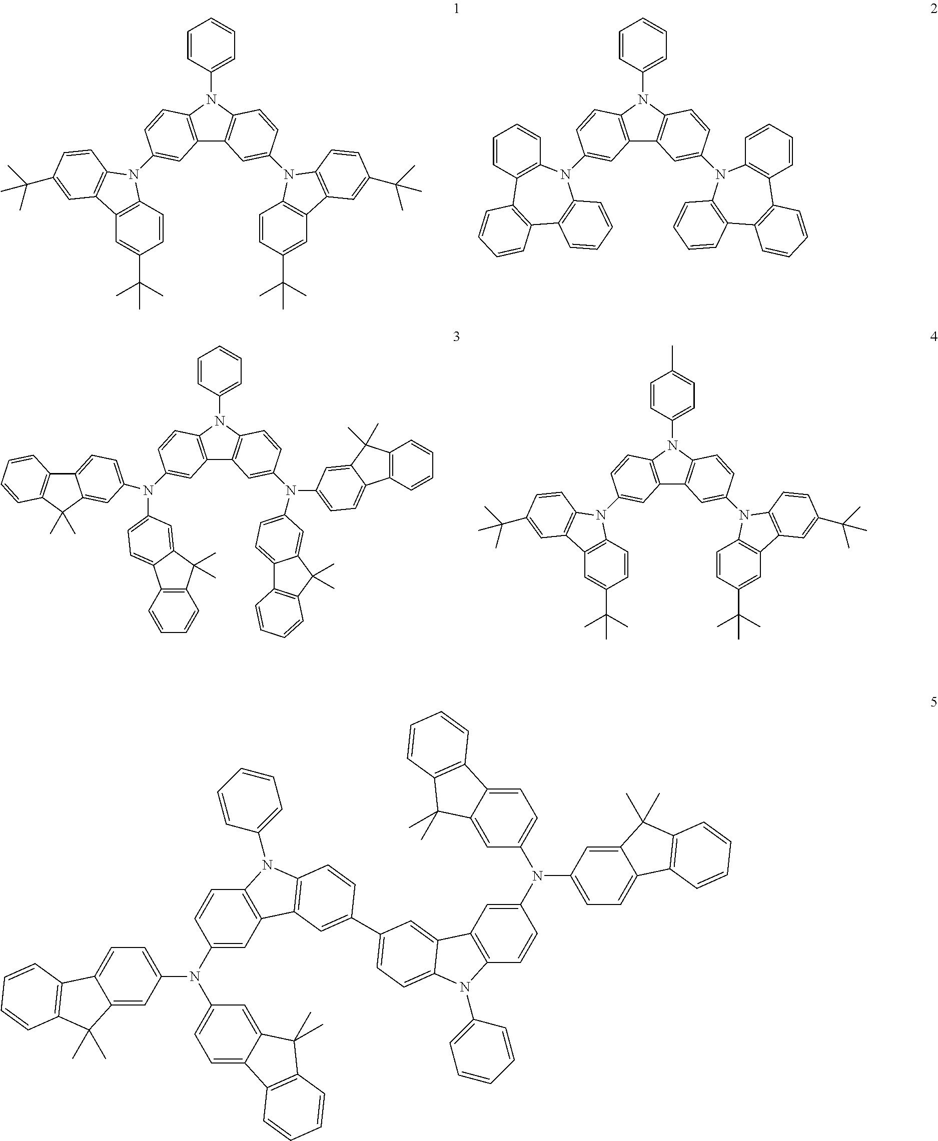

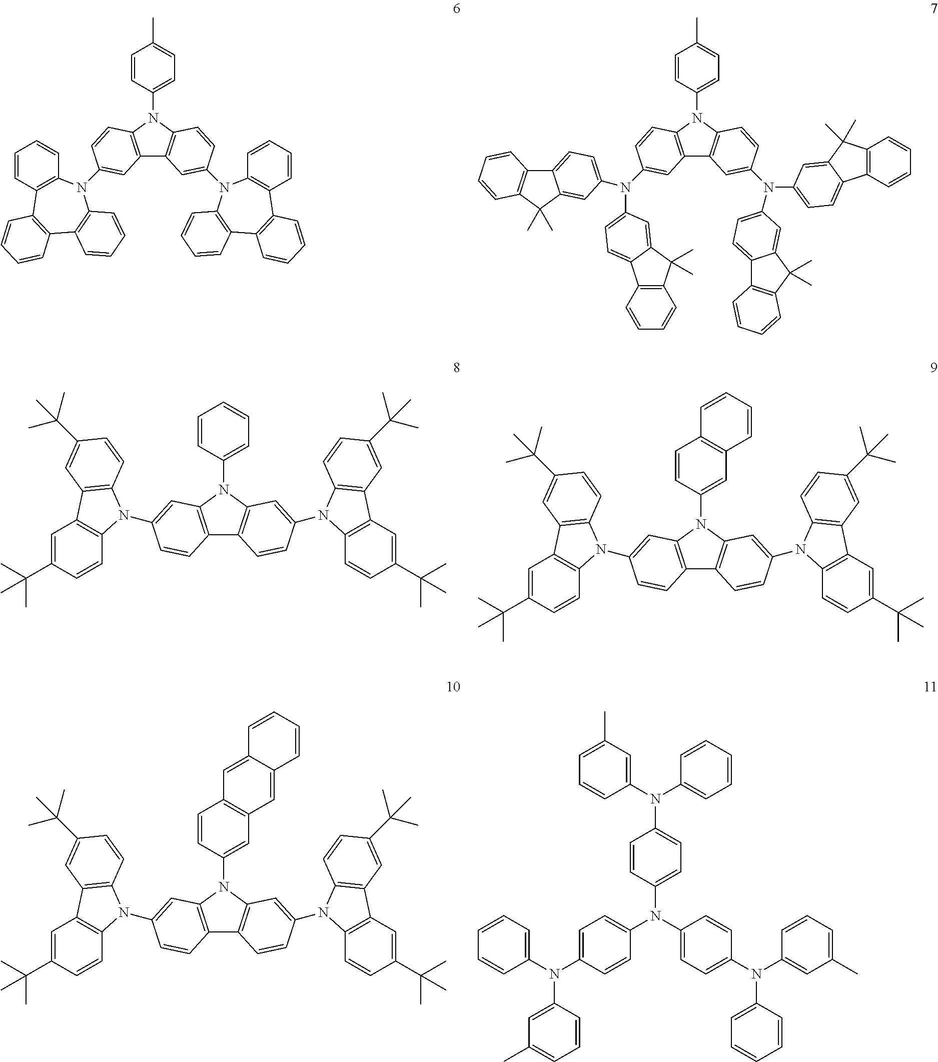

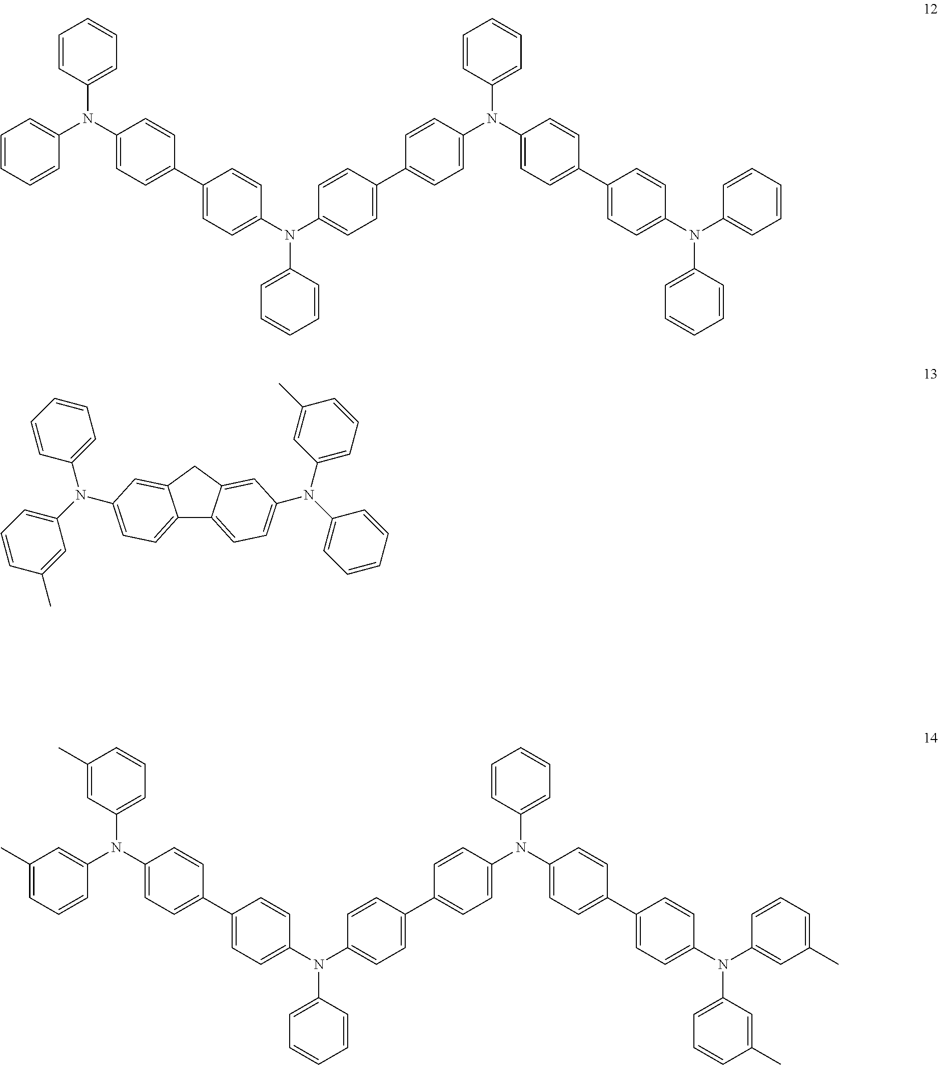

### [Chemical Formula 45]

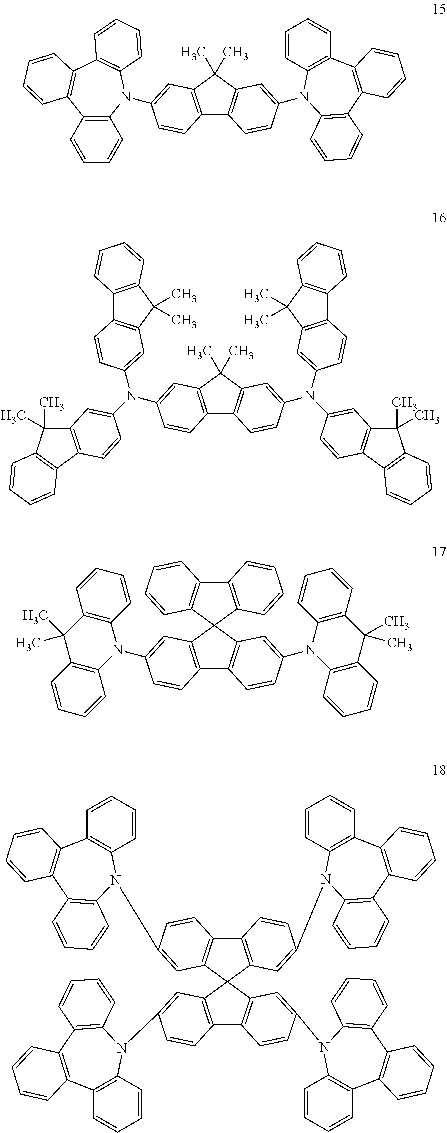

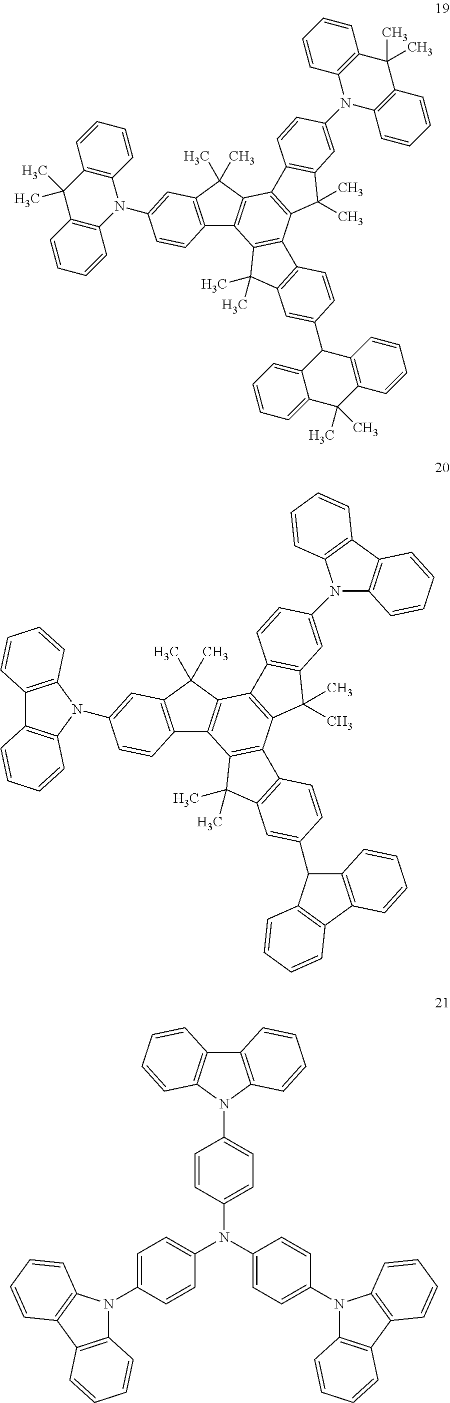 [SMILES: CC1(C)c2ccccc2C(c2ccc3c(c2)C(C)(C)c2c-3c3c(c4c2-c2ccc(N5c6ccccc6C(C)(C)c6ccccc65)cc2C4(C)C)-c2ccc(N4c5ccccc5C(C)(C)c5ccccc54)cc2C3(C)C)c2ccccc21 ; CC1(C)c2cc(C3c4ccccc4-c4ccccc43)ccc2-c2c1c1c(c3c2C(C)(C)c2cc(-n4c5ccccc5c5ccccc54)ccc2-3)C(C)(C)c2cc(-n3c4ccccc4c4ccccc43)ccc2-1 ; c1ccc2c(c1)c1ccccc1n2-c1ccc(N(c2ccc(-n3c4ccccc4c4ccccc43)cc2)c2ccc(-n3c4ccccc4c4ccccc43)cc2)cc1]

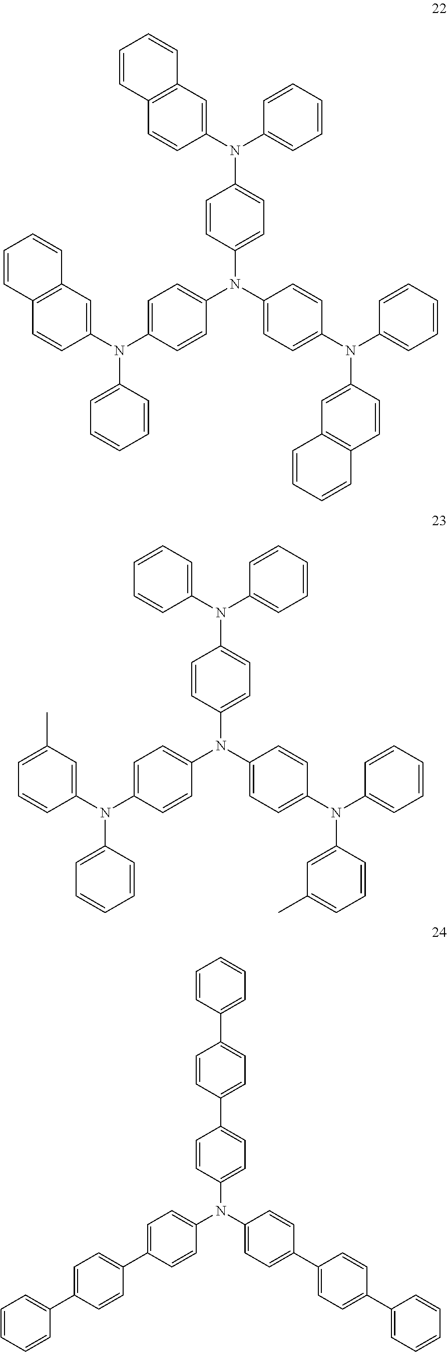 [SMILES: c1ccc(N(c2ccc(N(c3ccc(N(c4ccccc4)c4ccc5ccccc5c4)cc3)c3ccc(N(c4ccccc4)c4ccc5ccccc5c4)cc3)cc2)c2ccc3ccccc3c2)cc1 ; Cc1cccc(N(c2ccccc2)c2ccc(N(c3ccc(N(c4ccccc4)c4ccccc4)cc3)c3ccc(N(c4ccccc4)c4cccc(C)c4)cc3)cc2)c1 ; c1ccc(-c2ccc(-c3ccc(N(c4ccc(-c5ccc(-c6ccccc6)cc5)cc4)c4ccc(-c5ccc(-c6ccccc6)cc5)cc4)cc3)cc2)cc1]

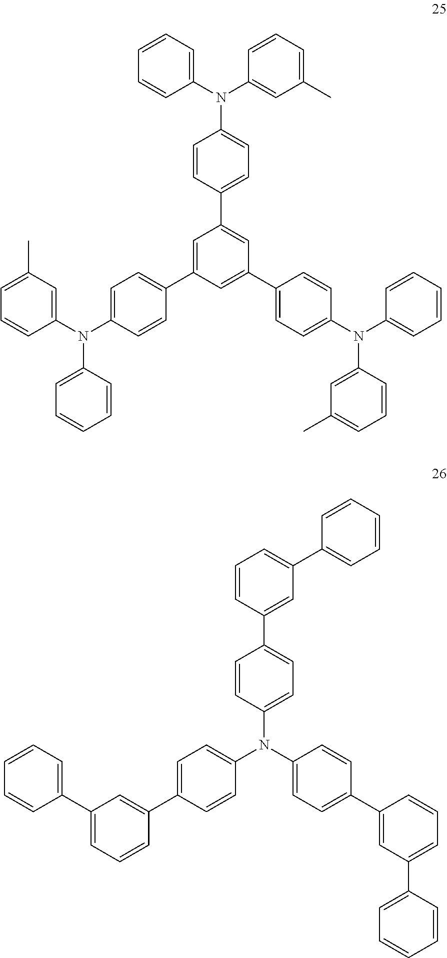 [SMILES: Cc1cccc(N(c2ccccc2)c2ccc(-c3cc(-c4ccc(N(c5ccccc5)c5cccc(C)c5)cc4)cc(-c4ccc(N(c5ccccc5)c5cccc(C)c5)cc4)c3)cc2)c1 ; c1ccc(-c2cccc(-c3ccc(N(c4ccc(-c5cccc(-c6ccccc6)c5)cc4)c4ccc(-c5cccc(-c6ccccc6)c5)cc4)cc3)c2)cc1]

### [Chemical Formula 46]

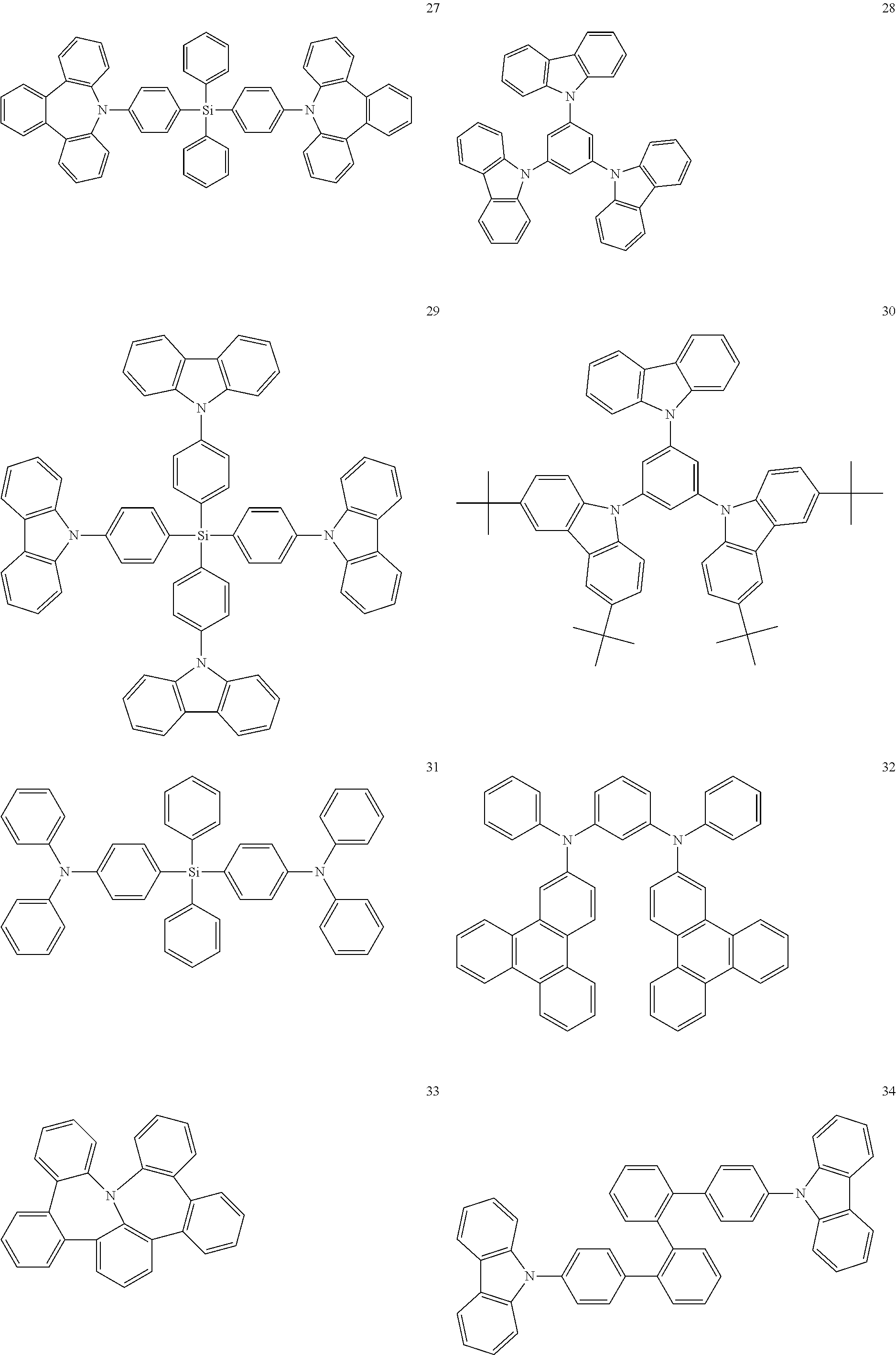

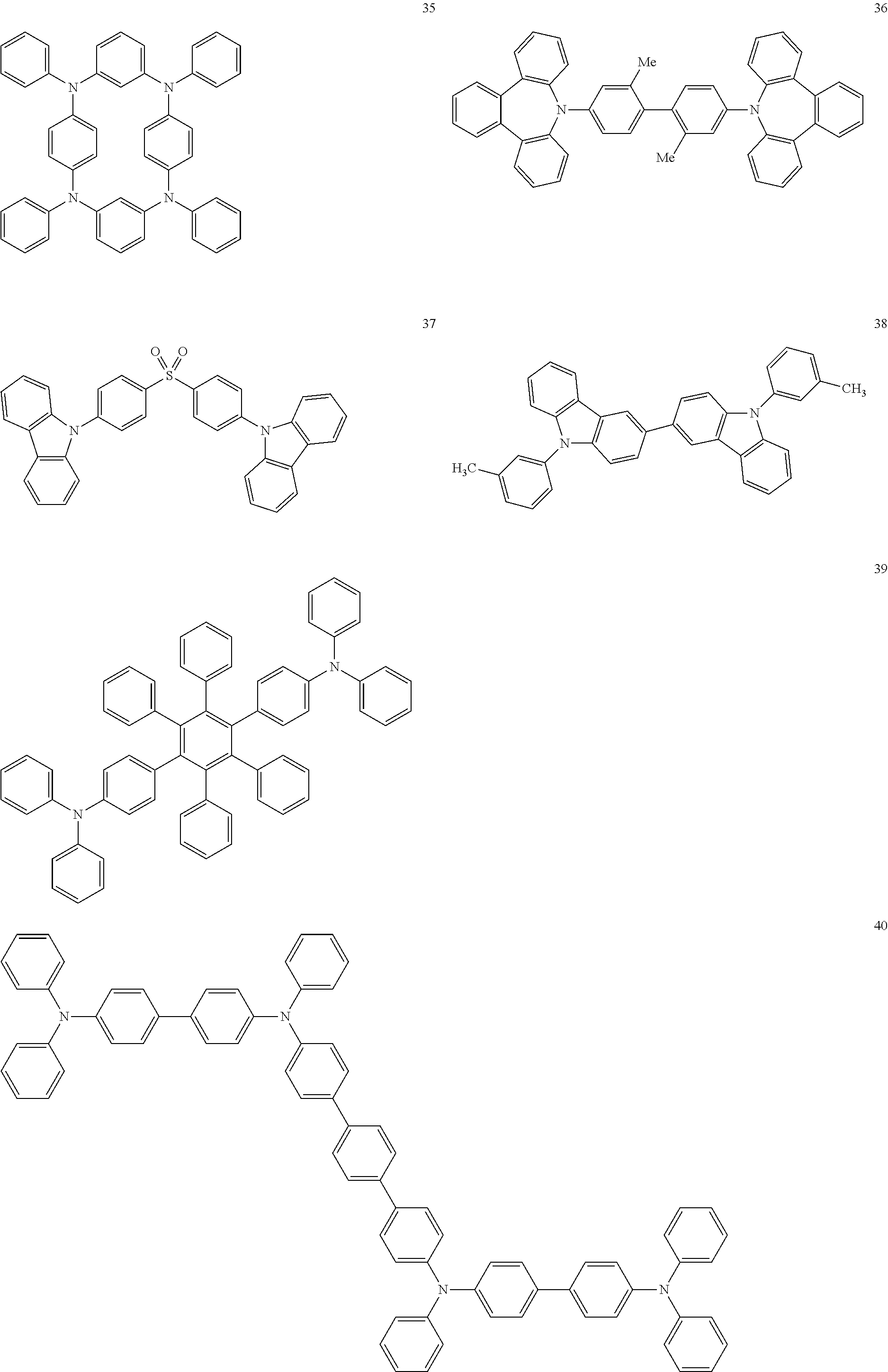

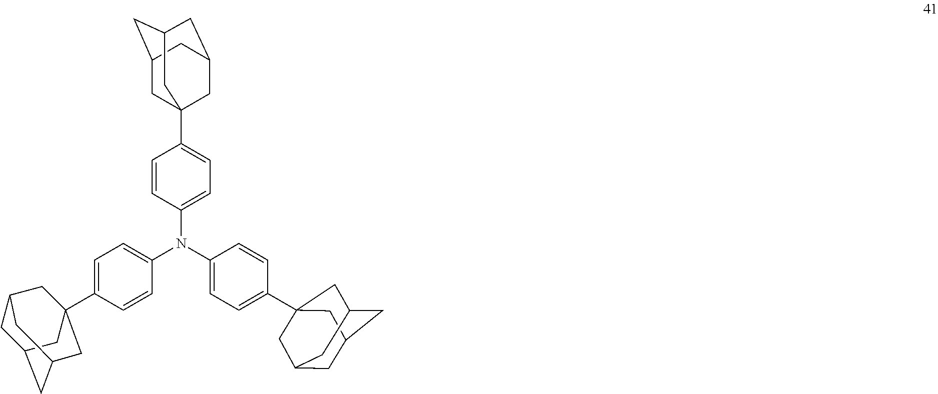 [SMILES: c1cc(C23CC4CC(CC(C4)C2)C3)ccc1N(c1ccc(C23CC4CC(CC(C4)C2)C3)cc1)c1ccc(C23CC4CC(CC(C4)C2)C3)cc1]

### [Chemical Formula 47]

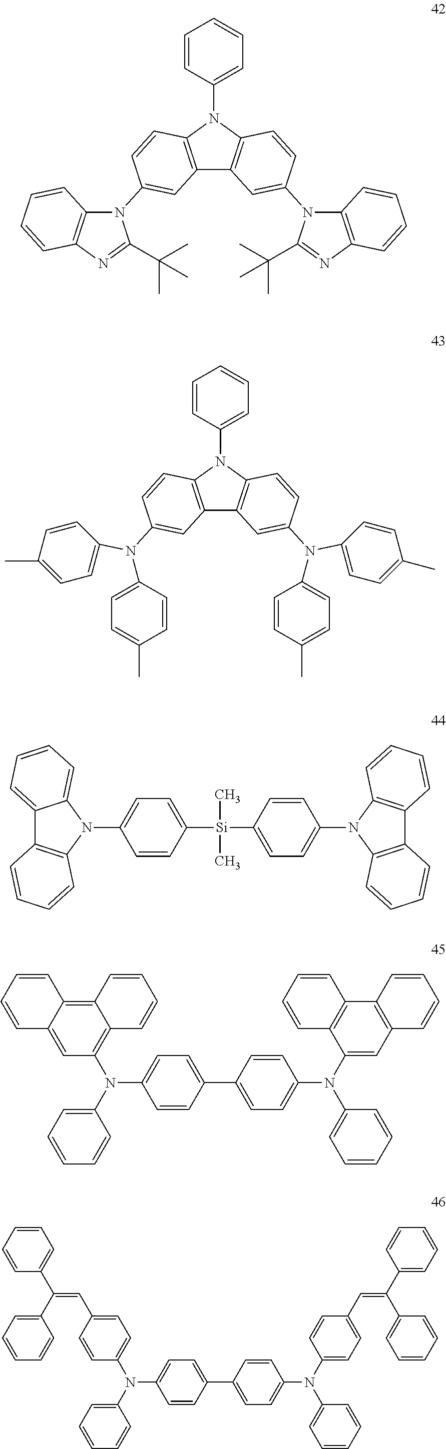

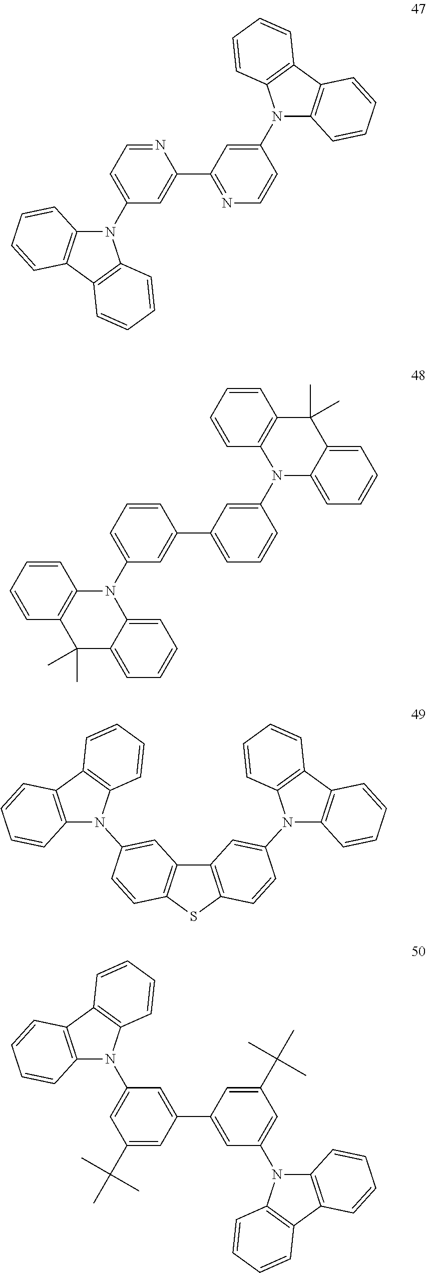 [SMILES: c1ccc2c(c1)c1ccccc1n2-c1ccnc(-c2cc(-n3c4ccccc4c4ccccc43)ccn2)c1 ; CC1(C)c2ccccc2N(c2cccc(-c3cccc(N4c5ccccc5C(C)(C)c5ccccc54)c3)c2)c2ccccc21 ; c1ccc2c(c1)c1ccccc1n2-c1ccc2sc3ccc(-n4c5ccccc5c5ccccc54)cc3c2c1 ; CC(C)(C)c1cc(-c2cc(-n3c4ccccc4c4ccccc43)cc(C(C)(C)C)c2)cc(-n2c3ccccc3c3ccccc32)c1]

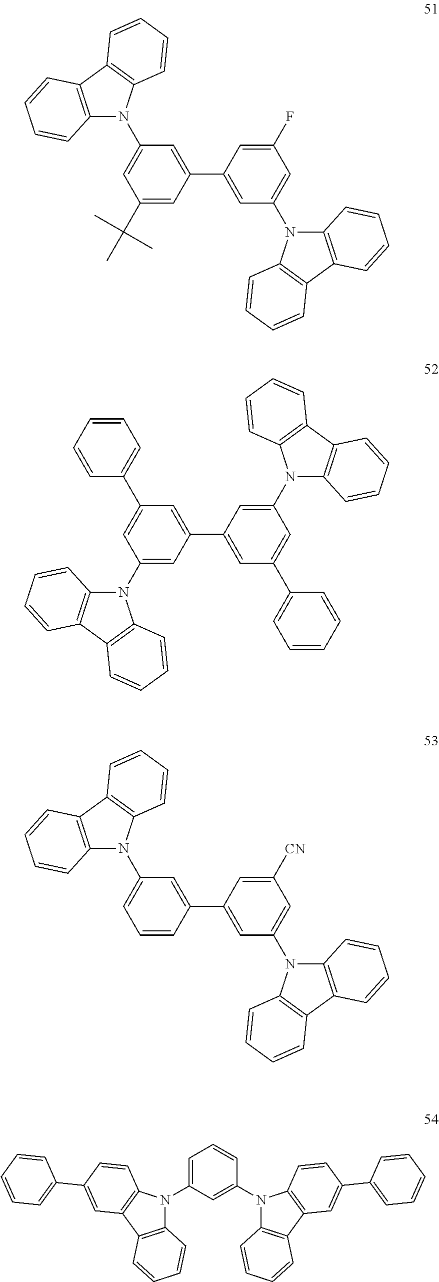 [SMILES: CC(C)(C)c1cc(-c2cc(F)cc(-n3c4ccccc4c4ccccc43)c2)cc(-n2c3ccccc3c3ccccc32)c1 ; c1ccc(-c2cc(-c3cc(-c4ccccc4)cc(-n4c5ccccc5c5ccccc54)c3)cc(-n3c4ccccc4c4ccccc43)c2)cc1 ; N#Cc1cc(-c2cccc(-n3c4ccccc4c4ccccc43)c2)cc(-n2c3ccccc3c3ccccc32)c1 ; c1ccc(-c2ccc3c(c2)c2ccccc2n3-c2cccc(-n3c4ccccc4c4cc(-c5ccccc5)ccc43)c2)cc1]

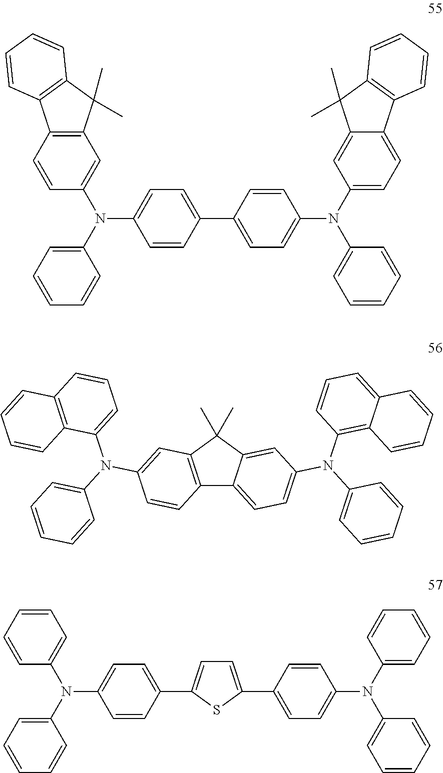 [SMILES: CC1(C)c2ccccc2-c2ccc(N(c3ccccc3)c3ccc(-c4ccc(N(c5ccccc5)c5ccc6c(c5)C(C)(C)c5ccccc5-6)cc4)cc3)cc21 ; CC1(C)c2cc(N(c3ccccc3)c3cccc4ccccc34)ccc2-c2ccc(N(c3ccccc3)c3cccc4ccccc34)cc21 ; c1ccc(N(c2ccccc2)c2ccc(-c3ccc(-c4ccc(N(c5ccccc5)c5ccccc5)cc4)s3)cc2)cc1]

### [Chemical Formula 48]

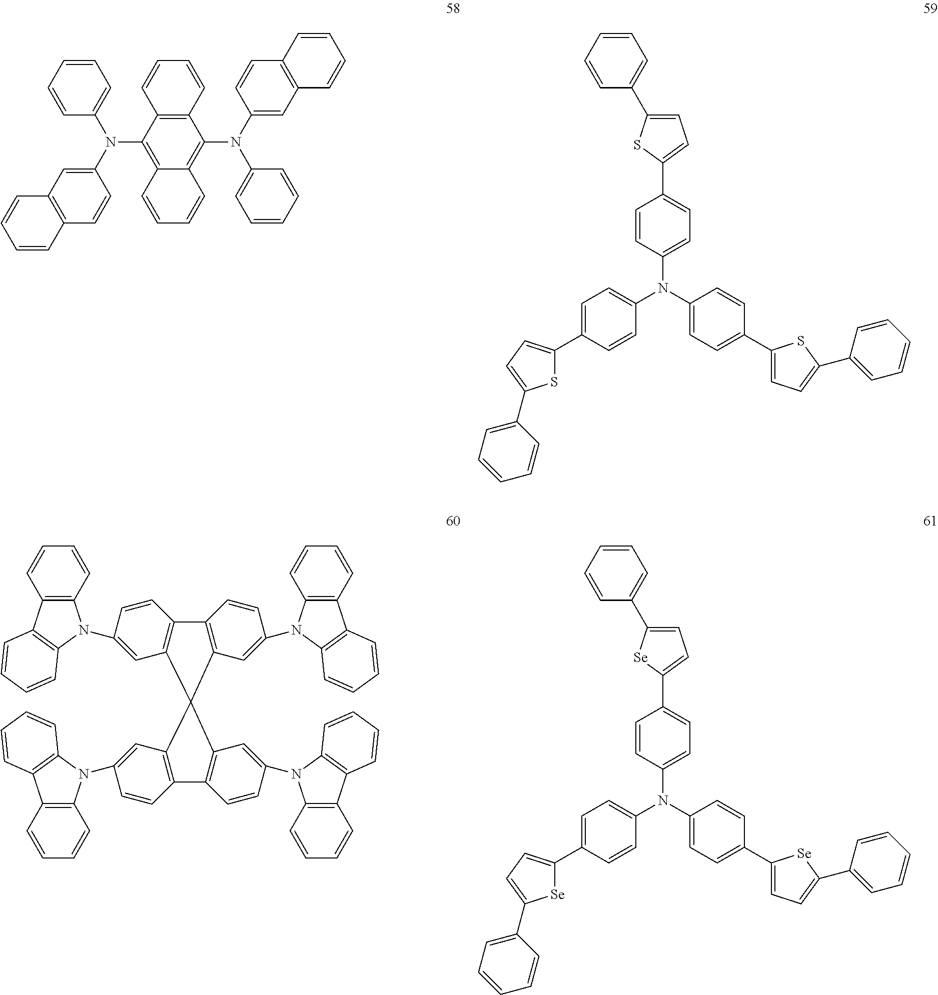 [SMILES: c1ccc(N(c2ccc3ccccc3c2)c2c3ccccc3c(N(c3ccccc3)c3ccc4ccccc4c3)c3ccccc23)cc1 ; c1ccc(-c2ccc(-c3ccc(N(c4ccc(-c5ccc(-c6ccccc6)s5)cc4)c4ccc(-c5ccc(-c6ccccc6)s5)cc4)cc3)s2)cc1 ; c1ccc2c(c1)c1ccccc1n2-c1ccc2c(c1)C1(c3cc(-n4c5ccccc5c5ccccc54)ccc3-2)c2cc(-n3c4ccccc4c4ccccc43)ccc2-c2ccc(-n3c4ccccc4c4ccccc43)cc21 ; c1ccc(-c2ccc(-c3ccc(N(c4ccc(-c5ccc(-c6ccccc6)[se]5)cc4)c4ccc(-c5ccc(-c6ccccc6)[se]5)cc4)cc3)[se]2)cc1]

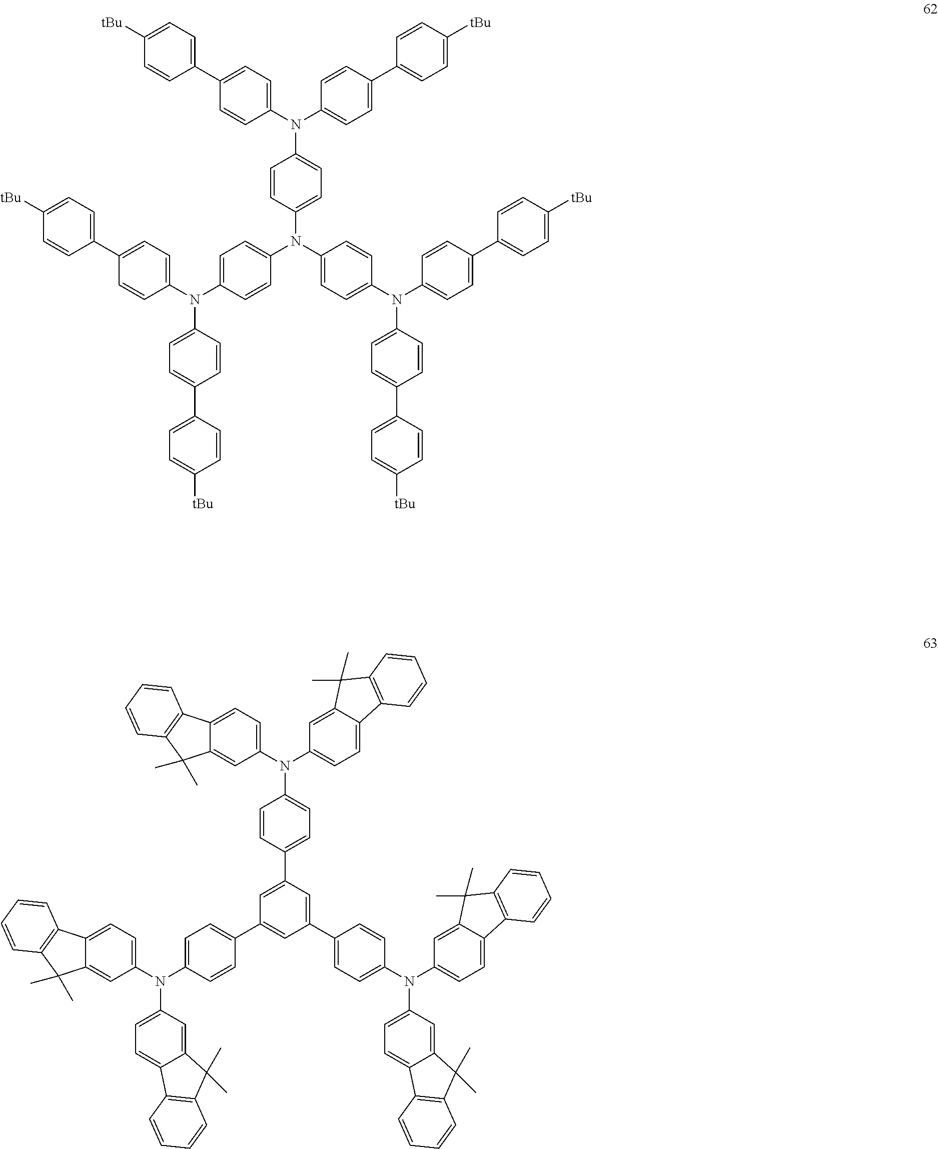 [SMILES: CC(C)(C)c1ccc(-c2ccc(N(c3ccc(-c4ccc(C(C)(C)C)cc4)cc3)c3ccc(N(c4ccc(N(c5ccc(-c6ccc(C(C)(C)C)cc6)cc5)c5ccc(-c6ccc(C(C)(C)C)cc6)cc5)cc4)c4ccc(N(c5ccc(-c6ccc(C(C)(C)C)cc6)cc5)c5ccc(-c6ccc(C(C)(C)C)cc6)cc5)cc4)cc3)cc2)cc1 ; CC1(C)c2ccccc2-c2ccc(N(c3ccc(-c4cc(-c5ccc(N(c6ccc7c(c6)C(C)(C)c6ccccc6-7)c6ccc7c(c6)C(C)(C)c6ccccc6-7)cc5)cc(-c5ccc(N(c6ccc7c(c6)C(C)(C)c6ccccc6-7)c6ccc7c(c6)C(C)(C)c6ccccc6-7)cc5)c4)cc3)c3ccc4c(c3)C(C)(C)c3ccccc3-4)cc21]

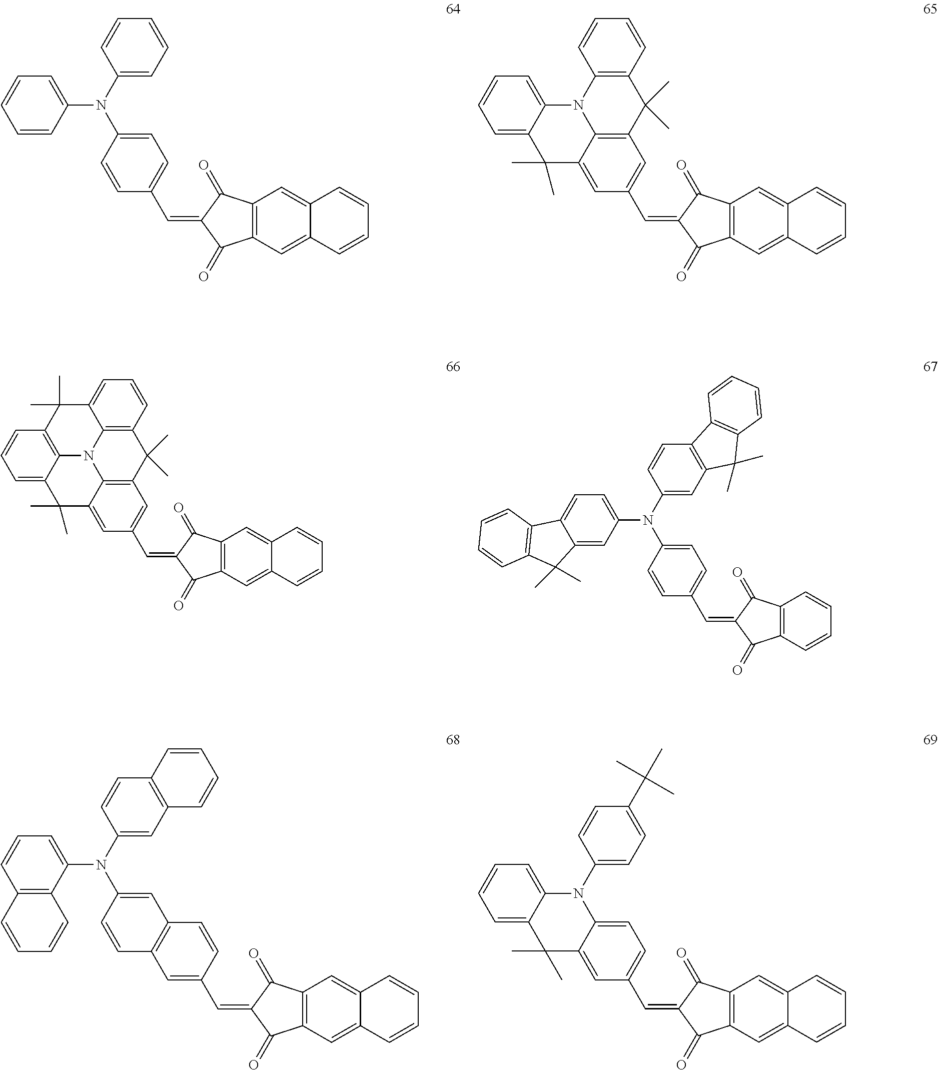 [SMILES: O=C1C(=Cc2ccc(N(c3ccccc3)c3ccccc3)cc2)C(=O)c2cc3ccccc3cc21 ; CC1(C)c2ccccc2N2c3ccccc3C(C)(C)c3cc(C=C4C(=O)c5cc6ccccc6cc5C4=O)cc1c32 ; CC1(C)c2cccc3c2N2c4c1cccc4C(C)(C)c1cc(C=C4C(=O)c5cc6ccccc6cc5C4=O)cc(c12)C3(C)C ; CC1(C)c2ccccc2-c2ccc(N(c3ccc(C=C4C(=O)c5ccccc5C4=O)cc3)c3ccc4c(c3)C(C)(C)c3ccccc3-4)cc21 ; O=C1C(=Cc2ccc3cc(N(c4ccc5ccccc5c4)c4cccc5ccccc45)ccc3c2)C(=O)c2cc3ccccc3cc21 ; CC(C)(C)c1ccc(N2c3ccccc3C(C)(C)c3cc(C=C4C(=O)c5cc6ccccc6cc5C4=O)ccc32)cc1]

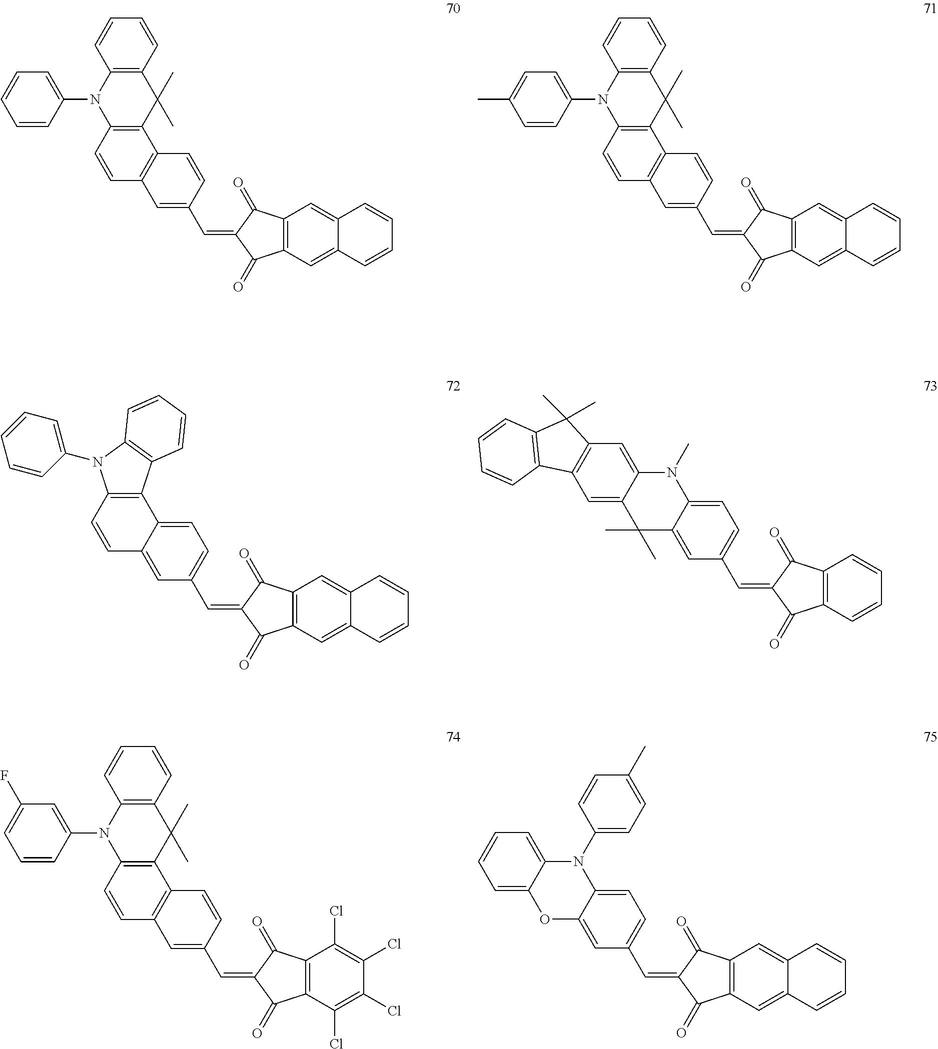 [SMILES: CC1(C)c2ccccc2N(c2ccccc2)c2ccc3cc(C=C4C(=O)c5cc6ccccc6cc5C4=O)ccc3c21 ; Cc1ccc(N2c3ccccc3C(C)(C)c3c2ccc2cc(C=C4C(=O)c5cc6ccccc6cc5C4=O)ccc32)cc1 ; O=C1C(=Cc2ccc3c(ccc4c3c3ccccc3n4-c3ccccc3)c2)C(=O)c2cc3ccccc3cc21 ; CN1c2ccc(C=C3C(=O)c4ccccc4C3=O)cc2C(C)(C)c2cc3c(cc21)C(C)(C)c1ccccc1-3 ; CC1(C)c2ccccc2N(c2cccc(F)c2)c2ccc3cc(C=C4C(=O)c5c(Cl)c(Cl)c(Cl)c(Cl)c5C4=O)ccc3c21 ; Cc1ccc(N2c3ccccc3Oc3cc(C=C4C(=O)c5cc6ccccc6cc5C4=O)ccc32)cc1]

### [Chemical Formula 49]

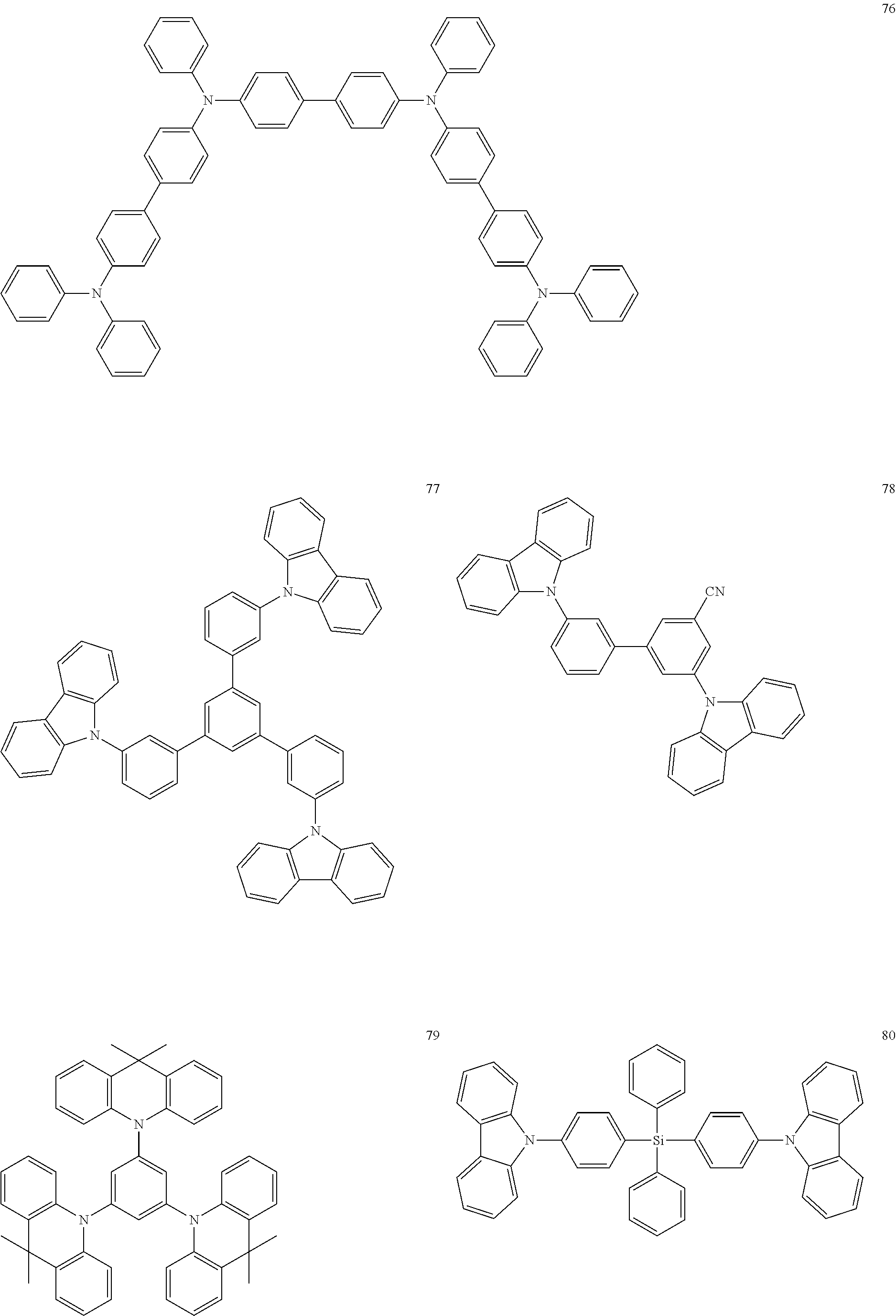 [SMILES: c1ccc(N(c2ccccc2)c2ccc(-c3ccc(N(c4ccccc4)c4ccc(-c5ccc(N(c6ccccc6)c6ccc(-c7ccc(N(c8ccccc8)c8ccccc8)cc7)cc6)cc5)cc4)cc3)cc2)cc1 ; c1cc(-c2cc(-c3cccc(-n4c5ccccc5c5ccccc54)c3)cc(-c3cccc(-n4c5ccccc5c5ccccc54)c3)c2)cc(-n2c3ccccc3c3ccccc32)c1 ; N#Cc1cc(-c2cccc(-n3c4ccccc4c4ccccc43)c2)cc(-n2c3ccccc3c3ccccc32)c1 ; CC1(C)c2ccccc2N(c2cc(N3c4ccccc4C(C)(C)c4ccccc43)cc(N3c4ccccc4C(C)(C)c4ccccc43)c2)c2ccccc21 ; c1ccc([Si](c2ccccc2)(c2ccc(-n3c4ccccc4c4ccccc43)cc2)c2ccc(-n3c4ccccc4c4ccccc43)cc2)cc1]

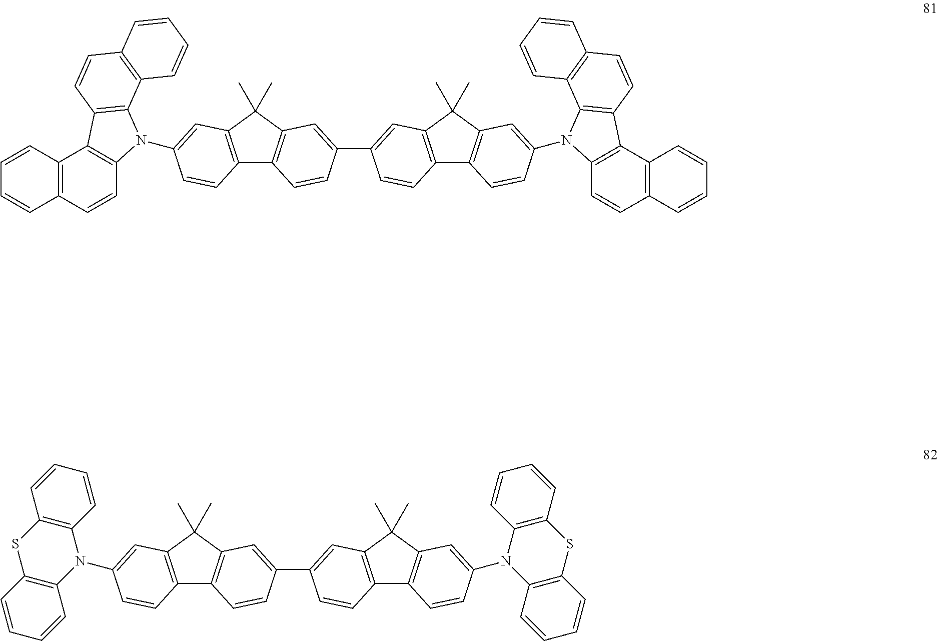 [SMILES: CC1(C)c2cc(-c3ccc4c(c3)C(C)(C)c3cc(-n5c6ccc7ccccc7c6c6ccc7ccccc7c65)ccc3-4)ccc2-c2ccc(-n3c4ccc5ccccc5c4c4ccc5ccccc5c43)cc21 ; CC1(C)c2cc(-c3ccc4c(c3)C(C)(C)c3cc(N5c6ccccc6Sc6ccccc65)ccc3-4)ccc2-c2ccc(N3c4ccccc4Sc4ccccc43)cc21]

Now, in particular, a description will be given of specific examples of the structure represented by the general formula (A-1) according to the present invention ((B-1) to (B-136)) and specific examples of the compound represented by the general formula (EB-3) or (EB-4). However, the invention is not limited to the specific examples below. In the following formulas (a) to (t), when the A₁₁ is not the same as A₁₂, R₂₀ is not the same as R′₂₀, and R₂₃, R₂₄ and R′₂₃, R′₂₄ are not the same, respectively, any combination other than the embodied structure is also allowed.

In examples of compounds described below, each reference character indicates a corresponding compound as follows: Me: methyl group, Et: ethyl group, i-Pr: isopropyl group, n-Bu: n-butyl group, t-Bu: tert-butyl group, Ph: phenyl group, 2-tol: 2-tolyl group, 3-tol: 3-tolyl group, 4-tol: 4-tolyl group, 1-Np: 1-naphthyl group, 2-Np: 2-naphthyl group, 2-An: 2-anthryl group, 2-Fn: 2-fluorenyl group.

### [Chemical Formula 50]

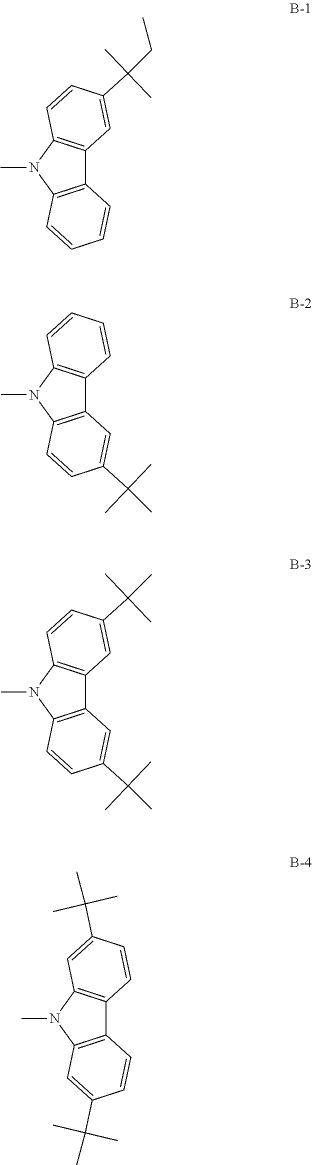

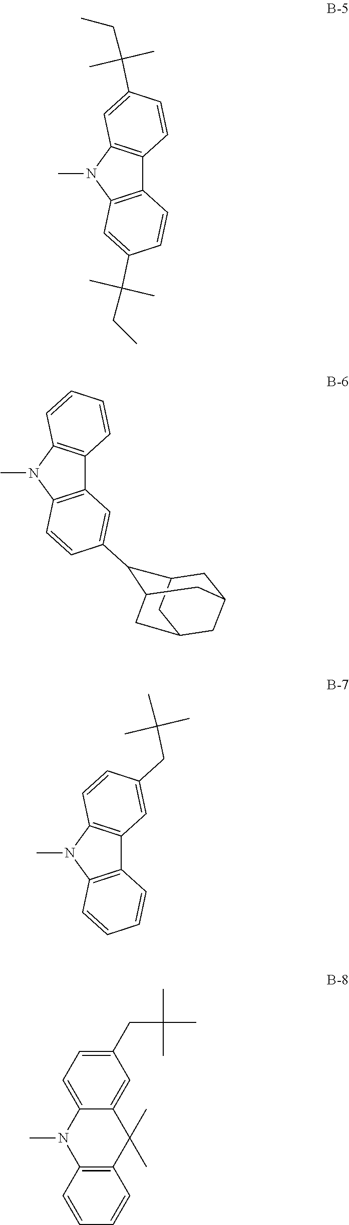

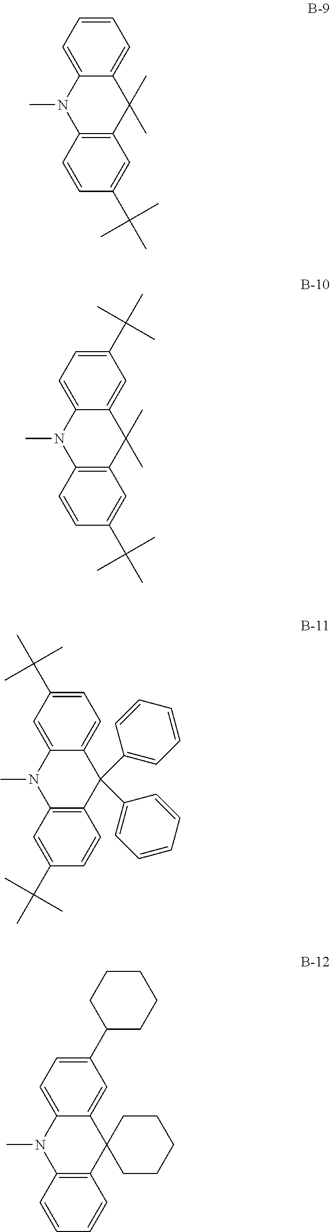

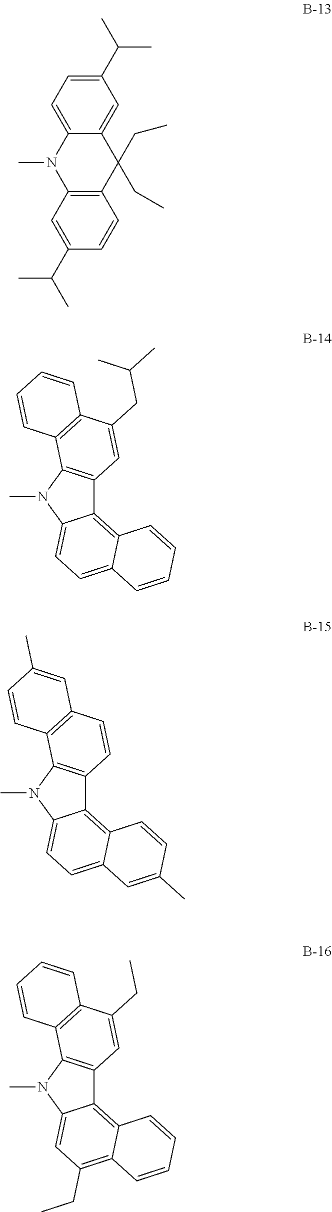

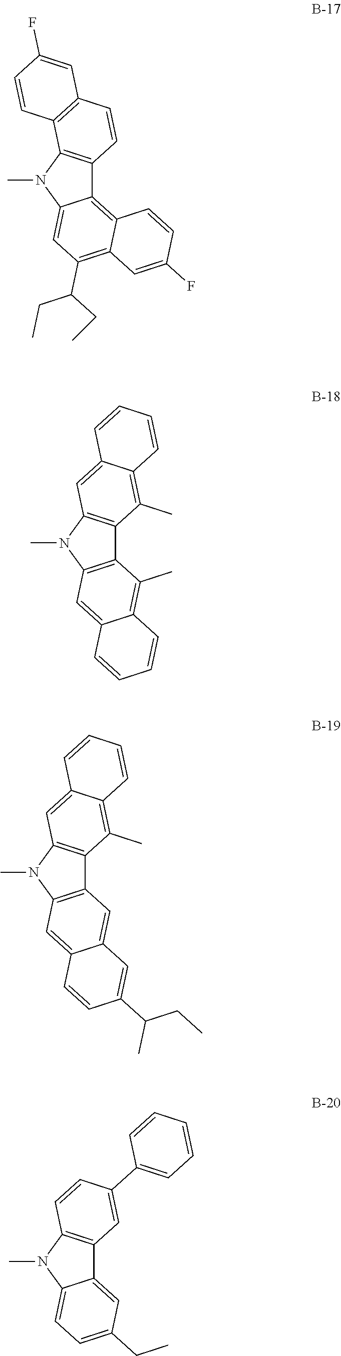

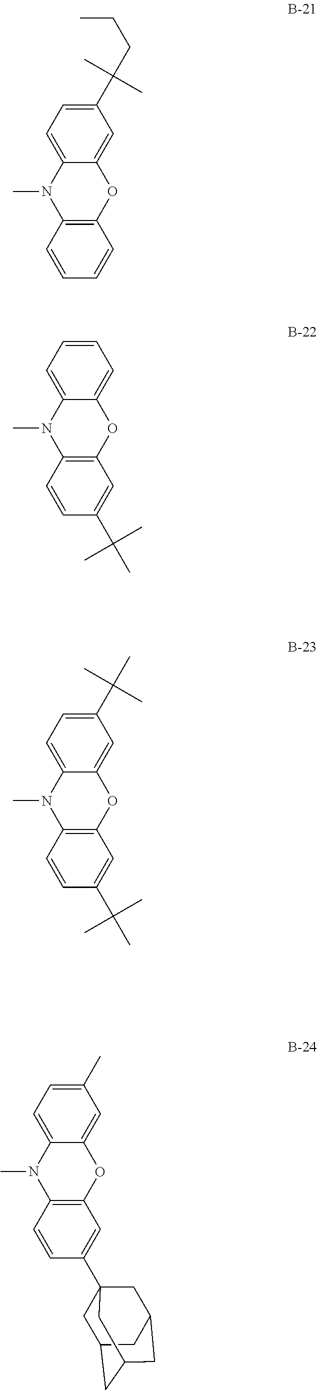

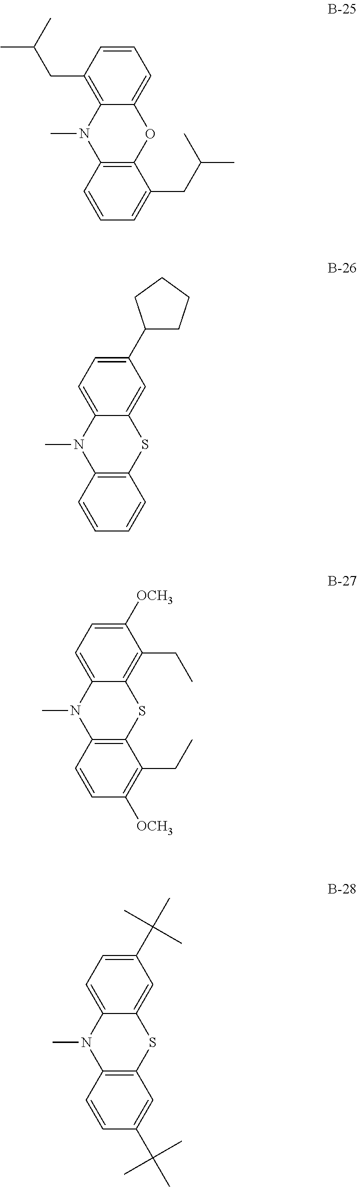

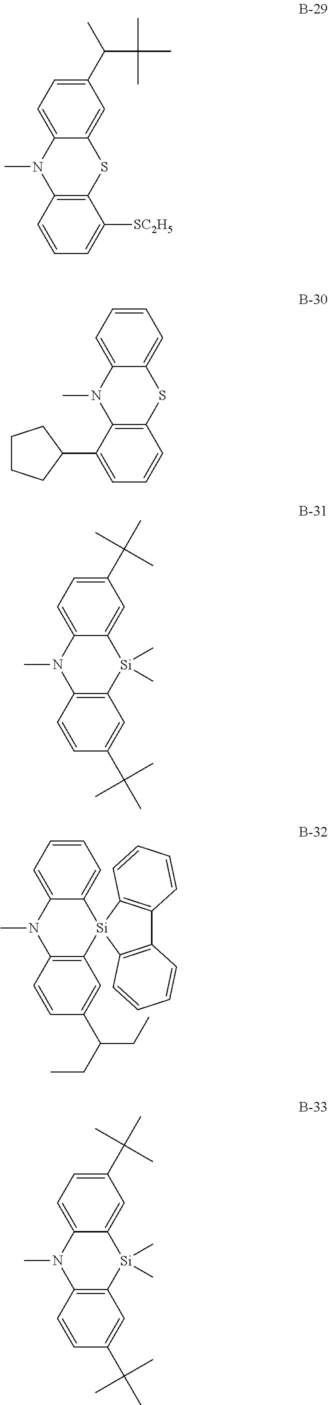

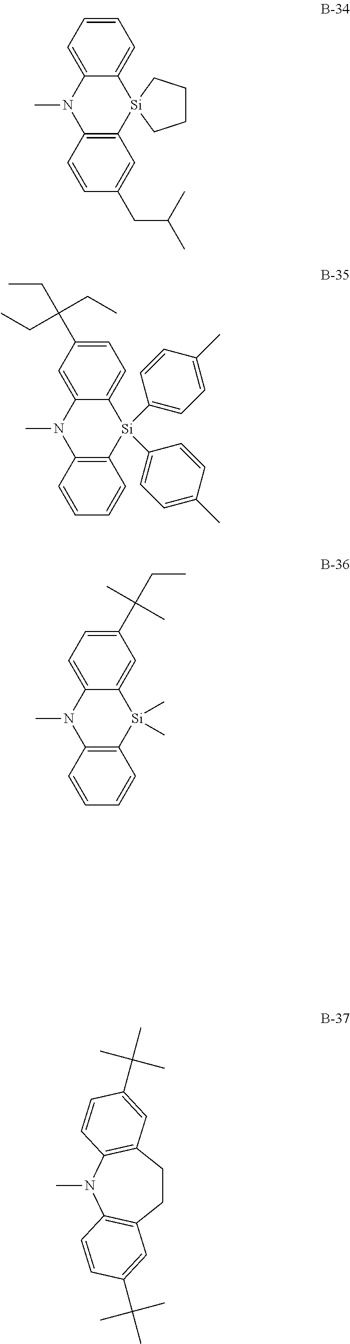

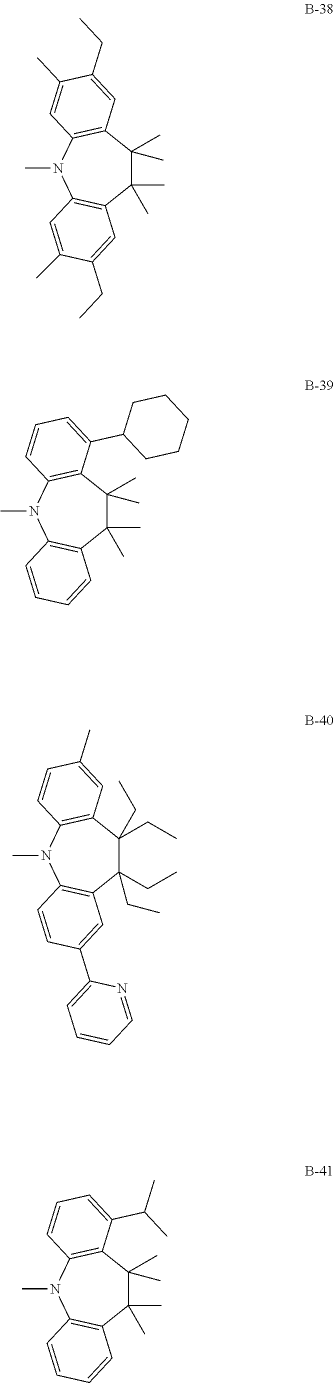

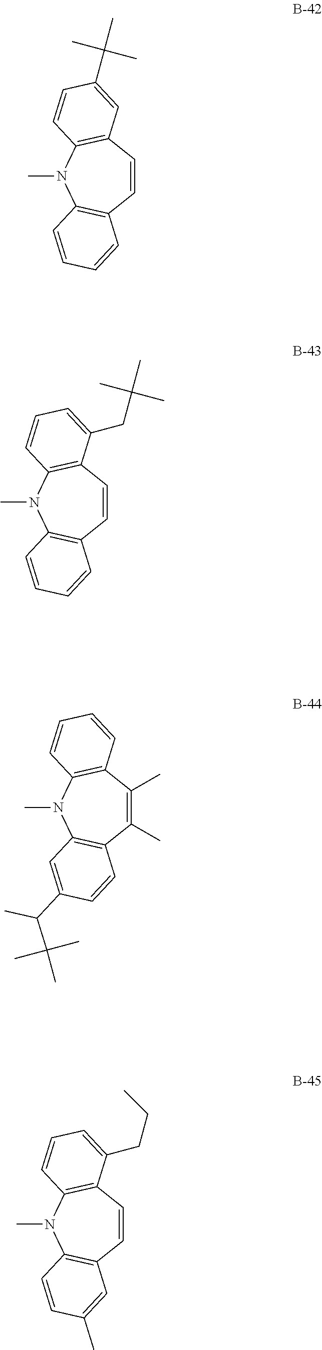

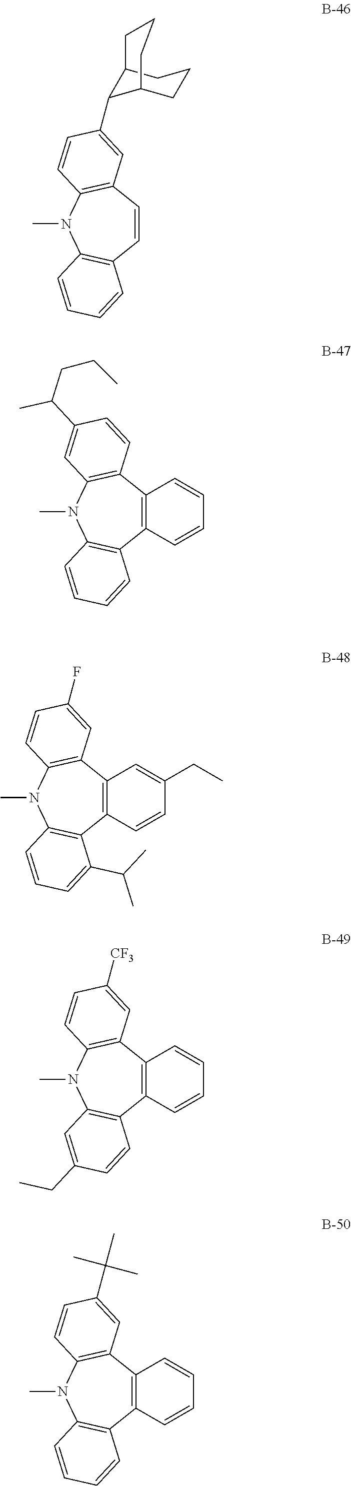

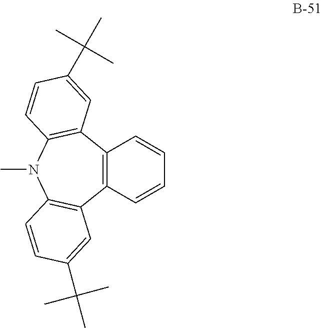 [SMILES: CN1c2ccc(C(C)(C)C)cc2-c2ccccc2-c2cc(C(C)(C)C)ccc21]

### [Chemical Formula 51]

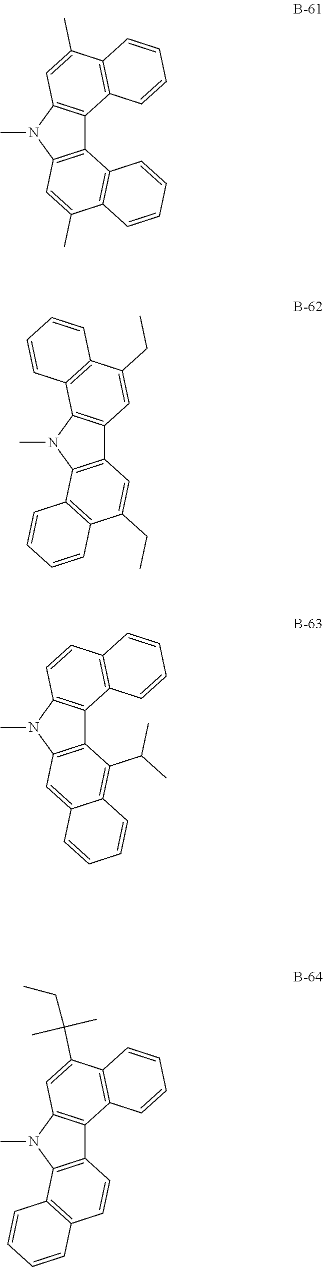

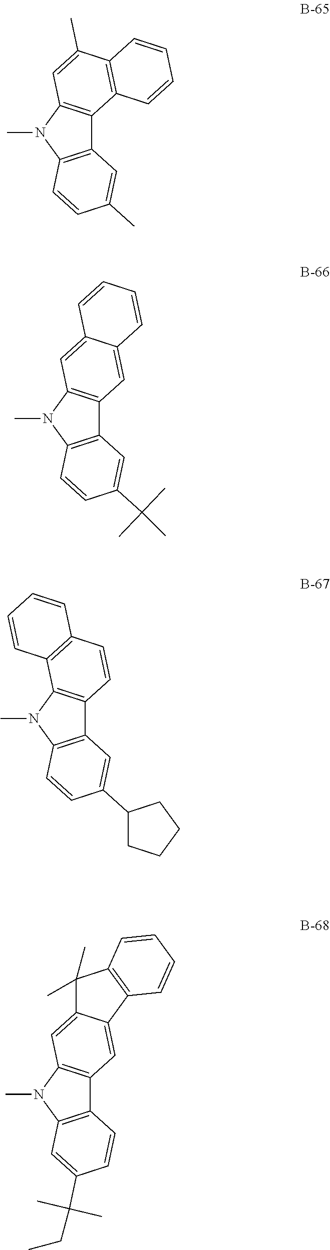

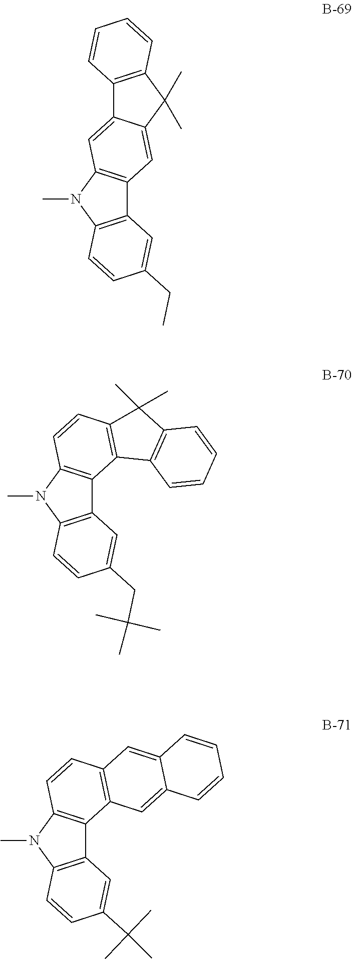

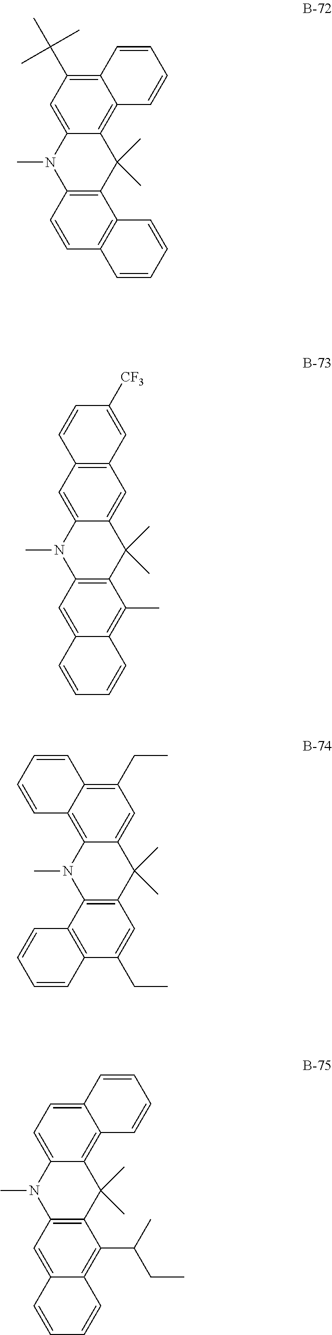

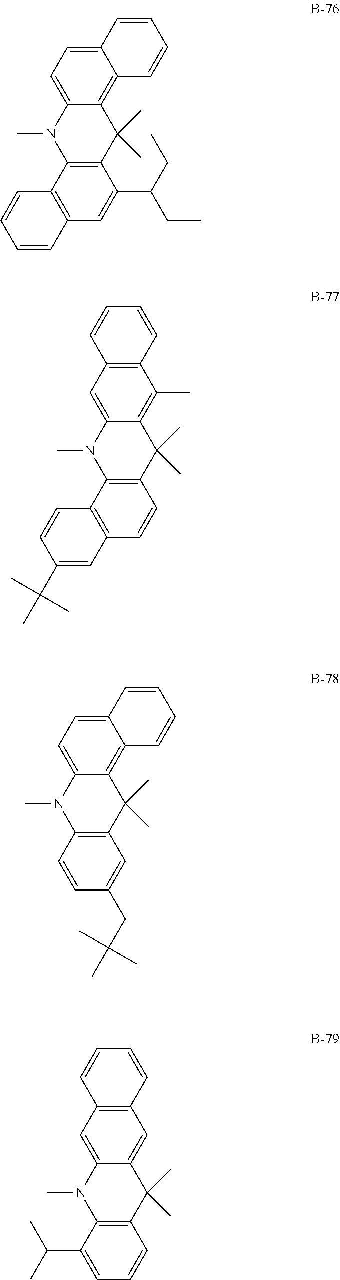

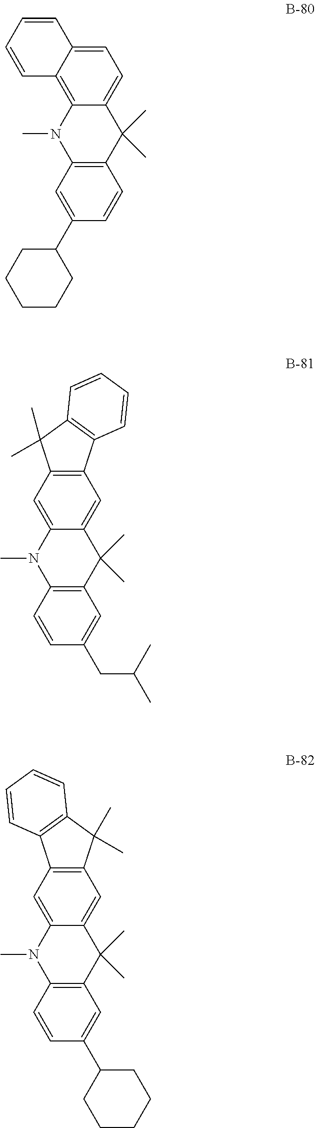

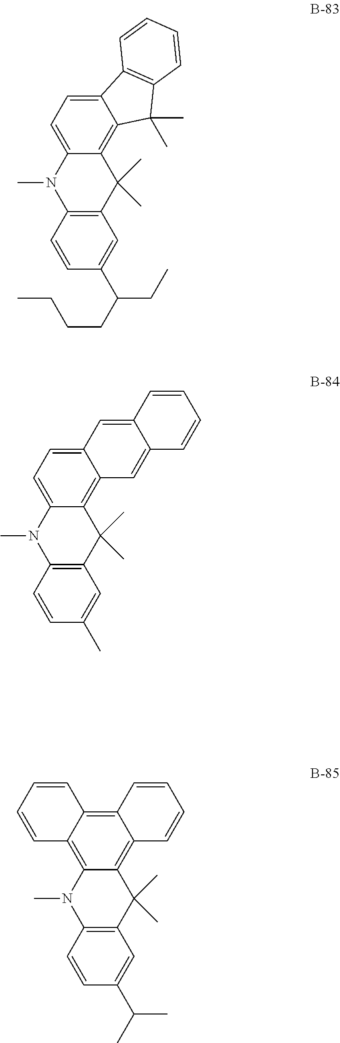

### [Chemical Formula 52]

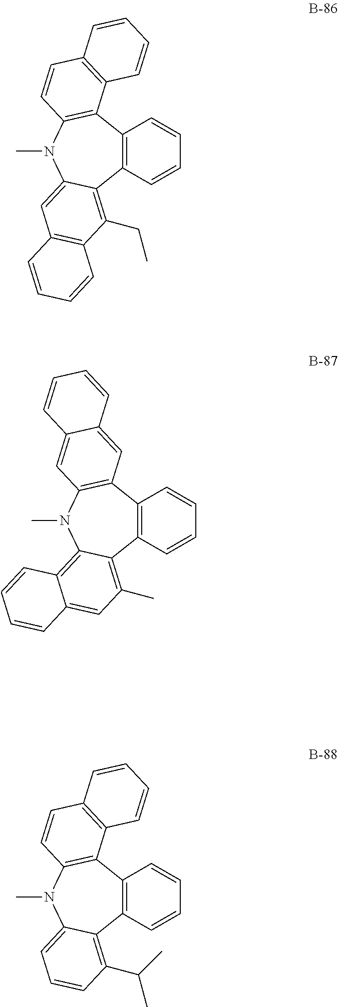 [SMILES: CCc1c2c(cc3ccccc13)N(C)c1ccc3ccccc3c1-c1ccccc1-2 ; Cc1cc2ccccc2c2c1-c1ccccc1-c1cc3ccccc3cc1N2C ; CC(C)c1cccc2c1-c1ccccc1-c1c(ccc3ccccc13)N2C]

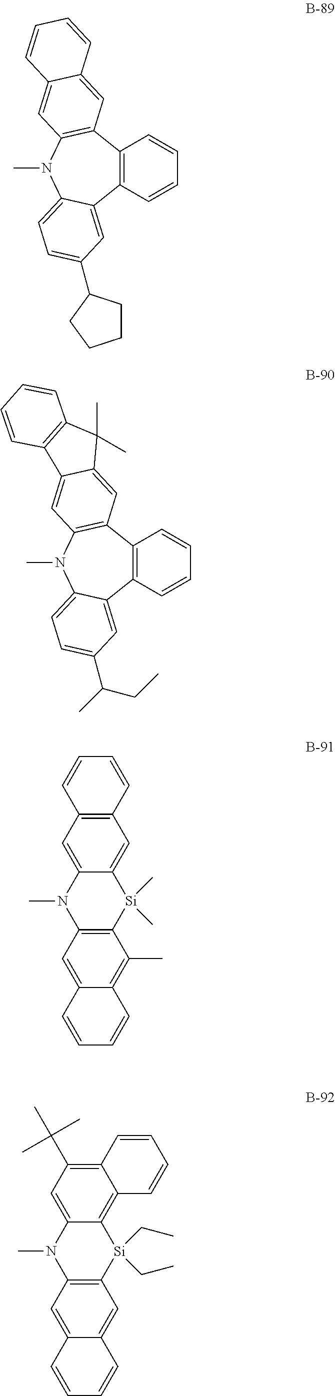

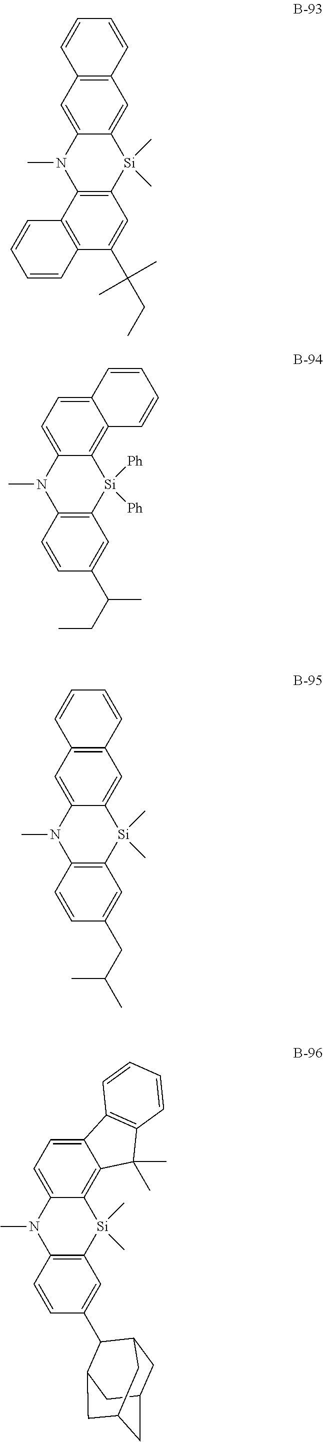

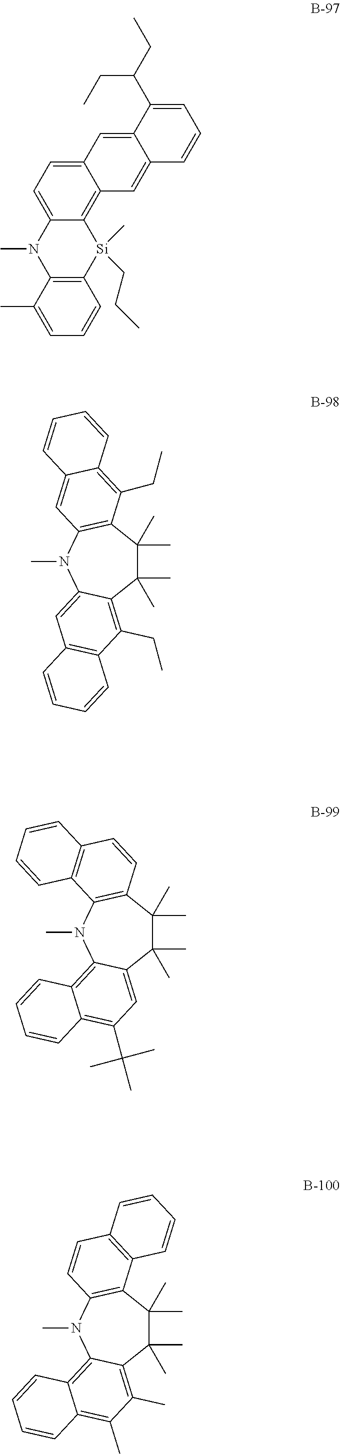

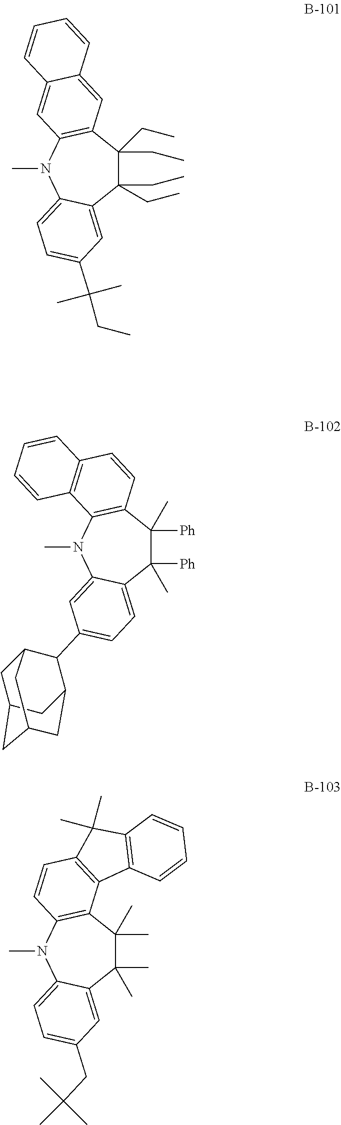

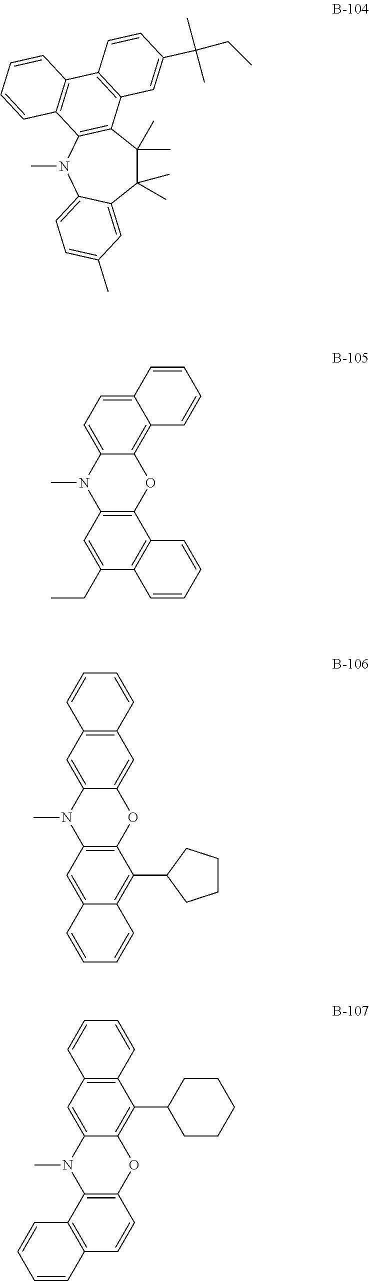

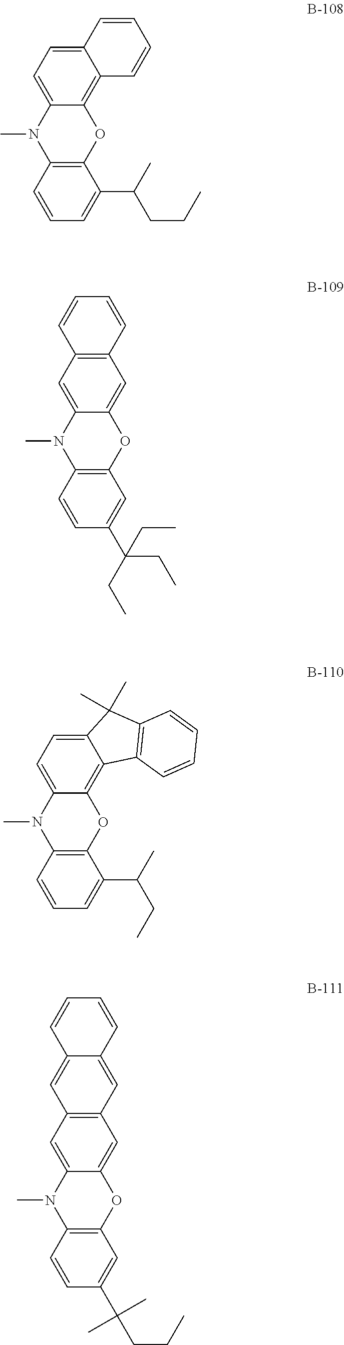

### [Chemical Formula 53]

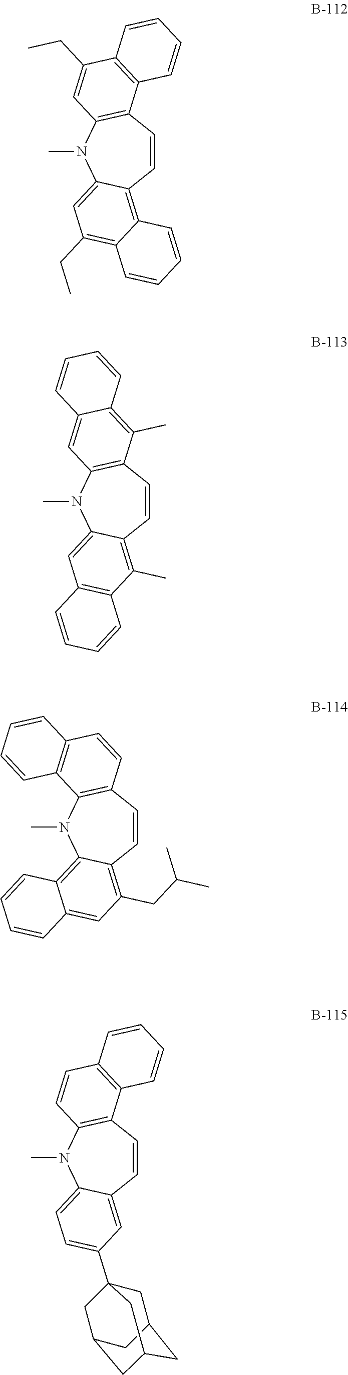

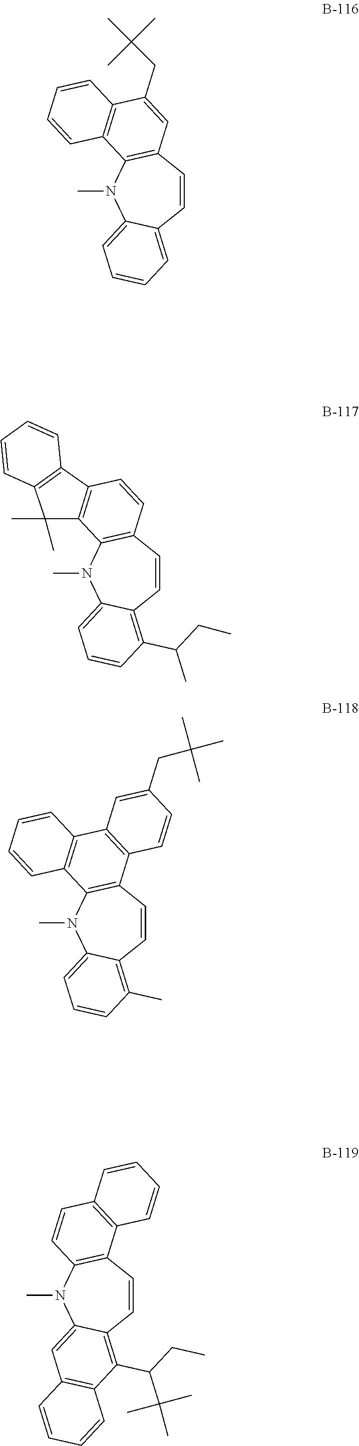

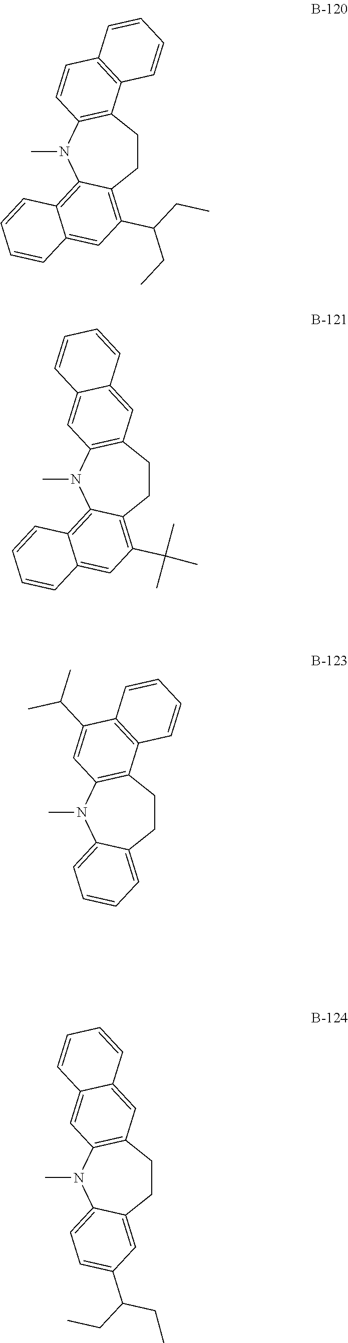

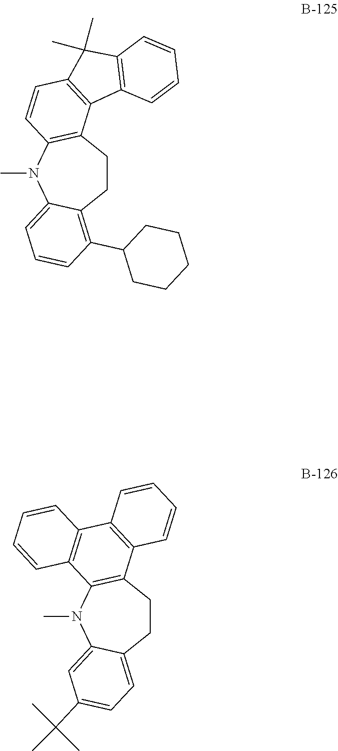 [SMILES: CN1c2cccc(C3CCCCC3)c2CCc2c1ccc1c2-c2ccccc2C1(C)C ; CN1c2cc(C(C)(C)C)ccc2CCc2c1c1ccccc1c1ccccc21]

### [Chemical Formula 54]

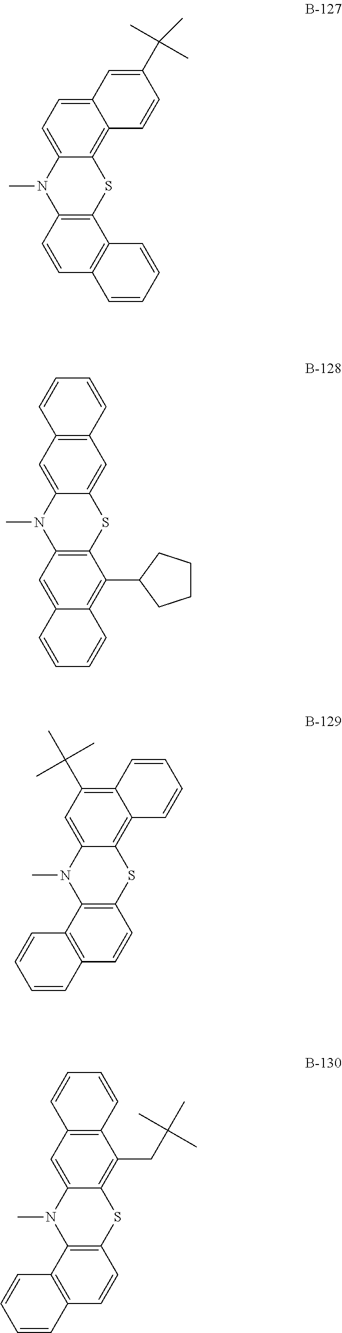

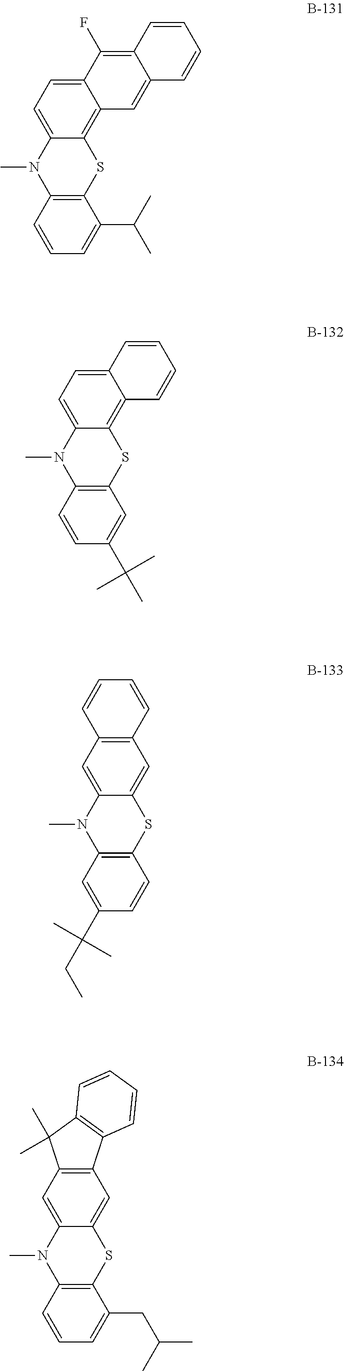 [SMILES: CC(C)c1cccc2c1Sc1c(ccc3c(F)c4ccccc4cc13)N2C ; CN1c2ccc(C(C)(C)C)cc2Sc2c1ccc1ccccc21 ; CCC(C)(C)c1ccc2c(c1)N(C)c1cc3ccccc3cc1S2 ; CC(C)Cc1cccc2c1Sc1cc3c(cc1N2C)C(C)(C)c1ccccc1-3]

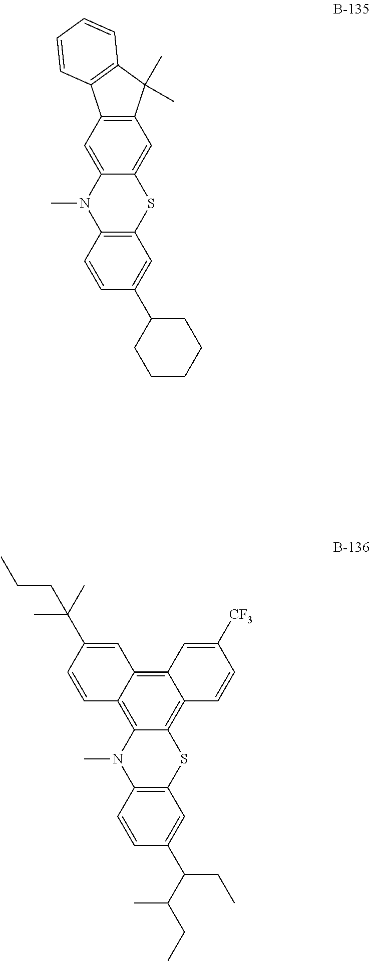 [SMILES: CN1c2ccc(C3CCCCC3)cc2Sc2cc3c(cc21)-c1ccccc1C3(C)C ; CCCC(C)(C)c1ccc2c3c(c4ccc(C(F)(F)F)cc4c2c1)Sc1cc(C(CC)C(C)CC)ccc1N3C]

### [Chemical Formula 55]

| | |
|---|---|
| | (a) |
| 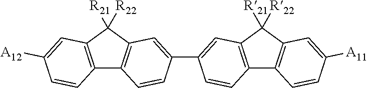 | |

| COM- | | | | | | |
|---|---|---|---|---|---|---|
| POUND | | | | | | |
| NUMBER | R₂₁ | R₂₂ | R′₂₁ | R′₂₂ | A₁₁ | A₁₂ |
| a-1 | Me | Me | Me | Me | B-1 | B-1 |
| a-2 | Me | Me | Me | Me | B-2 | B-2 |
| a-3 | Me | Me | Me | Me | B-3 | B-3 |
| a-4 | Me | Me | Me | Me | B-8 | B-8 |
| a-5 | Me | Me | Me | Me | B-9 | B-9 |
| a-6 | Me | Me | Me | Me | B-10 | B-10 |
| a-7 | Me | Me | Me | Me | B-14 | B-14 |
| a-8 | Me | Me | Me | Me | B-21 | B-21 |
| a-9 | Me | Me | Me | Me | B-23 | B-23 |
| a-10 | Me | Me | Me | Me | B-31 | B-33 |
| a-11 | Me | Me | Me | Me | B-42 | B-42 |
| a-12 | H | H | H | H | B-43 | B-43 |
| a-13 | H | H | H | Me | B-47 | B-47 |
| a-14 | Et | Et | Et | Et | B-48 | B-48 |
| a-15 | n-Bu | n-Bu | n-Bu | n-Bu | B-31 | B-33 |
| a-16 | Ph | Ph | Ph | Ph | B-4 | B-4 |
| a-17 | Me | Me | Me | Ph | B-5 | B-5 |
| a-18 | i-Pr | i-Pr | i-Pr | i-Pr | B-17 | B-17 |
| a-19 | 2-MeOEt | 2-MeOEt | 2-MeOEt | 2-MeOEt | B-1 | B-2 |
| a-20 | Me | Me | Me | Me | B-26 | B-26 |
| a-21 | Et | Et | Ph | Ph | B-6 | B-9 |
| a-22 | Me | Me | Me | Me | B-8 | B-10 |
| a-23 | Me | Me | Me | Me | B-1 | B-8 |
| a-24 | Me | Me | Me | Me | B-30 | B-10 |
| a-25 | Me | Et | Me | Ph | B-1 | B-20 |
| a-26 | Me | Me | Me | Me | B-61 | B-61 |
| a-27 | Me | Me | Me | Me | B-64 | B-64 |
| a-28 | Me | Me | Me | Me | B-65 | B-65 |
| a-29 | Me | Me | Me | Me | B-69 | B-69 |
| a-30 | Me | Me | Me | Me | B-71 | B-71 |
| a-31 | Me | Me | Me | Me | B-72 | B-72 |
| a-32 | Me | Me | Me | Me | B-74 | B-74 |
| a-33 | Me | Me | Me | Me | B-76 | B-76 |
| a-34 | Me | Me | Me | Me | B-78 | B-78 |
| a-35 | Me | Me | Me | Me | B-81 | B-81 |
| a-36 | Me | Me | Me | Me | B-84 | B-84 |
| a-37 | H | H | H | H | B-86 | B-86 |
| a-38 | H | H | H | Me | B-89 | B-89 |
| a-39 | Et | Et | Et | Et | B-93 | B-93 |
| a-40 | n-Bu | n-Bu | n-Bu | n-Bu | B-98 | B-101 |
| a-41 | Ph | Ph | Ph | Ph | B-102 | B-105 |
| a-42 | Me | Me | Me | Ph | B-106 | B-106 |
| a-43 | i-Pr | i-Pr | i-Pr | i-Pr | B-107 | B-110 |
| a-44 | 2-MeOEt | 2-MeOEt | 2-MeOEt | 2-MeOEt | B-112 | B-115 |
| a-45 | Me | Me | Me | Me | B-116 | B-119 |
| a-46 | Et | Et | Ph | Ph | B-120 | B-124 |
| a-47 | Me | Me | Me | Me | B-127 | B-131 |
| a-48 | Me | Me | Me | Me | B-132 | B-136 |
| a-49 | Me | Me | Me | Me | B-128 | B-128 |
| a-50 | Me | Et | Me | Ph | B-1 | B-61 |

### [Chemical Formula 56]

| | |
|---|---|
| | (b) |
| 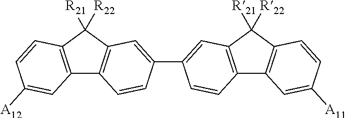 | |

| COM- | | | | | | |
|---|---|---|---|---|---|---|
| POUND | | | | | | |
| NUMBER | R₂₁ | R₂₂ | R′₂₁ | R′₂₂ | A₁₁ | A₁₂ |
| b-1 | Me | Me | Me | Me | B-1 | B-1 |
| b-2 | Me | Me | Me | Me | B-2 | B-2 |
| b-3 | Me | Me | Me | Me | B-3 | B-3 |
| b-4 | Me | Me | Me | Me | B-8 | B-8 |
| b-5 | Me | Me | Me | Me | B-9 | B-9 |
| b-6 | H | H | H | H | B-43 | B-43 |
| b-7 | H | H | H | Me | B-47 | B-47 |
| b-8 | Et | Et | Et | Et | B-48 | B-48 |
| b-9 | n-Bu | n-Bu | n-Bu | n-Bu | B-5 | B-6 |
| b-10 | Ph | Ph | Ph | Ph | B-11 | B-11 |
| b-11 | Me | Me | Me | Ph | B-15 | B-15 |
| b-12 | i-Pr | i-Pr | i-Pr | i-Pr | B-17 | B-17 |
| b-13 | 2-MeOEt | 2-MeOEt | 2-MeOEt | 2-MeOEt | B-23 | B-23 |
| b-14 | Et | Et | Ph | Ph | B-26 | B-26 |
| b-15 | Me | Et | Me | Ph | B-32 | B-32 |
| b-16 | Me | Me | Me | Me | B-62 | B-62 |
| b-17 | Me | Me | Me | Me | B-65 | B-65 |
| b-18 | Me | Me | Me | Me | B-73 | B-73 |
| b-19 | Me | Me | Me | Me | B-77 | B-77 |
| b-20 | Me | Me | Me | Me | B-86 | B-86 |
| b-21 | H | H | H | H | B-83 | B-83 |
| b-22 | H | H | H | Me | B-90 | B-90 |
| b-23 | Et | Et | Et | Et | B-103 | B-103 |
| b-24 | n-Bu | n-Bu | n-Bu | n-Bu | B-113 | B-113 |
| b-25 | Ph | Ph | Ph | Ph | B-116 | B-118 |
| b-26 | Me | Me | Me | Ph | B-126 | B-126 |
| b-27 | i-Pr | i-Pr | i-Pr | i-Pr | B-130 | B-130 |
| b-28 | 2-MeOEt | 2-MeOEt | 2-MeOEt | 2-MeOEt | B-133 | B-133 |
| b-29 | Et | Et | Ph | Ph | B-92 | B-92 |
| b-30 | Me | Et | Me | Ph | B-95 | B-95 |

### [Chemical Formula 57]

| | |
|---|---|
| | (c) |
| 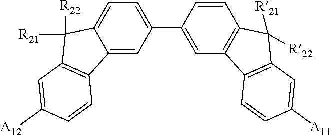 | |

| COM- | | | | | | |
|---|---|---|---|---|---|---|
| POUND | | | | | | |
| NUMBER | R₂₁ | R₂₂ | R′₂₁ | R′₂₂ | A₁₁ | A₁₂ |
| c-1 | Me | Me | Me | Me | B-1 | B-1 |
| c-2 | Me | Me | Me | Me | B-2 | B-2 |
| c-3 | Me | Me | Me | Me | B-3 | B-3 |
| c-4 | Me | Me | Me | Me | B-8 | B-8 |
| c-5 | Me | Me | Me | Me | B-9 | B-9 |
| c-6 | H | H | H | H | B-10 | B-10 |
| c-7 | H | H | H | Me | B-51 | B-51 |
| c-8 | Et | Et | Et | Et | B-46 | B-44 |
| c-9 | n-Bu | n-Bu | n-Bu | n-Bu | B-37 | B-37 |
| c-10 | Ph | Ph | Ph | Ph | B-38 | B-38 |
| c-11 | Me | Me | Me | Ph | B-33 | B-35 |
| c-12 | i-Pr | i-Pr | i-Pr | i-Pr | B-27 | B-27 |
| c-13 | 2-MeOEt | 2-MeOEt | 2-MeOEt | 2-MeOEt | B-24 | B-24 |
| c-14 | Et | Et | Ph | Ph | B-7 | B-7 |
| c-15 | Me | Et | Me | Ph | B-7 | B-24 |
| c-16 | Me | Me | Me | Me | B-63 | B-63 |
| c-17 | Me | Me | Me | Me | B-67 | B-67 |
| c-18 | Me | Me | Me | Me | B-75 | B-75 |
| c-19 | Me | Me | Me | Me | B-78 | B-78 |
| c-20 | Me | Me | Me | Me | B-87 | B-87 |
| c-21 | H | H | H | H | B-91 | B-91 |
| c-22 | H | H | H | Me | B-99 | B-99 |
| c-23 | Et | Et | Et | Et | B-108 | B-108 |
| c-24 | n-Bu | n-Bu | n-Bu | n-Bu | B-111 | B-111 |
| c-25 | Ph | Ph | Ph | Ph | B-114 | B-114 |
| c-26 | Me | Me | Me | Ph | B-121 | B-121 |
| c-27 | i-Pr | i-Pr | i-Pr | i-Pr | B-125 | B-125 |
| c-28 | 2-MeOEt | 2-MeOEt | 2-MeOEt | 2-MeOEt | B-129 | B-129 |
| c-29 | Et | Et | Ph | Ph | B-94 | B-94 |
| c-30 | Me | Et | Me | Ph | B-109 | B-109 |

### [Chemical Formula 58]

| | |
|---|---|
| | (d) |
| 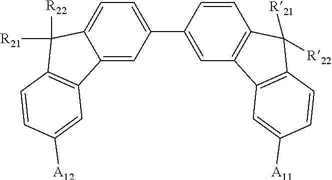 | |

| COM- | | | | | | |
|---|---|---|---|---|---|---|
| POUND | | | | | | |
| NUMBER | R₂₁ | R₂₂ | R′₂₁ | R′₂₂ | A₁₁ | A₁₂ |
| d-1 | Me | Me | Me | Me | B-1 | B-1 |
| d-2 | Me | Me | Me | Me | B-2 | B-2 |
| d-3 | Me | Me | Me | Me | B-3 | B-3 |
| d-4 | Me | Me | Me | Me | B-8 | B-8 |
| d-5 | Me | Me | Me | Me | B-9 | B-9 |
| d-6 | H | H | H | H | B-10 | B-10 |
| d-7 | H | H | H | Me | B-12 | B-12 |
| d-8 | Et | Et | Et | Et | B-18 | B-18 |
| d-9 | n-Bu | n-Bu | n-Bu | n-Bu | B-25 | B-25 |
| d-10 | Ph | Ph | Ph | Ph | B-31 | B-31 |
| d-11 | Me | Me | Me | Ph | B-34 | B-34 |
| d-12 | i-Pr | i-Pr | i-Pr | i-Pr | B-39 | B-39 |
| d-13 | 2-MeOEt | 2-MeOEt | 2-MeOEt | 2-MeOEt | B-49 | B-49 |
| d-14 | Et | Et | Ph | Ph | B-16 | B-22 |
| d-15 | Me | Et | Me | Ph | B-3 | B-10 |
| d-16 | Me | Me | Me | Me | B-61 | B-61 |
| d-17 | Me | Me | Me | Me | B-70 | B-70 |
| d-18 | Me | Me | Me | Me | B-72 | B-72 |
| d-19 | Me | Me | Me | Me | B-79 | B-79 |
| d-20 | Me | Me | Me | Me | B-88 | B-88 |
| d-21 | H | H | H | H | B-96 | B-96 |
| d-22 | H | H | H | Me | B-100 | B-100 |
| d-23 | Et | Et | Et | Et | B-117 | B-117 |
| d-24 | n-Bu | n-Bu | n-Bu | n-Bu | B-125 | B-125 |
| d-25 | Ph | Ph | Ph | Ph | B-131 | B-131 |
| d-26 | Me | Me | Me | Ph | B-134 | B-134 |
| d-27 | i-Pr | i-Pr | i-Pr | i-Pr | B-135 | B-135 |
| d-28 | 2-MeOEt | 2-MeOEt | 2-MeOEt | 2-MeOEt | B-14 | B-74 |
| d-29 | Et | Et | Ph | Ph | B-25 | B-86 |
| d-30 | Me | Et | Me | Ph | B-101 | B-10 |

### [Chemical Formula 59]

| | |
|---|---|
| | (e) |
| 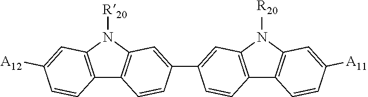 | |

| COMPOUND | | | | |
|---|---|---|---|---|
| NUMBER | R₂₀ | R′₂₀ | A₁₁ | A₁₂ |
| e-1 | Ph | Ph | B-1 | B-1 |
| e-2 | Ph | Ph | B-2 | B-2 |
| e-3 | Ph | Ph | B-3 | B-3 |
| e-4 | Ph | Ph | B-8 | B-8 |
| e-5 | Ph | Ph | B-9 | B-9 |
| e-6 | Ph | Ph | B-10 | B-10 |
| e-7 | Ph | Ph | B-14 | B-14 |
| e-8 | Ph | Ph | B-20 | B-20 |
| e-9 | Ph | Ph | B-21 | B-21 |
| e-10 | 2-tol | 2-tol | B-1 | B-1 |
| e-11 | 3-tol | 3-tol | B-2 | B-2 |
| e-12 | 4-tol | 4-tol | B-3 | B-3 |
| e-13 | 2-Np | 2-Np | B-8 | B-8 |
| e-14 | 1-Np | 1-Np | B-9 | B-9 |
| e-15 | 2-An | 2-An | B-10 | B-10 |
| e-16 | 2-Fn | 2-Fn | B-4 | B-4 |
| e-17 | Me | Me | B-28 | B-28 |
| e-18 | i-Pr | i-Pr | B-36 | B-36 |
| e-19 | Et | Et | B-40 | B-40 |
| e-20 | Ph | 2-tol | B-45 | B-50 |
| e-21 | 3-tol | Ph | B-8 | B-9 |
| e-22 | 2-Fn | Ph | B-8 | B-10 |
| e-23 | t-Bu | t-Bu | B-1 | B-1 |
| e-24 | t-Bu | t-Bu | B-3 | B-3 |
| e-25 | 2-Np | Ph | B-1 | B-8 |

### [Chemical Formula 60]

(f)

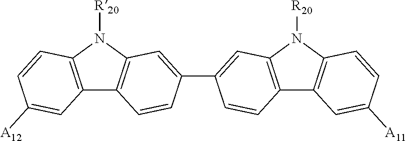

| COMPOUND | | | | |
|---|---|---|---|---|
| NUMBER | R₂₀ | R′₂₀ | A₁₁ | A₁₂ |
| f-1 | Ph | Ph | B-1 | B-1 |
| f-2 | Ph | Ph | B-2 | B-2 |
| f-3 | Ph | Ph | B-3 | B-3 |
| f-4 | Ph | Ph | B-8 | B-8 |
| f-5 | 2-tol | 2-tol | B-9 | B-9 |
| f-6 | 3-tol | 3-tol | B-10 | B-10 |
| f-7 | 4-tol | 4-tol | B-3 | B-3 |
| f-8 | 2-Np | 2-Np | B-8 | B-8 |
| f-9 | 1-Np | 1-Np | B-9 | B-9 |
| f-10 | 2-An | 2-An | B-10 | B-10 |
| f-11 | 2-Fn | 2-Fn | B-14 | B-14 |
| f-12 | Me | Me | B-21 | B-21 |
| f-13 | i-Pr | i-Pr | B-29 | B-29 |
| f-14 | Et | Et | B-41 | B-41 |
| f-15 | Ph | 2-tol | B-45 | B-50 |
| f-16 | 3-tol | Ph | B-9 | B-2 |
| f-17 | 2-Fn | Ph | B-8 | B-3 |
| f-18 | t-Bu | t-Bu | B-3 | B-4 |
| f-19 | 2-Np | Ph | B-1 | B-9 |
| f-20 | Ph | Ph | B-63 | B-63 |
| f-21 | Ph | Ph | B-68 | B-68 |
| f-22 | Ph | Ph | B-71 | B-71 |
| f-23 | Ph | Ph | B-74 | B-74 |
| f-24 | 2-tol | 2-tol | B-76 | B-76 |
| f-25 | 3-tol | 3-tol | B-80 | B-80 |
| f-26 | 4-tol | 4-tol | B-83 | B-83 |
| f-27 | 2-Np | 2-Np | B-87 | B-87 |
| f-28 | 1-Np | 1-Np | B-93 | B-93 |
| f-29 | 2-An | 2-An | B-97 | B-97 |
| f-30 | 2-Fn | 2-Fn | B-100 | B-100 |
| f-31 | Me | Me | B-104 | B-104 |
| f-32 | i-Pr | i-Pr | B-111 | B-111 |
| f-33 | Et | Et | B-113 | B-113 |
| f-34 | Ph | 2-tol | B-118 | B-118 |
| f-35 | 3-tol | Ph | B-124 | B-124 |
| f-36 | 2-Fn | Ph | B-127 | B-127 |
| f-37 | t-Bu | t-Bu | B-34 | B-114 |
| f-38 | 2-Np | Ph | B-45 | B-105 |

### [Chemical Formula 61]

(g)

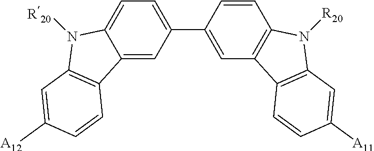

| COMPOUND | | | | |
|---|---|---|---|---|
| NUMBER | R₂₀ | R′₂₀ | A₁₁ | A₁₂ |
| g-1 | Ph | Ph | B-2 | B-2 |
| g-2 | Ph | Ph | B-3 | B-3 |
| g-3 | Ph | Ph | B-9 | B-9 |
| g-4 | Ph | Ph | B-10 | B-10 |
| g-10 | 2-tol | 2-tol | B-9 | B-9 |
| g-11 | 3-tol | 3-tol | B-14 | B-14 |
| g-12 | 4-tol | 4-tol | B-10 | B-10 |
| g-13 | 2-Np | 2-Np | B-8 | B-8 |
| g-14 | 1-Np | 1-Np | B-9 | B-9 |
| g-15 | 2-An | 2-An | B-10 | B-10 |
| g-16 | 2-Fn | 2-Fn | B-21 | B-21 |
| g-17 | Me | Me | B-26 | B-26 |
| g-18 | i-Pr | i-Pr | B-31 | B-31 |
| g-19 | Et | Et | B-37 | B-37 |
| g-20 | Ph | 2-tol | B-43 | B-43 |
| g-21 | 3-tol | Ph | B-48 | B-48 |
| g-22 | 2-Fn | Ph | B-22 | B-22 |
| g-24 | t-Bu | t-Bu | B-28 | B-28 |
| g-25 | 2-Np | Ph | B-1 | B-9 |
| g-26 | Ph | Ph | B-64 | B-54 |
| g-27 | Ph | Ph | B-67 | B-67 |
| g-28 | Ph | Ph | B-71 | B-71 |
| g-29 | Ph | Ph | B-75 | B-75 |
| g-30 | 2-tol | 2-tol | B-78 | B-78 |
| g-31 | 3-tol | 3-tol | B-81 | B-81 |
| g-32 | 4-tol | 4-tol | B-85 | B-85 |
| g-33 | 2-Np | 2-Np | B-88 | B-88 |
| g-34 | 1-Np | 1-Np | B-91 | B-91 |
| g-35 | 2-An | 2-An | B-95 | B-95 |
| g-36 | 2-Fn | 2-Fn | B-98 | B-98 |
| g-37 | Me | Me | B-101 | B-101 |
| g-38 | i-Pr | i-Pr | B-102 | B-102 |
| g-39 | Et | Et | B-106 | B-106 |
| g-40 | Ph | 2-tol | B-109 | B-109 |
| g-41 | 3-tol | Ph | B-114 | B-114 |
| g-42 | 2-Fn | Ph | B-116 | B-116 |
| g-43 | t-Bu | t-Bu | B-120 | B-120 |
| g-44 | 2-Np | Ph | B-123 | B-123 |
| g-45 | Ph | 2-tol | B-127 | B-127 |
| g-46 | Ph | Ph | B-131 | B-131 |
| g-47 | Ph | 2-tol | B-132 | B-132 |
| g-48 | 3-tol | Ph | B-135 | B-35 |
| g-49 | 2-Fn | Ph | B-22 | B-122 |
| g-50 | t-Bu | t-Bu | B-28 | B-128 |

### [Chemical Formula 62]

(h)

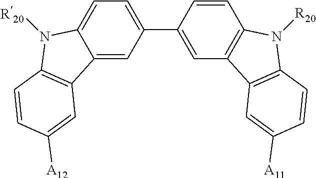

| COMPOUND | | | | |
|---|---|---|---|---|
| NUMBER | R₂₀ | R′₂₀ | A₁₁ | A₁₂ |
| h-1 | Ph | Ph | B-2 | B-2 |
| h-2 | Ph | Ph | B-3 | B-3 |
| h-3 | Ph | Ph | B-9 | B-9 |
| h-4 | Ph | Ph | B-10 | B-10 |
| h-5 | 2-tol | 2-tol | B-9 | B-9 |
| h-6 | 3-tol | 3-tol | B-14 | B-14 |
| h-7 | 4-tol | 4-tol | B-10 | B-10 |
| h-8 | 2-Np | 2-Np | B-8 | B-8 |
| h-9 | 1-Np | 1-Np | B-9 | B-9 |
| h-10 | 2-An | 2-An | B-10 | B-10 |
| h-11 | 2-Fn | 2-Fn | B-21 | B-21 |
| h-12 | Me | Me | B-26 | B-26 |
| h-13 | i-Pr | i-Pr | B-31 | B-31 |
| h-14 | Et | Et | B-38 | B-38 |
| h-15 | Ph | 2-tol | B-42 | B-42 |
| h-16 | 3-tol | Ph | B-51 | B-51 |
| h-17 | 2-Fn | Ph | B-3 | B-4 |
| h-18 | t-Bu | t-Bu | B-5 | B-5 |
| h-19 | 2-Np | Ph | B-3 | B-10 |
| h-20 | Ph | Ph | B-65 | B-65 |
| h-21 | Ph | Ph | B-68 | B-68 |
| h-22 | Ph | Ph | B-79 | B-79 |
| h-23 | Ph | Ph | B-80 | B-80 |
| h-24 | 2-tol | 2-tol | B-86 | B-86 |
| h-25 | 3-tol | 3-tol | B-89 | B-89 |
| h-26 | 4-tol | 4-tol | B-103 | B-103 |
| h-27 | 2-Np | 2-Np | B-105 | B-105 |
| h-28 | 1-Np | 1-Np | B-107 | B-107 |
| h-29 | 2-An | 2-An | B-115 | B-115 |
| h-30 | 2-Fn | 2-Fn | B-119 | B-119 |
| h-31 | Me | Me | B-125 | B-125 |
| h-32 | i-Pr | i-Pr | B-128 | B-128 |
| h-33 | Et | Et | B-133 | B-133 |
| h-34 | Ph | 2-tol | B-42 | B-142 |
| h-35 | 3-tol | Ph | B-51 | B-115 |
| h-36 | 2-Fn | Ph | B-13 | B-67 |
| h-37 | t-Bu | t-Bu | B-5 | B-65 |
| h-38 | 2-Np | Ph | B-3 | B-73 |

### [Chemical Formula 63]

(i)

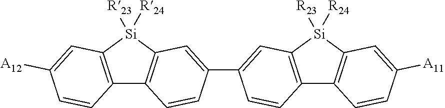

| COMPOUND | | | | | | |
|---|---|---|---|---|---|---|
| NUMBER | R₂₃ | R₂₄ | R′₂₃ | R′₂₄ | A₁₁ | A₁₂ |
| i-1 | Me | Me | Me | Me | B-1 | B-1 |
| i-2 | Me | Me | Me | Me | B-2 | B-2 |
| i-3 | Me | Me | Me | Me | B-3 | B-3 |
| i-4 | Me | Me | Me | Me | B-8 | B-8 |
| i-5 | Me | Me | Me | Me | B-9 | B-9 |
| i-6 | Me | Me | Me | Me | B-10 | B-10 |
| i-7 | Me | Me | Me | Me | B-14 | B-14 |
| i-8 | Me | Me | Me | Me | B-22 | B-22 |
| i-9 | Me | Me | Me | Me | B-27 | B-27 |
| i-10 | Me | Me | Me | Me | B-33 | B-33 |
| i-11 | Me | Me | Me | Me | B-42 | B-42 |
| i-12 | H | H | H | H | B-43 | B-43 |
| i-13 | H | H | H | Me | B-44 | B-44 |
| i-14 | Et | Et | Et | Et | B-45 | B-45 |
| i-15 | n-Bu | n-Bu | n-Bu | n-Bu | B-31 | B-33 |
| i-16 | Ph | Ph | Ph | Ph | B-4 | B-4 |
| i-17 | Me | Me | Me | Ph | B-5 | B-5 |
| i-18 | i-Pr | i-Pr | i-Pr | i-Pr | B-17 | B-17 |
| i-19 | 2-MeOEt | 2-MeOEt | 2-MeOEt | 2-MeOEt | B-1 | B-2 |
| i-20 | 3-tol | Me | 3-tol | Me | B-1 | B-3 |
| i-21 | Et | Et | Ph | Ph | B-8 | B-9 |
| i-22 | 4-tol | Ph | 4-tol | Me | B-8 | B-10 |
| i-23 | Me | Me | Me | Me | B-1 | B-8 |
| i-24 | 2-tol | Me | 2-tol | Me | B-30 | B-10 |
| i-25 | Me | Et | Me | Ph | B-1 | B-20 |
| i-26 | Me | Me | Me | Me | B-65 | B-65 |
| i-27 | Me | Me | Me | Me | B-67 | B-67 |
| i-28 | Me | Me | Me | Me | B-62 | B-62 |
| i-29 | Me | Me | Me | Me | B-72 | B-72 |
| i-30 | Me | Me | Me | Me | B-77 | B-77 |
| i-31 | Me | Me | Me | Me | B-84 | B-84 |
| i-32 | Me | Me | Me | Me | B-85 | B-85 |
| i-33 | Me | Me | Me | Me | B-86 | B-86 |
| i-34 | Me | Me | Me | Me | B-90 | B-90 |
| i-35 | Me | Me | Me | Me | B-94 | B-94 |
| i-36 | Me | Me | Me | Me | B-96 | B-96 |
| i-37 | H | H | H | H | B-103 | B-103 |
| i-38 | H | H | H | Me | B-108 | B-108 |
| i-39 | Et | Et | Et | Et | B-110 | B-110 |
| i-40 | n-Bu | n-Bu | n-Bu | n-Bu | B-117 | B-117 |
| i-41 | Ph | Ph | Ph | Ph | B-121 | B-121 |
| i-42 | Me | Me | Me | Ph | B-126 | B-126 |
| i-43 | i-Pr | i-Pr | i-Pr | i-Pr | B-129 | B-129 |
| i-44 | 2-MeOEt | 2-MeOEt | 2-MeOEt | 2-MeOEt | B-130 | B-130 |
| i-45 | 3-tol | Me | 3-tol | Me | B-133 | B-133 |
| i-46 | Et | Et | Ph | Ph | B-134 | B-134 |
| i-47 | 4-tol | Ph | 4-tol | Me | B-136 | B-136 |
| i-48 | Me | Me | Me | Me | B-1 | B-71 |
| i-49 | 2-tol | Me | 2-tol | Me | B-30 | B-90 |
| i-50 | Me | Et | Me | Ph | B-1 | B-66 |

### [Chemical Formula 64]

(j)

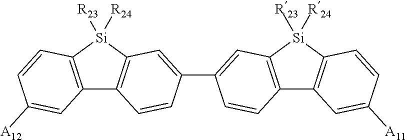

| COMPOUND | | | | | | |
|---|---|---|---|---|---|---|
| NUMBER | R₂₃ | R₂₄ | R′₂₃ | R′₂₄ | A₁₁ | A₁₂ |
| j-1 | Me | Me | Me | Me | B-1 | B-1 |
| j-2 | Me | Me | Me | Me | B-2 | B-2 |
| j-3 | Me | Me | Me | Me | B-3 | B-3 |
| j-4 | Me | Me | Me | Me | B-8 | B-8 |
| j-5 | Me | Me | Me | Me | B-9 | B-9 |
| j-6 | Me | Me | Me | Me | B-10 | B-10 |
| j-7 | Me | Me | Me | Me | B-14 | B-14 |
| j-8 | Me | Me | Me | Me | B-21 | B-21 |
| j-9 | Me | Me | Me | Me | B-31 | B-31 |
| j-10 | Me | Me | Me | Me | B-33 | B-33 |
| j-11 | Me | Me | Me | Me | B-42 | B-42 |
| j-12 | H | H | H | H | B-43 | B-43 |
| j-13 | H | H | H | Me | B-44 | B-44 |
| j-14 | Et | Et | Et | Et | B-45 | B-45 |
| j-15 | n-Bu | n-Bu | n-Bu | n-Bu | B-31 | B-31 |
| j-16 | Ph | Ph | Ph | Ph | B-4 | B-4 |
| j-17 | Me | Me | Me | Ph | B-5 | B-5 |
| j-18 | i-Pr | i-Pr | i-Pr | i-Pr | B-18 | B-18 |
| j-19 | 2-MeOEt | 2-MeOEt | 2-MeOEt | 2-MeOEt | B-1 | B-2 |
| j-20 | 3-tol | Me | 3-tol | Me | B-4 | B-3 |
| j-21 | Et | Et | Ph | Ph | B-8 | B-9 |
| j-22 | 4-tol | Ph | 4-tol | Me | B-8 | B-10 |
| j-23 | Me | Me | Me | Me | B-4 | B-5 |
| j-24 | 2-tol | Me | 2-tol | Me | B-31 | B-10 |
| j-25 | Me | Et | Me | Ph | B-3 | B-20 |
| j-26 | Me | Me | Me | Me | B-61 | B-61 |
| j-27 | Me | Me | Me | Me | B-64 | B-64 |
| j-28 | Me | Me | Me | Me | B-66 | B-66 |
| j-29 | Me | Me | Me | Me | B-69 | B-69 |
| j-30 | Me | Me | Me | Me | B-71 | B-71 |
| j-31 | Me | Me | Me | Me | B-72 | B-72 |
| j-32 | Me | Me | Me | Me | B-74 | B-74 |
| j-33 | Me | Me | Me | Me | B-76 | B-76 |
| j-34 | Me | Me | Me | Me | B-78 | B-78 |
| j-35 | Me | Me | Me | Me | B-81 | B-81 |
| j-36 | Me | Me | Me | Me | B-84 | B-84 |
| j-37 | H | H | H | H | B-86 | B-85 |
| j-38 | H | H | H | Me | B-89 | B-89 |
| j-39 | Et | Et | Et | Et | B-93 | B-93 |
| j-40 | n-Bu | n-Bu | n-Bu | n-Bu | B-98 | B-101 |
| j-41 | Ph | Ph | Ph | Ph | B-61 | B-61 |
| j-42 | Me | Me | Me | Ph | B-64 | B-64 |
| j-43 | i-Pr | i-Pr | i-Pr | i-Pr | B-66 | B-66 |
| j-44 | 2-MeOEt | 2-MeOEt | 2-MeOEt | 2-MeOEt | B-91 | B-91 |
| j-45 | 3-tol | Me | 3-tol | Me | B-99 | B-99 |
| j-46 | Et | Et | Ph | Ph | B-108 | B-108 |
| j-47 | 4-tol | Ph | 4-tol | Me | B-111 | B-111 |
| j-48 | Me | Me | Me | Me | B-114 | B-114 |
| j-49 | 2-tol | Me | 2-tol | Me | B-121 | B-121 |
| j-50 | Me | Et | Me | Ph | B-125 | B-125 |

### [Chemical Formula 65]

(k)

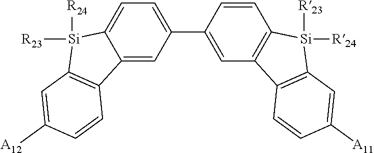

| COMPOUND | | | | | | |
|---|---|---|---|---|---|---|
| NUMBER | R₂₃ | R₂₄ | R′₂₃ | R′₂₄ | A₁₁ | A₁₂ |
| k-1 | Me | Me | Me | Me | B-1 | B-1 |
| k-2 | Me | Me | Me | Me | B-2 | B-2 |
| k-3 | Me | Me | Me | Me | B-3 | B-3 |
| k-4 | Me | Me | Me | Me | B-8 | B-8 |
| k-5 | Me | Me | Me | Me | B-9 | B-9 |
| k-6 | Me | Me | Me | Me | B-10 | B-10 |
| k-7 | Me | Me | Me | Me | B-14 | B-14 |
| k-8 | Me | Me | Me | Me | B-25 | B-25 |
| k-9 | Me | Me | Me | Me | B-22 | B-22 |
| k-10 | Me | Me | Me | Me | B-29 | B-29 |
| k-11 | Me | Me | Me | Me | B-33 | B-33 |
| k-12 | H | H | H | H | B-42 | B-42 |
| k-13 | H | H | H | Me | B-45 | B-45 |
| k-14 | Et | Et | Et | Et | B-50 | B-50 |
| k-15 | n-Bu | n-Bu | n-Bu | n-Bu | B-31 | B-31 |
| k-16 | Ph | Ph | Ph | Ph | B-3 | B-3 |
| k-17 | Me | Me | Me | Ph | B-9 | B-9 |
| k-18 | i-Pr | i-Pr | i-Pr | i-Pr | B-17 | B-18 |
| k-19 | 2-MeOEt | 2-MeOEt | 2-MeOEt | 2-MeOEt | B-3 | B-2 |
| k-20 | 3-tol | Me | 3-tol | Me | B-4 | B-3 |
| k-21 | Et | Et | Ph | Ph | B-8 | B-9 |
| k-22 | 4-tol | Ph | Ph | Me | B-8 | B-10 |
| k-23 | Me | Me | Me | Me | B-4 | B-5 |
| k-24 | 3-tol | Me | 2-tol | Me | B-31 | B-10 |
| k-25 | Me | Et | Me | Ph | B-3 | B-21 |
| k-26 | Me | Me | Me | Me | B-64 | B-84 |
| k-27 | Me | Me | Me | Me | B-67 | B-87 |
| k-28 | Me | Me | Me | Me | B-71 | B-71 |
| k-29 | Me | Me | Me | Me | B-75 | B-75 |
| k-30 | Me | Me | Me | Me | B-78 | B-78 |
| k-31 | Me | Me | Me | Me | B-81 | B-81 |
| k-32 | Me | Me | Me | Me | B-85 | B-85 |
| k-33 | Me | Me | Me | Me | B-88 | B-88 |
| k-34 | Me | Me | Me | Me | B-91 | B-91 |
| k-35 | Me | Me | Me | Me | B-95 | B-95 |
| k-36 | Me | Me | Me | Me | B-98 | B-98 |
| k-37 | H | H | H | H | B-101 | B-101 |
| k-38 | H | H | H | Me | B-102 | B-102 |
| k-39 | Et | Et | Et | Et | B-106 | B-106 |
| k-40 | n-Bu | n-Bu | n-Bu | n-Bu | B-109 | B-109 |
| k-41 | Ph | Ph | Ph | Ph | B-111 | B-111 |
| k-42 | Me | Me | Me | Ph | B-112 | B-112 |
| k-43 | i-Pr | i-Pr | i-Pr | i-Pr | B-116 | B-116 |
| k-44 | 2-MeOEt | 2-MeOEt | 2-MeOEt | 2-MeOEt | B-123 | B-123 |
| k-45 | 3-tol | Me | 3-tol | Me | B-126 | B-126 |
| k-46 | Et | Et | Ph | Ph | B-127 | B-131 |
| k-47 | 4-tol | Ph | Ph | Me | B-45 | B-50 |
| k-48 | Me | Me | Me | Me | B-8 | B-9 |
| k-49 | 3-tol | Me | 2-tol | Me | B-8 | B-10 |
| k-50 | Me | Et | Me | Ph | B-72 | B-17 |

### [Chemical Formula 66]

(l)

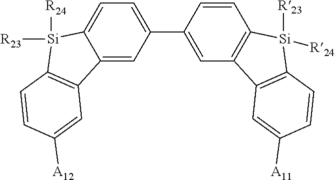

| COMPOUND | | | | | | |
|---|---|---|---|---|---|---|
| NUMBER | R₂₃ | R₂₄ | R′₂₃ | R′₂₄ | A₁₁ | A₁₂ |
| l-1 | Me | Me | Me | Me | B-1 | B-1 |
| l-2 | Me | Me | Me | Me | B-2 | B-2 |
| l-3 | Me | Me | Me | Me | B-3 | B-3 |
| l-4 | Me | Me | Me | Me | B-8 | B-8 |
| l-5 | Me | Me | Me | Me | B-9 | B-9 |
| l-6 | Me | Me | Me | Me | B-10 | B-10 |
| l-7 | Me | Me | Me | Me | B-14 | B-14 |
| l-8 | Me | Me | Me | Me | B-22 | B-22 |
| l-9 | Me | Me | Me | Me | B-27 | B-27 |
| l-10 | Me | Me | Me | Me | B-33 | B-33 |
| l-11 | Me | Me | Me | Me | B-42 | B-42 |
| l-12 | H | H | H | H | B-43 | B-43 |
| l-13 | H | H | H | Me | B-44 | B-44 |
| l-14 | Et | Et | Et | Et | B-45 | B-45 |
| l-15 | n-Bu | n-Bu | n-Bu | n-Bu | B-31 | B-33 |
| l-16 | Ph | Ph | Ph | Ph | B-4 | B-4 |
| l-17 | Me | Me | Me | Ph | B-5 | B-5 |
| l-18 | i-Pr | i-Pr | i-Pr | i-Pr | B-17 | B-17 |
| l-19 | 2-MeOEt | 2-MeOEt | 2-MeOEt | 2-MeOEt | B-1 | B-2 |
| l-20 | 3-tol | Me | 3-tol | Me | B-1 | B-3 |
| l-21 | Et | Et | Ph | Ph | B-8 | B-9 |
| l-22 | 4-tol | Ph | 4-tol | Me | B-8 | B-10 |
| l-23 | Me | Me | Me | Me | B-1 | B-8 |
| l-24 | 2-tol | Me | 2-tol | Me | B-30 | B-10 |
| l-25 | Me | Et | Me | Ph | B-1 | B-20 |
| l-26 | Me | Me | Me | Me | B-62 | B-62 |
| l-27 | Me | Me | Me | Me | B-65 | B-65 |
| l-28 | Me | Me | Me | Me | B-73 | B-73 |
| l-29 | Me | Me | Me | Me | B-77 | B-77 |
| l-30 | Me | Me | Me | Me | B-86 | B-86 |
| l-31 | Me | Me | Me | Me | B-83 | B-83 |
| l-32 | Me | Me | Me | Me | B-90 | B-90 |
| l-33 | Me | Me | Me | Me | B-93 | B-93 |
| l-34 | Me | Me | Me | Me | B-98 | B-101 |
| l-35 | Me | Me | Me | Me | B-102 | B-105 |
| l-36 | Me | Me | Me | Me | B-106 | B-106 |
| l-37 | H | H | H | H | B-107 | B-110 |
| l-38 | H | H | H | Me | B-112 | B-115 |
| l-39 | Et | Et | Et | Et | B-116 | B-118 |
| l-40 | n-Bu | n-Bu | n-Bu | n-Bu | B-120 | B-124 |
| l-41 | Ph | Ph | Ph | Ph | B-127 | B-131 |
| l-42 | Me | Me | Me | Ph | B-132 | B-136 |
| l-43 | i-Pr | i-Pr | i-Pr | i-Pr | B-128 | B-128 |
| l-44 | 2-MeOEt | 2-MeOEt | 2-MeOEt | 2-MeOEt | B-1 | B-61 |
| l-45 | 3-tol | Me | 3-tol | Me | B-1 | B-71 |
| l-46 | Et | Et | Ph | Ph | B-30 | B-90 |
| l-47 | 4-tol | Ph | 4-tol | Me | B-1 | B-66 |
| l-48 | Me | Me | Me | Me | B-5 | B-66 |
| l-49 | 2-tol | Me | 2-tol | Me | B-70 | B-71 |
| l-50 | Me | Et | Me | Ph | B-80 | B-81 |

### [Chemical Formula 67]

(m)

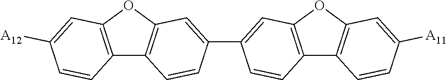

| COMPOUND | | |
|---|---|---|
| NUMBER | A₁₁ | A₁₂ |
| m-1 | B-1 | B-1 |
| m-2 | B-2 | B-2 |
| m-3 | B-3 | B-3 |
| m-4 | B-8 | B-8 |
| m-5 | B-9 | B-9 |
| m-6 | B-10 | B-10 |
| m-7 | B-14 | B-14 |
| m-8 | B-25 | B-25 |
| m-9 | B-22 | B-22 |
| m-10 | B-29 | B-29 |
| m-11 | B-33 | B-33 |
| m-12 | B-42 | B-42 |
| m-13 | B-45 | B-45 |
| m-14 | B-50 | B-50 |
| m-15 | B-31 | B-31 |
| m-16 | B-3 | B-3 |
| m-17 | B-9 | B-9 |
| m-18 | B-17 | B-18 |
| m-19 | B-3 | B-2 |
| m-20 | B-4 | B-3 |
| m-21 | B-63 | B-63 |
| m-22 | B-68 | B-68 |
| m-23 | B-71 | B-71 |
| m-24 | B-74 | B-74 |
| m-25 | B-76 | B-76 |
| m-26 | B-80 | B-80 |
| m-27 | B-83 | B-83 |
| m-28 | B-88 | B-88 |
| m-29 | B-96 | B-96 |
| m-30 | B-100 | B-100 |
| m-31 | B-117 | B-117 |
| m-32 | B-125 | B-125 |
| m-33 | B-131 | B-131 |
| m-34 | B-134 | B-134 |
| m-35 | B-135 | B-135 |
| m-36 | B-14 | B-74 |
| m-37 | B-25 | B-86 |
| m-38 | B-101 | B-10 |
| m-39 | B-6 | B-66 |
| m-40 | B-16 | B-73 |

### [Chemical Formula 68]

(n)

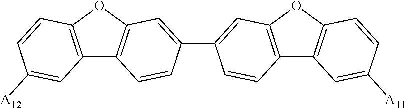

| COMPOUND | | |
|---|---|---|
| NUMBER | A₁₁ | A₁₂ |
| n-1 | B-2 | B-2 |
| n-2 | B-3 | B-3 |
| n-3 | B-9 | B-9 |
| n-4 | B-10 | B-10 |
| n-10 | B-9 | B-9 |
| n-11 | B-14 | B-14 |
| n-12 | B-10 | B-10 |
| n-13 | B-8 | B-8 |
| n-14 | B-9 | B-9 |
| n-15 | B-10 | B-10 |
| n-16 | B-21 | B-21 |
| n-17 | B-26 | B-26 |
| n-18 | B-31 | B-31 |
| n-19 | B-38 | B-38 |
| n-20 | B-42 | B-42 |
| n-21 | B-51 | B-51 |
| n-22 | B-3 | B-4 |
| n-24 | B-5 | B-5 |
| n-25 | B-3 | B-10 |
| n-26 | B-64 | B-64 |
| n-27 | B-67 | B-67 |
| n-28 | B-71 | B-71 |
| n-29 | B-75 | B-75 |
| n-30 | B-78 | B-78 |
| n-31 | B-81 | B-81 |
| n-32 | B-85 | B-85 |
| n-33 | B-88 | B-88 |
| n-34 | B-91 | B-91 |
| n-35 | B-95 | B-95 |
| n-36 | B-98 | B-98 |
| n-37 | B-101 | B-101 |
| n-38 | B-104 | B-104 |
| n-39 | B-109 | B-109 |
| n-40 | B-111 | B-111 |
| n-41 | B-112 | B-112 |
| n-42 | B-116 | B-116 |
| n-43 | B-123 | B-123 |
| n-44 | B-126 | B-126 |
| n-45 | B-127 | B-131 |
| n-46 | B-45 | B-50 |
| n-47 | B-8 | B-9 |
| n-48 | B-8 | B-10 |
| n-49 | B-72 | B-17 |
| n-50 | B-3 | B-68 |

### [Chemical Formula 69]

(o)

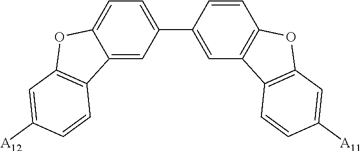

| COMPOUND | | |
|---|---|---|
| NUMBER | A₁₁ | A₁₂ |
| o-1 | B-2 | B-2 |
| o-2 | B-3 | B-3 |
| o-3 | B-9 | B-9 |
| o-4 | B-10 | B-10 |
| o-10 | B-9 | B-9 |
| o-11 | B-14 | B-14 |
| o-12 | B-10 | B-10 |
| o-13 | B-8 | B-8 |
| o-14 | B-9 | B-9 |
| o-15 | B-10 | B-10 |
| o-16 | B-21 | B-21 |
| o-17 | B-26 | B-26 |
| o-18 | B-31 | B-31 |
| o-19 | B-37 | B-37 |
| o-20 | B-43 | B-43 |
| o-21 | B-48 | B-48 |
| o-22 | B-22 | B-22 |
| o-24 | B-28 | B-28 |
| o-25 | B-1 | B-9 |
| o-26 | B-62 | B-62 |
| o-27 | B-66 | B-66 |
| o-28 | B-73 | B-73 |
| o-29 | B-77 | B-77 |
| o-30 | B-82 | B-82 |
| o-31 | B-84 | B-84 |
| o-32 | B-85 | B-85 |
| o-33 | B-86 | B-86 |
| o-34 | B-87 | B-87 |
| o-35 | B-89 | B-89 |
| o-36 | B-93 | B-93 |
| o-37 | B-98 | B-101 |
| o-38 | B-102 | B-105 |
| o-39 | B-106 | B-106 |
| o-40 | B-107 | B-110 |
| o-41 | B-112 | B-115 |
| o-42 | B-116 | B-119 |
| o-43 | B-120 | B-124 |
| o-44 | B-127 | B-131 |
| o-45 | B-132 | B-136 |
| o-46 | B-128 | B-128 |
| o-47 | B-1 | B-61 |
| o-48 | B-66 | B-70 |
| o-49 | B-72 | B-75 |
| o-50 | B-67 | B-76 |

### [Chemical Formula 70]

(p)

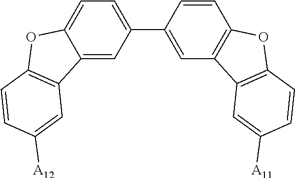

| COMPOUND | | |
|---|---|---|
| NUMBER | A₁₁ | A₁₂ |
| p-1 | B-1 | B-1 |
| p-2 | B-2 | B-2 |
| p-3 | B-3 | B-3 |
| p-4 | B-8 | B-8 |
| p-5 | B-9 | B-9 |
| p-6 | B-10 | B-10 |
| p-7 | B-14 | B-14 |
| p-8 | B-22 | B-22 |
| p-9 | B-27 | B-27 |
| p-10 | B-33 | B-33 |
| p-11 | B-42 | B-42 |
| p-12 | B-43 | B-43 |
| p-13 | B-44 | B-44 |
| p-14 | B-45 | B-45 |
| p-15 | B-31 | B-33 |
| p-16 | B-4 | B-4 |
| p-17 | B-5 | B-5 |
| p-18 | B-17 | B-17 |
| p-19 | B-1 | B-2 |
| p-20 | B-1 | B-3 |
| p-21 | B-8 | B-9 |
| p-22 | B-8 | B-10 |
| p-23 | B-1 | B-8 |
| p-24 | B-30 | B-10 |
| p-25 | B-1 | B-20 |
| p-26 | B-63 | B-63 |
| p-27 | B-68 | B-68 |
| p-28 | B-71 | B-71 |
| p-29 | B-74 | B-74 |
| p-30 | B-76 | B-76 |
| p-31 | B-80 | B-80 |
| p-32 | B-83 | B-83 |
| p-33 | B-87 | B-87 |
| p-34 | B-93 | B-93 |
| p-35 | B-95 | B-95 |
| p-36 | B-98 | B-98 |
| p-37 | B-101 | B-101 |
| p-38 | B-102 | B-102 |
| p-39 | B-106 | B-106 |
| p-40 | B-109 | B-109 |
| p-41 | B-111 | B-111 |
| p-42 | B-112 | B-112 |
| p-43 | B-116 | B-116 |
| p-44 | B-123 | B-123 |
| p-45 | B-126 | B-126 |
| p-46 | B-127 | B-131 |
| p-47 | B-45 | B-50 |
| p-48 | B-8 | B-9 |
| p-49 | B-8 | B-10 |
| p-50 | B-72 | B-17 |

### [Chemical Formula 71]

(q)

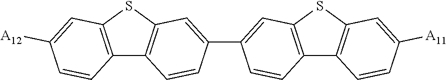

| COMPOUND | | |
|---|---|---|
| NUMBER | A₁₁ | A₁₂ |
| q-1 | B-1 | B-1 |
| q-2 | B-2 | B-2 |
| q-3 | B-3 | B-3 |
| q-4 | B-8 | B-8 |
| q-5 | B-9 | B-9 |
| q-6 | B-10 | B-10 |
| q-7 | B-14 | B-14 |
| q-8 | B-22 | B-22 |
| q-9 | B-27 | B-27 |
| q-10 | B-33 | B-33 |
| q-11 | B-42 | B-42 |
| q-12 | B-43 | B-43 |
| q-13 | B-44 | B-44 |
| q-14 | B-45 | B-45 |
| q-15 | B-31 | B-33 |
| q-16 | B-4 | B-4 |
| q-17 | B-5 | B-5 |
| q-18 | B-17 | B-17 |
| q-19 | B-1 | B-2 |
| q-20 | B-1 | B-3 |
| q-21 | B-8 | B-9 |
| q-22 | B-8 | B-10 |
| q-23 | B-1 | B-8 |
| q-24 | B-30 | B-10 |
| q-25 | B-1 | B-20 |
| q-26 | B-64 | B-64 |
| q-27 | B-67 | B-67 |
| q-28 | B-71 | B-71 |
| q-29 | B-75 | B-75 |
| q-30 | B-78 | B-78 |
| q-31 | B-81 | B-81 |
| q-32 | B-85 | B-85 |
| q-33 | B-88 | B-88 |
| q-34 | B-91 | B-91 |
| q-35 | B-95 | B-95 |
| q-36 | B-98 | B-98 |
| q-37 | B-100 | B-100 |
| q-38 | B-101 | B-101 |
| q-39 | B-102 | B-102 |
| q-40 | B-104 | B-104 |
| q-41 | B-106 | B-106 |
| q-42 | B-109 | B-109 |
| q-43 | B-111 | B-111 |
| q-44 | B-113 | B-113 |
| q-45 | B-114 | B-114 |
| q-46 | B-118 | B-118 |
| q-47 | B-124 | B-124 |
| q-48 | B-127 | B-127 |
| q-49 | B-34 | B-114 |
| q-50 | B-45 | B-105 |

### [Chemical Formula 72]

(r)

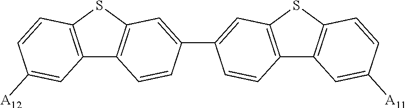

| COMPOUND | | |
|---|---|---|
| NUMBER | A₁₁ | A₁₂ |
| r-1 | B-1 | B-1 |
| r-2 | B-2 | B-2 |
| r-3 | B-3 | B-3 |
| r-4 | B-8 | B-8 |
| r-5 | B-9 | B-9 |
| r-6 | B-10 | B-10 |
| r-7 | B-14 | B-14 |
| r-8 | B-21 | B-21 |
| r-9 | B-23 | B-23 |
| r-10 | B-31 | B-33 |
| r-11 | B-42 | B-42 |
| r-12 | B-43 | B-43 |
| r-13 | B-47 | B-47 |
| r-14 | B-48 | B-48 |
| r-15 | B-31 | B-33 |
| r-16 | B-4 | B-4 |
| r-17 | B-5 | B-5 |
| r-18 | B-17 | B-17 |
| r-19 | B-1 | B-2 |
| r-20 | B-1 | B-3 |
| r-21 | B-8 | B-9 |
| r-22 | B-8 | B-10 |
| r-23 | B-1 | B-8 |
| r-24 | B-30 | B-10 |
| r-25 | B-1 | B-20 |
| r-26 | B-64 | B-64 |
| r-27 | B-67 | B-67 |
| r-28 | B-71 | B-71 |
| r-29 | B-75 | B-75 |
| r-30 | B-78 | B-78 |
| r-31 | B-81 | B-81 |
| r-32 | B-85 | B-85 |
| r-33 | B-88 | B-88 |
| r-34 | B-91 | B-91 |
| r-35 | B-95 | B-95 |
| r-36 | B-98 | B-98 |
| r-37 | B-101 | B-101 |
| r-38 | B-102 | B-102 |
| r-39 | B-106 | B-106 |
| r-40 | B-109 | B-109 |
| r-41 | B-111 | B-111 |
| r-42 | B-112 | B-112 |
| r-43 | B-116 | B-116 |
| r-44 | B-123 | B-123 |
| r-45 | B-126 | B-126 |
| r-46 | B-127 | B-131 |
| r-47 | B-45 | B-50 |
| r-48 | B-8 | B-9 |
| r-49 | B-8 | B-10 |
| r-50 | B-72 | B-17 |

### [Chemical Formula 73]

(s)

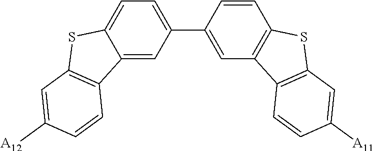

| COMPOUND | | |
|---|---|---|
| NUMBER | A₁₁ | A₁₂ |
| s-1 | B-1 | B-1 |
| s-2 | B-2 | B-2 |
| s-3 | B-3 | B-3 |
| s-4 | B-8 | B-8 |
| s-5 | B-9 | B-9 |
| s-6 | B-10 | B-10 |
| s-7 | B-51 | B-51 |
| s-8 | B-46 | B-44 |
| s-9 | B-37 | B-37 |
| s-10 | B-38 | B-38 |
| s-11 | B-33 | B-35 |
| s-12 | B-27 | B-27 |
| s-13 | B-24 | B-24 |
| s-14 | B-7 | B-7 |
| s-15 | B-7 | B-24 |
| s-16 | B-63 | B-63 |
| s-17 | B-68 | B-68 |
| s-18 | B-71 | B-71 |
| s-19 | B-76 | B-76 |
| s-20 | B-80 | B-80 |
| s-21 | B-83 | B-83 |
| s-22 | B-87 | B-87 |
| s-23 | B-93 | B-93 |
| s-24 | B-94 | B-94 |
| s-25 | B-99 | B-99 |
| s-26 | B-108 | B-108 |
| s-27 | B-114 | B-114 |
| s-28 | B-121 | B-121 |
| s-29 | B-125 | B-125 |
| s-30 | B-129 | B-129 |

### [Chemical Formula 74]

(t)

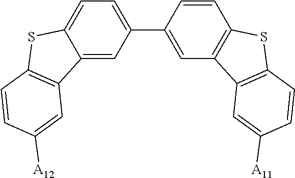

| COMPOUND | | |
|---|---|---|
| NUMBER | A₁₁ | A₁₂ |
| t-1 | B-1 | B-1 |
| t-2 | B-2 | B-2 |
| t-3 | B-3 | B-3 |
| t-4 | B-8 | B-8 |
| t-5 | B-9 | B-9 |
| t-6 | B-10 | B-10 |
| t-7 | B-14 | B-14 |
| t-8 | B-20 | B-20 |
| t-9 | B-21 | B-21 |
| t-10 | B-1 | B-1 |
| t-11 | B-2 | B-2 |
| t-12 | B-3 | B-3 |
| t-13 | B-8 | B-8 |
| t-14 | B-9 | B-9 |
| t-15 | B-10 | B-10 |
| t-16 | B-4 | B-4 |
| t-17 | B-28 | B-28 |
| t-18 | B-36 | B-36 |
| t-19 | B-40 | B-40 |
| t-20 | B-45 | B-50 |
| t-21 | B-8 | B-9 |
| t-22 | B-8 | B-10 |
| t-23 | B-1 | B-1 |
| t-24 | B-3 | B-3 |
| t-25 | B-1 | B-8 |
| t-26 | B-62 | B-62 |
| t-27 | B-66 | B-66 |
| t-28 | B-73 | B-73 |
| t-29 | B-77 | B-77 |
| t-30 | B-82 | B-82 |
| t-31 | B-84 | B-84 |
| t-32 | B-85 | B-85 |
| t-33 | B-86 | B-86 |
| t-34 | B-90 | B-90 |
| t-35 | B-97 | B-97 |
| t-36 | B-99 | B-99 |
| t-37 | B-104 | B-104 |
| t-38 | B-109 | B-109 |
| t-39 | B-111 | B-111 |
| t-40 | B-112 | B-112 |
| t-41 | B-102 | B-102 |
| t-42 | B-106 | B-106 |
| t-43 | B-109 | B-109 |
| t-44 | B-111 | B-111 |
| t-45 | B-112 | B-112 |
| t-46 | B-116 | B-116 |
| t-47 | B-123 | B-123 |
| t-48 | B-126 | B-126 |
| t-49 | B-127 | B-131 |
| t-50 | B-45 | B-50 |

### [Chemical Formula 75]

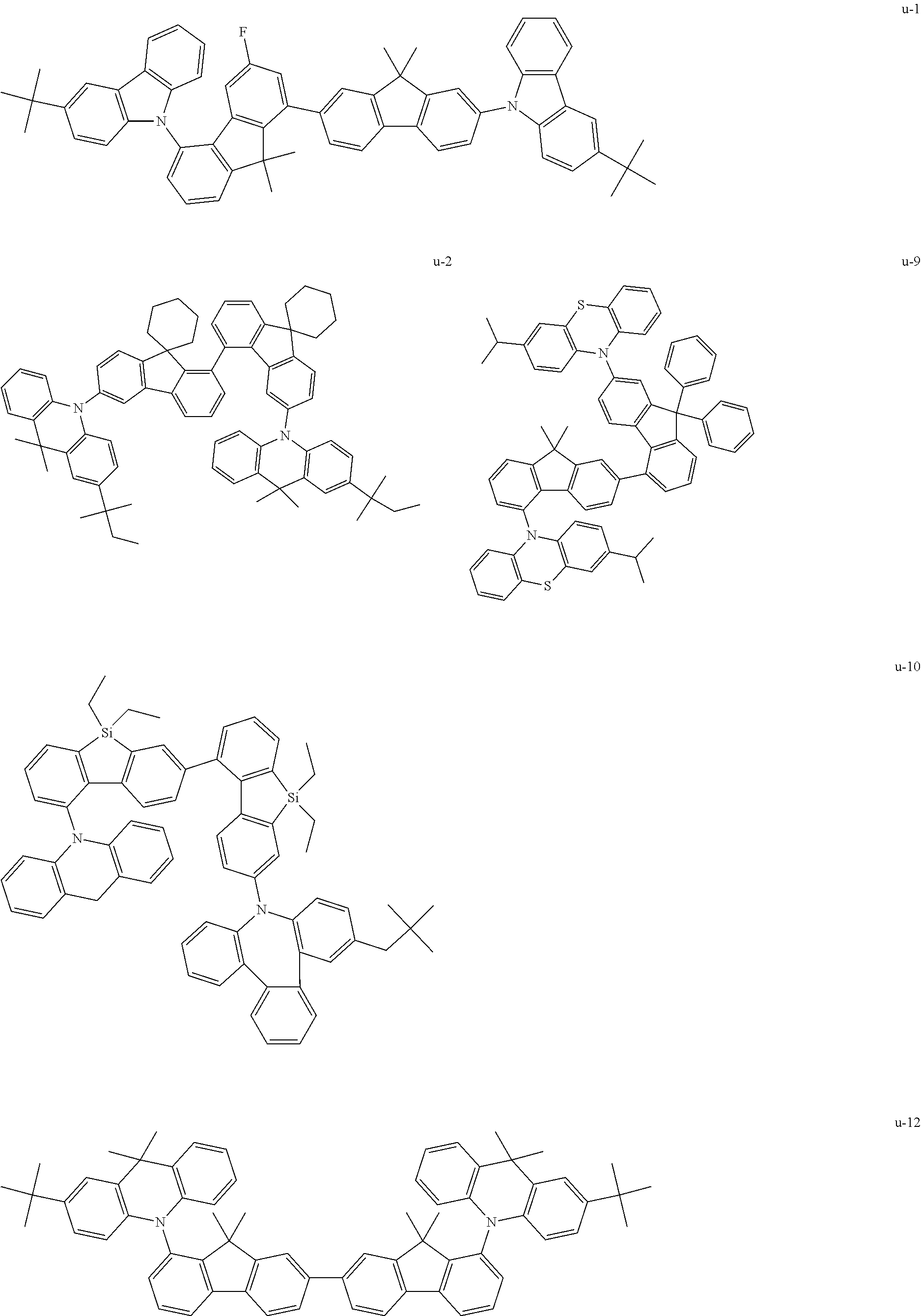

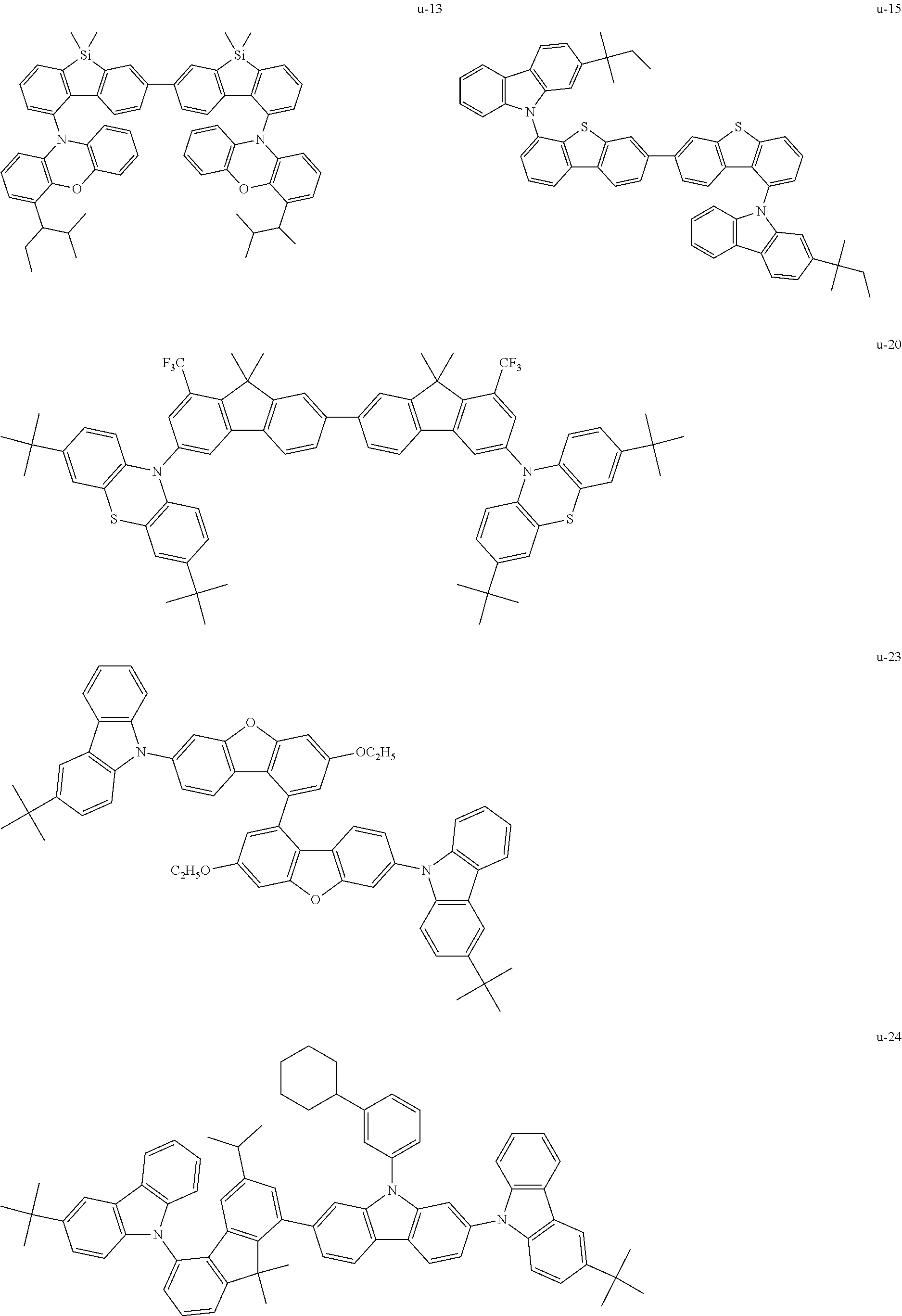

### [Chemical Formula 76]

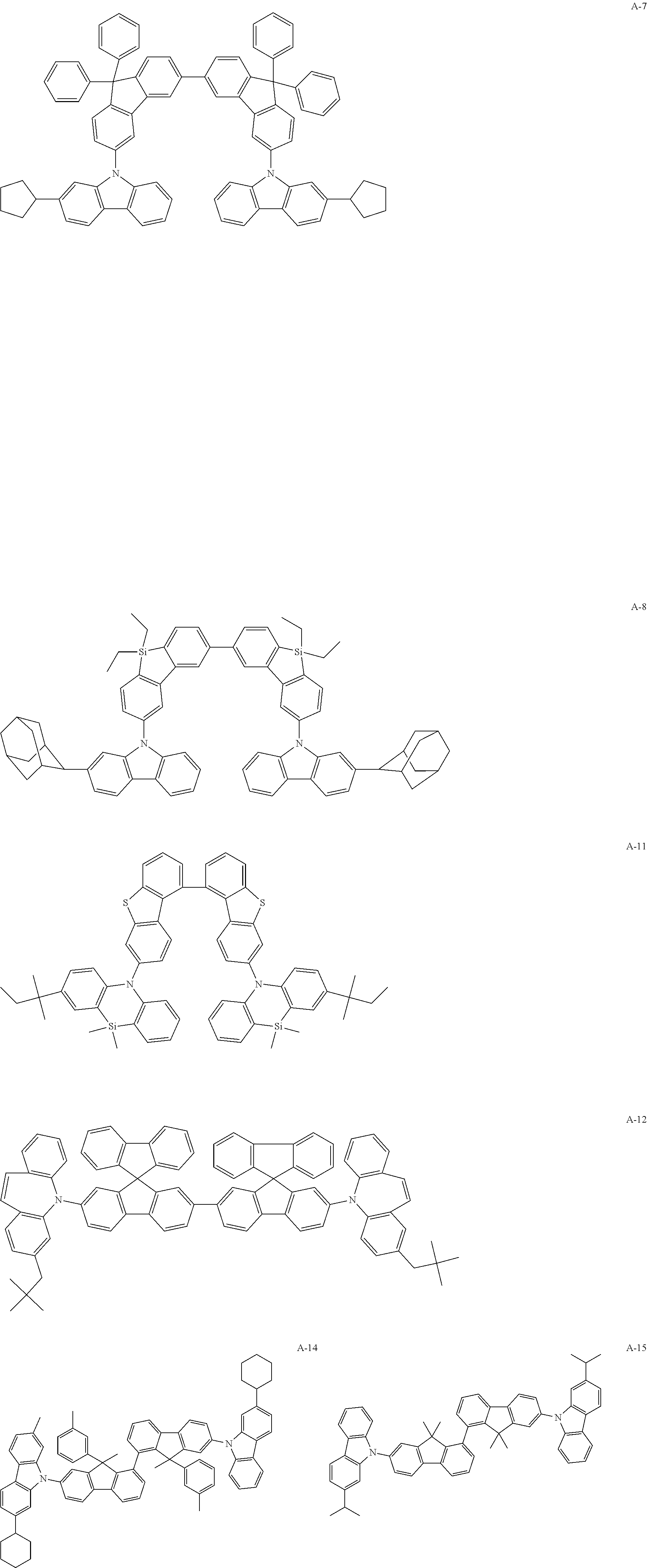

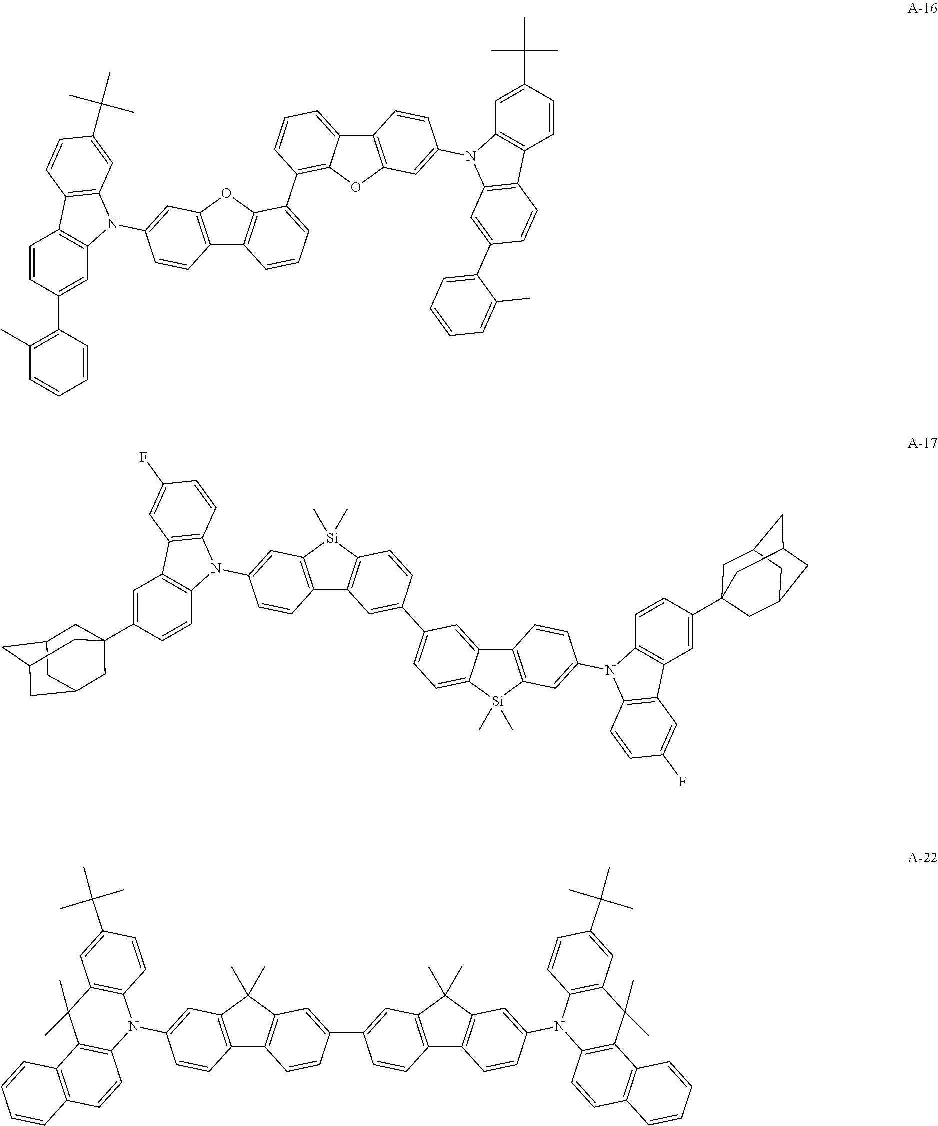 [SMILES: Cc1ccccc1-c1ccc2c3ccc(C(C)(C)C)cc3n(-c3ccc4c(c3)oc3c(-c5cccc6c5oc5cc(-n7c8cc(-c9ccccc9C)ccc8c8ccc(C(C)(C)C)cc87)ccc56)cccc34)c2c1 ; C[Si]1(C)c2ccc(-c3ccc4c(c3)-c3ccc(-n5c6ccc(F)cc6c6cc(C78CC9CC(CC(C9)C7)C8)ccc65)cc3[Si]4(C)C)cc2-c2ccc(-n3c4ccc(F)cc4c4cc(C56CC7CC(CC(C7)C5)C6)ccc43)cc21 ; CC(C)(C)c1ccc2c(c1)C(C)(C)c1c(ccc3ccccc13)N2c1ccc2c(c1)C(C)(C)c1cc(-c3ccc4c(c3)C(C)(C)c3cc(N5c6ccc(C(C)(C)C)cc6C(C)(C)c6c5ccc5ccccc65)ccc3-4)ccc1-2]

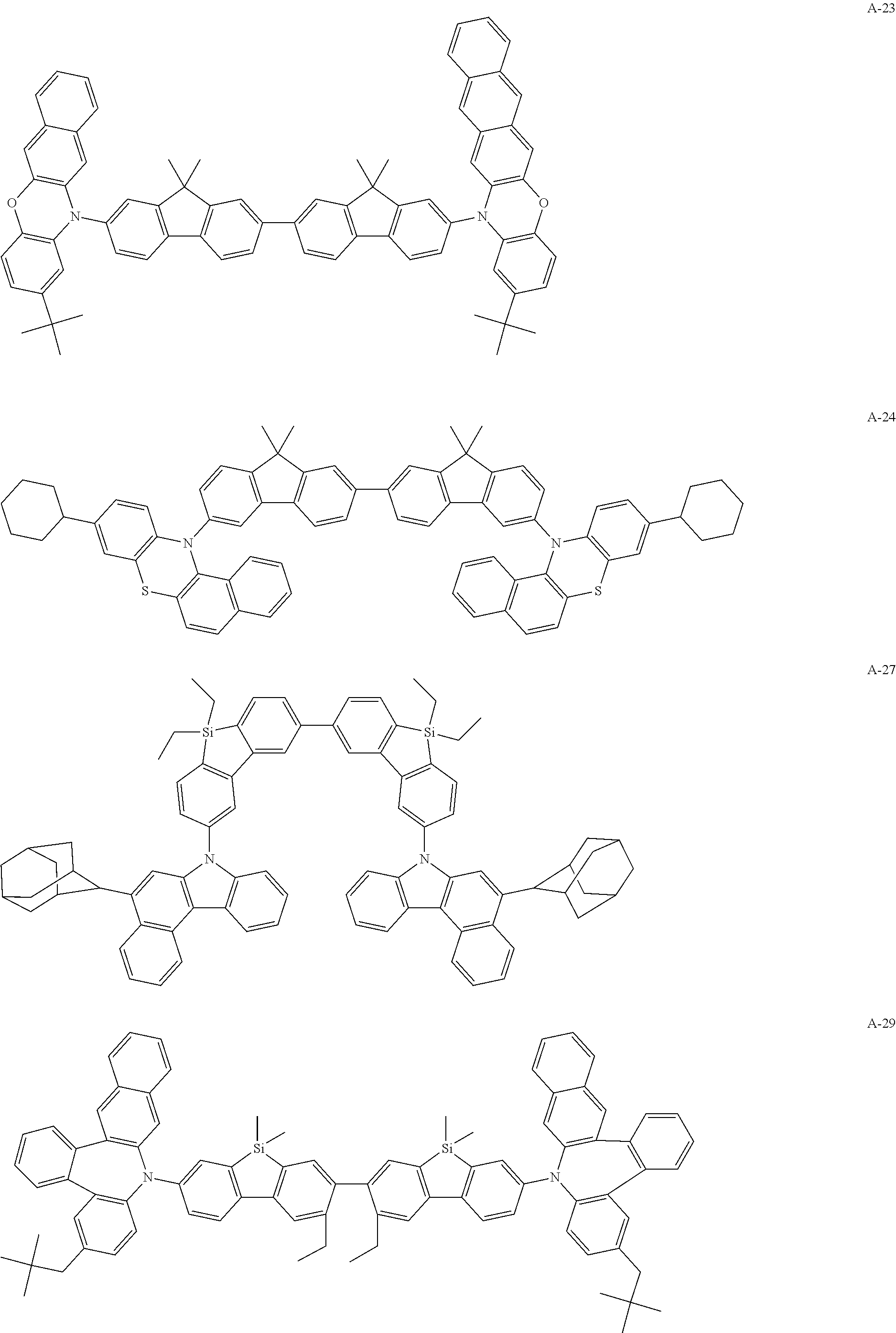

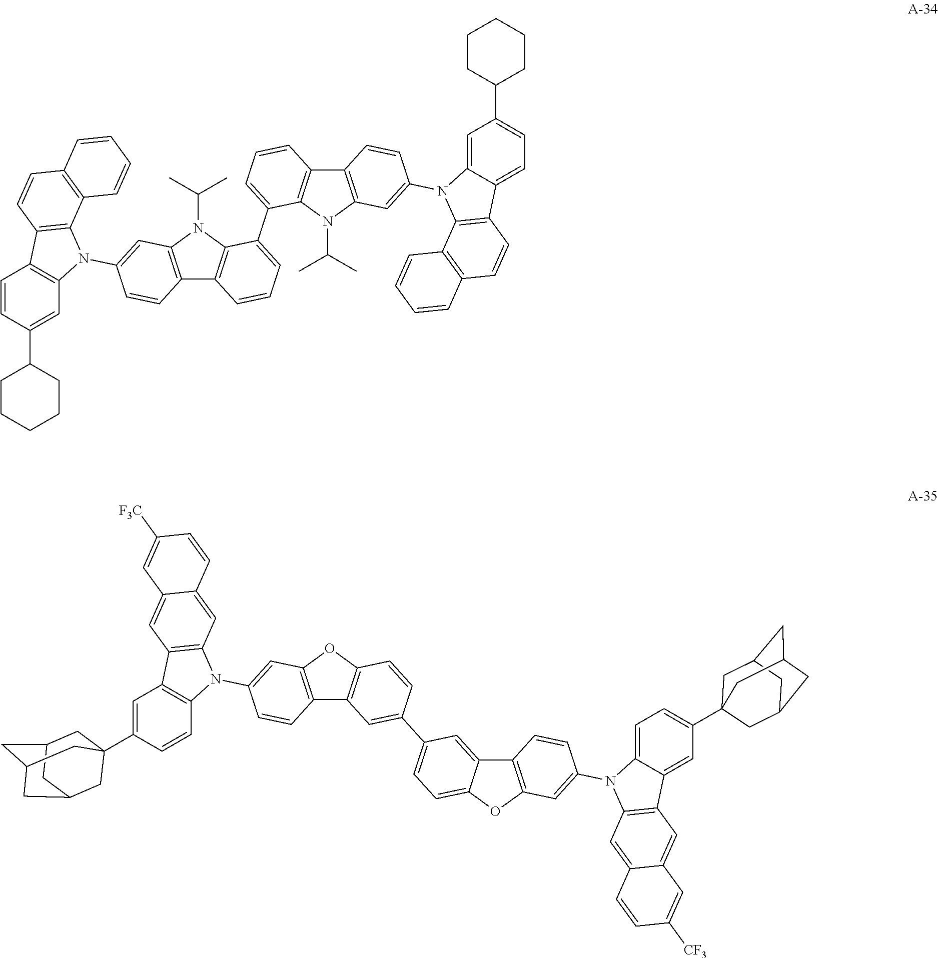 [SMILES: CC(C)n1c2cc(-n3c4cc(C5CCCCC5)ccc4c4ccc5ccccc5c43)ccc2c2cccc(-c3cccc4c5ccc(-n6c7cc(C8CCCCC8)ccc7c7ccc8ccccc8c76)cc5n(C(C)C)c34)c21 ; FC(F)(F)c1ccc2cc3c(cc2c1)c1cc(C24CC5CC(CC(C5)C2)C4)ccc1n3-c1ccc2c(c1)oc1ccc(-c3ccc4oc5cc(-n6c7ccc(C89CC%10CC(CC(C%10)C8)C9)cc7c7cc8cc(C(F)(F)F)ccc8cc76)ccc5c4c3)cc12]

A molecular weight of a compound represented by the general formula (EB-1), (EB-2), (EB-3) or (EB-4) is, preferably, 500 to 2000, more preferably 500 to 1500, even more preferably 700 to 1500, among them, preferably 800 to 1500, particularly preferably 900 to 1500, and most preferably 940 to 1500. By setting the molecular weight to not less than 500 nor more than 2000, the vapor deposition of the material can be performed, thereby improving the heat resistance.

It is preferred that when using these compounds in an organic photoelectric conversion element, an imaging element, and an organic electroluminescent element, impurities such as halide ions and metal ions are reduced in terms of the element performance.

The compound represented by the general formula (EB-1), (EB-2), (EB-3) or (EB-4) can be synthesized by applying known methods. After the synthesis, preferably, the compound is used as the organic material for deposition in the present embodiment to perform deposition, whereby the electron blocking layer is formed.

### [Substituent Group W]

Examples of the substituent group W include a halogen atom, an alkyl group (including a cycloalkyl group, a bicycloalkyl group, and a tricycloalkyl group), an alkenyl group (including a cycloalkenyl group, a bicycloalkenyl group), an alkynyl group, an aryl group, a heterocyclic group (it may be called a hetero ring group), a cyano group, a hydroxy group, a nitro group, a carboxy group, an alkoxy group, an aryloxy group, a silyloxy group, a heterocyclic oxy group, an acyloxy group, a carbamoyloxy group, an alkoxycarbonyloxy group, an aryloxycarbonyloxy group, an amino group (including an anilino group), an ammonio group, an acylamino group, an aminocarbonyl group, an alkoxycarbonylamino group, an aryloxycarbonylamino group, a sulfamoylamino group, an alkyl- or arylsulfonyl amino group, a mercapto group, an alkylthio group, an arylthio group, a heterocyclic thio group, a sulfamoyl group, a sulfo group, an alkyl- or aryl-sulfinyl group, an alkyl- or arylsulfonyl group, an acyl group, an aryloxy carbonyl group, an alkoxycarbonyl group, a carbamoyl group, an aryl and heterocyclic ring azo group, an imide group, a phosphino group, a phosphinyl group, a phosphinyloxy group, a phosphinylamino group, a phosphono group, a silyl group, a hydrazino group, a ureido group, a boronic acid group (—B(OH)₂), a phosphato group (—OPO(OH)₂), a sulfato group (—OSO₃H), and other known substituents.

More in detail, W represents the following (1) to (48) and the like.

### (1) Halogen Atom

For example, a fluorine atom, a chlorine atom, a bromine atom, an iodine atom.

### (2) Alkyl Group

W represents a linear, branched, or cyclic, substituted or unsubstituted alkyl group. They are intended to encompass (2-a) to (2-e).

### (2-a) Alkyl Group

Preferably an alkyl group having 1 to 30 carbon atoms (e.g. methyl, ethyl, n-propyl, isopropyl, t-butyl, n-octyl, eicosyl, 2-chloroethyl, 2-cyanoethyl, 2-ethylhexyl)

### (2-b) Cycloalkyl Group

Preferably, a substituted or unsubstituted cycloalkyl group having 3 to 30 carbon atoms (for example, cyclohexyl, cyclopentyl, 4-n-dodecyl cyclohexyl)

### (2-c) Bicycloalkyl Group

Preferably, a substituted or unsubstituted bicycloalkyl group having 5 to 30 carbon atoms (for example, bicyclo[1,2,2]heptane-2-yl, bicyclo[2,2,2]octane-3-yl)

### (2-d) Ticycloalkyl Group

Preferably, a substituted or unsubstituted tricycloalkyl group having 7 to 30 carbon atoms (for example, 1-adamantyl)

(2-e) Polycyclic Cycloalkyl Group with More Ring Structures

Note that the alkyl groups in the substituent group described below (e.g. an alkyl group of an alkylthio group) represent an alkyl group under this concept, but further include an alkenyl group and an alkynyl group.

### (3) Alkenyl Group

W represents a linear, branched, or cyclic, substituted or unsubstituted alkenyl group. They are intended to include the (3-a) to (3-c).

### (3-a) Alkenyl Group

Preferably, a substituted or unsubstituted alkenyl group having 2 to 30 carbon atoms (e.g., vinyl, allyl, prenyl, geranyl, oleyl)

### (3-b) Cycloalkenyl Group

Preferably, a substituted or unsubstituted cycloalkenyl group having 3 to 30 carbon atoms (for example, 2-cyclopentene-1-yl, 2-cyclohexen-1-yl)

### (3-c) Bicycloalkenyl Group

A substituted or unsubstituted bicycloalkenyl group, preferably a substituted or unsubstituted bicycloalkenyl group having 5 to 30 carbon atoms (for example, bicyclo [2,2,1]hept-2-en-1-yl, bicyclo[2 2,2]oct-2-en-4-yl)

### (4) Alkynyl Group

Preferably, a substituted or unsubstituted alkynyl group having 2 to 30 carbon atoms (e.g., ethynyl, propargyl, trimethylsilylethynyl group)

### (5) Aryl Group

Preferably, a substituted or unsubstituted aryl group having 6 to 30 carbon atoms (such as phenyl, p-tolyl, naphthyl, m-chlorophenyl, o-hexadecanoylaminophenyl, ferrocenyl)

### (6) Heterocyclic Group

Preferably, a monovalent group obtained by removing one hydrogen atom from a 5- or 6-membered, substituted or unsubstituted, aromatic or non-aromatic heterocyclic compound, more preferably a 5- or 6-membered aromatic heterocyclic group having 2 to 50 carbon atoms (e.g., 2-furyl, 2-thienyl, 2-pyrimidinyl, 2-benzothiazolyl, 2-carbazolyl, 3-carbazolyl, and 9-carbazolyl. Note that the heterocyclic group may be cationic heterocyclic group, such as 1-methyl-2-pyridinio, or 1-methyl-2-quinolinio)

### (7) Cyano Group

### (8) Hydroxy Group

### (9) Nitro Group

### (10) Carboxy Group

### (11) Alkoxy Group

Preferably, a substituted or unsubstituted alkoxy group having 1 to 30 carbon atoms (e.g. methoxy, ethoxy, isopropoxy, t-butoxy, n-octyloxy, 2-methoxyethoxy)

(12) Aryloxy Group Preferably, a substituted or unsubstituted aryloxy group having 6 to 30 carbon atoms (for example, phenoxy, 2-methylphenoxy, 4-t-butylphenoxy, 3-nitrophenoxy, 2-tetradecanoyl aminophenoxy)

### (13) Silyloxy Group

Preferably, a silyloxy group having 3 to 20 carbon atoms (for example, trimethylsilyloxy, t-butyldimethylsilyloxy)

### (14) Heterocyclic Oxy Group

Preferably, a substituted or unsubstituted heterocyclic oxy group having 2 to 30 carbon atoms (for example, 1-phenyl-tetrazole-5-oxy, 2-tetrahydropyranyloxy)

### (15) Acyloxy Group

Preferably, a formyloxy group, a substituted or unsubstituted alkyl carbonyl oxy group having 2 to 30 carbon atoms, a substituted or unsubstituted aryl carbonyl oxy group having 6 to 30 carbon atoms (for example, formyloxy, acetyloxy, pivaloyloxy, stearoyloxy, benzoyloxy, p-methoxyphenylcarbonyloxy)

### (16) Carbamoyloxy Group

Preferably, a substituted or unsubstituted, carbamoyl oxy group having 1 to 30 carbon atoms (e.g., N,N-dimethylcarbamoyloxy, N, N-diethylcarbamoyloxy, morpholino carbonyloxy, N, N-di-n-octyl aminocarbonyl oxy, N-n-octylcarbamoyl oxy)

### (17) Alkoxycarbonyloxy Group

Preferably, a substituted or unsubstituted alkoxycarbonyloxy group having 2 to 30 carbon atoms (for example, methoxycarbonyloxy, ethoxycarbonyloxy, t-butoxycarbonyloxy, n-octyl carbonyloxy)

### (18) Aryloxycarbonyloxy Group

Preferably, a substituted or unsubstituted aryloxycarbonyloxy group having 7 to 30 carbon atoms (eg, phenoxycarbonyloxy, p-methoxy phenoxycarbonyloxy, p-n-hexadecyloxy phenoxycarbonyloxy)

### (19) Amino Group

Preferably, an amino group, a substituted or unsubstituted alkylamino group having 1 to 30 carbon atoms, a substituted or unsubstituted anilino group having 6 to 30 carbon atoms (e.g., amino, methylamino, dimethylamino, anilino, N-methyl-anilino, diphenyl amino)

### (20) Ammonio Group

Preferably, an ammonio group, a substituted or unsubstituted alkyl having 1 to 30 carbon atoms, aryl, heterocyclic ring substituted ammonio group (e.g., trimethylammonio, triethyl ammonio, diphenylmethyl ammonio)

### (21) Acylamino Group

Preferably, a formylamino group, a substituted or unsubstituted alkyl carbonyl amino group having 1 to 30 carbon atoms, a substituted or unsubstituted aryl carbonyl amino group having 6 to 30 carbon atoms (e.g., formylamino, acetylamino, pivaloylamino, lauroyl amino, benzoylamino, 3,4,5-tri-n-octyloxyphenylcarbonylamino)

### (22) Amino Carbonyl Amino Group

Preferably, a substituted or unsubstituted aminocarbonylamino having 1 to 30 carbon atoms (for example, carbamoylamino, N, N-dimethylamino-carbonyl amino, N, N-diethylamino carbonylamino, morpholinocarbonylamino)

### (23) Alkoxycarbonyl Amino Group

Preferably, a substituted or unsubstituted alkoxycarbonylamino group having 2 to 30 carbon atoms (for example, methoxycarbonylamino, ethoxycarbonylamino, t-butoxycarbonylamino, n-octadecyl oxycarbonylamino, N-methyl methoxycarbonylamino)

### (24) Aryloxy Carbonyl Amino Group

Preferably, a substituted or unsubstituted aryloxycarbonylamino group having 7 to 30 carbon atoms (for example, phenoxycarbonylamino, p-chloro phenoxycarbonylamino, m-n-octyloxy phenoxycarbonylamino)

### (25) Sulfamoylamino Group

Preferably, a substituted or unsubstituted sulfamoylamino group having 0 to 30 carbon atoms (for example, sulfamoylamino, N,N-dimethylamino-sulfonyl amino, N-n-octyl aminosulfonylamino)

### (26) Alkyl or Aryl Sulfonyl Amino Group

Preferably, a substituted or unsubstituted alkylsulfonylamino having 1 to 30 carbon atoms, a substituted or unsubstituted arylsulfonylamino having 6 to 30 carbon atoms (for example, methylsulfonylamino, butylsulfonylamino, phenylsulfonylamino, 2,3,5-trichlorophenyl sulfonylamino, p-methylphenyl sulfonylamino)

### (27) Mercapto Group

### (28) Alkylthio Group

Preferably, a substituted or unsubstituted alkylthio group having 1 to 30 carbon atoms (for example methylthio, ethylthio, n-hexadecylthio)

### (29) Arylthio Group

Preferably, a substituted or unsubstituted arylthio having 6 to 30 carbon atoms (for example, phenylthio, p-chlorophenylthio, m-methoxyphenylthio)

### (30) Heterocyclic Thio Group

Preferably, a substituted or unsubstituted heterocyclic thio group having 2 to 30 carbon atoms (for example, 2-benzothiazolylthio, 1-phenyl tetrazole-5-ylthio)

### (31) Sulfamoyl Group

Preferably, a substituted or unsubstituted sulfamoyl group having 0 to 30 carbon atoms (for example, N-ethyl sulfamoyl, N-(3-dodecyloxypropyl) sulfamoyl, N,N-dimethyl-sulfamoyl, N-acetylsulfamoyl, N-benzo ylsulfamoyl, N—(N′-phenylcarbamoyl) sulfamoyl)

### (32) Sulfo Group

### (33) Alkyl or Aryl Sulfinyl Group

Preferably, a substituted or unsubstituted alkyl sulfinyl group having 1 to 30 carbon atoms, a substituted or unsubstituted aryl sulfinyl group having 6 to 30 carbon atoms (eg, methylsulfinyl, ethylsulfinyl, phenylsulfinyl, p-methylphenyl sulfinyl

### (34) Alkyl or Arylsulfonyl Group

Preferably, a substituted or unsubstituted alkylsulfonyl group having 1 to 30 carbon atoms, a substituted or unsubstituted arylsulfonyl group having 6 to 30 carbon atoms, for example, methylsulfonyl, ethylsulfonyl, phenylsulfonyl, p-methylphenyl sulfonyl)

### (35) Acyl Group

Preferably, a formyl group, a substituted or unsubstituted alkyl carbonyl group having 2 to 30 carbon atoms, a substituted or unsubstituted aryl carbonyl group having 7 to 30 carbon atoms, a substituted or unsubstituted heterocyclic carbonyl group having 4 to 30 carbon atoms which is attached to a carbonyl group with the carbon atoms (e.g., acetyl, pivaloyl, 2-chloroacetyl, stearoyl, benzoyl, p-n-octyloxyphenyl carbonyl, 2-pyridylcarbonyl, 2-furyl carbonyl)

### (36) Aryloxy Carbonyl Group

Preferably, a substituted or unsubstituted aryloxy carbonyl group having 7 to 30 carbon atoms (for example, phenoxycarbonyl, o-chlorophenoxycarbonyl, m-nitrophenoxycarbonyl, p-t-butyl-phenoxycarbonyl)

### (37) Alkoxycarbonyl Group

Preferably, a substituted or unsubstituted alkoxycarbonyl group having 2 to 30 carbon atoms (eg, methoxycarbonyl, ethoxycarbonyl, t-butoxycarbonyl, n-octadecyl oxycarbonyl)

### (38) Carbamoyl Group

Preferably, a substituted or unsubstituted carbamoyl having 1 to 30 carbon atoms (for example, carbamoyl, N-methylcarbamoyl, N,N-dimethylcarbamoyl, N,N-di-n-octylcarbamoyl, N-(methylsulfonyl) carbamoyl)

### (39) Aryl and Heterocyclic Azo Group

Preferably, a substituted or unsubstituted aryl azo group having 6 to 30 carbon atoms, a substituted or unsubstituted heterocyclic azo group having 3 to 30 carbon atoms (for example, phenylazo, p-chlorophenyl azo, 5-ethylthio-1,3,4-thiadiazole-2-ylazo)

### (40) Imide Group

Preferably, N-succinimide, N-phthalimide

### (41) Phosphino Group

Preferably, a substituted or unsubstituted phosphino group having 2 to 30 carbon atoms (for example, a dimethylphosphino, a diphenylphosphino, a methyl phenoxy phosphino)

### (42) Phosphinyl Group

Preferably, a substituted or unsubstituted phosphinyl group having 2 to 30 carbon atoms (for example, a phosphinyl, a dioctyloxyphosphinyl, a diethoxyphosphinyl)

### (43) Phosphinyloxy Group

Preferably, a substituted or unsubstituted phosphinyloxy group having 2 to 30 carbon atoms (for example, diphenoxyphosphoryloxy, dioctyloxyphosphinyl phosphinyloxy)

### (44) Phosphinylamino Group

Preferably, a substituted or unsubstituted phosphinylamino group having 2 to 30 carbon atoms (for example, dimethoxy phosphinyl amino, dimethylamino phosphinylamino)

### (45) Phospho Group

### (46) Silyl Group

Preferably, a substituted or unsubstituted silyl group having 3 to 30 carbon atoms (for example, trimethylsilyl, triethylsilyl, triisopropylsilyl, t-butyldimethylsilyl, phenyl dimethylsilyl)

### (47) Hydrazino Group

Preferably a substituted or unsubstituted hydrazino group having 0 to 30 carbon atoms (for example, trimethyl hydrazino)

### (48) Ureido Group

Preferably a substituted or unsubstituted ureido group having 0 to 30 carbon atoms (for example N, N-dimethyl-ureido)

Among the substituent groups W described above, those having a hydrogen atom may be substituted with the above group by removing this hydrogen atom. Examples of such substituent groups, —CONHSO₂— group (a sulfonyl carbamoyl group, a carbonyl sulfamoyl group), —CONHCO-group (a carbonyl carbamoyl group), —SO₂NHSO₂— group (sulfonyl sulfamoyl group). More specifically, an alkylcarbonylaminosulfonyl group (for example, acetylamino sulfonyl), an arylcarbonyl aminosulfonyl group (e.g., a benzoyl amino sulfonyl group), an alkylsulfonyl aminocarbonyl groups (for example, methylsulfonyl aminocarbonyl), arylsulfonylamino carbonyl group (for example, p-methyl phenyl sulfonyl amino carbonyl).

### [Ring R]

The rings R include an aromatic or non-aromatic hydrocarbon ring, a heterocyclic ring, a polycyclic condensed ring combined with these. For example, the rings include a benzene ring, naphthalene ring, anthracene ring, phenanthrene ring, a fluorene ring, a triphenylene ring, a naphthacene ring, a biphenyl ring, a pyrrole ring, a furan ring, a thiophene ring, an imidazole ring, an oxazole ring, a thiazole ring, a pyridine ring, a pyrazine ring, a pyrimidine ring, a pyridazine ring, an indolizine ring, an indole ring, a benzofuran ring, a benzothiophene ring, an isobenzofuran ring, a quinolizine ring, a quinoline ring, a phthalazine ring, naphthyridine ring, a quinoxaline ring, a quinoxazoline ring, an isoquinoline ring, a carbazole ring, a phenanthridine ring, an acridine ring, a phenanthroline ring, a thianthrene ring, a chromene ring, a xanthene ring, a phenoxathiin ring, a phenothiazine ring, and a phenazine ring. Ring R may further have a substituent group of the substituent W.

### [Imaging Element]

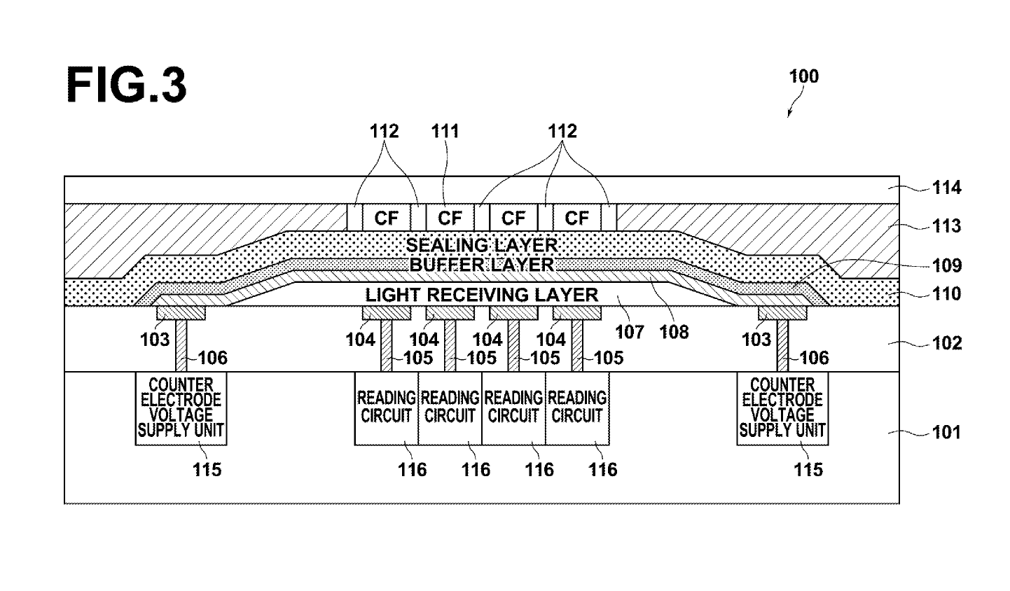

Next, the configuration of the imaging element (light sensor) 100 including the photoelectric conversion element 1 will be described with reference to FIG. 3. FIG. 3 is an exemplary cross sectional view showing a schematic configuration of an imaging element for explaining one embodiment of the present invention. The imaging element is used by being mounted on an imaging device, such as a digital camera or a digital video camera, an electronic endoscope, an imaging module, such as a cellular phone.

The imaging element 100 includes a plurality of the organic photoelectric conversion elements 1 with the configuration shown in FIG. 1, and a circuit board on which the reading circuit for reading a signal corresponding to charges generated in the photoelectric conversion layer of each organic photoelectric conversion element. On the same upper surface of the circuit substrate, the organic photoelectric conversion elements 1 are arranged in one-dimensional or two-dimensional manner.

The imaging element 100 includes a substrate 101, an insulating layer 102, a connection electrode 103, a pixel electrode 104, a connection portion 105, a connection portion 106, a light receiving layer 107, a counter electrode 108, a buffer layer 109, a sealing layer 110, a color filter (CF) 111, a partition 112, a light shielding layer 113, a protective layer 114, a counter electrode voltage supply unit 115 and a reading circuit 116.

The pixel electrode 104 has the same function as the lower electrode 20 of the organic photoelectric conversion element 1 shown in FIG. 1. The counter electrode 108 has the same function as the upper electrode 40 of the organic photoelectric conversion element 1 shown in FIG. 1. The light receiving layer 107 has the same configuration as the light receiving layer 30 provided between the lower electrode 20 and the upper electrode 40 of the organic photoelectric conversion element 1 shown in FIG. 1. The sealing layer 110 has the same function as the sealing layer 50 of the organic photoelectric conversion element 1 shown in FIG. 1. The pixel electrode 104, a part of the counter electrode 108 opposed to this, the light receiving layer 107 sandwiched between these electrodes, and parts of the buffer layer 109 and sealing layer 110 facing the pixel electrode 104 configure the organic photoelectric conversion element.

The substrate 101 is a semiconductor substrate, such as a glass substrate or Si. On the substrate 101, an insulating layer 102 is formed. The surface of the insulating layer 102 has a plurality of pixel electrodes 104 and a plurality of connection electrodes 103 formed thereon.

The light receiving layer 107 is a common layer to all the organic photoelectric conversion element that is provided to cover them over a plurality of pixel electrodes 104.

The counter electrode 108 is a common one electrode to all the organic photoelectric conversion elements provided on the light receiving layer 107. The counter electrode 108 is formed over the top of the connection electrode 103 disposed outside the light receiving layer 107, and is electrically connected to the connection electrode 103.

The connection portion 106 is buried in the insulating layer 102, and is a plug or the like for electrically connecting the connection electrode 103 to the counter electrode voltage supply unit 115. The counter electrode voltage supply unit 115 is formed in the substrate 101, and a predetermined voltage is applied to the counter electrode 108 via the connection portion 106 and a connection electrode 103. If the voltage to be applied to the counter electrode 108 is higher than the power supply voltage of the imaging element, the power supply voltage is increased by a boost circuit such as a charge pump to supply the above predetermined voltage.

The reading circuits 116 are provided on the substrate 101 corresponding to the respective pixel electrodes 104, and is adapted to read a signal corresponding to the charge captured in the corresponding pixel electrode 104. A reading circuit 116 is configured of, for example a CCD, MOS circuit, or a TFT circuit and the like, and are blocked by the light blocking layer (not shown) disposed in the insulating layer 102. The reading circuit 116 is electrically connected via the connection portion 105 to the corresponding pixel electrode 104.

The buffer layer 109 is formed over the counter electrode 108 to cover the counter electrode 108. The sealing layer 110 is formed over the buffer layer 109 to cover the buffer layer 109. The color filter 111 is formed in a position facing the respective pixel electrodes 104 on the sealing layer 110. The partition 112 is provided between the color filter 111s, and is intended to improve the light transmission efficiency of the color filter 111.

The light shielding layer 113 is formed in the region on the sealing layer 110 other than the region where the color filter 111 and the partition 112 are formed, thereby preventing the light from entering the light receiving layer 107 formed in regions other than the effective pixel regions. The protective layer 114 is formed over the color filter 111, the partition 112, and the light shielding layer 113 to protect the entire imaging element 100.

In the thus-configured imaging element 100, when light is incident, the light is incident on the light receiving layer 107, where the charge is generated. Holes of the generated charges are collected by the pixel electrode 104, and a voltage signal corresponding to the amount is output to the outside of the imaging element 100 by the reading circuit 116.

A manufacturing method of the imaging element 100 is as follows.

The connection portions 105 and 106, a plurality of connecting electrodes 103, a plurality of pixel electrodes 104, and the insulating layer 102 are formed over the circuit substrate on which the counter electrode voltage supply unit 115 and the reading circuit 116 are formed. The pixel electrodes 104 are arranged on the surface of the insulating layer 102, for example, in a square grid pattern.

Then, on the plurality of pixel electrodes 104, the light receiving layer 107, the counter electrode 108, the buffer layer 109, and the sealing layer 110 are formed in this order. Method of forming the light receiving layer 107, counter electrode 108, and sealing layer 110 have been noted in the description of the photoelectric conversion element 1. The buffer layer 109 is formed, for example, by vacuum resistance heating deposition method. Then, after forming the color filter 111, the partition 112, and the light shielding layer 113, the protective layer 114 are formed to complete the imaging element 100.

### “Organic Electroluminescent Element”

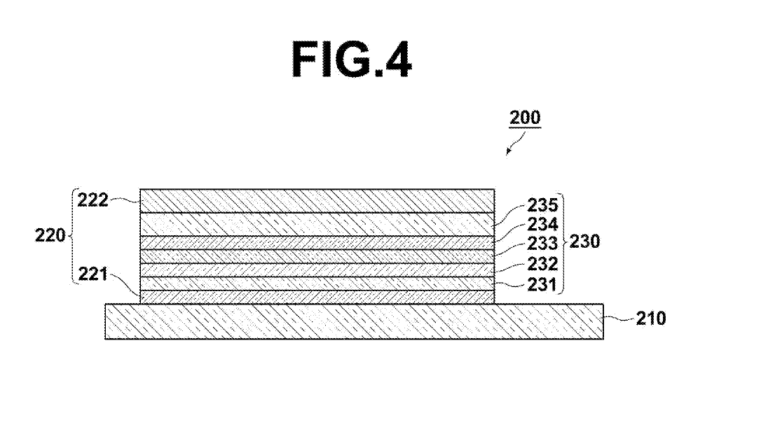

In the above, the embodiment has been described in which a photoelectric conversion element suitable as an imaging element, as well as the imaging element include an organic layer deposited by using the organic material for deposition of the present invention. The organic material 60 for deposition of the present invention can also be preferably used for deposition of the organic layer in the organic electroluminescent element and in the photoelectric conversion element suitable as the organic electroluminescent element. The organic electroluminescent element will be described below with reference to FIG. 4.

FIG. 4 is a schematic cross sectional view of the organic electroluminescent element 200 in one embodiment of the organic electroluminescent element of the present invention. The organic electroluminescent element 200 shown in FIG. 4 is a light emitting element comprising a plurality of the photoelectric conversion elements in the present invention, in which an organic layer 230 is sandwiched between a pair of electrodes 220 (anode 221 and cathode 222) over a support substrate 210. Specifically, a hole injection layer 231, a hole transport layer 232, an electron blocking layer 233, a light emitting layer (photoelectric conversion layer) 234, and an electron transport layer 235 are stacked in this order between the anode 221 and cathode 222. The hole injection layer 231, the hole transport layer 232, the electron blocking layer 233, the light-emitting layer (photoelectric conversion layer) 234, and the electron transport layer 235 serve as the organic layer 230. The light is emitted from the light emitting layer 230 by applying a voltage between the pair of electrodes 220, so that the light can be taken out of the end surface on the transparent electrode (for example, the anode 221) side from which the light is to be taken out.

Layers sandwiched between the pair of electrodes 220 are all made of an organic layer in this embodiment. At least one layer may be an organic layer.

In the organic electroluminescent element 200, at least one layer of the organic layers 230 may be deposited using the organic material 60 for deposition described above. However, even in the organic electroluminescent element 200, as many layers as possible are preferably made using the organic material 60 for deposition.

The organic material 60 for deposition may be any one of a luminescent material, a host material, an electron transport material, a hole transport material, an electron blocking material, and a hole blocking material; preferably, a luminescent material, a host material, a hole transport material, an electron blocking material; and more preferably a luminescent material, a host material, and a hole transport material.

The layer structure of the organic layer 230 is not particularly limited, and can be appropriately selected according to the use or aim of the organic electroluminescent element. Preferably, the layer structure of the organic layer 230 is formed over either of the pair of the electrodes 220. In this case, the organic layer 230 is formed over the entire surface or one surface of either of the pair of electrodes 220.

The shape, size, thickness, and the like of the organic layer are not specifically limited, but can be selected depending on the aim as appropriate.

The structure of the organic layer in the organic electroluminescent element 200 include the following structure, in addition to the structure shown in FIG. 4. However, the invention is not limited to these structures.

Anode/hole transport layer/light emitting layer/electron transport layer/cathode

Anode/hole transport layer/light emitting layer/hole blocking layer/electron transport layer/cathode

Anode/hole transport layer/light emitting layer/hole blocking layer/electron transport layer/electron injection layer/cathode

Anode/hole injection layer/hole transport layer/light emission layer/hole blocking layer/electron transport layer/cathode

Anode/hole transport layer/electron blocking layer/light emitting layer/electron transport layer/cathode

Anode/hole transport layer/electron blocking layer/light emitting layer/electron transport layer/electron injection layer/cathode

Anode/hole injection layer/hole transport layer/electron blocking layer/light emitting layer/electron transport layer/electron injection layer/cathode

Anode/hole injection layer/hole transport layer/electron blocking layer/light emitting layer/hole blocking layer/electron transport layer/electron injection layer/cathode

Anode/hole injection layer/hole transport layer/electron blocking layer/light emitting layer/hole blocking layer/electron injection layer/cathode

Anode/hole injection layer/hole transport layer/electron blocking layer/light emitting layer/hole blocking layer/electron transport layer/cathode

Anode/hole injection layer/hole transport layer/light emitting layer/blocking layer/electron transport layer/electron injection layer/cathode

Anode/hole injection layer/hole transport layer/electron blocking layer/light emitting layer/hole blocking layer/electron transport layer/electron injection layer/cathode

Anode/hole injection layer/hole transport layer/light emitting layer/electron transport layer/electron injection layer/cathode

In the following, respective elements constituting the organic electroluminescent device 200 will be described in detail.

### <Substrate>

The substrate 210 is preferably a substrate that does not scatter or attenuate light emitted from the organic layer 230. When the substrate is made of organic material, the substrate preferably has excellent heat resistance, dimensional stability, solvent resistance, electrical insulation, and workability.

### <Anode>

The anode 221 is usually sufficient to have a function as an electrode supplying holes to the organic layer 230. The shape, structure, and size of the anode are not particularly limited, and can select material from the known electrode materials as appropriate, depending on application and aim of the light emitting element. As described above, the anode is usually provided as a transparent anode.

### <Cathode>

A cathode 222 is usually sufficient to have a function as an electrode for supplying electrons to the organic layer 230. The shape, structure, and size of the cathode are not particularly limited, and can select material from the known electrode materials as appropriate, depending on application and aim of the light emitting element.

As to the substrate 210, anode 221, and cathode 222, the matters described in paragraphs [0070]-[0089] of Japanese Unexamined Patent Publication No. 2008-270736 can also be applied to the present application.

### <Organic Layer>

The organic layer 230, as described above, include the hole injection layer 231, the hole transport layer 232, the electron blocking layer 233, the light emitting layer (photoelectric conversion layer) 234, and the electron transport layer 235. These organic layers can be formed, for example, by physical vapor deposition, sputtering, and a dry film forming method such as chemical vapor deposition.

A light emitting layer 234, when an electric field is applied thereto, receives holes from the anode 221, hole injection layer 231, or hole transport layer 232, and also receives electrons from the cathode 222, the electron injection layer (not shown), or the electron transport layer 235, and thus the light emitting layer 234 is a layer having a function of emitting light by providing the site of recombination between the holes and electrons.

The light emitting layer 234 may consist of only a light emitting material, and may be configured as a mixed layer of a host material and a luminescent material. As the light emitting material, a fluorescent material or a phosphorescent material can be used, and the dopant may be of one kind or two or more kinds.

The host material is preferably a charge transport material. The host material may also be of one kind or two or more kinds, for example, may be a mixture of an electron transport host material and a hole transport host material. Furthermore, the light emitting layer 234 may contain material that does not emit light (binder material) without the charge transport property.

Further, the light emitting layer 234 may be a single layer or a multilayer of two or more layers. The respective light emitting layers may emit lights in different colors.

(Fluorescent Material)

Examples of fluorescent materials include for example, benzoxazole derivatives, benzimidazole derivatives, benzothiazole derivatives, styryl benzene derivatives, polyphenyl derivatives, diphenyl butadiene derivatives, tetraphenyl butadiene derivatives, naphthalimide derivatives, coumarin derivatives, condensed aromatic compounds, perinone derivatives, oxadiazole derivatives, oxazine derivatives, aldazine derivatives, pyralidine derivatives, cyclopentadiene derivatives, bisstyrylanthracene derivatives, quinacridone derivatives, pyrrolopyridine derivatives, thiadiazolopyridine derivatives, cyclopentadiene derivatives, styrylamine derivatives, diketo pyrrolopyrrole derivatives, aromatic dimethylidyne compounds, various complexes represented by complexes of 8-quinolinol derivatives and complexes of pyromethene derivatives, polythiophene, polyphenylene, polymer compounds such as polyphenylene vinylene, and compounds such as organic silane derivatives and the like.

(Phosphorescent Material)

Examples of the phosphorescent material include phosphorescent compounds disclosed in the following patent documents: e.g. U.S. Pat. Nos. 6,303,238, 6,097,147, International Patent Publication Nos. 2000/057676, 2000/070655, 2001/008230, 2001/039234, 2001/041512, 2002/002714, 2002/015645, 2002/044189, 2005/019373, Japanese Unexamined Patent Publication Nos. 2001-247859, 2002-302671, 2002-117978, 2003-133074, 2002-235076, 2003-123982, 2002-170684, European Patent Publication No. 1211257, Japanese Unexamined Patent Publication Nos. 2002-226495, 2002-234894, 2001-247859, 2001-298470, 2002-173674, 2002-203678, 2002-203679, 2004-357791, 2006-256999, 2007-019462, 2007-084635, 2007-096259 and the like. Among them, more preferably, examples of light emitting dopants include an Ir complex, a Pt complex, a Cu complex, a Re complex, a W complex, a Rh complex, a Ru complex, a Pd complex, an Os complex, an Eu complex, a Tb complex, a Gd complex, a Dy complex, and a Ce complexes. Particularly preferably, examples of the light emitting dopants are an Ir complex, a Pt complex, and a Re complex. Among them, the Ir complex, the Pt complex, or the Re complex containing a coordination form of at least one of a metal-carbon bond, a metal-nitrogen bond, a metal-oxygen bond, and a metal-sulfur bond are preferable. Further, in terms of the light emission efficiency, driving durability, chromaticity, etc., the Ir complex, the Pt complex, or the Re complex containing a tridentate or higher multidentate ligand are particularly preferable.

The content of the luminescent material with respect to the total mass of the light emitting layer 234 is preferably in a range of 0.1 mass % or more to 50 mass % or less, more preferably in the range of 1 mass % or more to 40 mass % or less, and most preferably, 5 mass % or more to 30 mass % or less. In particular, within the range of 5 mass % or more to 30 mass % or less, the chromaticity of light emission from the organic electroluminescent element 200 is less likely to depend on the concentration of addition of the light emitting material.

(Host Material)

The host material is a compound that mainly implants and transports charges in the light emitting layer. The host material itself is a compound that does not substantially emit light. The term “not substantially emit light” as used in the present specification means that a light emission amount from the compound that does not substantially emit light to the total amount of light emission from the entire element is 5% or less, more preferably 3% or less, and even more preferably 1% or less.

The light emitting layer 234 preferably includes the host material. As the host material, a hole transport host material, an electron transport host material, or both thereof, which is so-called bipolar host material, is used. The bipolar host material is preferable.

The concentration of the host material in the light emitting layer 234 is not particularly limited, but the host material is preferably a main component (a component whose content is the most) in a light emitting layer 234. The concentration of the host material is more preferably not less than 50 mass % nor more than 99.9 mass %, even more preferably not less than 50 mass % nor more than 99.8 mass %, particularly preferably not less than 60 mass % nor more than 99.7 mass %, and most preferably not less than 70 mass % nor more than 95 mass %.

A glass transition temperature Tg of the host material is preferably not less than 60° C. nor more than 500° C., more preferably not less than 90° C. nor more than 250° C., and even more preferably not less than 130° C. nor more than 250° C. Among them, the Tg is still more preferably not less than 175° C. nor more than 250° C., particularly preferably, not less than 200° C. nor more than 250° C., and most preferably not less than 220° C. nor more than 250° C.

In the light emitting layer 234, it is preferable that the lowest triplet excitation energy (T₁ energy) of the host material is higher than that of the T₁ energy of the light emitting material from the viewpoint of the light emission efficiency and driving durability.

The host material may contain the following compounds in its partial structure. For example, pyrrole, indole, carbazole (e.g. CBP (4,4′-di(9-carbazolyl)biphenyl)), azaindole, azacarbazole, triazole, oxazole, oxadiazole, pyrazole, imidazole, thiophene, polyarylalkane, pyrazoline, pyrazolone, phenylenediamine, arylamine, amino-substituted chalcone, styryl anthracene, fluorenone, hydrazone, stilbene, silazane, an aromatic tertiary amine compound, a styrylamine compound, a porphyrin compound, a polysilane compound, poly (N-vinyl carbazole), an aniline copolymer, a thiophene oligomer, a conductive high molecular oligomer such as polythiophene, organic silane, carbon film, pyridine, pyrimidine, triazine, anthraquinodimethane, anthrone, diphenylquinone, thiopyrandioxide, carbodiimide, fluorenylidene methane, distyrylpyrazine, fluorine-substituted aromatic compounds, a heterocyclic tetracarboxylic acid anhydride such as naphthalene perylene, phthalocyanine, a metal complex of 8-quinolinone derivative, a metal phthalocyanine, or various metal complexes, typified by metal complexes containing benzoxazole or benzothiazole as a ligand, and derivatives thereof (which may have a substituent group or a condensed ring), and materials to be described by way of example in the following sections about the hole injection layer, hole transport layer, electron injection layer, and electron transport layer.

Further, as a host material, for example, the compound described in paragraphs [0113] to [0161] of Japanese Unexamined Patent Publication No. 2002-100476, and the compound described in paragraphs [0087] to [0098] of Japanese Unexamined Patent Publication No. 2004-214179 can be suitably used, but the host material is not limited thereto.

The thickness of the light emitting layer 234 is not particularly limited, but generally, is preferably from 1 nm to 500 nm, more preferably from 5 nm to 200 nm, and even more preferably from 10 nm to 100 nm

### —Hole Injection Layer, Hole Transport Layer—

The hole injection layer 231, and hole transport layer 232 are provided between the anode 221 and the light emitting layer 234, and are layers having a function of receiving holes from the anode 221 or the anode 221 side to transport the holes to the cathode 222 side. Specifically, the hole injection layer 231 and the hole transport layer 232 are preferably layers that include a carbazole derivative, a triazole derivative, an oxazole derivative, an oxadiazole derivative, an imidazole derivative, a polyarylalkane derivative, a pyrazoline derivative, a pyrazolone derivative, a phenylenediamine derivative, an arylamine derivative, an amino-substituted chalcone derivative, a styryl anthracene derivative, a fluorenone derivative, a hydrazone derivative, a stilbene derivative, a silazane derivative, an aromatic tertiary amine compound, a styrylamine compound, a porphyrin compound, an organic silane derivative, carbon, etc.

The thickness of the hole injection layer 231, and the thickness of the hole transport layer 232 each are preferably equal to or less than 500 nm from the viewpoint of lowering the driving voltage.

The thickness of the hole transport layer 232 is preferably from 1 nm to 500 nm, more preferably from 5 nm to 200 nm, and even more preferably from 5 nm to 100 nm. The thickness of the hole injection layer 231 is preferably from 0.1 nm to 500 nm, more preferably from 0.5 nm to 300 nm, and even more preferably from 1 nm to 200 nm.

The hole injection layer 231, and the hole transport layer 232 may be a single layer structure made of one or two or more kinds of the materials described above, or may be a multi-layered structure consisting of a plurality of layers made of the same composition or different compositions.

### —Electron Injection Layer, Electron Transport Layer—

An electron injection layer (not shown), and the electron transport layer 235 are provided between the cathode 222 and the light emitting layer 234, and is a layer having a function of receiving electrons from the cathode 222 or cathode 222 side to transfer the electrons to the anode 221 side. Specifically, the electron injection layer and the electron transport layer 235 are preferably layers that include a triazole derivative, an oxazole derivative, an oxadiazole derivative, an imidazole derivative, a fluorenone derivative, an anthraquinodimethane derivative, an anthrone derivative, a diphenylquinone derivative, a thiopyran dioxide derivative, a carbodiimide derivative, a fluorenylidene methane derivative, a distyrylpyrazine derivative, a naphthalene, an aromatic tetracarboxylic anhydride such as perylene, a phthalocyanine derivative, a phenanthrene derivative, a phenanthroline derivative, various kinds of complexes typified by a complex of a 8-quinolinol derivative, a complex containing a metal phthalocyanine, a benzoxazole or a benzothiazole as a ligand, an organic silane derivative, and the like.

The thickness of the electron injection layer and the thickness of the electron transport layer 235 each are preferably equal to or less than 100 nm from the viewpoint of lowering the driving voltage.

The thickness of the electron transport layer 235 is preferably from 1 nm to 100 nm, more preferably from 5 nm to 50 nm, and even more preferably from 10 nm to 30 nm. The thickness of the electron injection layer is preferably from 0.1 nm to 100 nm, more preferably from 0.2 nm to 80 nm, and even more preferably from 0.5 nm to 50 nm

The electron injection layer and the electron transport layer 235 may be a single layer structure made of one or two or more kinds of materials described above, or a multi-layer structure composed of a plurality of layers made of the same composition or different compositions.

### —Hole Blocking Layer—

The hole blocking layer (not shown) is provided between the cathode 222 and the light emitting layer 234, and is a layer having a function of preventing the holes transported from the anode 221 side to the light emitting layer 234 from passing through the cathode 222 side. Although not illustrated in FIG. 4, a hole blocking layer can be provided as an organic layer adjacent to the light emitting layer 234 on the cathode 222 side.

Examples of the organic compound constituting the hole blocking layer includes an aluminum complex, such as aluminum (III) bis(2-methyl-8-quinolinato)4-phenyl-phenolate (abbreviated as BAlq), carbazole derivatives, triazole derivatives, phenanthroline derivatives, such as 2,9-dimethyl-4,7-diphenyl-1,10-phenanthroline (abbreviated as BCP), and the like.

The thickness of the hole blocking layer is preferably from 1 nm to 500 nm, more preferably from 5 nm to 200 nm, and even more preferably from 10 nm to 100 nm.

The hole blocking layer may be a single layer structure made of one or two or more kinds of the materials described above, or a multi-layer structure composed of a plurality of layers made of the same composition or different compositions.

### —Electron Blocking Layer—

An Electron blocking layer 233 is provided between the anode 221 and the light emitting layer 234, and is a layer having a function of preventing electrons transported from the cathode 222 side to the light emitting layer 234 from passing through the anode 221 side. For example, the above-mentioned examples of the hole transport material can be applied as an example of the organic compound forming the electron blocking layer 233.

The thickness of the electron blocking layer 233 is preferably from 1 nm to 500 nm, more preferably from 5 nm to 200 nm, and even more preferably from 10 nm to 100 nm

The electron blocking layer 233 may be a single layer structure made of one or two or more kinds of layers of the material described above, or a multi-layer structure composed of a plurality of layers made of the same composition or different compositions.

### <Protective Layer>

The entire organic electroluminescent element 200 may be protected by the protective layer. As to the protective layer, the matters described in paragraphs [0169]-[0170] of Japanese Unexamined Patent Publication No. 2008-270736 can also be applied to the present application.

### <Sealing Container>

The entire organic electroluminescent element 200 may be sealed by using a sealing container. As to the sealing container, the matters described in paragraph [0171] of Japanese Unexamined Patent Publication No. 2008-270736 can also be applied to the present application.

(Drive)

The organic electroluminescent element 200 can emit light by applying a direct current (which may contain an alternating current component if necessary) voltage (usually of 2 volts to 15 volts), or direct current between the anode 221 and the cathode 222.

As a driving method of the organic electroluminescent element 200, driving methods disclosed in the following references can be applied: Japanese Unexamined Patent Publication No. 2(1990)-148687, 6(1994)-301355, 5(1993)-029080, 7(1995)-134558, 8(1996)-234685, 8(1996)-241047, Japanese Patent No. 2784615, U.S. Pat. No. 5,828,429, and the specification of U.S. Pat. No. 6,023,308.

(Use of Organic Electroluminescent Element)

The organic electroluminescent element 200 can be suitably applied to display devices, displays, backlights, electrophotography, illumination sources, recording light sources, exposure light sources, reading light sources, signs, signboards, interior, or optical communication or the like. In particular, the organic electroluminescent element can be preferably applied to a device that drives in an area with high light emission brightness, such as an illumination device, or a display device.

The invention will be further described in detail below by using Examples, but is not limited thereto.

Examples and Comparative Examples of the invention will be described below. In the Examples and Comparative examples shown below, the following compounds (exemplary compounds) 1 to 34 were used for studies. These exemplary compounds 1 to 34 are newly denoted by reference numbers 1 to 34 for better understanding of the examples, and thus do not match with the compounds 1 to 34 already mentioned as the specific examples of (EB-1) to (EB-4).

### [Chemical Formula 77]

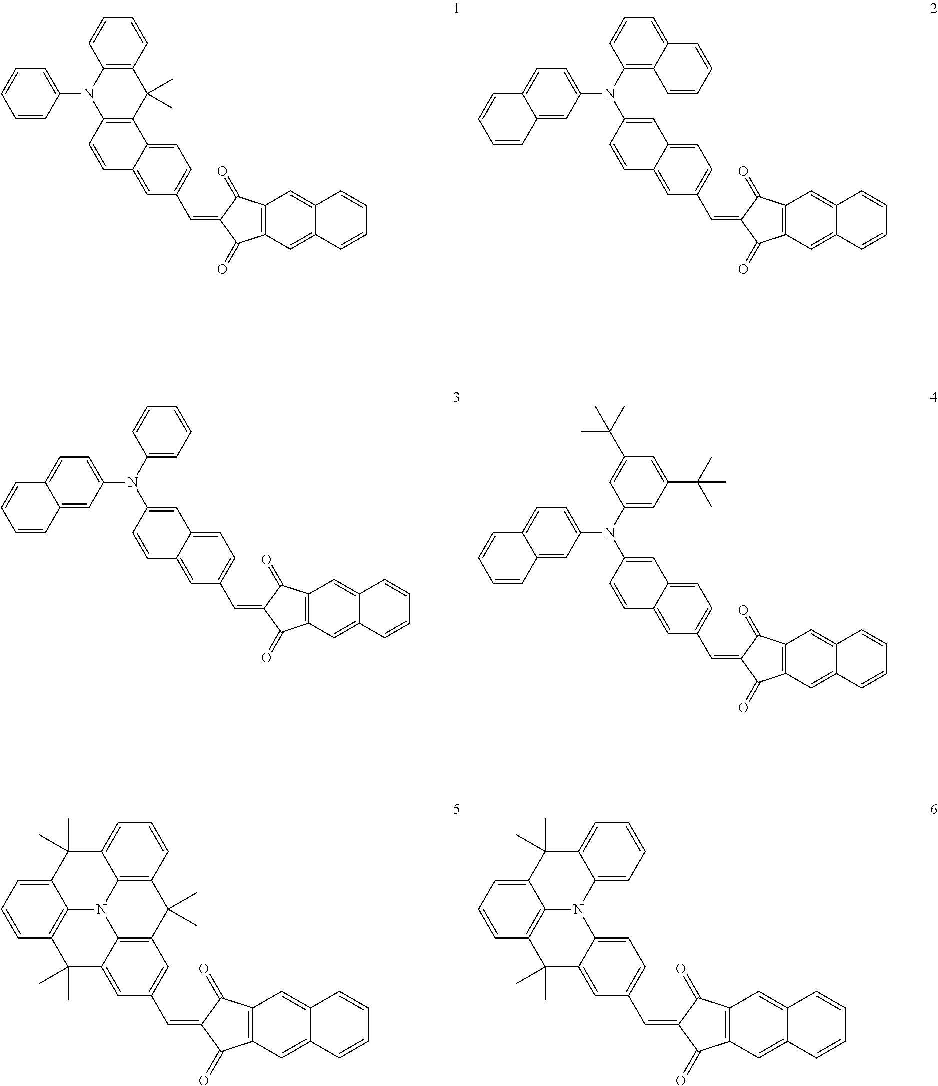 [SMILES: CC1(C)c2ccccc2N(c2ccccc2)c2ccc3cc(C=C4C(=O)c5cc6ccccc6cc5C4=O)ccc3c21 ; O=C1C(=Cc2ccc3cc(N(c4ccc5ccccc5c4)c4cccc5ccccc45)ccc3c2)C(=O)c2cc3ccccc3cc21 ; O=C1C(=Cc2ccc3cc(N(c4ccccc4)c4ccc5ccccc5c4)ccc3c2)C(=O)c2cc3ccccc3cc21 ; CC(C)(C)c1cc(N(c2ccc3ccccc3c2)c2ccc3cc(C=C4C(=O)c5cc6ccccc6cc5C4=O)ccc3c2)cc(C(C)(C)C)c1 ; CC1(C)c2cccc3c2N2c4c1cccc4C(C)(C)c1cc(C=C4C(=O)c5cc6ccccc6cc5C4=O)cc(c12)C3(C)C ; CC1(C)c2ccccc2N2c3ccc(C=C4C(=O)c5cc6ccccc6cc5C4=O)cc3C(C)(C)c3cccc1c32]

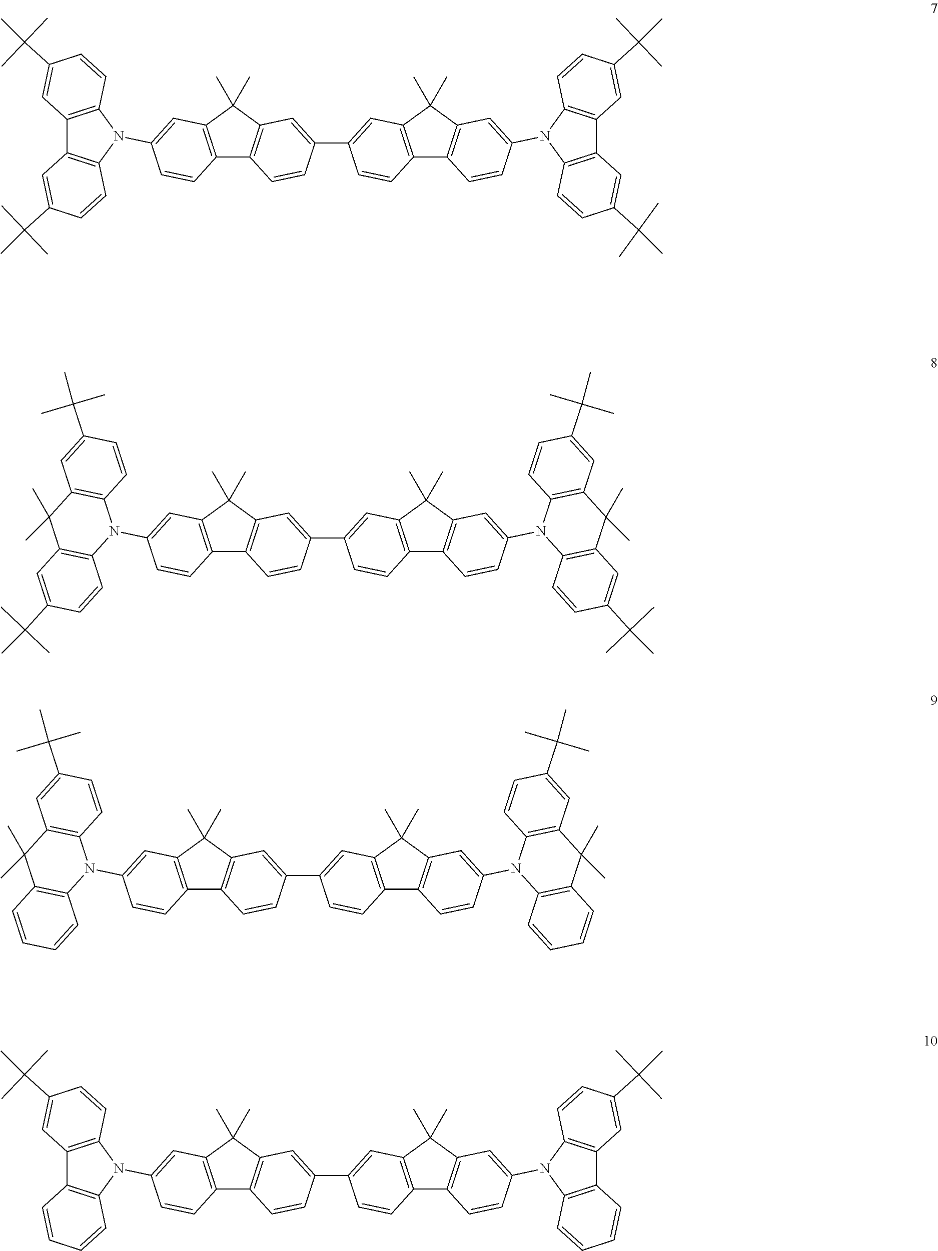

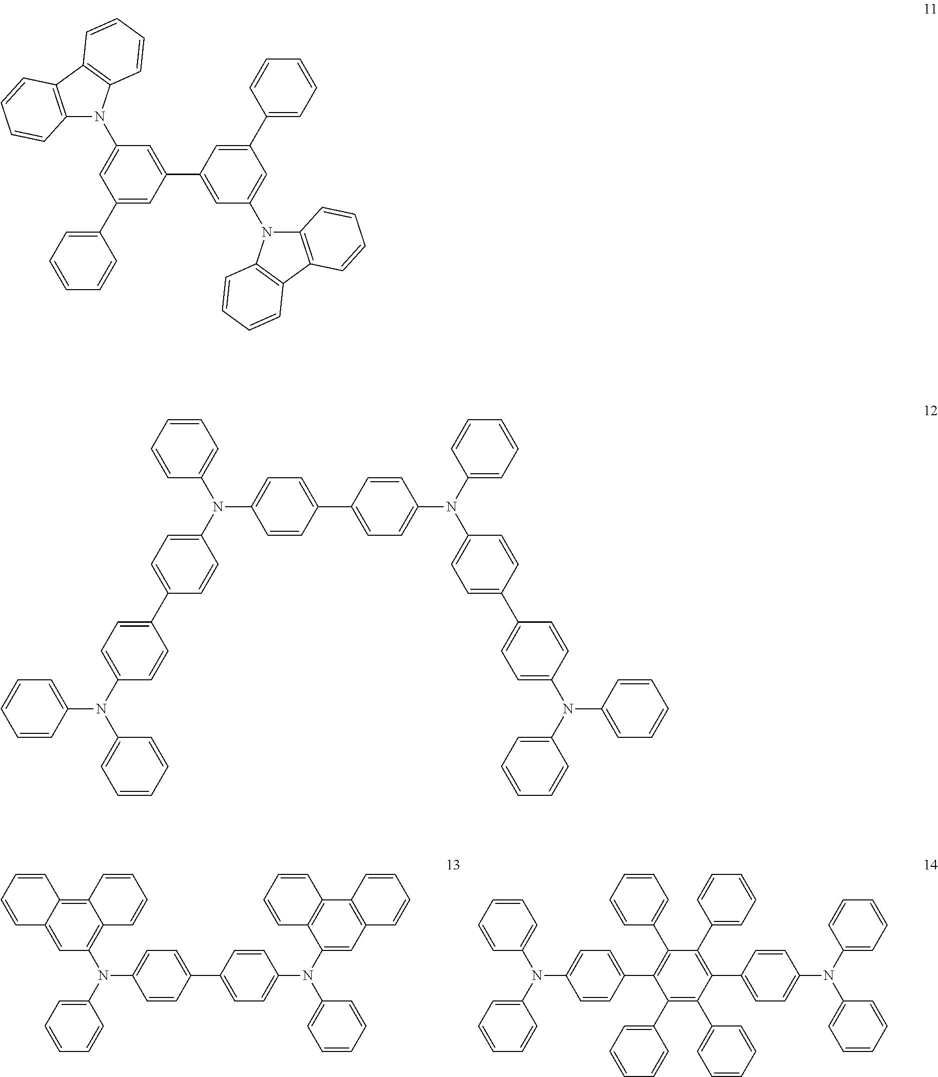 [SMILES: c1ccc(-c2cc(-c3cc(-c4ccccc4)cc(-n4c5ccccc5c5ccccc54)c3)cc(-n3c4ccccc4c4ccccc43)c2)cc1 ; c1ccc(N(c2ccccc2)c2ccc(-c3ccc(N(c4ccccc4)c4ccc(-c5ccc(N(c6ccccc6)c6ccc(-c7ccc(N(c8ccccc8)c8ccccc8)cc7)cc6)cc5)cc4)cc3)cc2)cc1 ; c1ccc(N(c2ccc(-c3ccc(N(c4ccccc4)c4cc5ccccc5c5ccccc45)cc3)cc2)c2cc3ccccc3c3ccccc23)cc1 ; c1ccc(-c2c(-c3ccccc3)c(-c3ccc(N(c4ccccc4)c4ccccc4)cc3)c(-c3ccccc3)c(-c3ccccc3)c2-c2ccc(N(c3ccccc3)c3ccccc3)cc2)cc1]

### [Chemical Formula 78]

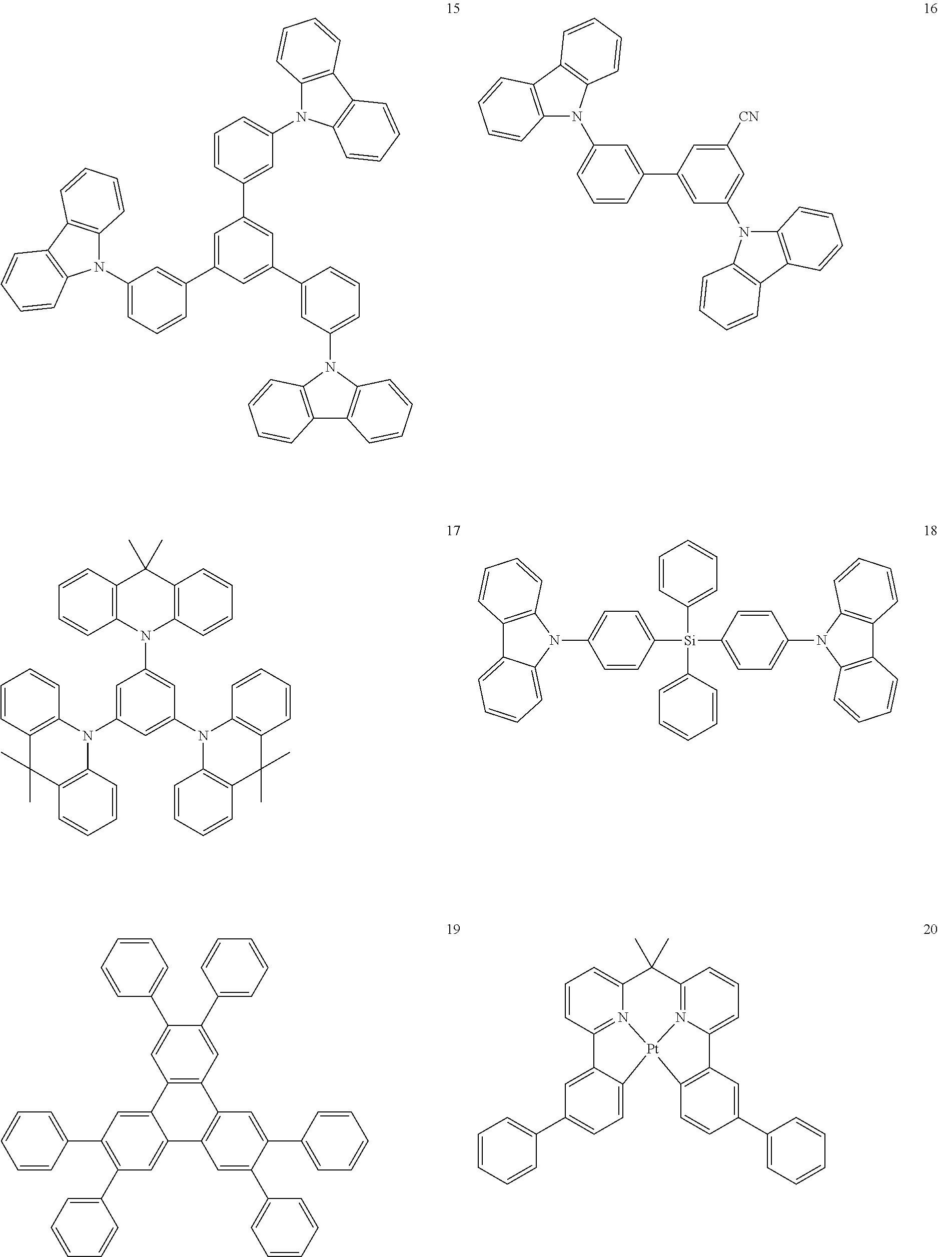

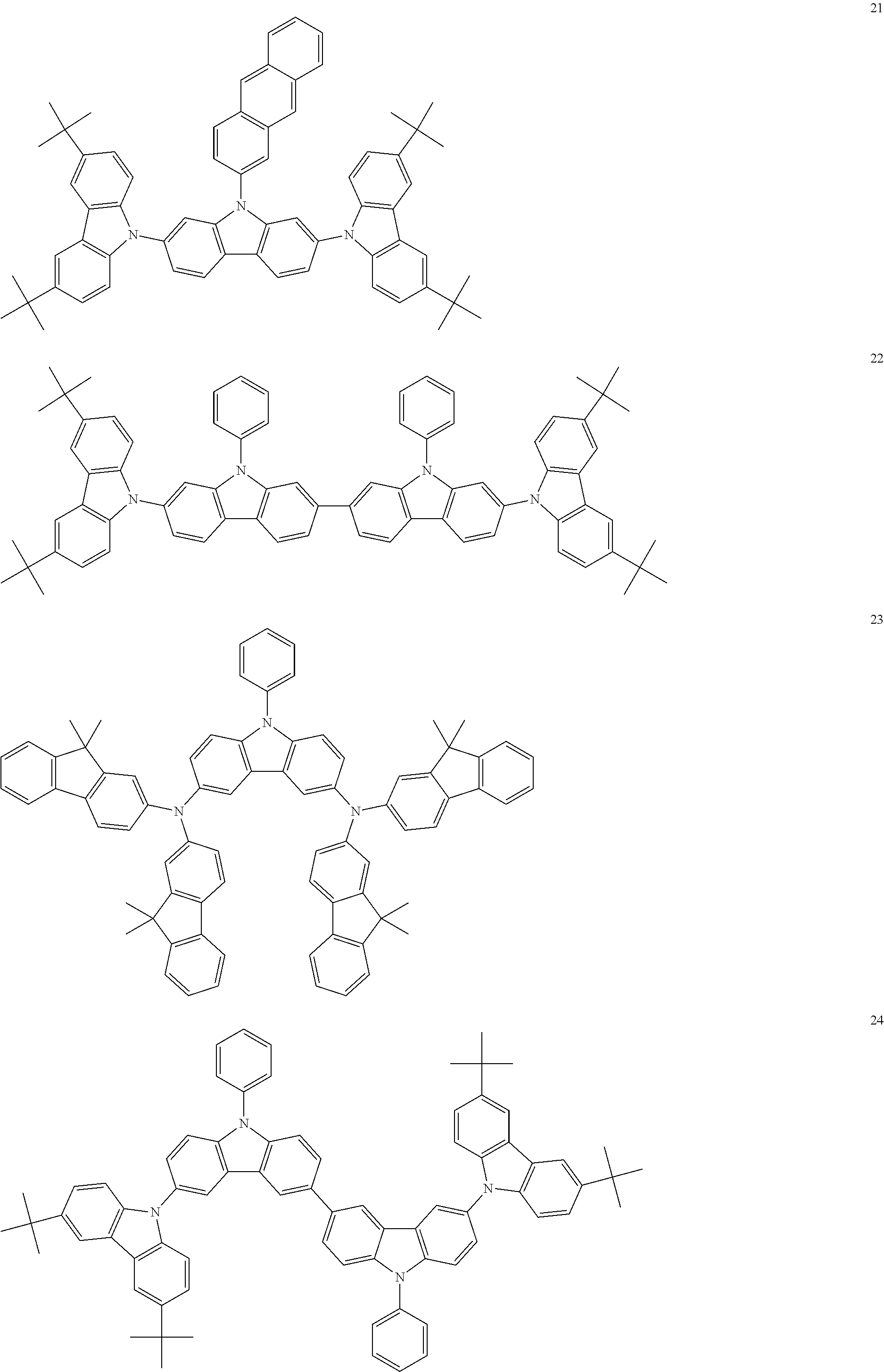

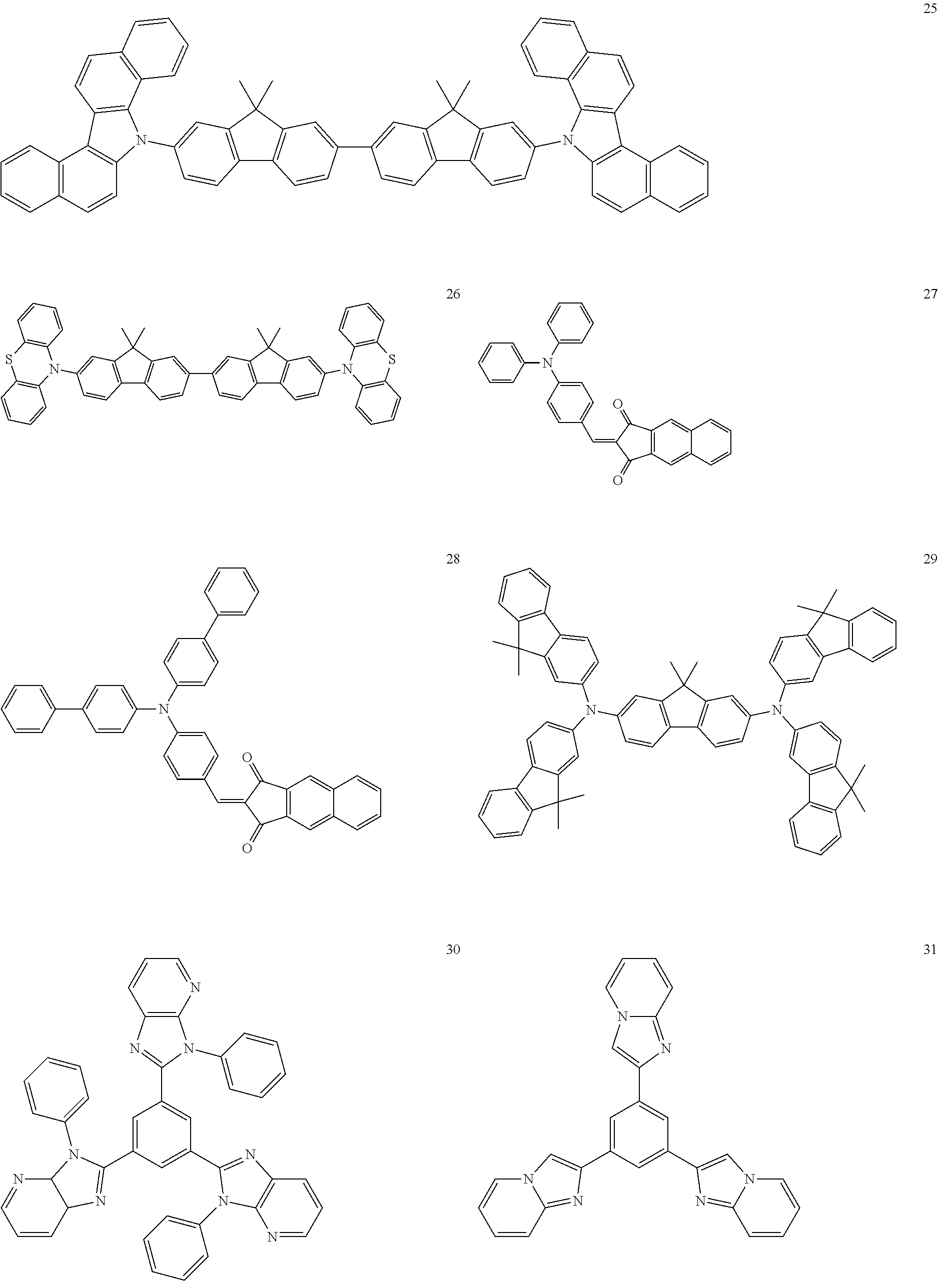

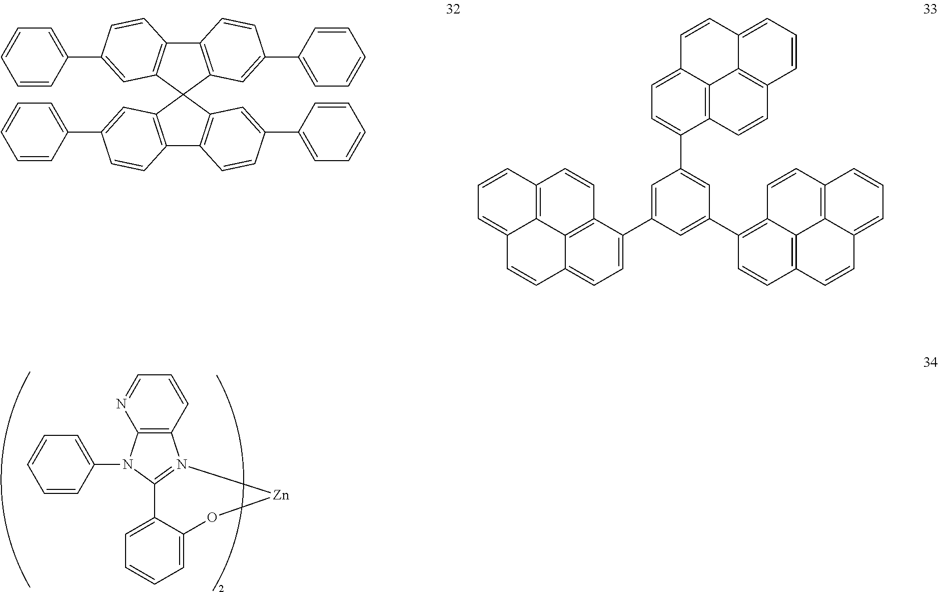

### Examples 1 to 6, Comparative Examples 1 to 3

### Synthesis of Organic Material for Deposition

First, the organic material for deposition of the above-mentioned exemplary compound 1 was synthesized. The synthesis of the exemplary compound 1 was carried out according to steps shown in the following reaction formula.

### [Chemical Formula 79]

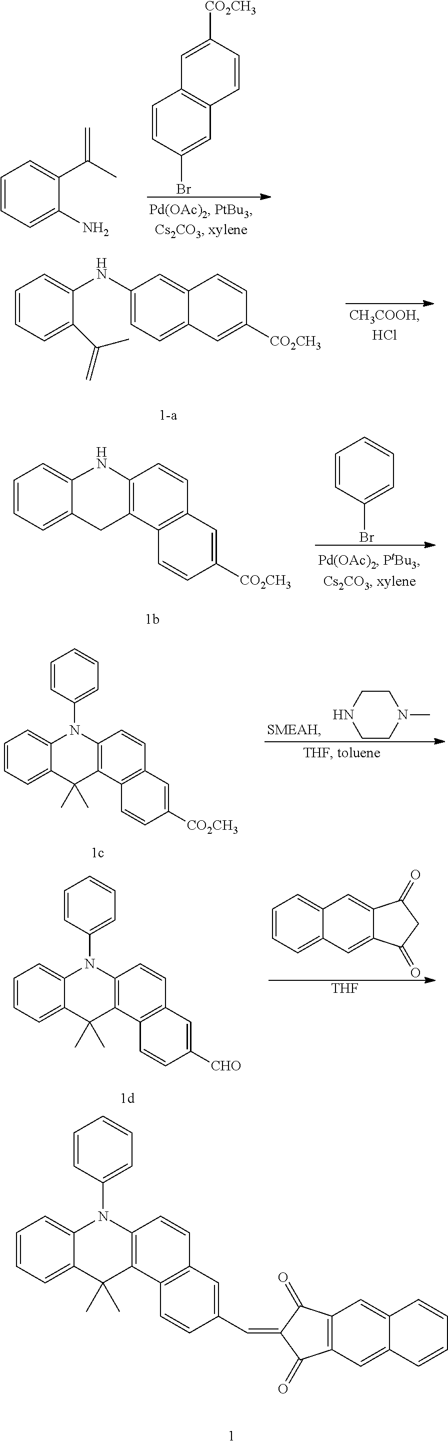

First, 2-iso-propenyl aniline, palladium acetate, tri (t-butyl)phosphine, cesium carbonate, and 6-bromo-2-naphthoic acid methyl were dissolved in xylene, and reacted together for 5 hours under a nitrogen atmosphere at the boiling point under reflux to yield a compound 1a. The compound 1a were added into a mixture solvent of acetic acid and hydrochloric acid, and stirred for 30 minutes at 60° C. to yield a compound 1b. The compound 1b, palladium acetate, tri (t-butyl) phosphine, cesium carbonate, and bromobenzene were dissolved in xylene, and reacted for 7 hours under a nitrogen atmosphere at the boiling point under reflux to yield a compound 1c. Under the nitrogen atmosphere, dihydrate hydride bis (2-methoxyethoxy) aluminum sodium (SMEAH) 70% toluene solution was added to THF, and cooled to 0° C. To the THF, N-methylpiperazine was added dropwise, and stirred for 30 minutes to prepare a reducing agent solution. The reducing agent solution was added dropwise to the THF solution containing the compound 1c at −40° C. under the nitrogen atmosphere. After the reaction solution was stirred for 4 hours at −20° C., the reaction was terminated with dilute hydrochloric acid to yield a compound 1d. The compound 1d and benzoin Dan dione were dissolved in the THF solution and refluxed for 3 hours. under a nitrogen atmosphere, and were then left until cooled, followed by suction filtration to produce a compound 1.

Then, to the thus-obtained reaction compound 1 (rough body) was subjected to a solvent removal step in such a manner that the solvent content was the amount corresponding to each of Examples 1 to 6 and Comparison Examples 1 to 3 shown in Table 1, thereby producing an organic material for deposition of the present invention containing the compound 1 as a principal component (hereinafter, a compound 1 for deposition). Specifically, by sublimation purification and recrystallization purification, the solvent amount of the compound 1 was adjusted. A residual solvent content contained in the organic material for deposition in each example was measured by NMR (analysis system manufactured by Bruker Corporation, AV400) and Karl Fischer measurement (manufactured by Hiranuma Sangyo Co., Ltd., AQ-2100). Powder purity was determined by HPLC (analysis system: manufactured by Shimadzu Corporation, LC-10A, column: Tosoh Corporation, TSKGel-80TS).

### <Deposition>

Then, using the compound 1 for deposition in each example, a vapor-deposited film was deposited in a thickness of 100 nm on a glass substrate by vacuum resistance heating deposition at a vapor-deposition rate of about 2 Å/s, by defining the time when the deposition becomes stable at this rate, as 0 minute. While the vapor-deposition rate was kept, a vapor-deposited film was deposited in a thickness of 100 nm over a new glass substrate every 60 minutes. In this way, in each example, the vapor-deposited film was deposited at each of 0 minute, 60 minutes and 120 minutes. The total thickness of vapor-deposited film undergoing continuous heating at the deposition time after 120 minutes has elapsed was about 16000 Å, including the part of film during raising the temperature.

### <Evaluation>

In each example, the film purity of the vapor-deposited film was measured by HPLC. The film purity was determined by calculation of a peak area ratio of HPLC (detection wavelength: 254 nm).

The evaluation results are shown in Table 1 below. As shown in the table, the vapor-deposited films of Examples 1-6 had an amount of change in film purity of less than 10% until the continuous deposition time reached 120 minutes without being affected by powder purity, and hence was confirmed to be suitable for mass production.

Furthermore, a solution obtained by dissolving the compound 1 d and benzoin Dan dione was changed to acetic acid, anisole, ethanol, dimethyl ether, or a mixed solvent thereof, and the residual solvent content was changed in the same way. However, the same results were obtained.

### <Preparation of Photoelectric Conversion Element>

Then, a photoelectric conversion element was prepared by using the compound 1 for deposition in each of the above examples, and then the photoelectric conversion element was evaluated for their properties.

On a glass substrate, the amorphous ITO (30 nm) was deposited for a plurality of substrates by sputtering, thereby forming pixel electrodes (lower electrodes). Thereon, an electron blocking layer made of an electron blocking layer material (EB-A) (which may include impurities) represented by the following formula was formed in a thickness of 100 nm by the vacuum resistance heating deposition by using the organic material for deposition of the invention in which the residual solvent content is reduced to 0.1 mol % or less by performing the solvent removal step.

### [Chemical Formula 80]

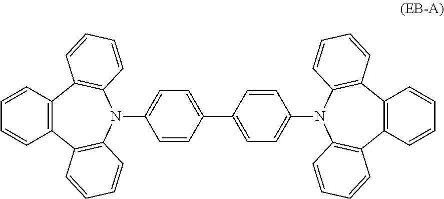 [SMILES: c1ccc2c(c1)-c1ccccc1N(c1ccc(-c3ccc(N4c5ccccc5-c5ccccc5-c5ccccc54)cc3)cc1)c1ccccc1-2]

Thereon, the compound 1 for deposition and fullerene C₆₀ were co-deposited by vacuum resistance heating deposition to form a photoelectric conversion layer having a thickness of 300 nm. In the co-deposition, the volume ratio of the compound 1 to that of the fullerene was set to 1:3, and the fabrication of the element was started by defining the time where the deposition rate becomes about 2 Å/s to be stable, as 0 minute. After forming the photoelectric conversion layer, the rate of the compound 1 was maintained, and C₆₀ had its temperature reduced to such a level that did not get adequate rate, and had the temperature kept. Thereafter, C₆₀ was heated again, and the vapor-deposited film (of 300 nm in thickness) was deposited while exchanging the substrate every 60 minutes.

Furthermore, an amorphous ITO (of 10 nm in thickness) was deposited as an upper electrode by sputtering to form the transparent electrode (upper electrode), thereby fabricating a photoelectric conversion element. On the upper electrode, a SiO film was formed by heating deposition as a sealing layer, followed by formation of an Al₂O₃ layer thereon by an ALCVD method.

[Evaluation]

For the photoelectric conversion elements obtained in the respective examples, the relative response speed upon application of an electric field of 2×10⁵ V/cm (rise time with respect to 0 to 99% signal strength), and the relative sensitivity (wavelength of 500-750 nm) were measured. The result is shown in Table 2. At the time of measurement of the photoelectric conversion performance of each element, the light entered from the upper electrode (transparent conductive film) side.

The relative sensitivity and the relative response speed were represented by a relative value with those at 0 minute set to 100 in Example 1 (see Table 2). As shown in Table 2, it was confirmed that in the photoelectric conversion element using the vapor deposition film of Examples 1-6 (Table 2), the changes in the relative response speed and sensitivity were remained within 5%, which was suitable for mass production.

TABLE 1

| | | | | | | | Ratio of film purity |
|---|---|---|---|---|---|---|---|
| | | | | | | | after 120 minutes |
| | | Solvent | Powder | Film purity(%) | | | to that after 0 minute |
| | | content | purity | After 0 | After 60 | After 120 | (Truncate the number |
| | Compound | (mol %) | (%) | minute | minutes | minutes | to 2 decimal places) |
| Example 1 | Compound 1 | 0.1 | 99.7 | 99.7 | 99.6 | 99.6 | 0.99 |
| Example 2 | Compound 1 | 0.5 | 99.7 | 99.7 | 99.4 | 99.3 | 0.99 |
| Example 3 | Compound 1 | 0.7 | 99.6 | 99.6 | 99.2 | 99.1 | 0.99 |
| Example 4 | Compound 1 | 1.2 | 99.5 | 99.5 | 99.7 | 99.0 | 0.99 |
| Example 5 | Compound 1 | 2.0 | 99.8 | 99.3 | 97.5 | 95.6 | 0.96 |
| Example 6 | Compound 1 | 2.8 | 99.6 | 99.1 | 94.9 | 90.3 | 0.91 |
| Comparative | Compound 1 | 3.1 | 99.7 | 94.2 | 87.0 | 81.7 | 0.86 |
| example 1 | | | | | | | |
| Comparative | Compound 1 | 3.5 | 99.6 | 90.3 | 84.0 | 76.0 | 0.84 |
| example 2 | | | | | | | |
| Comparative | Compound 1 | 3.9 | 99.7 | 87.6 | 82.2 | 71.1 | 0.81 |
| example 3 | | | | | | | |

TABLE 2

| | | Solvent | Powder | Relative sensitivity | | | Relative response speed | | |
|---|---|---|---|---|---|---|---|---|---|
| | | content | purity | After 0 | After 60 | After 120 | After 0 | After 60 | After 120 |
| | Compound | (mol %) | (%) | minute | minutes | minutes | minute | minutes | minutes |
| Example 1 | Compound 1 | 0.1 | 99.7 | 100 | 100 | 100 | 100 | 100 | 100 |
| Example 2 | Compound 1 | 0.5 | 99.7 | 100 | 100 | 100 | 100 | 100 | 100 |
| Example 3 | Compound 1 | 0.7 | 99.6 | 100 | 100 | 100 | 100 | 100 | 100 |
| Example 4 | Compound 1 | 1.2 | 99.5 | 100 | 100 | 100 | 100 | 100 | 100 |
| Example 5 | Compound 1 | 2.0 | 99.8 | 100 | 100 | 100 | 100 | 100 | 100 |
| Example 6 | Compound 1 | 2.8 | 99.6 | 100 | 100 | 98.5 | 100 | 100 | 99 |
| Comparative | Compound 1 | 3.1 | 99.7 | 100 | 98 | 95 | 100 | 98 | 94.5 |
| example 1 | | | | | | | | | |
| Comparative | Compound 1 | 3.5 | 99.6 | 98.5 | 97.3 | 93 | 98 | 97.5 | 93 |
| example 2 | | | | | | | | | |
| Comparative | Compound 1 | 3.9 | 99.7 | 98 .2 | 96.1 | 90 | 98 | 95 | 90 |
| example 3 | | | | | | | | | |

### Examples 7 to 47 and Comparative Examples 4 to 39

Like Examples and Comparative Examples of the compound 1, as to the compounds 2 to 34, the deposition of a vapor deposited film (100 nm) was started using the compound by the vacuum resistance heating deposition over the glass substrate by defining the time when the vapor deposition rate becomes about 2 Å/s as 0 minute. Then, the rate was maintained, and a new vapor deposited film (100 nm) was deposited by the vacuum resistance heating deposition every 60 minutes, and then the vapor deposited films were evaluated for film purity.

### [Synthesis of Compound 2]

The above exemplary compounds 2 can be fabricated by the following reaction formula.

### [Chemical Formula 81]

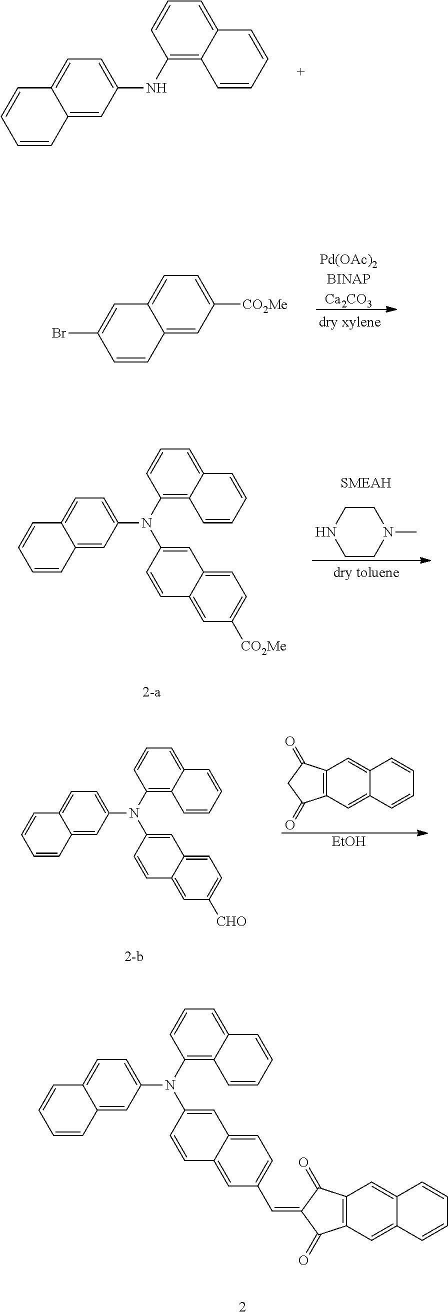

To dehydrated xylene, 1,2′-dinaphthylamine (manufactured by Tokyo Kasei Kogyo Co., Ltd.), 6-bromo-2-methyl-naphthoic acid (manufactured by Wako Pure Chemical Industries, Ltd.), palladium acetate, BINAP (2,2′-bis(diphenylphosphino)-1,1′-binaphthyl), and cesium carbonate were added, followed by reflux of the mixture for 4 hours. The reaction mixture was purified with a silica gel column to give a compound 2-a. To dehydrated toluene was added SMEAH (sodium bis(2-metoxyetoxy) aluminumhydride toluene solution (about 70%)(manufactured by Wako Pure Chemical Industries, Ltd.), followed by cooling an internal temperature thereof to 0° C. in an ice bath, and then a solution of 1-methyl piperazine dissolved in the dehydrated toluene was added dropwise thereto. Then, the compound 2-a was dissolved in the dehydrated toluene, followed by cooling an internal temperature thereof to −40° C. in a dry ice bath, and to this was added dropwise the SMEAH toluene solution previously adjusted, followed by stirring for 4.5 hours, and quenching with concentrated hydrochloric acid. The reaction mixture was purified by a silica gel column to give a compound 2-b. The compound 2 was synthesized using the compound 2-b according to the known method (U.S. Patent Application Publication No. 20050065351).

### [Synthesis of Compound 3]

An exemplary compound 3 was synthesized in the same way as Example 2 except that 1,2′-dinaphthylamine in Example 2 was changed to N-phenyl-2-naphthylamine (manufactured by Tokyo Kasei Kogyo Co., Ltd.).

### [Synthesis of Compound 4]

An exemplary compound 4 can be prepared by the following reaction formula.

### [Chemical Formula 82]

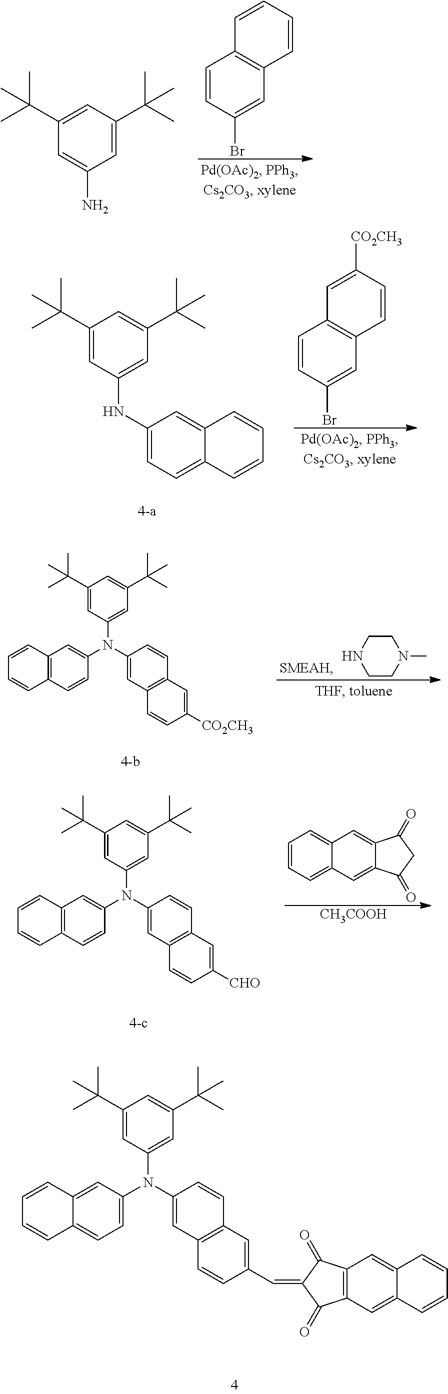

3,5-di-tert-butyl aniline, palladium acetate, triphenylphosphine, cesium carbonate, and 2-bromonaphthalene were dissolved in xylene, and reacted together for 7 hours under a nitrogen atmosphere at the boiling point under reflux to yield a compound 4-a. The compound 4-a, palladium acetate, triphenylphosphine, cesium carbonate, and 6-bromo-2-naphthoic acid methyl were dissolved in xylene, and reacted together for 10 hours under a nitrogen atmosphere at the boiling point under reflux to yield a compound 4-b. Under the nitrogen atmosphere, dihydrate hydride bis (2-methoxyethoxy) aluminum sodium (SMEAH) 70% toluene solution was added to THF, and cooled to 0° C. To the THF, N-methylpiperazine was added dropwise, and stirred for 30 minutes to prepare a reducing agent solution. The reducing agent solution was added dropwise to the THF solution containing the compound 4-b at −40° C. under the nitrogen atmosphere. After the reaction solution was stirred for 4 hours at −20° C., the reaction was terminated with dilute hydrochloric acid to yield a compound 4-c. The compound 4-c and benzoin Dan dione were added to acetic acid solvent under the nitrogen atmosphere, and refluxed for 3 hours, and then left until cooled, followed by suction filtration to perform recystalization with N,N-dimethylacetamide. Then, the suction filtration was performed to yield a compound 4.

### [Synthesis of Compound 5]

An exemplary compound 5 can be prepared by the following reaction formula.

### [Chemical Formula 83]

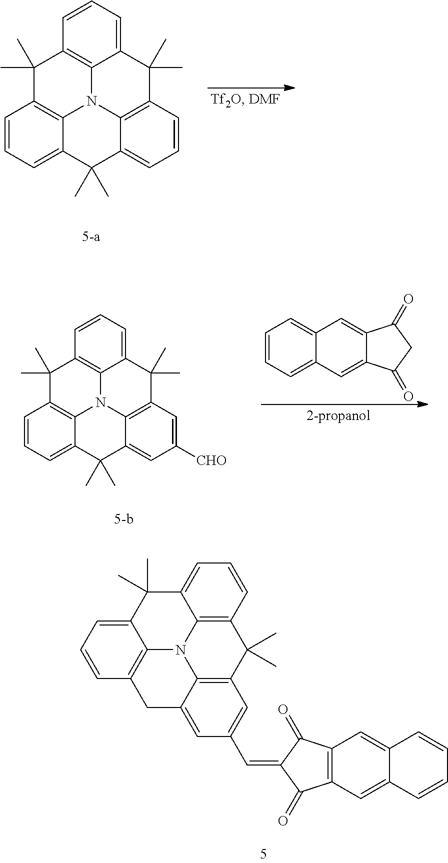

The compound 5-a was synthesized by a method disclosed in Org. Lett. 2009, Vol. 11, 1-4. The compound 5-a was dissolved in dehydrated N, N-dimethylformamide, to which trifluoromethanesulfonic acid anhydride was added dropwise. The solution was heated under a nitrogen atmosphere at 90° C., following stirring for one hour to give a compound 5-b. Under a nitrogen atmosphere, a compound 5-b and benzoin Dan dione were added to the 2-propanol solvent, and refluxed for 3 hours. After being left until cooled, suction filtration was carried out to give compound 5.

### [Synthesis of Compound 6]

An exemplary compound 6 can be prepared by the following reaction formula.

### [Chemical Formula 84]

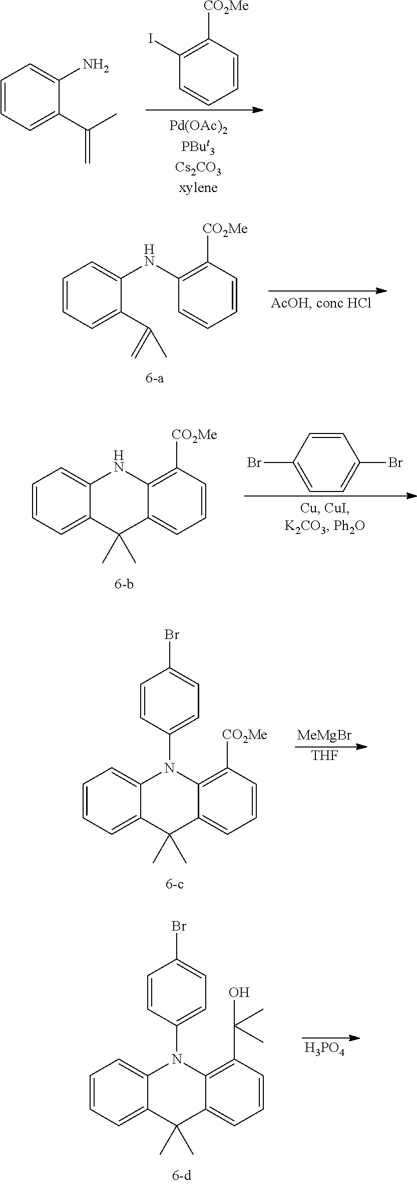

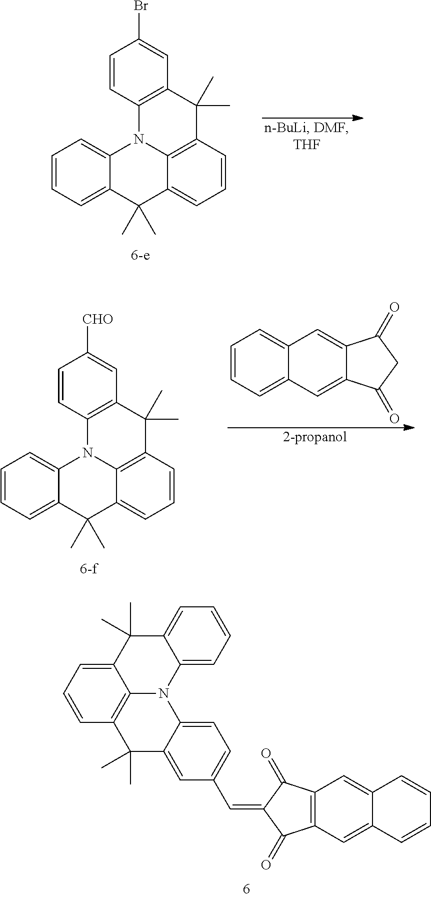

Isopropenyl aniline, Orutoyodo methyl benzoate, palladium acetate, tri(t-butyl) phosphine, and the cesium carbonate were dissolved in xylene, and reacted together for 5 hours under a nitrogen atmosphere under reflux to yield a compound 6-a. The compound 6-a was dissolved in a mixed solvent of acetic acid and concentrated hydrochloric acid, followed by stirring for 1 hour at 60° C. to yield a compound 6-b. The compound 6-b, p-dibromobenzene, copper powder, copper iodide, and potassium carbonate were added to the diphenyl ether, followed by reflux for 5 hours to yield a compound 6-c. The compound 6-c was dissolved in dehydrated tetrahydrofuran, to which a 3M methyl Grignard reagent (ethyl ether solution) was added dropwise. Thereafter, the solution was heated to the reflux temperature, and stirred for one hour to yield a compound 6-d. The compound 6-d was added to phosphoric acid, followed by stirring for 2 hours at 90° C. to give a compound 6-e. The compound 6-e was dissolved in dehydrated tetrahydrofuran, and then cooled to −40° C. using a dry ice bath, followed by adding of n-butyl lithium (1.6M in hexane) dropwise, and stirring for 15 minutes. To this, N,N-dimethylformamide was added dropwise and then the dry ice bath was removed. By addition of 1M dilute hydrochloric acid, a compound 6-f was obtained. Under the nitrogen atmosphere, to a 2-propanol solvent was added the compound 6-f and benzoin Dan dione, followed by reflux for 3 hours. After being left until cooled, the solution was filtered by suction filtration to give a compound 6.

### (Synthesis of Compound 7)

An exemplary Compound 7 can be prepared by the following reaction formula.

### [Chemical Formula 85]

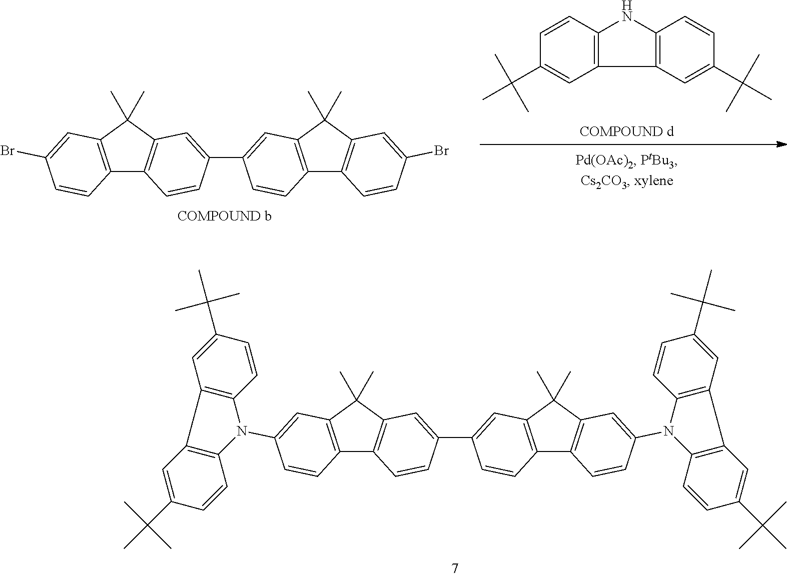 [SMILES: CC1(C)c2cc(Br)ccc2-c2ccc(-c3ccc4c(c3)C(C)(C)c3cc(Br)ccc3-4)cc21 ; CC(C)(C)c1ccc2[nH]c3ccc(C(C)(C)C)cc3c2c1 ; CC(C)(C)c1ccc2c(c1)c1cc(C(C)(C)C)ccc1n2-c1ccc2c(c1)C(C)(C)c1cc(-c3ccc4c(c3)C(C)(C)c3cc(-n5c6ccc(C(C)(C)C)cc6c6cc(C(C)(C)C)ccc65)ccc3-4)ccc1-2]

A compound b (1.10 g, 2.02 mmol) and palladium acetate (22.7 mg, 0.101 mmol), tri (t-butyl) phosphine (61.3 mg, 0.303 mmol), cesium carbonate (2.63 g, 8.08 mmol), and the compound d (1.24 g, 4.44 mmol) were dissolved in 11 ml of xylene, and allowed to react for 4 hours at a boiling point under reflux in nitrogen atmosphere. To the reaction mixture were added ethyl acetate and water to separate an organic phase. The organic phase was washed with water and saturated sodium chloride solution, and condensed under reduced pressure. The thus-obtained reaction mixture was purified by recrystallization to thereby yield a compound 7.

### (Synthesis of Compound 8)

An exemplary Compound 8 can be prepared by the following reaction formula.

### [Chemical Formula 86]

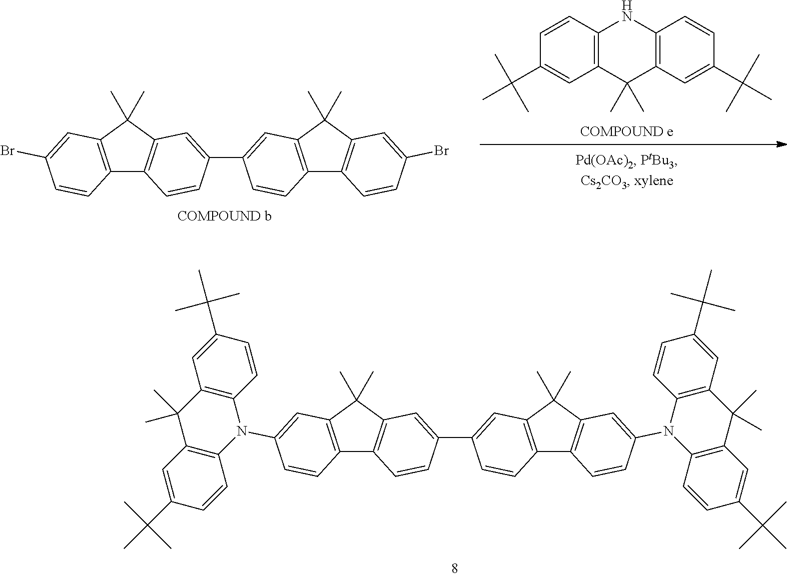 [SMILES: CC1(C)c2cc(Br)ccc2-c2ccc(-c3ccc4c(c3)C(C)(C)c3cc(Br)ccc3-4)cc21 ; CC(C)(C)c1ccc2c(c1)C(C)(C)c1cc(C(C)(C)C)ccc1N2 ; CC(C)(C)c1ccc2c(c1)C(C)(C)c1cc(C(C)(C)C)ccc1N2c1ccc2c(c1)C(C)(C)c1cc(-c3ccc4c(c3)C(C)(C)c3cc(N5c6ccc(C(C)(C)C)cc6C(C)(C)c6cc(C(C)(C)C)ccc65)ccc3-4)ccc1-2]

The compound b (1.10 g, 2.02 mmol) and palladium acetate (22.7 mg, 0.101 mmol), tri (t-butyl) phosphine (61.3 mg, 0.303 mmol), cesium carbonate (2.63 g, 8.08 mmol), and the compound e (1.36 g, 4.24 mmol) were dissolved in 10 ml of xylene, and allowed to react for 4 hours at a boiling point under reflux in nitrogen atmosphere. To the reaction mixture were added ethyl acetate and water to separate an organic phase. The organic phase was washed with water and saturated sodium chloride solution, and condensed under reduced pressure. The thus-obtained reaction mixture was purified by recrystallization to thereby yield a compound 8.

### (Synthesis of Compound 9)

An exemplary compound 9 can be prepared by the following reaction formula.

### [Chemical Formula 87]

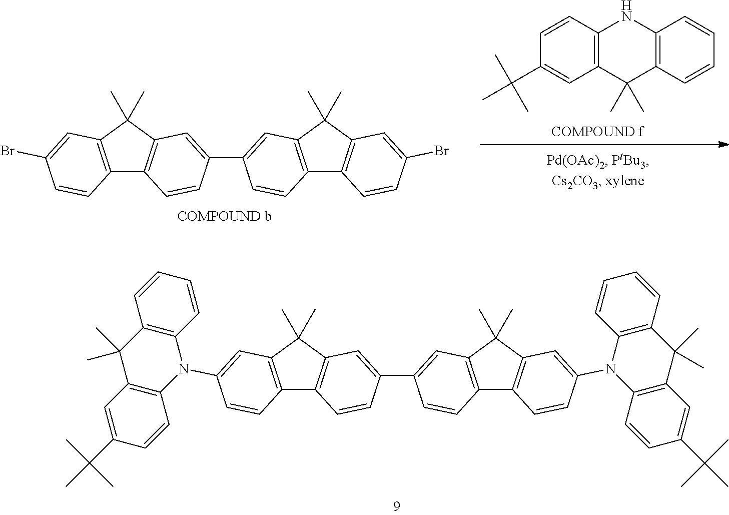 [SMILES: CC1(C)c2cc(Br)ccc2-c2ccc(-c3ccc4c(c3)C(C)(C)c3cc(Br)ccc3-4)cc21 ; CC(C)(C)c1ccc2c(c1)C(C)(C)c1ccccc1N2 ; CC(C)(C)c1ccc2c(c1)C(C)(C)c1ccccc1N2c1ccc2c(c1)C(C)(C)c1cc(-c3ccc4c(c3)C(C)(C)c3cc(N5c6ccccc6C(C)(C)c6cc(C(C)(C)C)ccc65)ccc3-4)ccc1-2]

The compound b (1.10 g, 2.02 mmol) and palladium acetate (22.7 mg, 0.101 mmol), tri (t-butyl)phosphine (61.3 mg, 0.303 mmol), cesium carbonate (2.63 g, 8.08 mmol), and a compound f (1.13 g, 4.24 mmol) were dissolved in 10 ml of xylene, and allowed to react for 4 hours at a boiling point under reflux in nitrogen atmosphere. To the reaction mixture were added ethyl acetate and water to separate an organic phase. The organic phase was washed with water and saturated sodium chloride solution, and condensed under reduced pressure. The thus-obtained reaction mixture was purified by recrystallization to thereby yield a compound 9.

### (Synthesis of Compound 10)

An exemplary compound 10 can be prepared by the following reaction formula.

### [Chemical Formula 88]

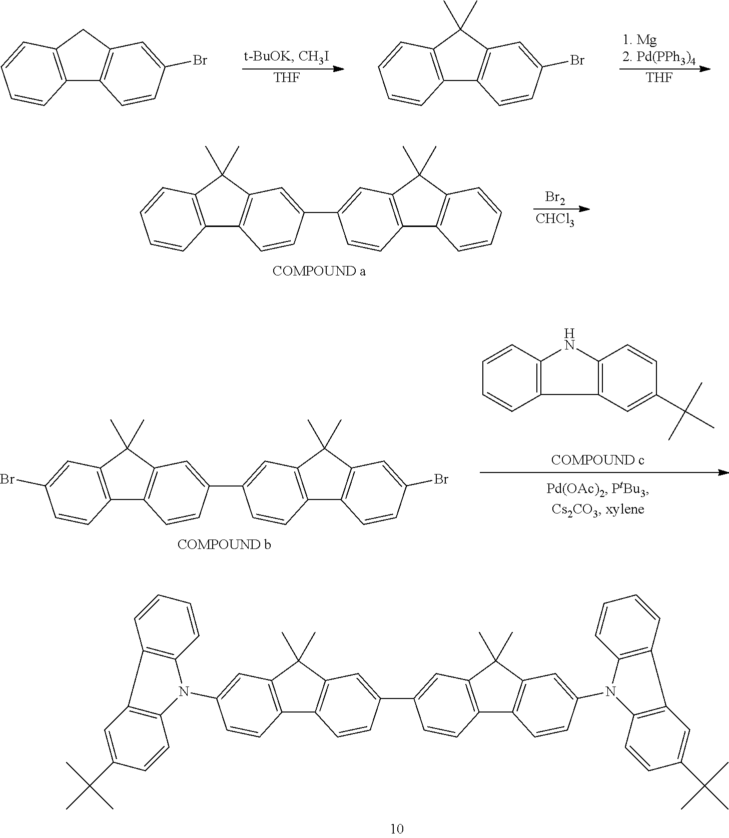

2-bromo-fluorene (89.0 g, 0.363 mol) was dissolved in tetrahydrofuran (THF) 1.31, and cooled to 5° C., and then potassium-tert-butoxide (102 g, 0.908 mol) was added thereto. Methyl iodide (565 ml, 0.908 mol) was added dropwise to the solution at 5° C. After the dropwise addition, the mixture was stirred at room temperature for 5 hours to give 2-bromo-9,9-dimethyl-fluorene in 87% yield. Under a nitrogen atmosphere, into 50 ml of THF, magnesium powder (3.51 g, 0.144 mol) was added, and refluxed at a boiling point. To the solution, 250 ml of THF solution containing 2-bromo-9,9-dimethyl-fluorene (75.0 g, 0.275 mol) was added dropwise, followed by stirring for one hour. Thereafter, tetrakis (triphenylphosphine) palladium (1.59 g, 1.38 mmol) was added to the solution, which was refluxed at a boiling point for 2 hours to give a compound a in 82% yield. To 500 ml of chloroform solution containing the compound a (43.8 g, 0.113 mol), was added dropwise bromine (39.8 g, 0.249 mol), followed by stirring for 3 hours to synthesize a compound b in 78% yield. The compound b (1.10 g, 2.02 mmol) and palladium acetate (22.7 mg, 0.101 mmol), tri (t-butyl) phosphine (61.3 mg, 0.303 mmol), cesium carbonate (2.63 g, 8.08 mmol), and the compound c (991 mg, 4.44 mmol) were dissolved in 11 ml of xylene, and allowed to react for 4 hours at a boiling point under reflux in nitrogen atmosphere. To the reaction mixture, were added ethyl acetate and water to separate an organic phase. The organic phase was washed with water and saturated sodium chloride solution, and then concentrated under reduced pressure. The obtained reaction mixture was purified by recrystallization to produce a compound 10.

### (Synthesis of Compound 11)

An exemplary compound 11 can be prepared by the following reaction formula.

### [Chemical Formula 89]

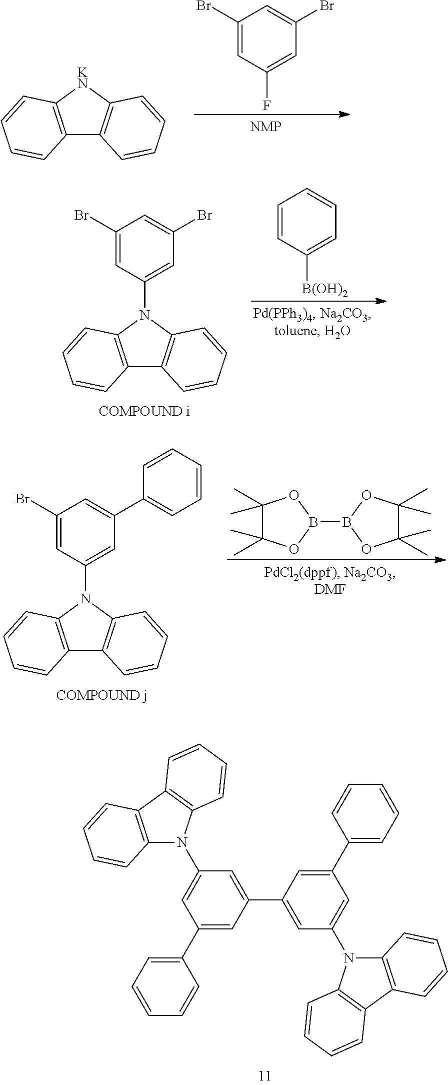

Carbazole potassium salt (17.6 g, 85.9 mol), 1,3-dibromo-5-fluorobenzene (24.0 g, 94.5 mol) were dissolved in 150 ml of 1-methyl-2-pyrrolidone, and stirred at 100° C. for 3 hours to give a compound i in 75% yield. The compound i (40.0 g, 99.7 mmol), phenylboronic acid (13.4 g, 110 mmol), tetrakis (triphenylphosphine) palladium (2.30 g, 1.99 mmol), sodium carbonate (21.1 g, 199 mmol) were dissolved in 500 ml toluene/200 ml H₂O/200 ml ethanol mixed solvent, and reacted for 2 hours at a boiling point under reflux in a nitrogen atmosphere to synthesize a compound j in 32% yield. The compound j (7.00 g, 17.6 mmol) and bis (pinacolato) diboron (2.23 g, 8.80 mmol), PdCl₂ (dppf) (719 mg, 0.88 mmol), sodium acetate (5.18 g, 52.8 mmol) were dissolved in 80 ml of DMF, and allowed to react for 3 hours at a boiling point under reflux in nitrogen atmosphere. To the reaction mixture were added ethyl acetate and water to separate the organic phase. The organic phase was washed with water and saturated sodium chloride solution, and concentrated under reduced pressure. The thus-obtained reaction mixture was purified by recrystallization to yield the exemplary compound 11.

### (Synthesis of Compound 15)

An exemplary compound 15 can be prepared by the following reaction formula.

### [Chemical Formula 90]

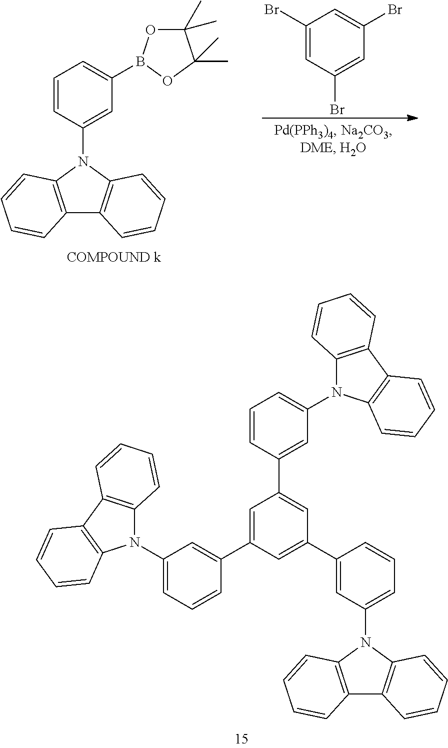 [SMILES: CC1(C)OB(c2cccc(-n3c4ccccc4c4ccccc43)c2)OC1(C)C ; Brc1cc(Br)cc(Br)c1 ; c1cc(-c2cc(-c3cccc(-n4c5ccccc5c5ccccc54)c3)cc(-c3cccc(-n4c5ccccc5c5ccccc54)c3)c2)cc(-n2c3ccccc3c3ccccc32)c1]

The compound k (7.00 g, 19.0 mol), 1,3,5-tribromobenzene (1.93 g, 6.13 mmol), tetrakis (triphenylphosphine) palladium (355 mg, 0.307 mmol), sodium carbonate (3.90 g, 36.8 mmol) were dissolved in a mixed solvent of 1,2-dimethoxyethane 300 ml/H₂O 80 ml, and reacted for 6 hours at a boiling point under reflux in nitrogen atmosphere. The reaction mixture was filtered and washed with ethyl acetate to produce white powder. The obtained white powder was purified by recrystallization to thereby yield an exemplary compound 15.

### (Synthesis of Exemplified Compound 20)

An exemplary Compound 20 can be prepared by the following reaction formula.

### [Chemical Formula 91]

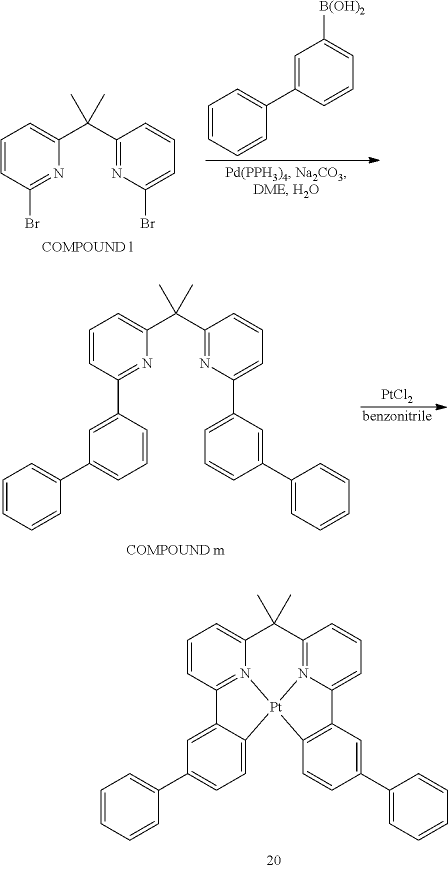

The compound 1 (2.50 g, 7.02 mol), 3-biphenyl boronic acid (2.93 g, 14.8 mmol), tetrakis (triphenylphosphine) palladium (406 mg, 0.351 mmol), and sodium carbonate (5.96 g, 56.2 mmol) were dissolved in a mixed solvent of 1,2-dimethoxyethane 40 ml/H₂O 40 ml, and reacted for 6 hours at a boiling point under reflux in nitrogen atmosphere, thereby producing a compound m in 72% yield. The compound m (1.76 g, 4.39 mmol) and platinum chloride (1.17 g, 4.39 mmol) were added to 14 ml of benzonitrile, and reacted for 5 hours at a boiling point under reflux in the nitrogen atmosphere. The reaction mixture was filtered and washed with ethyl acetate, so that the thus-obtained powder in orange color was purified by recrystallization in a solvent containing benzonitrile to give the exemplary compound 20.

### (Synthesis of Exemplary Compound 21)

An exemplary compound 21 can be prepared by the following reaction formula.

### [Chemical Formula 92]

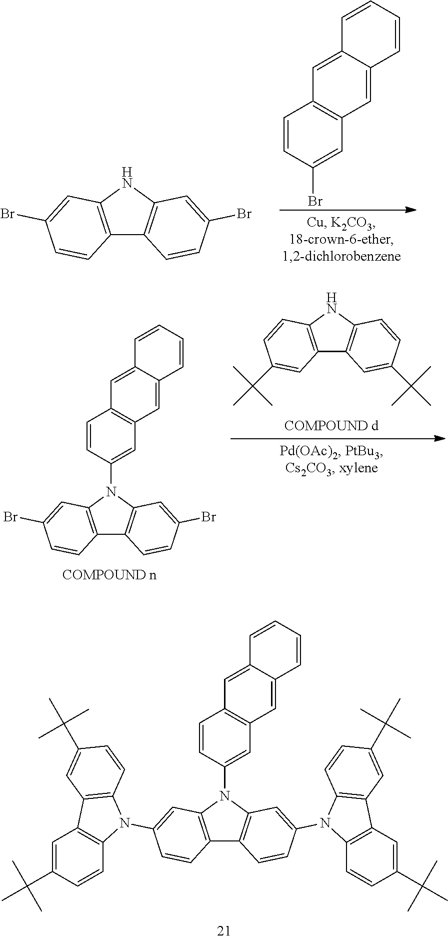 [SMILES: Brc1ccc2c(c1)[nH]c1cc(Br)ccc12 ; Brc1ccc2cc3ccccc3cc2c1 ; Brc1ccc2c3ccc(Br)cc3n(-c3ccc4cc5ccccc5cc4c3)c2c1 ; CC(C)(C)c1ccc2[nH]c3ccc(C(C)(C)C)cc3c2c1 ; CC(C)(C)c1ccc2c(c1)c1cc(C(C)(C)C)ccc1n2-c1ccc2c3ccc(-n4c5ccc(C(C)(C)C)cc5c5cc(C(C)(C)C)ccc54)cc3n(-c3ccc4cc5ccccc5cc4c3)c2c1]

According to Journal of Organic Chemistry, Vol. 70, section 5014 to 5019, 2005, 2,7-dibromocarbazole was synthesized. 3.5 g of the sample, 8.3 g of 2-bromo-anthracene, 0.8 g of copper powder, 3 g of potassium carbonate, 20 ml of 1,2-dichlorobenzene, and 1.4 g of 18-crown-6-ether were heated under reflux, and stirred for 6 hours under a nitrogen atmosphere. After cooling the reaction solution to room temperature, the reaction solution was purified by silica gel column chromatography with toluene-hexane mixed solvent to obtain 1.7 g of a compound n. The sample was reacted with the compound d to give the exemplary compound 21.

### (Synthesis of Exemplary Compound 22)

An exemplary compound 22 can be prepared by the following reaction formula.

### [Chemical Formula 93]

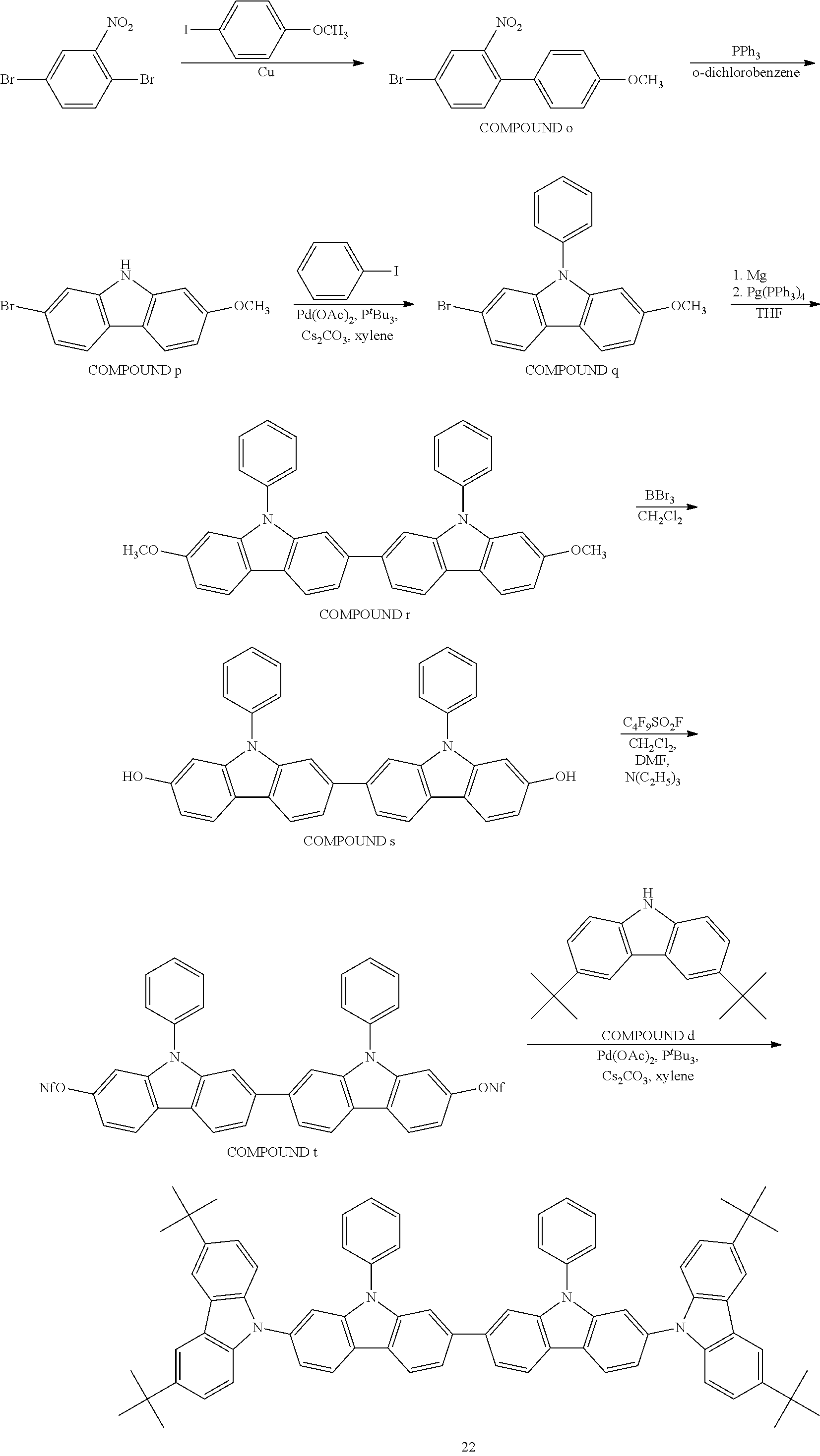

To 4 iodoanisole (25.1 g, 0.107 mol) were added 1,4-dibromo-2-nitrobenzene (23.2 g, 0.0825 mol) and copper powder (15.6 g, 0.248 mol), followed by stirring at 175° C. for 3 hours to thereby give a compound o in 44% yield. The compound o (11.1 g, 36.0 mmol) and triphenylphosphine (23.6 g, 90.0 mmol) were dissolved in 70 ml of o-dichlorobenzene, and reacted for 5 hours at a boiling point under reflux in a nitrogen atmosphere to thereby yield a compound p in 89% yield. The compound p (4.4 g, 15.9 mmol) and palladium acetate (89.4 mg, 0.398 mmol), tri (t-butyl) phosphine (241 mg, 119 mmol), cesium carbonate (15.5 g, 47.7 mmol), and iodo toluene (16.2 g, 79.5 mmol) were dissolved in 86 ml of xylene, and reacted in a nitrogen atmosphere for 3 hours at a boiling point under reflux, thereby synthesizing a compound q (in 52% yield).

Into 2 ml of THF under a nitrogen atmosphere, magnesium (103 mg, 4.24 mmol) was added, and refluxed at a boiling point. To this solution, the compound q (2.90 g, 8.23 mmol) in 8 ml of THF solution was added dropwise, and stirred for one hour. Then, tetrakis (triphenylphosphine) palladium (47.6 mg, 0.0412 mmol) was added to the solution, and refluxed for 2 hours at a boiling point to thereby give a compound r in 52% yield. The compound r (1.20 g, 2.20 mmol) was dissolved in 50 ml of methylene chloride. To the solution, 5.5 ml of 1 mol/IBBr₃ methylene chloride solution was added dropwise at 0° C. in a nitrogen atmosphere, and allowed to react for 3 hours at room temperature.

After the reaction quench, ethyl acetate and water were added to the reaction mixture to separate an organic phase. The organic phase was washed with water and saturated sodium chloride solution, and condensed under reduced pressure. The condensed reaction mixture (compound s) was dissolved in 30 ml of a methylene chloride and N, N′-dimethylformamide mixed solvent (1:1), and to the solution was added triethylamine (0.92 ml, 6.60 mmol). Under a nitrogen atmosphere at 5° C., perfluoro butane sulfonyl fluoride (1.16 ml, 6.60 mmol) was added dropwise to the solution, and allowed to react at room temperature for 3 hours to thereby give a compound t in 46% yield. The compound t (1.00 g, 0.925 mmol), palladium acetate with (11.3 mg, 0.0463 mmol), tri(t-butyl)phosphine (28.1 mg, 0.139 mmol), cesium carbonate (1.21 g, 3.70 mmol), and the compound d (567 mg, 2.03 mmol) were dissolved in 9 ml of xylene, and allowed to react for 4 hours at a boiling point under reflux in nitrogen atmosphere.

To the reaction mixture was added ethyl acetate and water to thereby separate an organic phase. The organic phase was washed with the water and saturated sodium chloride, and condensed under reduced pressure. The thus-obtained reaction mixture was purified by recrystallization to obtain the exemplary compound 22.

### (Synthesis of Exemplary Compound 23)

An exemplary compound 23 can be prepared by the following reaction formula.

### [Chemical Formula 94]

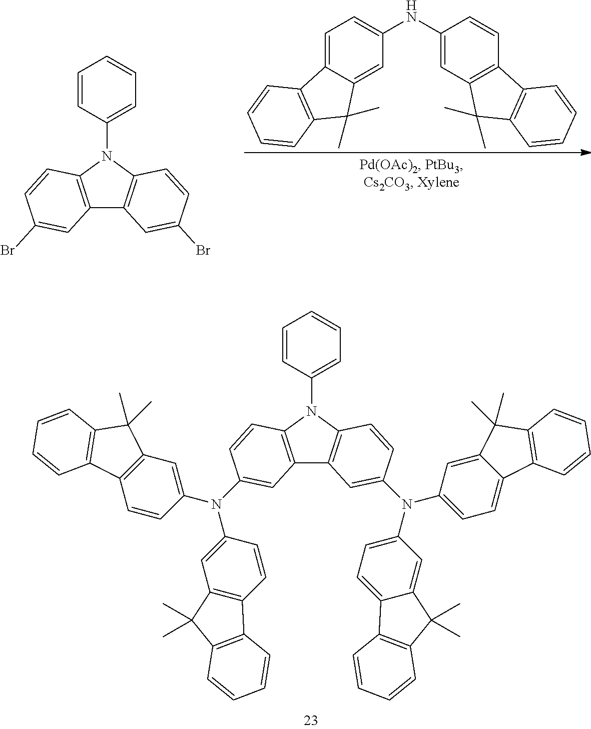 [SMILES: Brc1ccc2c(c1)c1cc(Br)ccc1n2-c1ccccc1 ; CC1(C)c2ccccc2-c2ccc(Nc3ccc4c(c3)C(C)(C)c3ccccc3-4)cc21 ; CC1(C)c2ccccc2-c2ccc(N(c3ccc4c(c3)C(C)(C)c3ccccc3-4)c3ccc4c(c3)c3cc(N(c5ccc6c(c5)C(C)(C)c5ccccc5-6)c5ccc6c(c5)C(C)(C)c5ccccc5-6)ccc3n4-c3ccccc3)cc21]

3,6-dibromo-9-phenyl carbazole (2.00 g, 4.99 mmol) and palladium acetate (60.8 mg, 0.249 mmol), tri(t-butyl) phosphine (151 mg, 0.747 mmol), cesium carbonate (6.51 g, 20.0 mmol), and bis (9,9′-dimethyl-fluorenyl-2-yl) amine (4.46 g, 110 mmol) were dissolved in 55 ml of xylene, and allowed to react for 5 hours at a boiling point under reflux in nitrogen atmosphere.

To the reaction mixture were added ethyl acetate and water to separate an organic phase. The organic phase was washed with water and saturated sodium chloride solution, and condensed under reduced pressure. The thus-obtained reaction mixture was purified by recrystallization to give the exemplary compound 23.

### (Synthesis of Compound 25)

An exemplary compound 25 can be prepared by the following reaction formula.

### [Chemical Formula 95]

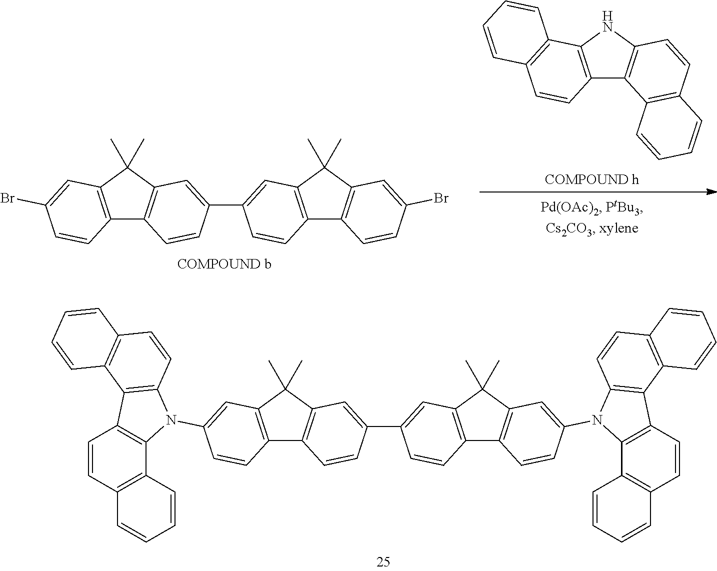 [SMILES: CC1(C)c2cc(Br)ccc2-c2ccc(-c3ccc4c(c3)C(C)(C)c3cc(Br)ccc3-4)cc21 ; c1ccc2c(c1)ccc1c2[nH]c2ccc3ccccc3c21 ; CC1(C)c2cc(-c3ccc4c(c3)C(C)(C)c3cc(-n5c6ccc7ccccc7c6c6ccc7ccccc7c65)ccc3-4)ccc2-c2ccc(-n3c4ccc5ccccc5c4c4ccc5ccccc5c43)cc21]

The compound b (1.11 g, 2.04 mmol) and palladium acetate (45.8 mg, 0.204 mmol), tri (t-butyl) phosphine (82.5 mg, 0.408 mmol), cesium carbonate (2.66 g, 8.16 mmol), and the compound h (1.20 g, 4.49 mmol) were dissolved in 10 ml of xylene, and allowed to react for 8 hours at a boiling point under reflux in nitrogen atmosphere. To the reaction mixture were added ethyl acetate and water to separate an organic phase. The organic phase was washed with water and saturated sodium chloride solution, and condensed under reduced pressure. The thus-obtained reaction mixture was purified by recrystallization to thereby yield the exemplary compound 25.

### (Synthesis of Compound 26)

An exemplary compound 26 can be prepared by the following reaction formula.

### [Chemical Formula 96]

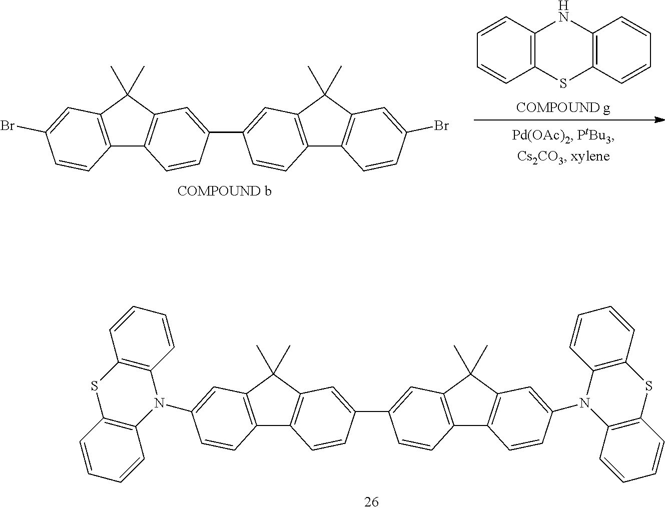 [SMILES: CC1(C)c2cc(Br)ccc2-c2ccc(-c3ccc4c(c3)C(C)(C)c3cc(Br)ccc3-4)cc21 ; c1ccc2c(c1)Nc1ccccc1S2 ; CC1(C)c2cc(-c3ccc4c(c3)C(C)(C)c3cc(N5c6ccccc6Sc6ccccc65)ccc3-4)ccc2-c2ccc(N3c4ccccc4Sc4ccccc43)cc21]

The compound b (1.10 g, 2.02 mmol) and palladium acetate (22.7 mg, 0.101 mmol), tri (t-butyl) phosphine (61.3 mg, 0.303 mmol), cesium carbonate (2.63 g, 8.08 mmol), and the compound g (845 mg, 4.24 mmol) were dissolved in 10 ml of xylene, and allowed to react for 4 hours at a boiling point under reflux in nitrogen atmosphere. To the reaction mixture were added ethyl acetate and water to separate an organic phase. The organic phase was washed with water and saturated sodium chloride solution, and condensed under reduced pressure. The thus-obtained reaction mixture was purified by recrystallization to thereby yield the exemplary compound 26.

### (Synthesis of Exemplified Compound 27)

An exemplary compound 27 can be prepared by the following reaction formula.

### [Chemical Formula 97]

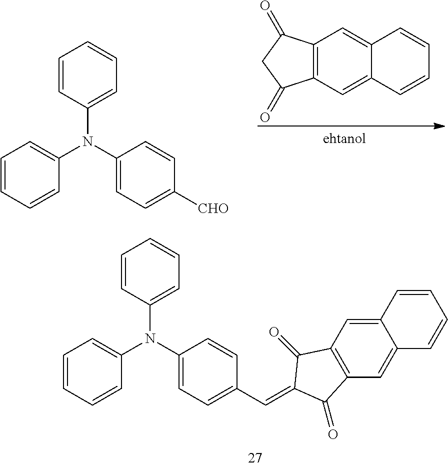

According to J. Med. Chem., Vol. 16, Section 1334-1339, 1973, Benz[f]indane-1,3-dione was synthesized. 2 g of the sample, and 3.1 g of 4-(N, N-diphenylamino) benzaldehyde were heated and stirred in 20 ml of ethanol for 6 hours under reflux, and then cooled to room temperature. The resulting crystals were filtered off and washed, followed by recrystallization from chloroform acetonitrile, thereby producing the exemplary compound 27.

### (Synthesis of Exemplified Compound 28)

An exemplary compound 28 can be prepared by the following reaction formula.

### [Chemical Formula 98]

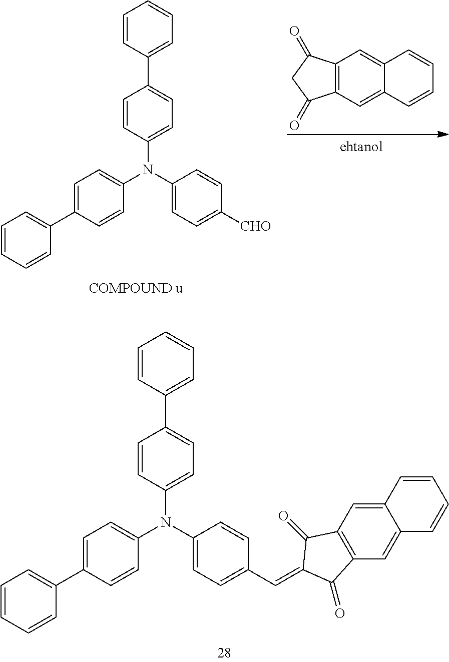 [SMILES: O=Cc1ccc(N(c2ccc(-c3ccccc3)cc2)c2ccc(-c3ccccc3)cc2)cc1 ; O=C1CC(=O)c2cc3ccccc3cc21 ; O=C1C(=Cc2ccc(N(c3ccc(-c4ccccc4)cc3)c3ccc(-c4ccccc4)cc3)cc2)C(=O)c2cc3ccccc3cc21]

According to J. Med. Chem., Vol. 17, Section 2088-2094, 1973, a compound u was synthesized. The compound u (2.0 g, 4.70 mmol) and Benz[f]indane-1,3-dione (1.01 g, 5.17 mmol) were heated and stirred in 15 ml, or 20 ml of ethanol under reflux for 6 hours, and allowed to cool to room temperature. The resulting crystals were filtered off and washed, followed by recrystallization from chloroform acetonitrile, thereby producing the exemplary compound 28.

Other compounds used in Examples and Comparative Examples were synthesized with reference to the above-mentioned method, or cited references, such as U.S. Patent Application Publication No. 20070293704, Chem. Lett., Vol. 35, 158-159, 2006, European Patent Publication No. 1559706, International Patent Publication No. 1999/040655, and the like.

The evaluation results are shown in Table 3 below. As shown in the table, it was confirmed that in the vapor deposited films of Examples 7 to 47, the changes in the film purity were less than 10% without being affected by the powder purity until the continuous deposition time reaches 120 minutes, which was suitable for mass production.

TABLE 3

| | | Solvent | Powder | Film purity(%) | | | Ratio of film purity |
|---|---|---|---|---|---|---|---|
| | | content | purity | After 0 | After 60 | After 120 | after 120 minutes |
| | Compound | (mol %) | (%) | minute | minutes | minutes | to that after 0 minute |
| Example 7 | Compound 2 | 0.3 | 99.7 | 99.7 | 99.5 | 99.4 | 0.99 |
| Example 8 | Compound 2 | 2.0 | 99.7 | 99.2 | 98.9 | 97.9 | 0.98 |
| Example 9 | Compound 3 | 1.8 | 99.5 | 99.3 | 99.0 | 98.2 | 0.98 |
| Example 10 | Compound 4 | 1.3 | 99.7 | 99.7 | 99.1 | 99.0 | 0.99 |
| Example 11 | Compound 5 | 1.0 | 99.8 | 99.8 | 99.6 | 99.2 | 0.99 |
| Example 12 | Compound 6 | 0.9 | 99.6 | 99.6 | 99.4 | 99.1 | 0.99 |
| Example 13 | Compound 6 | 2.8 | 99.6 | 99.1 | 94.9 | 90.4 | 0.91 |
| Example 14 | Compound 7 | 0.2 | 99.4 | 99.4 | 99.4 | 99.4 | 1.00 |
| Example 15 | Compound 7 | 1.2 | 99.5 | 99.5 | 99.3 | 99.2 | 0.99 |
| Example 16 | Compound 7 | 3.0 | 99.4 | 98.8 | 95.8 | 90.1 | 0.91 |
| Example 17 | Compound 8 | 0.5 | 99.0 | 99.0 | 99.0 | 98.9 | 0.99 |
| Example 18 | Compound 8 | 1.6 | 99.0 | 99.0 | 98.9 | 98.6 | 0.99 |
| Example 19 | Compound 9 | 1.4 | 99.3 | 99.3 | 99.2 | 99.0 | 0.99 |
| Example 20 | Compound 10 | 0.8 | 99.2 | 99.2 | 99.1 | 98.9 | 0.99 |
| Example 21 | Compound 10 | 2.1 | 99.2 | 99.1 | 98.0 | 97.0 | 0.97 |
| Example 22 | Compound 11 | 0.4 | 99.6 | 99.6 | 99.6 | 99.5 | 0.99 |
| Example 23 | Compound 12 | 1.6 | 98.9 | 98.9 | 98.3 | 98.0 | 0.99 |
| Example 24 | Compound 13 | 0.6 | 99.7 | 99.7 | 99.5 | 99.3 | 0.99 |
| Example 25 | Compound 14 | 2.3 | 98.6 | 98.4 | 97.1 | 94.0 | 0.95 |
| Example 26 | Compound 15 | 1.0 | 99.6 | 99.6 | 99.6 | 99.4 | 0.99 |
| Example 27 | Compound 16 | 2.0 | 99.6 | 99.5 | 98.5 | 96.2 | 0.96 |
| Example 28 | Compound 17 | 1.0 | 99.8 | 99.8 | 99.7 | 99.5 | 0.99 |
| Example 29 | Compound 18 | 1.1 | 99.7 | 99.7 | 99.6 | 99.3 | 0.99 |
| Example 30 | Compound 19 | 1.5 | 99.6 | 99.6 | 99.3 | 98.9 | 0.99 |
| Example 31 | Compound 20 | 1.4 | 99.2 | 99.2 | 99.0 | 98.6 | 0.99 |
| Example 32 | Compound 21 | 0.4 | 99.6 | 99.6 | 99.6 | 99.5 | 0.99 |
| Example 33 | Compound 21 | 2.4 | 99.0 | 98.7 | 98.8 | 96.9 | 0.98 |
| Example 34 | Compound 22 | 2.6 | 98.8 | 98.4 | 97.0 | 95.3 | 0.96 |
| Example 35 | Compound 23 | 2.0 | 98.9 | 98.5 | 97.0 | 95.0 | 0.96 |
| Example 36 | Compound 24 | 1.1 | 98.9 | 98.6 | 98.4 | 98.2 | 0.99 |
| Example 37 | Compound 25 | 1.3 | 98.6 | 98.5 | 98.3 | 98.0 | 0.99 |
| Example 38 | Compound 26 | 0.9 | 98.5 | 98.5 | 98.3 | 98.2 | 0.99 |
| Example 39 | Compound 27 | 0.4 | 99.5 | 99.5 | 99.4 | 99.2 | 0.99 |
| Example 40 | Compound 27 | 2.7 | 99.4 | 99.1 | 94.9 | 90.2 | 0.91 |
| Example 41 | Compound 28 | 0.6 | 99.2 | 99.1 | 99.1 | 99.0 | 0.99 |
| Example 42 | Compound 29 | 1.8 | 99.1 | 98.8 | 97.4 | 95.4 | 0.96 |
| Example 43 | Compound 30 | 1.0 | 99.3 | 99.2 | 99.0 | 98.7 | 0.99 |
| Example 44 | Compound 31 | 1.2 | 99.0 | 99.0 | 99.0 | 98.7 | 0.99 |
| Example 45 | Compound 32 | 0.9 | 99.5 | 99.5 | 99.2 | 99.0 | 0.99 |
| Example 46 | Compound 33 | 0.5 | 99.4 | 99.4 | 99.3 | 99.2 | 0.99 |
| Example 47 | Compound 34 | 0.8 | 99.1 | 99.1 | 99.0 | 98.9 | 0.99 |

TABLE 4

| | | Solvent | Powder | Film purity(%) | | | Ratio of film purity |
|---|---|---|---|---|---|---|---|
| | | content | purity | After 0 | After 60 | After 120 | after 120 minutes |
| | Compound | (mol %) | (%) | minute | minutes | minutes | to that after 0 minute |
| Comparative | Compound 2 | 3.4 | 99.6 | 91.1 | 84.0 | 75.0 | 0.83 |
| example 4 | | | | | | | |
| Comparative | Compound 2 | 4.0 | 99.5 | 87.0 | 81.1 | 70.1 | 0.80 |
| example 5 | | | | | | | |
| Comparative | Compound 3 | 3.5 | 99.5 | 90.1 | 84.0 | 73.6 | 0.81 |
| example 6 | | | | | | | |
| Comparative | Compound 4 | 3.6 | 99.5 | 90.2 | 82.5 | 74.0 | 0.82 |
| example 7 | | | | | | | |
| Comparative | Compound 5 | 3.3 | 99.7 | 90.3 | 85.2 | 77.8 | 0.86 |
| example 8 | | | | | | | |
| Comparative | Compound 6 | 3.4 | 99.6 | 90.1 | 84.9 | 77.7 | 0.86 |
| example 9 | | | | | | | |
| Comparative | Compound 6 | 4.4 | 99.6 | 88.4 | 79.4 | 67.0 | 0.75 |
| example 10 | | | | | | | |
| Comparative | Compound 7 | 3.4 | 99.4 | 89.8 | 84.0 | 78.1 | 0.86 |
| example 11 | | | | | | | |
| Comparative | Compound 8 | 3.3 | 99.0 | 90.0 | 80.1 | 75.8 | 0.84 |
| example 12 | | | | | | | |
| Comparative | Compound 9 | 5.0 | 99.2 | 87.9 | 75.0 | 63.0 | 0.71 |
| example 13 | | | | | | | |
| Comparative | Compound 10 | 4.8 | 99.2 | 88.1 | 76.0 | 64.1 | 0.72 |
| example 14 | | | | | | | |
| Comparative | Compound 11 | 3.7 | 99.6 | 90.9 | 82.5 | 74.4 | 0.81 |
| example 15 | | | | | | | |
| Comparative | Compound 12 | 5.2 | 98.8 | 84.0 | 71.3 | 60.2 | 0.71 |
| example 16 | | | | | | | |
| Comparative | Compound 13 | 3.6 | 99.6 | 90.2 | 81.2 | 70.4 | 0.78 |
| example 17 | | | | | | | |
| Comparative | Compound 14 | 3.7 | 98.6 | 89.9 | 78.0 | 69.8 | 0.77 |
| example 18 | | | | | | | |
| Comparative | Compound 15 | 3.7 | 99.6 | 90.0 | 80.3 | 72.5 | 0.80 |
| example 19 | | | | | | | |
| Comparative | Compound 16 | 3.4 | 99.6 | 89.7 | 79.9 | 70.0 | 0.78 |
| example 20 | | | | | | | |
| Comparative | Compound 17 | 4.0 | 99.8 | 88.6 | 77.7 | 69.7 | 0.78 |
| example 21 | | | | | | | |
| Comparative | Compound 18 | 4.2 | 99.7 | 90.0 | 80.1 | 68.8 | 0.76 |
| example 22 | | | | | | | |
| Comparative | Compound 19 | 3.7 | 99.5 | 89.9 | 81.2 | 70.1 | 0.77 |
| example 23 | | | | | | | |
| Comparative | Compound 20 | 3.4 | 99.2 | 88.7 | 81.4 | 72.5 | 0.81 |
| example 24 | | | | | | | |
| Comparative | Compound 21 | 4.0 | 99.5 | 87.9 | 80.4 | 72.2 | 0.82 |
| example 25 | | | | | | | |
| Comparative | Compound 21 | 3.6 | 99.0 | 89.5 | 81.0 | 71.5 | 0.79 |
| example 26 | | | | | | | |
| Comparative | Compound 22 | 4.1 | 98.7 | 88.7 | 80.3 | 70.7 | 0.79 |
| example 27 | | | | | | | |
| Comparative | Compound 23 | 3.5 | 98.7 | 89.5 | 82.0 | 73.0 | 0.81 |
| example 28 | | | | | | | |
| Comparative | Compound 24 | 3.4 | 98.8 | 89.4 | 81.9 | 73.1 | 0.81 |
| example 29 | | | | | | | |
| Comparative | Compound 25 | 4.2 | 98.6 | 87.8 | 77.5 | 68.9 | 0.78 |
| example 30 | | | | | | | |
| Comparative | Compound 26 | 4.3 | 98.5 | 87.6 | 77.8 | 68.5 | 0.78 |
| example 31 | | | | | | | |
| Comparative | Compound 27 | 3.3 | 99.4 | 89.7 | 82.3 | 73.1 | 0.81 |
| example 32 | | | | | | | |
| Comparative | Compound 28 | 4.0 | 99.2 | 87.5 | 77.4 | 68.6 | 0.78 |
| example 33 | | | | | | | |
| Comparative | Compound 29 | 3.9 | 99.1 | 87.6 | 79.8 | 70.0 | 0.79 |
| example 34 | | | | | | | |
| Comparative | Compound 30 | 3.8 | 99.3 | 88.0 | 80.2 | 70.4 | 0.80 |
| example 35 | | | | | | | |
| Comparative | Compound 31 | 3.5 | 99.0 | 90.4 | 82.4 | 74.4 | 0.82 |
| example 36 | | | | | | | |
| Comparative | Compound 32 | 3.6 | 99.5 | 91.2 | 82.9 | 74.9 | 0.82 |
| example 37 | | | | | | | |
| Comparative | Compound 33 | 3.9 | 99.3 | 88.1 | 78.6 | 69.2 | 0.78 |
| example 38 | | | | | | | |
| Comparative | Compound 34 | 3.5 | 99.0 | 89.8 | 82.3 | 73.3 | 0.81 |
| example 39 | | | | | | | |

### <Preparation of Photoelectric Conversion Element>

Next, using the compound 7 for deposition in the above each example, a photoelectric conversion element was fabricated in the same way as in Example 1, and evaluated for its properties.

On a glass substrate, amorphous ITO (30 nm) were deposited for a plurality of substrates by sputtering, thereby making pixel electrodes (lower electrodes). Thereon, an electron blocking layer was formed by the vacuum resistance heating deposition in a thickness of 100 nm using the compound 7 for deposition on the same conditions as Examples 15 and 16 and Comparative Example 11 by defining the time when the deposition rate becomes about 2 Å/s to be stable as 0 minute. After formation of the electron blocking layer, a vapor deposited film (100 nm) was deposited by exchanging the substrate every 60 minutes while the rate of the compound 7 was maintained.

The compound 6 for deposition and fullerene C₆₀ were co-deposited by vacuum resistance heating deposition to form the photoelectric conversion layer having a thickness of 300 nm. In the co-deposition, the volume ratio of the compound 6 to the fullerene was 1:3.

Furthermore, an amorphous ITO (of 10 nm in thickness) was deposited as the upper electrode by sputtering to form the transparent electrode (upper electrode), thereby fabricating the photoelectric conversion element. On the upper electrode, after the SiO film is formed by heating deposition as a sealing layer, followed by formation of an Al₂O₃ layer thereon by an ALCVD method.

### [Evaluation]

For the photoelectric conversion elements obtained in the respective examples, the relative response speed upon application of an electric field of 2×10⁵ V/cm (rise time with respect to 0 to 99% signal strength), and the relative sensitivity (wavelength 500-750 nm) were measured. The result is shown in Table 5. At the time of measurement of the photoelectric conversion performance of each element, the light entered from the upper electrode (transparent conductive film) side.

The relative sensitivity and the relative response speed were represented by a relative value with those at 0 minute set to 100 in Example 15. As shown in Table 5, it was confirmed that in the photoelectric conversion element using the vapor deposition film of Examples 15 and 16 (Table 4), the changes in the relative response speed and sensitivity were remained within 5%, which was suitable for mass production.

TABLE 5

| | | Solvent | Powder | Relative sensitivity | | | Relative response speed | | |
|---|---|---|---|---|---|---|---|---|---|
| | | content | purity | After 0 | After 60 | After 120 | After 0 | After 60 | After 120 |
| | Compound | (mol %) | (%) | minute | minutes | minutes | minute | minutes | minutes |
| Example 15 | Compound 7 | 1.2 | 99.5 | 100 | 100 | 100 | 100 | 100 | 100 |
| Example 16 | Compound 7 | 3.0 | 99.4 | 100 | 100 | 99 | 100 | 100 | 98 |
| Comparative | Compound 7 | 3.4 | 99.4 | 100 | 99 | 94 | 100 | 98 | 94 |
| example 11 | | | | | | | | | |

The organic material for deposition and the deposition method using the same according to the present invention can be preferably applied to deposition of organic layers of organic photoelectric conversion elements, including an organic imaging element, such as a digital camera, a camera for a cell phone, or a camera for an endoscope; an organic light emitting element mounted on an organic EL display or an organic EL illumination; an organic thin film transistor mounted on an electron paper, a wireless tag, or the like; an optical sensor, and the like.

What is claimed is:

1. An organic material for deposition that is used for dry deposition of an organic layer configuring an organic photoelectric conversion element, wherein
the organic material contains an organic composition of the organic layer as a principal component, and
a residual solvent content of the organic material for deposition is equal to or less than 3 mol %.

2. The organic material for deposition according to claim 1, wherein
a vapor pressure of the residual solvent having a degree of vacuum of 1·10⁻³ Pa or less is higher than a sublimation pressure of the organic composition.

3. The organic material for deposition according to claim 1, wherein
a ratio of a film purity of the organic layer obtained after the continuous dry deposition for two hours to that of the organic layer in an initial stage of the deposition is 0.9 or more.

4. The organic material for deposition according to claim 1, wherein, when the dry deposition is continuously performed and a total thickness of the organic layer reaches 16000 Å, the ratio of the film purity of the organic layer to that of the organic layer in an initial stage of the deposition is 0.9 or more.

5. The organic material for deposition according to claim 1, wherein when the dry deposition is continuously performed and a total thickness of the organic layer reaches 16000 Å, the ratio of the film purity of the organic layer to a purity of the organic layer for deposition is 0.9 or more.

6. A deposition method according to claim 1, wherein the dry deposition method is a vacuum resistance heating deposition method.

7. The organic material for deposition according to claim 1, wherein the organic layer is a photoelectric conversion layer or an electron blocking layer.

8. The organic material for deposition according to claim 7, wherein the organic composition is an organic compound represented by the following general formula (D-I):
[Chemical Formula 1] 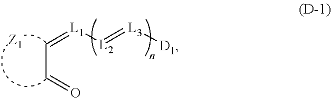
in the general formula (D-I), Z₁ represents a group of atoms required to form a 5- or 6-membered ring; L₁, L₂ and L₃ independently represent a non-substituted methine group, or a substituted methine group; D₁ represents a group of atoms; and n represents an integer number of 0 or more.

9. The organic material for deposition according to claim 8, wherein the D₁ is represented by the following general formula (D-II):
[Chemical Formula 6] 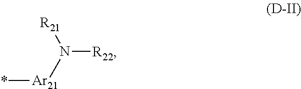
in the general formula (D-II), R₂₁ and R₂₂ each independently represent a hydrogen atom or a substituent group; Ar₂₁ represents an aromatic hydrocarbon ring group or an aromatic heterocyclic group; * represents a binding position; and Ar₂₁ and R₂₁, Ar₂₁ and R₂₂, and R₂₁ and R₂₂ respectively are optionally bonded to each other to form a ring.

10. The organic material for deposition according to claim 8, wherein the compound represented by the general formula (D-I) is represented by the following general formula (D-III):
[Chemical Formula 11] 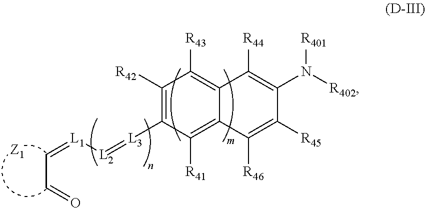
in the general formula (D-III), Z₁ represents a group of atoms requested to form a 5 or 6-membered ring; L₁, L₂ and L₃ each independently represent an unsubstituted methine group or a substituted methine group; n represents an integer number of 0 or more; m represents 0 or 1; R₄₁ to R₄₆ each independently represent a hydrogen atom or a substituent group; R₄₂ and R₄₃, R₄₃ and R₄₄, R₄₅ and R₄₆, and R₄₁ and R₄₆ respectively optionally form a ring independently; R₄₀₁ and R₄₀₂ each represent a single bond, or a divalent or trivalent coupling group; R₄₀₁ and any one of R₄₁ to R₄₆, R₄₀₁ and R₄₀₂, and R₄₀₂ and any one of R₄₁ to R₄₆ respectively are optionally bonded to each other to form a ring.

11. The organic material for deposition according to claim 8, wherein the compound represented by the general formula (D-I) is represented by the following general formula (D-IV):
[Chemical Formula 12] 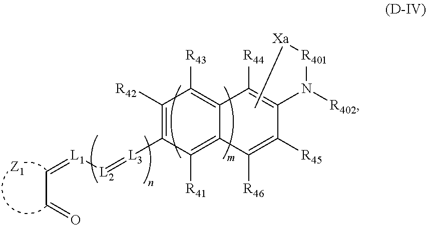
in the general formula (D-IV), Z₁ represents a group of atoms required to form a 5 or 6-membered ring; L₁, L₂ and L₃ each independently represent an unsubstituted methine group or a substituted methine group; n represents an integer number of 0 or more; m represents 0 or 1; R₄₁ to R₄₆ independently represent a hydrogen atom or a substituent group; R₄₂ and R₄₃, R₄₃ and R₄₄, R₄₅ and R₄₆, and R₄₁ and R₄₆ respectively optionally form a ring independently; R₄₀₁ represents a single bond or a divalent coupling group, and R₄₀₂ independently represents a hydrogen atom or a substitutent group; Xa represents a single bond, an oxygen atom, a sulfur atom, an alkylene group, a silylene group, an alkenylene group, a cycloalkylene group, a cycloalkenylene group, an arylene group, a divalent heterocyclic group, or an imino group, in which these optionally further have a substituent group to be bonded as any one of R₄₁ to R₄₆; and R₄₀₁ and R₄₀₂, and R₄₀₂ and any one of R₄₁ to R₄₆ respectively are optionally bonded to each other to form a ring.

12. The organic material for deposition according to claim 8, wherein a structure formed by the Z₁ and an oxygen atom is represented by the following general formula (D-V):
[Chemical Formula 3] 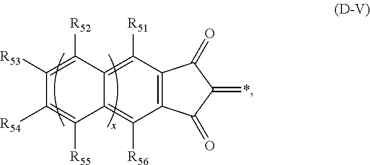
in the general formula (D-V), R₅₁ to R₅₆ each independently represent a hydrogen atom or a substituent group; Any adjacent two of R₅₁ to R₅₆ are optionally bonded to each other to form a ring; * represents the binding position with the L₁; and x represents 0 or 1.

13. The organic material for deposition according to claim 7, wherein an organic composition used in the organic layer is an organic compound represented by the following general formula (EB-1):
[Chemical Formula 29] 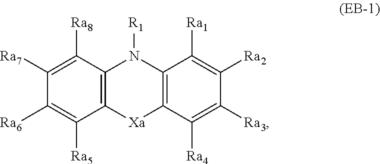
in the general formula, R₁ represents an alkyl group, an aryl group, or a heterocyclic group, which optionally has a substituent group; Ra₁ to Ra₈ each independently represent a hydrogen atom or substituent; at least two of R₁ and Ra₁ to Ra₈ are optionally bonded to each other to form a ring; and Xa represents a single bond, an oxygen atom, a sulfur atom, or an alkylene group, a silylene group, an alkenylene group, a cycloalkylene group, a cycloalkenylene group, an arylene group, a divalent heterocyclic group, or an imino group, which optionally has a substituent group.

14. The organic material for deposition according to claim 13, wherein a compound represented by the general formula (EB-1) is a compound represented by the following general formula (EB-3):
[Chemical Formula 35] 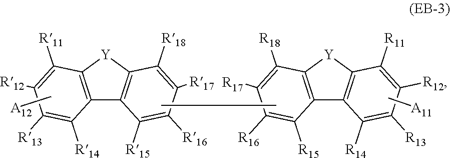
in the general formula (EB-3), R₁₁ to R₁₈, and R′₁₁ to R′₁₈ independently represent a hydrogen atom, a halogen atom, an alkyl group, an aryl group, a heterocyclic group, a hydroxyl group, an amino group or a mercapto group, which optionally has a further substituent group; any one of R₁₅ to R₁₈ is coupled to any one of R′₁₅ to R′₁₈ to form a single bond; A₁₁ and A₁₂ each independently represent a substituent group represented by the following general formula (A-1), and which substitutes as any one of R₁₁ to R₁₄, and any one of R′₁₁ to R′₁₄; and Y each independently represents a carbon atom, a nitrogen atom, an oxygen atom, a sulfur atom, or a silicon atom, which optionally has a further substituent group,
[Chemical Formula 31] 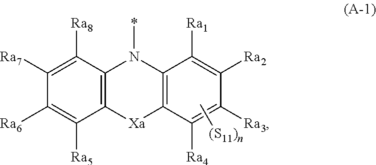
in the general formula (A-1), Ra₁ to Ra₈ each independently represent a hydrogen atom, a halogen atom, an alkyl group, an aryl group, a heterocyclic group, or an alkoxy group, which optionally has a further substituent group; at least two of Ra₁ to Ra₈ are optionally bonded to each other to form a ring; * represents a binding position; Xa represents a single bond, an oxygen atom, a sulfur atom, or an alkylene group, a silylene group, an alkenylene group, a cycloalkylene group, a cycloalkenylene group, an arylene group, a divalent heterocyclic group, or an imino group, which optionally has a substituent group; S₁₁ each independently indicates the following substituent group (S₁₁), and substitutes as any one of Ra₁ to Ra₈. n each independently represents an integer number of 1 to 4,
[Chemical Formula 32] 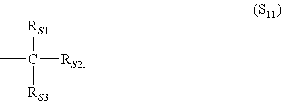
Rs₁ to Rs₃ each independently represent a hydrogen atom or an alkyl group; and at least two of Rs₁ to Rs₃ are optionally bonded to each other to form a ring.

15. The organic material for deposition according to claim 14, wherein a compound represented by the general formula (EB-3) is a compound represented by the following general formula (EB-4):
[Chemical Formula 43] 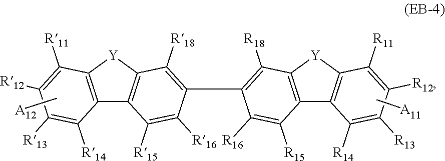
in the general formula (EB-4), R₁₁ to R₁₆, R₁₈, R′₁₁ to R′₁₆, and R′₁₈ each independently represent a hydrogen atom, a halogen atom, an alkyl group, an aryl group, a heterocyclic group, a hydroxyl group, an amino group, or a mercapto group, which optionally has a further substituent group; A₁₁ and A₁₂ each independently represent a substituent group represented by the general formula (A-1), and substitute as any one of R₁₁ to R₁₄, and any one of R′₁₁ to R′₁₄; and Y respectively independently represents a carbon atom, a nitrogen atom, an oxygen atom, a sulfur atom, or a silicon atom, which optionally has a further substituent group.

16. The organic material for deposition according to claim 13, wherein in the general formulas (EB-3) and (EB-4), a substituent group represented by the general formula (A-1) is independently substituted by each of the R₁₂ and R′₁₂.

17. The organic material for deposition according to claim 14, wherein n in the general formula (A-1) is 1 or 2.

18. The organic material for deposition according to claim 14, wherein at least one of Ra₁ and Ra₆ in the general formula (A-1) is independently substituted by the substitutent group (S₁₁).

19. The organic material for deposition according to claim 14, wherein Y in the general formulas (EB-3) and (EB-4) represents —N(R₂₀)—, in which R₂₀ represents an alkyl group, an aryl group, or a heterocyclic group.

20. The organic material for deposition according to claim 14, wherein Y in the general formulas (EB-3) and (EB-4) represents —C(R₂₁)(R₂₂)—, in which R₂₁ and R₂₂ each independently represent an alkyl group, an aryl group, or a heterocyclic group.

21. The organic material for deposition according to claim 7, wherein the organic material for deposition is material represented by the following general formula (EB-2):
[Chemical Formula 30] 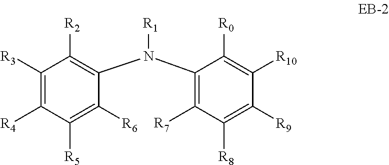
(in the formula, R₁ represents an alkyl group, an aryl group, or a heterocyclic group, which optionally has a substituent group; and R₀ and R₂ to R₁₀ each independently represent a hydrogen atom or a substituent group.)

22. The organic material for deposition according to claim 21, wherein in the general formula (EB-2), R₁ which optionally has a substitute group is an aryl group.

23. A method for forming an organic layer constituting an organic photoelectric conversion element by a dry deposition method, the method comprising the steps of:
preparing an organic material for deposition containing an organic composition of the organic layer as a principal component;
removing a solvent contained in the organic material for deposition such that a solvent content is 3 mol % or less; and
depositing the organic layer by the dry deposition method using the organic material for deposition whose residual solvent content is set to 3 mol % or less by the solvent removal step.

24. The method for forming an organic layer according to claim 23, wherein the dry deposition method is a vacuum resistance heating deposition method.

25. The method for forming an organic layer according to claim 23, wherein the organic layer is a photoelectric conversion layer or an electron blocking layer.

26. The method for forming an organic layer according to claim 25, wherein the organic layer is a photoelectric conversion layer or an electron blocking layer, and the organic composition is an organic compound represented by the following general formula (D-I) or (EB-1):
[Chemical Formula 1] 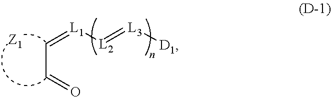
in the general formula (D-I), Z₁ represents a group of atoms required to form a 5- or 6-membered ring; L₁, L₂ and L₃ independently represent an unsubstituted methine group, or a substituted methine group; D₁ represents a group of atoms; and n represents an integer number of 0 or more.
[Chemical Formula 29] 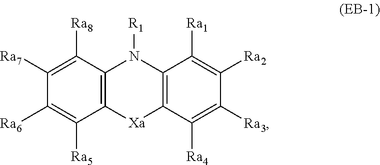
in the general formula, R₁ represents an alkyl group, an aryl group, or a heterocyclic group, which optionally has a substituent group; Ra₁ to Ra₈ each independently represent a hydrogen atom or a substituent group; at least two of R₁ and Ra₁ to Ra₈ are optionally bonded to each other to form a ring; and Xa represents a single bond, an oxygen atom, a sulfur atom, or an alkylene group, a silylene group, an alkenylene group, a cycloalkylene group, a cycloalkenylene group, an arylene group, a divalent heterocyclic group, or an imino group, which optionally has a substituent group.

27. A method for manufacturing an organic photoelectric conversion element that includes a pair of electrodes, and a light receiving layer or a light emitting layer including at least a photoelectric conversion layer sandwiched between the pair of electrodes, wherein
the light receiving layer is deposited by the deposition method according to claim 23.

28. An organic photoelectric conversion element that includes a pair of electrodes, and a light receiving layer or a light emitting layer including at least a photoelectric conversion layer sandwiched between the pair of electrodes, wherein
at least one of the pair of electrodes is a transparent electrode, and
the light receiving layer or the light emitting layer is deposited by a dry deposition using the organic material for deposition according to claim 7.

29. An optical sensor, comprising:
a plurality of the photoelectric conversion elements according to claim 28; and
a circuit substrate on which a signal reading circuit for reading a signal corresponding to a charge generated in the photoelectric conversion layer of the photoelectric conversion element is formed.

30. An imaging element, comprising:
a plurality of the photoelectric conversion elements according to claim 28; and
a circuit substrate on which a signal reading circuit for reading a signal corresponding to a charge generated in the photoelectric conversion layer of the photoelectric conversion element is formed.

31. A light emitting element comprising a plurality of the photoelectric conversion elements according to claim 28, wherein light is emitted from the light emitting layer by applying a voltage to between the pair of electrodes, and the light is taken out of an end surface on a side of the transparent electrode.
